SEQUENCE LISTING (294 sequences)

SEQ ID NO: 1 - protein, 344 aa, Synthetic construct
MNKPKFKAAAVQAAPVFLDLDKTIDKTRALMEKAARNGAELIAFPETWLPGYPWYAWLYAPAMWVPVYTRRYFDNSLEFGTPQKHRIRKDAKDNNIYVSMGLSEREGESLYISQWFIDRDGDTIAERRKLKPTHVERTIFGEGDGSDLAVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELHEGLPAGEEGLIYAEIDLGLISVSKAVNDPAGHYSRPDVTQLIHDKAPRRAVVDTAPPSDPAPAVERPHGADEPALPAMAEL

SEQ ID NO: 2 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaaaaagcggcgcgcaacggcgcggaactgattgcgtttccggaaacctggttaccgggctatccgtggtatgcgtggctgtatgcgccggcaatgtgggtgccggtgtatacccgccgctattttgataacagcctggaatttggcaccccgcagaaacatcgcattcgcaaagatgcgaaagataacaacatttatgtgagcatgggcctgagcgaacgcgaaggcgaaagcctgtacatttctcagtggtttattgatcgcgatggcgataccattgcggaacgccgcaaactgaaaccgacccatgtggaacgcaccatttttggcgaaggcgatggcagcgatctggcggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 3 - protein, 344 aa, Synthetic construct
MNKPKFKAAAVQAAPVFLDLDKTIDKTIALMEEAARNGAELIAFPETWLPGYPWYAWLDAPAMWLMQYTQRYFDNSLEYGTPQKARIRKDAKDNNIYVVMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYEAYAYALGPDVNTSASRIYAVEGQCFVLAPCATVSKGMVDEMCTTDGQKALLKTGGGYARIFGPDGRQLSEGLPAGEEGLIYAEIDLGLISVSKAVGDPAGHYSRPDVTQLIHDKAPRRAVVDTAPPSDPAPAVERPHGADEPALPAMAEL

SEQ ID NO: 4 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaatgcagtatacccagcgctattttgataacagcctggaatatggcaccccgcagaaagcgcgcattcgcaaagatgcgaaagataacaacatttatgtggtgatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcaactgttagcaaaggcatggtggatgagatgtgcaccaccgatggccagaaagcgctgttaaaaaccggcggcggctatgcgcgcatttttggcccggatggccgccagttaagcgaaggcctgccggccggcgaagaaggcctgatttatgcggaaattgatctgggcctgattagcgtgagcaaagcggtgggcgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 5 - protein, 344 aa, Synthetic construct
MNKPKFKAAAVQAAPVFLDLDKSIDKTEALMEDAARNGAELIAFPETWLPGYPWYAWLDAPAMWLPQYTRRYFDNSLEYGTPQAERIRKDAKDNNITVSMGLSEREGGSLYISQWFIDRDGDTIATRRKLKPTHVERTIFGEGDGSDLAVIDLRLGRVGGLCCWEHLQPLSKYAMYAQNEQVHVAAWPSFSMYEAYAYALGPEVNTSASRIYAVEGQCFVLAPCATVSKEMVDEMCTTDLQKALLKVGGAYARIFGPDGRQLHEGLPTGEEGLIYAEIDLGLISVSKAVGDPAGHYSRPDVTQLIHNKAPRRAVVDTAPPSDPAPAVERPHGADEPALPAMAEL

SEQ ID NO: 6 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagaccgaagcgctgatggaagatgcggcgcgcaacggcgcggaactgattgcgtttccggaaacctggttaccgggctatccgtggtatgcgtggctggatgcgccggcaatgtggttaccgcagtatacccgccgctattttgataacagcctggaatatggcaccccgcaggcggaacgcattcgcaaagatgcgaaagataacaacattaccgtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgattgatctgcgtttaggtcgcgttggcggcttatgctgctgggaacatctgcagccgctgagcaaatatgcgatgtatgcgcagaacgaacaggtgcatgtggcggcgtggccgagctttagcatgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaagtgggcggcgcgtatgcgcgcatttttggcccggatggccgccagttacatgaaggcctgccgaccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 7 - protein, 344 aa, Synthetic construct
MNKPKFKAAAVQAAPVLLDLDKTIDKTEALMEDAARNGAELIAFPETWLPGYPWYAWLDSPAMWMPQYTQRYFDNSLEYGTPQAKRIRKDAKDNNIVVSMGLSEREGASLYISQWFIDRDGDTIAQRRKLKPTHVERTIFGEGDGSDLAVHDTRLGRVGGLCCWEHLQPLSKYAMYAQNEQVHVAAWPSFSLYEAYAYALGPEVNTSASRIYAVEGQCFVLAPCATVSKGMVDEMCTTDLQKALLKTGGGYASIFGPDGRQLHEGLPTGEEGLIYAEIDLGLISVSKAVNDPAGHYSRPDVTQLIHNKAPRRAVVDTAPPSDPAPAVERPHGADEPALPAMAEL

SEQ ID NO: 8 - DNA, 1032 bp, Synthetic construct
  source 1..1032: mol_type = other DNA
atgaacaaaccgaaatttaaagcggcggcggtgcaggcggcgccggtgctgctggatttagacaaaaccattgataagaccgaagcgctgatggaagatgcggcgcgcaacggcgcggaactgattgcgtttccggaaacctggttaccgggctatccgtggtatgcgtggctggatagcccggcaatgtggatgccgcagtatacccagcgctattttgataacagcctggaatatggcaccccgcaggcgaaacgcattcgcaaagatgcgaaagataacaacattgtggtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgcatgatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaaggcatggtggatgagatgtgcaccaccgatctgcagaaagcgctgttaaaaaccggcggcggctatgcgagcatttttggcccggatggccgccagttacatgaaggcctgccgaccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 9 - protein, 344 aa, Synthetic construct
MNKPKFKAAAVQAAPVFLDLDKTIDKTIALAEDAARNGAELIAFPETWLPGYPWYAWLDAPAMWGPQYTQRYFDNSLEYGTPQFERIRKAAKDNNIVVSMGLSEREGGSLYISQWFIDRDGDTIAKRRKLKPTHVERTIFGEGDGSDLAVHDTRLGRVGGLCCWEHLQPLSKYAMYAQNEQVHVAAWPSFSIYEAYAYALGPIVNTSASRIYAVEGQCFVLAPCATVSKSMVDEMCTTDSQKALLTTGGGYARIFGPDGRSLHEGLPAGEEGLILAEIDLGLISVSKTVGDPAGHYSRPDVTQLIHNKAPRRAVVDTAPPSDPAPAVERPHGADEPALPAMAEL

SEQ ID NO: 10 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctggcggaagatgcggcgcgcaacggcgcggaactgattgcgtttccggaaacctggttaccgggctatccgtggtatgcgtggctggatgcgccggcaatgtggggcccgcagtatacccagcgctattttgataacagcctggaatatggcaccccgcagtttgaacgcattcgcaaagcggcgaaagataacaacattgtggtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgcatgatacccgtttaggtcgcgttggcggcttatgctgctgggaacatctgcagccgctgagcaaatatgcgatgtatgcgcagaacgaacaggtgcatgtggcggcgtggccgagctttagcatttatgaagcgtatgcgtatgcgttaggcccgattgtgaacaccagcgcgagcagaatttatgcggtggaaggccagtgctttgtgctggcgccgtgcgcaactgttagcaaaagcatggtggatgagatgtgcaccaccgatagccagaaagcgctgttaaccaccggcggcggctatgcgcgcatttttggcccggatggccgcagcttacatgaaggcctgccggccggcgaagaaggcctgattctggcggaaattgatctgggcctgattagcgtgagcaaaaccgtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 11 - protein, 344 aa, Synthetic construct
MNKPKFKAAAVQAAPVFLDLDKTIDKTIALMEDAARNGAELIAFPETWLPGYPWYAWLDAPAMWLPQYTKRYFDNSLEYGTPQKERIRKAAKNNNIVVSMGLSEREGASLYISQWFIDRDGDTIAQRRKLKPTHVERTIFGEGDGSDLAVWDIRLGRVGGLCCWEHLQPLSKYAMYAQNEQVHVAAWPSFSVYRAYAYALGPIVNTSASRIYAVEGQCFVLAPCAVVSKEMVDEMCTTDLHKALLKTGGGYARIFGPDGRQLHEGLPAGEEGLLYAEIDLGLISVSKAVGDPAGHYSRPDVTQLIHNKAPRRAVVDTAPPSDPAPAVERPHGAPEPALPAMAEL

SEQ ID NO: 12 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtataccaaacgctattttgataacagcctggaatatggcaccccgcagaaagaacgcattcgcaaagcggcgaaaaacaacaacattgtggtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaacgaacaggtgcatgtggcggcgtggccgagctttagcgtgtatcgcgcgtatgcgtatgcgttaggcccgattgtgaacaccagcgcgagcagaatttatgcggtggaaggccagtgctttgtgctggcgccgtgcgcagtggttagcaaagaaatggtggatgagatgtgcaccaccgatctgcataaagcgctgttaaaaaccggcggcggctatgcgcgcatttttggcccggatggccgccagttacatgaaggcctgccggccggcgaagaaggcctgctgtatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcaccggaaccggcattaccggcgatggcggaactg

SEQ ID NO: 13 - protein, 344 aa, Synthetic construct
MNKPKFKAAAVQAAPVLLDLDKTIDKTIALMERAARNGAELIAFPETWLPGYPWYAWLDAPAMWLPQYTQRYFDNSLEIGTPQAERIRKAAKDNNITVSMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYEAYAYALGAEVNTSASRIYAVEGQCFVLAPCATVSKGMVDEMCGTDGRKALLKTGGVYARIFGPDGRALVEGLPAGEEGLIYAEIDLGLISVSKAVADPAGHYSRPDVTQLIHNKAPRRAVVDTAPPSDPAPAVERPHGADEPALPAMAEL

SEQ ID NO: 14 - DNA, 1032 bp, Synthetic construct
  source 1..1032: mol_type = other DNA
atgaacaaaccgaaatttaaagcggcggcggtgcaggcggcgccggtgctgctggatttagacaaaaccattgataagaccattgcgctgatggaacgcgcggcgcgcaacggcgcggaactgattgcgtttccggaaacctggttaccgggctatccgtggtatgcgtggctggatgcgccggcaatgtggttaccgcagtatacccagcgctattttgataacagcctggaaattggcaccccgcaggcggaacgcattcgcaaagcggcgaaagataacaacattaccgtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcgcggaagtgaacaccagcgcgagcagaatttatgcggtggaaggccagtgctttgtgctggcgccgtgcgcaactgttagcaaaggcatggtggatgagatgtgcggcaccgatggccgcaaagcgctgttaaaaaccggcggcgtgtatgcgcgcatttttggcccggatggccgcgcgttagtggaaggcctgccggccggcgaagaaggcctgatttatgcggaaattgatctgggcctgattagcgtgagcaaagcggtggcggatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 15 - protein, 344 aa, Synthetic construct
MNKPKFKAAAVQAAPVFLDLDKTIDKTEALMEDAARNGAELIAFPETWLPGYPWYAWLDAPAMWLPMYTQRYFDNSLEYGTPQAERIRKAAKDNNITVSMGLSEREGASLYISQWFIDDDGDTIAKRRKLKPTHVERTIFGEGDGSDLAVADIRLGRVGGLCCWEHLQPLSKYAMYAQDEQVHVAAWPSFSLYEAYAYALGPEVNTSASRIYAVEGQCFVLAPCATVSKEMVDEMCTTDLHKALLKTGGGYAGIFGPDGRQLHEGLPAGEEGLIYAEIDLGLISVSKSVGDPAGHYSRPDVTQLIHNKAPRRRVVDTAAPSDPAPAVERPHGADEPALPAMAEL

SEQ ID NO: 16 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaagatgcggcgcgcaacggcgcggaactgattgcgtttccggaaacctggttaccgggctatccgtggtatgcgtggctggatgcgccggcaatgtggttaccgatgtatacccagcgctattttgataacagcctggaatatggcaccccgcaggcggaacgcattcgcaaagcggcgaaagataacaacattaccgtgagcatgggcctgagcgaacgcgaaggcgcgagcctgtacatttctcagtggtttattgatgatgatggcgataccattgcgaaacgccgcaaactgaaaccgacccatgtggaacgcaccatttttggcgaaggcgatggcagcgatctggcggtggcggatattcgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcataaagcgctgttaaaaaccggcggcggctatgcgggcatttttggcccggatggccgccagttacatgaaggcctgccggccggcgaagaaggcctgatttatgcggaaattgatctgggcctgattagcgtgagcaaaagcgtgggcgatccggcgggtcattatagccgcccagatgtgacccagttaattcataacaaagcgccgcgccgtcgcgttgttgatactgcagcgccatctgacccagcaccggcggtggaacgtccacatggtgcagatgaaccggcattaccggcgatggcggaactg

SEQ ID NO: 17 - protein, 344 aa, Synthetic construct
MNKPKFKAASVQAAPVFLDLDKTIDKTLALMEDAARNGAELIAFPETWLPGYPWYAWLGAPAMWLPQYTQRYFDNSLEYGTPQAERIRKTAKDNNIVVSMGLSEREGGSLYISQWFIDRDGDTIAQRRKLKPTHVERTIFGEGDGSDLAVADTRLGRVGGLCCWEHLQPLSKYAMYAQHEQVHVAAWPSFSLYVAYAYALGPDVNTSASRIYAVEGQCFVLAPCATVSKEMVDEMCTTDLQKALLLTGGCYARIFGPDGRQLHEGLAAGEEGLIYAEIDLGLISVSKAVGDPAGHYSRPDVTQLIHHKASRRAVVDTAPPSDPAPAVERPHGADEPALPAMAEL

SEQ ID NO: 18 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaagatgcggcgcgcgcgggcgcggaactgattgcgtttccggaaacctggttaccgggctatccgtggtatgcgtggctggatgcgccggcaatgtggttaccggtgtatacccagcgctattttgataacagcctggaatatggcaccccgcaggcggaacgcattcgcaaagatgcgaaagataacaacatttatgtgagcatgggcctgagcgaacgcgaaggcgaaagcctgtacatttctcagtggtttattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagcatgaacaggtgcatgtggcggcgtggccgagctttagcctgtatgaagcgtatgcgtatgcgttaggcggcgaagtgaacaccagcgcgagcagaatttatgcggtggaaggcagctgctttgtgctggcgccgtgcgcaactgttagcaaaggcatggtggatgagatgtgcaccaccgatctgcataaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgaccgatccggcgggtcattatagccgcccagatgtgacccagttaattcataacaaacgcccgcgccgtgcagttgttgatactgcaccaccatctgacccagcaccggcggtggaacgtccacatggtgcagatgaaccggcattaccggcgatggcggaactg

SEQ ID NO: 19 - protein, 344 aa, Synthetic construct
MNKPKFKAAAVQAAPVFLDLDKTIDKTEALMEDAARAGAELIAFPETWLPGYPWYAWLDAPAMWLPVYTQRYFDNSLEYGTPQAERIRKDAKDNNIYVSMGLSEREGESLYISQWFIDRDGDTIATRRKLKPTHVERTIFGEGDGSDLAVWDTRLGRVGGLCCWEHLQPLSKYAMYAQHEQVHVAAWPSFSLYEAYAYALGGEVNTSASRIYAVEGSCFVLAPCATVSKGMVDEMCTTDLHKALLKTGGGYARIFGPDGRQLHEGLPAGEEGLIYAEIDLGLISVSKAVTDPAGHYSRPDVTQLIHNKRPRRAVVDTAPPSDPAPAVERPHGADEPALPAMAEL

SEQ ID NO: 20 - DNA, 1032 bp, Synthetic construct; identical to SEQ ID NO: 18
  source 1..1032: mol_type = other DNA

SEQ ID NO: 21 - protein, 344 aa, Synthetic construct
MNKPKFKAAAVQAAPVFLDLDKTIDKTIALMEKAARNGAELIAFPETWLPGYPWYAWLDAPAMWLPQYTQRYFDNSLEYGTPQDERIRKDAKDNNIHVSTGLSEREGGSLYISQWFIDRDGDTIARRRKLKPTHVERTIFGEGDGSDLAVWDTRLGRVGGLCCWEHLQPLSKYAMYAQNEQVHVAAWPSFSAYRAYAYALGPIVNTSASRIYAVEGQCFVLAPCATVSKEMSDEMCTTDLKKALLKTGGGYATIFGPDGRQLHEGLPAGEEGLIYAEIDLGLISVSKSVGDPAGHYSRPDVTQLIHNKAPRRAVVDQAPPSDPAPAVERPHGADEPALPAMAEL

SEQ ID NO: 22 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggatgaacgcattcgcaaagatgcgaaagataacaacattcatgtgagcaccggcctgagcgaacgcgaaggcggcagcctgtacatttctcagtggtttattgatcgcgatggcgataccattgcgcgccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcgcgtatcgcgcgtatgcgtatgcgttaggcccgattgtgaacaccagcgcgagcagaatttatgcggtggaaggccagtgctttgtgctggcgccgtgcgcaactgttagcaaagaaatgagcgatgagatgtgcaccaccgatctgaaaaaagcgctgttaaaaaccggcggcggctatgcgaccatttttggcccggatggccgccagttacatgaaggcctgccggccggcgaagaaggcctgatttatgcggaaattgatctgggcctgattagcgtgagcaaaagcgtgggcgatccggcgggtcattatagccgcccagatgtgacccagttaattcataacaaagcgccgcgccgtgcagttgttgatcaggcaccaccatctgacccagcaccggcggtggaacgtccacatggtgcagatgaaccggcattaccggcgatggcggaactg

SEQ ID NO: 23 - protein, 344 aa, Synthetic construct
MNKPKFKAAAVQAAPVFLDLDKTIDKTIALMEDAARNGAELIAFPETWFPGYPWYAWLTAPAMWLPQYTQRYFDNSLEFGTPQAERIRKDAKDNNIVVSMGLSEREGESLYISQWFIDRDGDTIAKRRKLKPTHVERTIFGEGDGSDLAVWDTRLGRVGGLCCWEHLQPLSKYAMYAQTEQVHVAAWPSFSLYVAYAYALGPDVNTSASRIYAVEGQCFVLAPCATVSKEMVDEMCTTDLHKALLKTGGGYARIFGPDGRLLHEGLPAGEEGLIFAEIDLGLISVSKAVGDPAGHYSRPDVTQLIHHKKPRRAVVDTAPPSDPAPRVERPHGADEPALPAMAEL

SEQ ID NO: 24 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggtttccgggctatccgtggtatgcgtggctgaccgcgccggcaatgtggttaccgcagtatacccagcgctattttgataacagcctggaatttggcaccccgcaggcggaacgcattcgcaaagatgcgaaagataacaacattgtggtgagcatgggcctgagcgaacgcgaaggcgaaagcctgtacatttctcagtggtttattgatcgcgatggcgataccattgcgaaacgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaccgaacaggtgcatgtggcggcgtggccgagctttagcctgtatgtggcgtatgcgtatgcgttaggcccggatgtgaacaccagcgcgagcagaatttatgcggtggaaggccagtgctttgtgctggcgccgtgcgcaactgttagcaaagaaatggtggatgagatgtgcaccaccgatctgcataaagcgctgttaaaaaccggcggcggctatgcgcgcatttttggcccggatggccgcctgttacatgaaggcctgccggccggcgaagaaggcctgatttttgcggaaattgatctgggcctgattagcgtgagcaaagcggtgggcgatccggcgggtcattatagccgcccagatgtgacccagttaattcatcataaaaaaccgcgccgtgcagttgttgatactgcaccaccatctgacccagcaccgcgcgtggaacgtccacatggtgcagatgaaccggcattaccggcgatggcggaactg

SEQ ID NO: 25 - protein, 344 aa, Synthetic construct
MNKPKFKAAAVQAAPVFLDLDKTIDKTIALMEDAARNGAELIAFPETWLPGYPWYAWLDAPAMWLPQYTARYFDNSLEIGTPQADRIRKAAKDNNIVVSMGLSEREGGSLYISQWFIDRDGDTIATRRKLKPTHVERTIFGEGDGSDLAVWDTRLGRVGGLCCWEHLQPLSKYAMYAQNEQVHVAAWPSFSMYDAYAYALGSIVNTSASRIYAVEGQCFVLAPCAIVSKEMVDEMCTTDDQKALLKTGGGYAVIFGPDGRQLHEGLPAGEEGLIYAEIDLGLISVSKAVNDPAGHYSRPDVTQLIHNKAPRRAVVDTAPPSDDAPTVERPHGADLPALPAMAEL

SEQ ID NO: 26 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtataccgcgcgctattttgataacagcctggaaattggcaccccgcaggcggatcgcattcgcaaagcggcgaaagataacaacattgtggtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcatgtatgatgcgtatgcgtatgcgttaggcagcattgtgaacaccagcgcgagcagaatttatgcggtggaaggccagtgctttgtgctggcgccgtgcgcaattgttagcaaagaaatggtggatgagatgtgcaccaccgatgatcagaaagcgctgttaaaaaccggcggcggctatgcggtgatttttggcccggatggccgccagttacatgaaggcctgccggccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacgatgcaccgaccgtggaacgtccacatggtgcagatctgccggcattaccggcgatggcggaactg

SEQ ID NO: 27 - protein, 344 aa, Synthetic construct
MNKPKFKAAAVQAAPVFLDLDKTIDKTVALMEKAARNGAELIAFPETWLPGYPWYAWLDAPAMWLPQYTQRYFDNSLEYGTPQAKRIRKAAKRNNITVSLGLSEREGGSLYISQWFIDRDGDTIAKRRKLKPTHVERTIFGEGDGSDLAVHDIRLGRVGGLCCWEHLQPLSKYAMYAQNEQVHVAAWPSFSLYKAYAYALGPEVNTSASRIYAVEGQCFVLAPCACVSKEMVDEMCTTDARKALLKTGGGYARIFGPDGRQLHEGLPAGEEGLIYAEIDLGLISVGKAVGDPAGHYSRPDVTQLIHNKAPRRAVVDTAQPSDPAPAVERPHGADEPALPAMAEL

SEQ ID NO: 28 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcgaaacgcattcgcaaagcggcgaaacgcaacaacattaccgtgagcctgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgcatgatattcgtttaggtcgcgttggcggcttatgctgctgggaacatctgcagccgctgagcaaatatgcgatgtatgcgcagaacgaacaggtgcatgtggcggcgtggccgagctttagcctgtataaagcgtatgcgtatgcgttaggcccggaagtgaacaccagcgcgagcagaatttatgcggtggaaggccagtgctttgtgctggcgccgtgcgcatgcgttagcaaagaaatggtggatgagatgtgcaccaccgatgcgcgcaaagcgctgttaaaaaccggcggcggctatgcgcgcatttttggcccggatggccgccagttacatgaaggcctgccggccggcgaagaaggcctgatttatgcggaaattgatctgggcctgattagcgtgggcaaagcggtgggcgatccggcgggtcattatagccgcccagatgtgacccagttaattcataacaaagcgccgcgccgtgcagttgttgatactgcacagccatctgacccagcaccggcggtggaacgtccacatggtgcagatgaaccggcattaccggcgatggcggaactg

SEQ ID NO: 29 - protein, 344 aa, Synthetic construct
MNKPKFKAAAVQAAPVFLDLDKTIDKTLALMEDAARNGAELIAFPETWLPGYPWYAWLDAPAMWLPRYIQRYFDNSLEYGTPQAARIRKAAKDNNIYISMGLSEREGSSLYISQWFIDRDGDTIAQRRKLKPTHVERTIFGEGDGSDLAVADTRLGRVGGLCCWEHLQPLSKYAMYAQKEQVHVAAWPSFSLYEAYAYALGPEVNESASRIYAVEGQCFVLAPCATVSKEMVDEMCTTDLRKALLKTGGVYARIFGPDGRSLHEGLPAGEEGLIYAEIDLGLITVSKAVGDPAGHYSRPDVTQLIFHKAPRRAVVDTAPPSDPAPAVERPHGADEPALPAMAEL

SEQ ID NO: 30 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaagatgcggcgcgcaacggcgcggaactgattgcgtttccggaaacctggttaccgggctatccgtggtatgcgtggctggatgcgccggcaatgtggttaccgcgctatattcagcgctattttgataacagcctggaatatggcaccccgcaggcggcgcgcattcgcaaagcggcgaaagataacaacatttatattagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtggcggatacccgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacgaaagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggcgtgtatgcgcgcatttttggcccggatggccgcagcttacatgaaggcctgccggccggcgaagaaggcctgatttatgcggaaattgatctgggcctgattaccgtgagcaaagcggtgggcgatccggcgggtcattatagccgcccagatgtgacccagttaatttttcataaagcgccgcgccgtgcagttgttgatactgcaccaccatctgacccagcaccggcggtggaacgtccacatggtgcagatgaaccggcattaccggcgatggcggaactg

SEQ ID NO: 31 - protein, 344 aa, Synthetic construct
MNKPKFKAAAVQAAPVFLDLDKTIDKTIALIEDAARNGAELIAFPETWLPGYPWYAWLDTPAMWAPQYTQRYFDNSLEFGTPQAERIRKDAKDNNIVVSMGLSEREGGSLYISQWFIDRDGDTIARRRKLKPTHVERTIFGEGDGSDLAVWDTRLGRVGGLCCWEHLQPLSKYAMYAQKEQVHVAAWPSFSAYEAYAYALGPEVNTSASRIYAVEGQCFVLAPCATVSKEMVDEMCTTDAKKALLKTGGVYARIFGPDGRQLHEGLPAEEEGLVYAEIDLGLISVSKAVSDPAGHYSRPDVTQLIHDKAPRRAVVDTAPPSDPAPAVERPHGADLPALPAMAEL

SEQ ID NO: 32 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgattgaagatgcggcgcgcaacggcgcggaactgattgcgtttccggaaacctggttaccgggctatccgtggtatgcgtggctggataccccggcaatgtgggcgccgcagtatacccagcgctattttgataacagcctggaatttggcaccccgcaggcggaacgcattcgcaaagatgcgaaagataacaacattgtggtgagcatgggcctgagcgaacgcgaaggcggcagcctgtacatttctcagtggtttattgatcgcgatggcgataccattgcgcgccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcgcgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatgcgaaaaaagcgctgttaaaaaccggcggcgtgtatgcgcgcatttttggcccggatggccgccagttacatgaaggcctgccggccgaagaagaaggcctggtgtatgcggaaattgatctgggcctgattagcgtgagcaaagcggtgagcgatccggcgggtcattatagccgcccagatgtgacccagttaattcatgataaagcgccgcgccgtgcagttgttgatactgcaccaccatctgacccagcaccggcggtggaacgtccacatggtgcagatctgccggcattaccggcgatggcggaactg

SEQ ID NO: 33 - protein, 344 aa, Synthetic construct
MNKPKFKAAAVQAAPVFLDLDKAIDKTIALMERAARNGAELIAFPETWLPGYPWYAWLDAPAMWLPMYTQRYFDNSLEYGTPQARRIRKAAKDNNITVSMGLSEREGGSLYIAQWFIDDDGDTLAQRRKLKPTHVERTIFGEGDGSDLAVWDTRLGRVGGLCCWEHLQPLSKYAMYAQHEQVHVAAWPSFSLYGAYAYALGPEVNTSASRAYAVEGQCFVLAPCALVSKEMVDEMCTTDGQKALLKTGGAYARIFGPDGRVLHEGLPAGEEGLIYAEIDLGLISVSKAVADPAGHYSRPDVTQLIHNKAPRRAVVDTAPPSDPAPAVERPHGADEPALPAMAEL

SEQ ID NO: 34 - DNA, 1032 bp, Synthetic construct
  source 1..1032: mol_type = other DNA
atgaacaaaccgaaatttaaagcggcggcggtgcaggcggcgccggtgtttctggatttagacaaagcgattgataagaccattgcgctgatggaacgcgcggcgcgcaacggcgcggaactgattgcgtttccggaaacctggttaccgggctatccgtggtatgcgtggctggatgcgccggcaatgtggttaccgatgtatacccagcgctattttgataacagcctggaatatggcaccccgcaggcgcgccgcattcgcaaagcggcgaaagataacaacattaccgtgagcatgggcctgagcgaacgcgaaggcggcagcctgtacattgcgcagtggtttattgatgatgatggcgataccctggcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagcatgaacaggtgcatgtggcggcgtggccgagctttagcctgtatggcgcgtatgcgtatgcgttaggcccggaagtgaacaccagcgcgagcagagcgtatgcggtggaaggccagtgctttgtgctggcgccgtgcgcactggttagcaaagaaatggtggatgagatgtgcaccaccgatggccagaaagcgctgttaaaaaccggcggcgcgtatgcgcgcatttttggcccggatggccgcgtgttacatgaaggcctgccggccggcgaagaaggcctgatttatgcggaaattgatctgggcctgattagcgtgagcaaagcggtggcggatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 35 - protein, 344 aa, Synthetic construct
MNKPKFKAAAVQAAPVFLDLDKTIDKTLALMEDAARNGAELIAFPETWLPGYPWYAWLYSPAMWLPQYTQRYFDNSLEYGTPQADRIRKDAKDNNIVVSMGLSEREGGSLYISQWFIDRDGDTIAARRKLKPTHVERTIFGEGDGSDLAVWDTRLGRVGGLCCWEHLQPLSKYAMYAQNEQVHVAAWPSFSVYEAYANALGPEVNTSASRIYAVEGGCFVLAPCASVSKGMVDEMCTTDVQKALLKTGGCYARIFGPDGRQLHEGLPAGEEGLIVAEIDLGLISVSKAVTDPAGHYSRPDVTQLLHNKAPRRAVVDTAPPSDPAPAVERPHGADEPALPAMAEL

SEQ ID NO: 36 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaagatgcggcgcgcaacggcgcggaactgattgcgtttccggaaacctggttaccgggctatccgtggtatgcgtggctgtatagcccggcaatgtggttaccgcagtatacccagcgctattttgataacagcctggaatatggcaccccgcaggcggatcgcattcgcaaagatgcgaaagataacaacattgtggtgagcatgggcctgagcgaacgcgaaggcggcagcctgtacatttctcagtggtttattgatcgcgatggcgataccattgcggcgcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcgtgtatgaagcgtatgcgaacgcgttaggcccggaagtgaacaccagcgcgagcagaatttatgcggtggaaggcggctgctttgtgctggcgccgtgcgcaagcgttagcaaaggcatggtggatgagatgtgcaccaccgatgtgcagaaagcgctgttaaaaaccggcggctgctatgcgcgcatttttggcccggatggccgccagttacatgaaggcctgccggccggcgaagaaggcctgattgtggcggaaattgatctgggcctgattagcgtgagcaaagcggtgaccgatccggcgggtcattatagccgcccagatgtgacccagttactgcataacaaagcgccgcgccgtgcagttgttgatactgcaccaccatctgacccagcaccggcggtggaacgtccacatggtgcagatgaaccggcattaccggcgatggcggaactg

SEQ ID NO: 37 - protein, 344 aa, Synthetic construct
MNKPKFKAAAVQAAPVFLDLDKTIDKTIALMEKAARNGAELIAFPETWLPGYPWYAWLDAPAMWLPQYTQRYFDNSLEVGTPQAERIRKDAKDNNIVVSMGLSEREGASLYISQWFIDRDGDTIARRRKLKPTHVERTIFGEGDGSDLSVWDARLGRVGGLCCWEHLQPLSKYAMYAQDEQVHVAAWPSFSIYKAYAYALGHEVNTSASRIYAVEGQCFVLAPCATVSKDMVDEMCTTDLQKALLKTGGGYARIFGPDGRSLHEGLPASEEGLVYAEIDLGLIGVSKAVGDPAGHYSRPDVTQLIHHKAPRRAVVDTAPPSDPAPAVERPHGADEPALPAMAEL

SEQ ID NO: 38 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaagtgggcaccccgcaggcggaacgcattcgcaaagatgcgaaagataacaacattgtggtgagcatgggcctgagcgaacgcgaaggcgcgagcctgtacatttctcagtggtttattgatcgcgatggcgataccattgcgcgccgccgcaaactgaaaccgacccatgtggaacgcaccatttttggcgaaggcgatggcagcgatctgagcgtgtgggatgcgcgtttaggtcgcgttggcggcttatgctgctgggaacatctgcagccgctgagcaaatatgcgatgtatgcgcaggatgaacaggtgcatgtggcggcgtggccgagctttagcatttataaagcgtatgcgtatgcgttaggccatgaagtgaacaccagcgcgagcagaatttatgcggtggaaggccagtgctttgtgctggcgccgtgcgcaactgttagcaaagatatggtggatgagatgtgcaccaccgatctgcagaaagcgctgttaaaaaccggcggcggctatgcgcgcatttttggcccggatggccgcagcttacatgaaggcctgccggccagcgaagaaggcctggtgtatgcggaaattgatctgggcctgattggcgtgagcaaagcggtgggcgatccggcgggtcattatagccgcccagatgtgacccagttaattcatcataaagcgccgcgccgtgcagttgttgatactgcaccaccatctgacccagcaccggcggtggaacgtccacatggtgcagatgaaccggcattaccggcgatggcggaactg

SEQ ID NO: 39 - protein, 344 aa, Synthetic construct
MNKPKFKAAAVQAAPVFLDLDKTIDKTIALMEDAARNGAELIAFPETWLPGYPWYAWLDAPAMWLPQYTHRYFDNSLEFGTPQADRIRKVAKDNNIVVSMGLSEREGGSLYISQWFIDRDGDTIAMRRKLKPTHAERTIFGEGDGSDLAVVDTRLGRVGGLCCWEHLQPLSKYAMYAQDEQVHVAAWPSFSLYEAYAYALGPEVNTHASRIYAVEGQCFVLAPCAIVSKEMVDEMCTTDLRKALLKVGGAYARIFGPDGRELHEGLPAGEEGLIYAEIDLGLISVSKAVGDPAGHYSRPDVTQLIHHKAPRRPVVDTAPPSDPAPAVERPHGADEAALPAMAEL

SEQ ID NO: 40 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccatcgctattttgataacagcctggaatttggcaccccgcaggcggatcgcattcgcaaagtggcgaaagataacaacattgtggtgagcatgggcctgagcgaacgcgaaggcggcagcctgtacatttctcagtggtttattgatcgcgatggcgataccattgcgatgcgccgcaaactgaaaccgacccatgcggaacgcaccatttttggcgaaggcgatggcagcgatctggcggtggtggatacccgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccggaagtgaacacccatgcgagcagaatttatgcggtggaaggccagtgctttgtgctggcgccgtgcgcaattgttagcaaagaaatggtggatgagatgtgcaccaccgatctgcgcaaagcgctgttaaaagtgggcggcgcgtatgcgcgcatttttggcccggatggccgcgaattacatgaaggcctgccggccggcgaagaaggcctgatttatgcggaaattgatctgggcctgattagcgtgagcaaagcggtgggcgatccggcgggtcattatagccgcccagatgtgacccagttaattcatcataaagcgccgcgccgtccggttgttgatactgcaccaccatctgacccagcaccggcggtggaacgtccacatggtgcagatgaagcggcattaccggcgatggcggaactg

SEQ ID NO: 41 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEVGTPQHDRIRKVAKDNNIVVVMGLSEREGGSLYISQWFIDRDGDTIAARRKLKPTHVERTIFGEGDGSDLAVIDLRLGRVGGLCCWEHLQPLSKYAMYAQTEQVHVAAWPSFSAYEGYAYALGPEVNTSASRTYAVEGQCFVLAPCALVSKEMVDEMCTTALQKALLKTGGCYARIFGPDGRQLHEGLPAGEEGLIYAEIDLGLISVSKAVGDPAGHYSRPDVTQLIINKAPRRAVVDTAPPSDPAPAVERPHGADEPALPAMAEL

SEQ ID NO: 42 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaagtgggcaccccgcagcatgatcgcattcgcaaagtggcgaaagataacaacattgtggtggtgatgggcctgagcgaacgcgaaggcggcagcctgtacatttctcagtggtttattgatcgcgatggcgataccattgcggcgcgccgcaaactgaaaccgacccatgtggaacgcaccatttttggcgaaggcgatggcagcgatctggcggtgattgatctgcgtttaggtcgcgttggcggcttatgctgctgggaacatctgcagccgctgagcaaatatgcgatgtatgcgcagaccgaacaggtgcatgtggcggcgtggccgagctttagcgcgtatgaaggctatgcgtatgcgttaggcccggaagtgaacaccagcgcgagcagaacctatgcggtggaaggccagtgctttgtgctggcgccgtgcgcactggttagcaaagaaatggtggatgagatgtgcaccaccgcgctgcagaaagcgctgttaaaaaccggcggctgctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattattaacaaagcgccgcgccgtgcagttgttgatactgcaccaccatctgacccagcaccggcggtggaacgtccacatggtgcagatgaaccggcattaccggcgatggcggaactg

SEQ ID NO: 43 - protein, 344 aa, Synthetic construct
MNKPKFKAAAVQAAPVFLDLDKTIDKTEALMEDAARNGAELIAFPETWLPGYPWYAWLDAPAMWLPQYTQRYFDNSLEIGTPQAKRIRKAAKDNNIWVSMGLSEREGASLYISQWFIDRDGDTIARRRKLKPTHVERTIFGEGDGSDLAVWDTRLGRVGTLCCWEHLQPLSKYAMYAQTEQVHVAAWPSFSLYKAYAYALGPIVNTSASRIYAVEGQCFVLAPCATVSKEMVDEMCTTDLRKALLKTGGGYARIFGPDGRVLHEGLPASEEGLIYAEIDLGMISVSKAVSDPAGHYSRPDVTQLIHDKAPRRAVVDFAPPSDPAPRVERPHGADEPALPAMAEL

SEQ ID NO: 44 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaaattggcaccccgcaggcgaaacgcattcgcaaagcggcgaaagataacaacatttgggtgagcatgggcctgagcgaacgcgaaggcgcgagcctgtacatttctcagtggtttattgatcgcgatggcgataccattgcgcgccgccgcaaactgaaaccgacccatgtggaacgcaccatttttggcgaaggcgatggcagcgatctggcggtgtgggatacccgtttaggtcgcgttggcaccttatgctgctgggaacatctgcagccgctgagcaaatatgcgatgtatgcgcagaccgaacaggtgcatgtggcggcgtggccgagctttagcctgtataaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggcggctatgcgcgcatttttggcccggatggccgcgtgttacatgaaggcctgccggccagcgaagaaggcctgatttatgcggaaattgatctgggcatgattagcgtgagcaaagcggtgagcgatccggcgggtcattatagccgcccagatgtgacccagttaattcatgataaagcgccgcgccgtgcagttgttgattttgcaccaccatctgacccagcaccgcgcgtggaacgtccacatggtgcagatgaaccggcattaccggcgatggcggaactg

SEQ ID NO: 45 - protein, 344 aa, Synthetic construct
MNKPKFKAAAVQAAPVFLDLDKTIDKTIALMEDAAREGAELIAFPETWLPGYPWYAWLTAPAMWLPQYTARYFDNSLEYGTPQARRIRKAAKDNNIVVSMGLSEREGGSLYISQWFIDRDGDTIAKRRKLKPTHVERTIFGEGDGSDLAVHDIRLGRVGGLCCWEHLQPLSKYAMYAQNEQVHVAAWPSFSMYEAYAYALGPDVNTSASRLYAVEGQCFVLAPCAIVSKEMVDEMCTTDDQKALLHTGGVYAVIFGPDGRELHEGLPHGEEGLIYAEIDLGLISVAKAVGDPAGHYSRPDVTQLIHNKAPRRAVVDTAPPSDPAPAVERPHGADEPALPAMAEL

SEQ ID NO: 46 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcgaaggcgcggaactgattgcgtttccggaaacctggttaccgggctatccgtggtatgcgtggctgaccgcgccggcaatgtggttaccgcagtataccgcgcgctattttgataacagcctggaatatggcaccccgcaggcgcgccgcattcgcaaagcggcgaaagataacaacattgtggtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgcatgatattcgtttaggtcgcgttggcggcttatgctgctgggaacatctgcagccgctgagcaaatatgcgatgtatgcgcagaacgaacaggtgcatgtggcggcgtggccgagctttagcatgtatgaagcgtatgcgtatgcgttaggcccggatgtgaacaccagcgcgagcagactgtatgcggtggaaggccagtgctttgtgctggcgccgtgcgcaattgttagcaaagaaatggtggatgagatgtgcaccaccgatgatcagaaagcgctgttacataccggcggcgtgtatgcggtgatttttggcccggatggccgcgaattacatgaaggcctgccgcatggcgaagaaggcctgatttatgcggaaattgatctgggcctgattagcgtggcgaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 47 - protein, 344 aa, synthetic construct
MNKPKFKAAAVQAAPVFLDLDKSIDKTRALMEDAARNGAELIAFPETWLPGYPWYAWLDAPAMWLPQYTKRYFDNSLEYGSPQADRIRKDAKDNNIVMSMGLSEREGSSLYISQWFIDRDGDTIAQRRKLKPTHVERTIFGEGDGSDLAVIDIRLGRVGGLCCWEHLQPLSKYAMYAQTEQVHVAAWPSFSIYDAYAYALGPEVNTSASRIYAVEGQCFVLAPCATVSKGMSDEMCTTDLQKALLKTGGVYATIFGPDGRQLHEGLPAGQEGLIYAEIDLGLISVSKAVGDPAGHYSRPDVTQLIHNKVPRRAVVDTAPPSDPAPAVERPHGADEPALPAMAEL

SEQ ID NO: 48 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagacccgcgcgctgatggaagatgcggcgcgcaacggcgcggaactgattgcgtttccggaaacctggttaccgggctatccgtggtatgcgtggctggatgcgccggcaatgtggttaccgcagtataccaaacgctattttgataacagcctggaatatggcagcccgcaggcggatcgcattcgcaaagatgcgaaagataacaacattgtgatgagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtgattgatattcgtttaggtcgcgttggcggcttatgctgctgggaacatctgcagccgctgagcaaatatgcgatgtatgcgcagaccgaacaggtgcatgtggcggcgtggccgagctttagcatttatgatgcgtatgcgtatgcgttaggcccggaagtgaacaccagcgcgagcagaatttatgcggtggaaggccagtgctttgtgctggcgccgtgcgcaactgttagcaaaggcatgagcgatgagatgtgcaccaccgatctgcagaaagcgctgttaaaaaccggcggcgtgtatgcgaccatttttggcccggatggccgccagttacatgaaggcctgccggccggccaggaaggcctgatttatgcggaaattgatctgggcctgattagcgtgagcaaagcggtgggcgatccggcgggtcattatagccgcccagatgtgacccagttaattcataacaaagtgccgcgccgtgcagttgttgatactgcaccaccatctgacccagcaccggcggtggaacgtccacatggtgcagatgaaccggcattaccggcgatggcggaactg

SEQ ID NO: 49 - protein, 344 aa, Synthetic construct
MNKPKFKAAAVQAAPVFLDLDKTIDKTIALMERAARNGAELIAFPETWLPGYPWYAWLDAPAMWLPRYTQRYFDNSLEYGTPQAERIRKVAKQNNIVVSMGLSEREGGSLYISQWFIDRDGDTIATRRKLKPTHVERTIFGEGDGSDLAVVDTRLGRVGGLCCWEHLQPLSKYAMYAQTEQVHVAAWPSFSMYKAYAYALGPDVNTSASRIYAVEGQCFVLAPCATVSKSMVDEMCTTDLKKALLKTGGAYARIFGPDGRSLSEGLPAGTEGLIFAEIDLGLISVSKAVGDPAGHYSRPDVTQLIHNKAPRRAVVDTAPPSDPAPEVERPHGTDEPALPAMAEL

SEQ ID NO: 50 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctggttaccgggctatccgtggtatgcgtggctggatgcgccggcaatgtggttaccgcgctatacccagcgctattttgataacagcctggaatatggcaccccgcaggcggaacgcattcgcaaagtggcgaaacagaacaacattgtggtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtggtggatacccgtttaggtcgcgttggcggcttatgctgctgggaacatctgcagccgctgagcaaatatgcgatgtatgcgcagaccgaacaggtgcatgtggcggcgtggccgagctttagcatgtataaagcgtatgcgtatgcgttaggcccggatgtgaacaccagcgcgagcagaatttatgcggtggaaggccagtgctttgtgctggcgccgtgcgcaactgttagcaaaagcatggtggatgagatgtgcaccaccgatctgaaaaaagcgctgttaaaaaccggcggcgcgtatgcgcgcatttttggcccggatggccgcagcttaagcgaaggcctgccggccggcaccgaaggcctgatttttgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggaagtggaacgtccacatggtaccgatgaaccggcattaccggcgatggcggaactg

SEQ ID NO: 51 - protein, 344 aa, Synthetic construct
MNKPKFKAAAVQAAPVLLDLDKAIDKTIALMEDAARNGAELIAFPETWLPGYPWYAWLGAPAMWLPQYIQRYFDNSLEFGTPQAERIRKDAKKNNIWVSMGLSEREGGSLYISQWFIDRDGDTIAQRRKLKPTHVERTIFGEGDGSDLAVWDTRLGRVGGLCCWEHLQPLSKYAMYAQHEQVHVAAWPSFSLYVAYAYALGPIVNTSASRIYAVEGQCFVLAPCALVSKEMVDEMCTTDLRKALLKTGGAYASIFGPDGRVLHEGLPHGEEGLIYAEIDLGLISVAKAVADPAGHYSRPDVTQLIHNKAPRRAVVDTAPPSDPAPAVERPHGADEPALPAMAEL

SEQ ID NO: 52 - DNA, 1032 bp, Synthetic construct
  source 1..1032: mol_type = other DNA
atgaacaaaccgaaatttaaagcggcggcggtgcaggcggcgccggtgctgctggatttagacaaagcgattgataagaccattgcgctgatggaagatgcggcgcgcaacggcgcggaactgattgcgtttccggaaacctggttaccgggctatccgtggtatgcgtggctgggcgcgccggcaatgtggttaccgcagtatattcagcgctattttgataacagcctggaatttggcaccccgcaggcggaacgcattcgcaaagatgcgaaaaaaaacaacatttgggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagcatgaacaggtgcatgtggcggcgtggccgagctttagcctgtatgtggcgtatgcgtatgcgttaggcccgattgtgaacaccagcgcgagcagaatttatgcggtggaaggccagtgctttgtgctggcgccgtgcgcactggttagcaaagaaatggtggatgagatgtgcaccaccgatctgcgcaaagcgctgttaaaaaccggcggcgcgtatgcgagcatttttggcccggatggccgcgtgttacatgaaggcctgccgcatggcgaagaaggcctgatttatgcggaaattgatctgggcctgattagcgtggcgaaagcggtggcggatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 53 - protein, 344 aa, Synthetic construct
MNKPKFKAAAVQAAPVFLDLDKTIDKTIALMEKAARNGAELIAFPETWLPGYPWYAWLYAPAMWLPQYTQRYFDNSLEYGTPQAHRIRKAAKDNNIHVSMGLSEREGASLYITQWHIDRDGDTIAQRRKLKPTHVERTIFGEGDGSDLAVYDPRLGRVGGLCCWEHLQPLSKYAMYAQKEQVHVAAWPSFSVYEAYAYALGPDVNTSASRIYAVEGQCFVLAPCATVSKEMVDEMCITDSQKALLKTGGGYARIFGPDGRELHEGLPASEEGLIYAEIDLGLISVSKAVSDPAGHYSRPDVTQLIFEKAPRRAVVDTAPPSDEAPAVERPHGADEPALPAMAEL

SEQ ID NO: 54 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctgtatgcgccggcaatgtggttaccgcagtatacccagcgctattttgataacagcctggaatatggcaccccgcaggcgcatcgcattcgcaaagcggcgaaagataacaacattcatgtgagcatgggcctgagcgaacgcgaaggcgcgagcctgtacattacccagtggcatattgatcgcgatggcgataccattgcgcagcgccgcaaactgaaaccgacccatgtggaacgcaccatttttggcgaaggcgatggcagcgatctggcggtgtatgatccgcgtttaggtcgcgttggcggcttatgctgctgggaacatctgcagccgctgagcaaatatgcgatgtatgcgcagaaagaacaggtgcatgtggcggcgtggccgagctttagcgtgtatgaagcgtatgcgtatgcgttaggcccggatgtgaacaccagcgcgagcagaatttatgcggtggaaggccagtgctttgtgctggcgccgtgcgcaactgttagcaaagaaatggtggatgagatgtgcattaccgatagccagaaagcgctgttaaaaaccggcggcggctatgcgcgcatttttggcccggatggccgcgaattacatgaaggcctgccggccagcgaagaaggcctgatttatgcggaaattgatctgggcctgattagcgtgagcaaagcggtgagcgatccggcgggtcattatagccgcccagatgtgacccagttaatttttgaaaaagcgccgcgccgtgcagttgttgatactgcaccaccatctgacgaagcaccggcggtggaacgtccacatggtgcagatgaaccggcattaccggcgatggcggaactg

SEQ ID NO: 55 - protein, 344 aa, Synthetic construct
MNKPKFKAAAVQAAPVFLDLDKTIDKTIALIERAARNGAELIAFPETWLPGYPWYAWLYAPAMWLPQYTQRYFDNSLEYGTPQAERIRKTAKDNNIVVSMGLSEREGESLYISQWFIDRDGDTAAQRRKLKPTHVERTIFGEGDGSDLAVWDLRLGRVGGLCCWEHLQPLSKYAMYAQNEQVHVAAWPSFSMYEAYAYALGPEVNTSASRIYAVEGQCFVLAPCALVSKDMVDEMCGTDLHKALLKTGGCYATIFGPDGRVLHEGLPAGEEGLIYAEIDLGMISVGKAVNDPAGHYSRPDVTQLIHHKAPRRAVVDTAPPSDPAPAVERPHGADDPALPAMAEL

SEQ ID NO: 56 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgattgaacgcgcggcgcgcaacggcgcggaactgattgcgtttccggaaacctggttaccgggctatccgtggtatgcgtggctgtatgcgccggcaatgtggttaccgcagtatacccagcgctattttgataacagcctggaatatggcaccccgcaggcggaacgcattcgcaaaaccgcgaaagataacaacattgtggtgagcatgggcctgagcgaacgcgaaggcgaaagcctgtacatttctcagtggtttattgatcgcgatggcgataccgcggcgcagcgccgcaaactgaaaccgacccatgtggaacgcaccatttttggcgaaggcgatggcagcgatctggcggtgtgggatctgcgtttaggtcgcgttggcggcttatgctgctgggaacatctgcagccgctgagcaaatatgcgatgtatgcgcagaacgaacaggtgcatgtggcggcgtggccgagctttagcatgtatgaagcgtatgcgtatgcgttaggcccggaagtgaacaccagcgcgagcagaatttatgcggtggaaggccagtgctttgtgctggcgccgtgcgcactggttagcaaagatatggtggatgagatgtgcggcaccgatctgcataaagcgctgttaaaaaccggcggctgctatgcgaccatttttggcccggatggccgcgtgttacatgaaggcctgccggccggcgaagaaggcctgatttatgcggaaattgatctgggcatgattagcgtgggcaaagcggtgaacgatccggcgggtcattatagccgcccagatgtgacccagttaattcatcataaagcgccgcgccgtgcagttgttgatactgcaccaccatctgacccagcaccggcggtggaacgtccacatggtgcagatgatccggcattaccggcgatggcggaactg

SEQ ID NO: 57 - protein, 344 aa, Synthetic construct
MNKPKFKAAAVQAAPVLLDLDKTIDKTEALMEDAARNGAELIAFPETYLPGYPWYAWLDAPAMWAPQYVQRYFDNSLEYETPQAHRIRKAAKDNNITVSMGLSEREGASLYISQWFIDRDGDTIAQRRKLKPTHVERTIFGEGDGSDLAVVDTRLGRIGGLCCWEHLQPLSKYAMYAQDEQVHVAAWPSFSLYEAYAYALGPIVNTSASRIYAVEGQCFVLAPCATVSKDMVDEMCTTDEHKALLKTGGGYARIFGPDGRPLHEGLPHGEEGLIYAEIDLGLISVSKAVNDPAGHYSRPDVTQLIHEKAPRRAVVDTAPPSDPAPAVERPHGADEPALPAMAEL

SEQ ID NO: 58 - DNA, 1032 bp, Synthetic construct
  source 1..1032: mol_type = other DNA
atgaacaaaccgaaatttaaagcggcggcggtgcaggcggcgccggtgctgctggatttagacaaaaccattgataagaccgaagcgctgatggaagatgcggcgcgcaacggcgcggaactgattgcgtttccggaaacctatttaccgggctatccgtggtatgcgtggctggatgcgccggcaatgtgggcgccgcagtatgtgcagcgctattttgataacagcctggaatatgaaaccccgcaggcgcatcgcattcgcaaagcggcgaaagataacaacattaccgtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtggtggatacccgtttaggtcgcattggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccgattgtgaacaccagcgcgagcagaatttatgcggtggaaggccagtgctttgtgctggcgccgtgcgcaactgttagcaaagatatggtggatgagatgtgcaccaccgatgaacataaagcgctgttaaaaaccggcggcggctatgcgcgcatttttggcccggatggccgcccgttacatgaaggcctgccgcatggcgaagaaggcctgatttatgcggaaattgatctgggcctgattagcgtgagcaaagcggtgaacgatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 59 - protein, 344 aa, Synthetic construct
MNKPKFKAAAVQAAPVFLDLDKTIDKTIALMEDAARNGAELIAFPETWLPGYPWYAWLDAPAMWLPMYTQRYFDNSLEYGTPQFERIRKSAKDNNIWVSMGLSEREGGSLYISQWFIDRDGDTIAQRRKLKPTHVERTIFGEGDGSDLAVWDTRLGRVGGLCCWEHLQPLSKYAMYAQKEQVHVAAWPSFSMYDAYAYALGPEVNTSASRMYAVEGTCFVLAPCALVSKEMVDEMCTTDSRKALLKTGGVYASIFGPDGRELHEGLPAGEEGLIYAEIDLGLISVSKAVGDPAGHYSRPDVTQLIFDKASRRAVVDTAAPSDPAPAVERPHGADEDALPAMAEL

SEQ ID NO: 60 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgatgtatacccagcgctattttgataacagcctggaatatggcaccccgcagtttgaacgcattcgcaaaagcgcgaaagataacaacatttgggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcatgtatgatgcgtatgcgtatgcgttaggcccggaagtgaacaccagcgcgagcagaatgtatgcggtggaaggcacctgctttgtgctggcgccgtgcgcactggttagcaaagaaatggtggatgagatgtgcaccaccgatagccgcaaagcgctgttaaaaaccggcggcgtgtatgcgagcatttttggcccggatggccgcgaattacatgaaggcctgccggccggcgaagaaggcctgatttatgcggaaattgatctgggcctgattagcgtgagcaaagcggtgggcgatccggcgggtcattatagccgcccagatgtgacccagttaatttttgataaagcgagccgccgtgcagttgttgatactgcagcgccatctgacccagcaccggcggtggaacgtccacatggtgcagatgaagatgcattaccggcgatggcggaactg

SEQ ID NO: 61 - protein, 344 aa, Synthetic construct
MNKPKFKIAAVQAAPVFLDLDKTIDKTIALMEDAARNGAELIAFPETFLPGYPWYAWLDAPAMWVPQYTRRYFDNSLEYGTPQARRIRKSAKDNNIVVSLGLSEREGESLYISQWHIDRDGDTIAQRRKLKPTHVERTIFGEGDGSDLAVRDTRLGRVGGLCCWEHLQPLSKYAMYAQNEQVHVAAWPSFSAYGAYAYALGPDVNTSASRIYAVEGQCFVLAPCATVSKSMVDEMCTTDLRKALLKTGGAYAGIFGPDGRQLHEGLPAGEEGLIYAEIDLGLITVSKAVGDPAGHYSRPDVTQLIHNKAPRRAVVDTAPPSDPAPAVERPHGADEPALPAMAEL

SEQ ID NO: 62 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctatttaccgggctatccgtggtatgcgtggctggatgcgccggcaatgtggggcccgcgctatacccagcgctattttgataacagcctggaatatggcaccccgcaggcggaacgcattcgcaaagatgcgaaagataacaacattgtgattagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcatgatccgcgtttaggtcgcgttggcggcttatgctgctgggaacatctgcagccgctgagcaaatatgcgatgtatgcgcagaacgaacaggtgcatgtggcggcgtggccgagctttagcgtgtatgaagcgtatgcgtatgcgttaggcccggatgtgaacaccggcgcgagcagaatttatgcggtggaaggccagtgctttgtgctggcgccgtgcgcaactgttagcaaagatatggtggatgagatgtgcaccaccgatggccataaagcgctgttaaaaaccggcggcgtgtatgcgaccattctgggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcactgccatctgacccagcaccggcggtggaacgtccacatggtgcagatgaaccggcattaccggcgatggcggaactg

SEQ ID NO: 63 - protein, 344 aa, Synthetic construct
MNKPKFKAAAVQAAPVFLDLDKTIDKTIALMEDAARNGAELIAFPETYLPGYPWYAWLDAPAMWGPRYTQRYFDNSLEYGTPQAERIRKDAKDNNIVISMGLSEREGGSLYISQWFIDRDGDTIAQRRKLKPTHVERTIFGEGDGSDLAVHDPRLGRVGGLCCWEHLQPLSKYAMYAQNEQVHVAAWPSFSVYEAYAYALGPDVNTGASRIYAVEGQCFVLAPCATVSKDMVDEMCTTDGHKALLKTGGVYATILGPDGRQLHEGLPAGEEGLIYAEIDLGLISVSKAVGDPAGHYSRPDVTQLIHNKAPRRAVVDTALPSDPAPAVERPHGADEPALPAMAEL

SEQ ID NO: 64 - DNA, 1032 bp, Synthetic construct; identical to SEQ ID NO: 62
  source 1..1032: mol_type = other DNA

SEQ ID NO: 65 - protein, 344 aa, Synthetic construct
MNKPKFKAAAVQAAPVFLDLDKTIDKTIALMEDAARNGAELIAFPETWLPGYPWYAWLDSPAMWLPQYTRRYFDNSLEYGTPQKERIRKAAKDNNIYVSMGLSEREGGSLYISQWFIDRDGDTIAARRKLKPTHVERTIFGEGDGSDLAVADTRLGRVGGLCCWEHLQPLSKYAMYAQNEQVHVAAWPSFSLYGAYAYALGPEVNTSASRIYAVEGQCFVLAPCAVVSKSMVDEMCTTPLQKALLKTGGVYAAIFGPDGRLLHEGLPAGKEGLIVAEIDLGLISVSKAVTDPAGHYSRPDVTQLIHEKAPRRAVVDTAPPSDPAPPVERPHGADEPALPAMAEL

SEQ ID NO: 66 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatagcccggcaatgtggttaccgcagtatacccgccgctattttgataacagcctggaatatggcaccccgcagaaagaacgcattcgcaaagcggcgaaagataacaacatttatgtgagcatgggcctgagcgaacgcgaaggcggcagcctgtacatttctcagtggtttattgatcgcgatggcgataccattgcggcgcgccgcaaactgaaaccgacccatgtggaacgcaccatttttggcgaaggcgatggcagcgatctggcggtggcggatacccgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgtatgcgttaggcccggaagtgaacaccagcgcgagcagaatttatgcggtggaaggccagtgctttgtgctggcgccgtgcgcagtggttagcaaaagcatggtggatgagatgtgcaccaccccgctgcagaaagcgctgttaaaaaccggcggcgtgtatgcggcgatttttggcccggatggccgcctgttacatgaaggcctgccggccggcaaagaaggcctgattgtggcggaaattgatctgggcctgattagcgtgagcaaagcggtgaccgatccggcgggtcattatagccgcccagatgtgacccagttaattcatgaaaaagcgccgcgccgtgcagttgttgatactgcaccaccatctgacccagcaccgccggtggaacgtccacatggtgcagatgaaccggcattaccggcgatggcggaactg

SEQ ID NO: 67 - protein, 344 aa, Synthetic construct
MNKPKFKAAAVQAAPVFLDLDKTIDKTIALMEDAARNGAELIAFPETWLPGYPWYAWLDAPAMWLPQYTARYFDNSLEYGTPQDHRIRKSAKDNNIVVSMGLSEREGGSLYISQWFIDADGDTIAQRRKLKPTHVERTIFGEGDGSDLAVWDLRLGRVGGLCCWEHLQPLSKYAMYAQHEQVHVAAWPSFSLYEAYAYALGPIVNTSASRIYAVEGQCFVLAPCASVSKEMVDEMCTEDVQKALLKTGGCYAAIFGPDGRQLHEGLPPGEEGLAYAEIDLGAISVSKAVTDPAGHYSRPDVTQLIHDKAPRRAVVDTAPPSDPAPAVERPHGADEPALPAMAEL

SEQ ID NO: 68 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtataccgcgcgctattttgataacagcctggaatatggcaccccgcaggatcatcgcattcgcaaaagcgcgaaagataacaacattgtggtgagcatgggcctgagcgaacgcgaaggcggcagcctgtacatttctcagtggtttattgatgcggatggcgataccattgcgcagcgccgcaaactgaaaccgacccatgtggaacgcaccatttttggcgaaggcgatggcagcgatctggcggtgtgggatctgcgtttaggtcgcgttggcggcttatgctgctgggaacatctgcagccgctgagcaaatatgcgatgtatgcgcagcatgaacaggtgcatgtggcggcgtggccgagctttagcctgtatgaagcgtatgcgtatgcgttaggcccgattgtgaacaccagcgcgagcagaatttatgcggtggaaggccagtgctttgtgctggcgccgtgcgcaagcgttagcaaagaaatggtggatgagatgtgcaccgaagatgtgcagaaagcgctgttaaaaaccggcggctgctatgcggcgatttttggcccggatggccgccagttacatgaaggcctgccgccgggcgaagaaggcctggcgtatgcggaaattgatctgggcgcgattagcgtgagcaaagcggtgaccgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 69 - protein, 344 aa, Synthetic construct
MNKPKFKAAAVQAAPVFLDLDKTIDKTVALMEDAARNGAELIAFPETWLPGYPWYAWLDTPAMWLPQYTQRYFDNSLEVGTPQAARIRKAAKDNNIVVSMGLSEREGSSLYISQWFIDSDGDTIATRRKLKPTHVERTIFGEGDGSDLAVWDARLGRVGGLCCWEHLQPLSKYAMYAQTEQVHVAAWPSFSAYVAYAYALGPEVNTSASRIYAVEGQCFVLAPCASVSKEMVDEMCTTDLHKALLKTGGGYARIFGPDGRQLSEGLPAGEEGLIYAEIDLGLISVSKAVGDPAGHYSRPDVTQLIHHKAPRRAVVDGAAPSDPAPAVERPHGADEPALPAMAEL

SEQ ID NO: 70 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaagatgcggcgcgcaacggcgcggaactgattgcgtttccggaaacctggttaccgggctatccgtggtatgcgtggctggataccccggcaatgtggttaccgcagtatacccagcgctattttgataacagcctggaagtgggcaccccgcaggcggcgcgcattcgcaaagcggcgaaagataacaacattgtggtgagcatgggcctgagcgaacgcgaaggcagcagcctgtacatttctcagtggtttattgatagcgatggcgataccattgcgacccgccgcaaactgaaaccgacccatgtggaacgcaccatttttggcgaaggcgatggcagcgatctggcggtgtgggatgcgcgtttaggtcgcgttggcggcttatgctgctgggaacatctgcagccgctgagcaaatatgcgatgtatgcgcagaccgaacaggtgcatgtggcggcgtggccgagctttagcgcgtatgtggcgtatgcgtatgcgttaggcccggaagtgaacaccagcgcgagcagaatttatgcggtggaaggccagtgctttgtgctggcgccgtgcgcaagcgttagcaaagaaatggtggatgagatgtgcaccaccgatctgcataaagcgctgttaaaaaccggcggcggctatgcgcgcatttttggcccggatggccgccagttaagcgaaggcctgccggccggcgaagaaggcctgatttatgcggaaattgatctgggcctgattagcgtgagcaaagcggtgggcgatccggcgggtcattatagccgcccagatgtgacccagttaattcatcataaagcgccgcgccgtgcagttgttgatggcgcagcgccatctgacccagcaccggcggtggaacgtccacatggtgcagatgaaccggcattaccggcgatggcggaactg

SEQ ID NO: 71 - protein, 344 aa, Synthetic construct
MNKPKFKAAAVQAAPVFLDLDKTIDKTIALMEDAARNGAELIAFPETFLPGYPWYAWLDAPAMWLPQYTRRYFDNSLTFGTPQAHRIRKAAKDNNIWVSMGLSEREGASLYISQWFIDRDGDTIATRRKLKPTHVERTIFGEGDGSDLAVRDTRLGRVGGLCCWEHLQPLSKYAMYAQDEQVHVAAWPSFSLYEAYAHALGPEVNTSASRIYAVEGQCFVLAPCAIVSKEMSDEMCTTDDKKALLKTGGAYARIFGPDGRVLHEGLPAGEEGLIYAEIDLGLISVSKAVGDPAGHYSRPDVTQLIHNKAPRRAVVDTAQPSDPAPAVERPHGADEPALPAMAEL

SEQ ID NO: 72 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctttttaccgggctatccgtggtatgcgtggctggatgcgccggcaatgtggttaccgcagtatacccgccgctattttgataacagcctgacctttggcaccccgcaggcgcatcgcattcgcaaagcggcgaaagataacaacatttgggtgagcatgggcctgagcgaacgcgaaggcgcgagcctgtacatttctcagtggtttattgatcgcgatggcgataccattgcgacccgccgcaaactgaaaccgacccatgtggaacgcaccatttttggcgaaggcgatggcagcgatctggcggtgcgcgatacccgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgcatgcgttaggcccggaagtgaacaccagcgcgagcagaatttatgcggtggaaggccagtgctttgtgctggcgccgtgcgcaattgttagcaaagaaatgagcgatgagatgtgcaccaccgatgataaaaaagcgctgttaaaaaccggcggcgcgtatgcgcgcatttttggcccggatggccgcgtgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcacagccatctgacccagcaccggcggtggaacgtccacatggtgcagatgaaccggcattaccggcgatggcggaactg

SEQ ID NO: 73 - protein, 344 aa, Synthetic construct
MNKPKFKAAAVQAAPVFLDLDKTIDKTIALMEDAARNGAELIAFPETFLPGYPWYAWLYAPAMWLPQYTDRYFDNSLEYGTPQAERIRKDAKDNNIVVSMGLSEREGESLYISQWFIDRDGDTIAQRRKLKPTHVERTIFGEGDGSDLAVVDTDLGRVGGLCCWEHLQPLSKYAMYAQHEQVHVAAWPSFSLYEAYAYALGPDVNTSASRIYAVEGGCFVLAPCAVVSKAMVDEMCTTDLQKALLKTGGCYAAIFGPDGRPLHEGLPAGEEGLIYAEIDLGAISVAKAVGDPAGHYSRPDVTQLIHEKAPRRAVVDTAPPSDPAPAVERPHGADEPALPAMAEL

SEQ ID NO: 74 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctttttaccgggctatccgtggtatgcgtggctgtatgcgccggcaatgtggttaccgcagtataccgatcgctattttgataacagcctggaatatggcaccccgcaggcggaacgcattcgcaaagatgcgaaagataacaacattgtggtgagcatgggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtggtggataccgatttaggtcgcgttggcggcttatgctgctgggaacatctgcagccgctgagcaaatatgcgatgtatgcgcagcatgaacaggtgcatgtggcggcgtggccgagctttagcctgtatgaagcgtatgcgtatgcgttaggcccggatgtgaacaccagcgcgagcagaatttatgcggtggaaggcggctgctttgtgctggcgccgtgcgcagtggttagcaaagcgatggtggatgagatgtgcaccaccgatctgcagaaagcgctgttaaaaaccggcggctgctatgcggcgatttttggcccggatggccgcccgttacatgaaggcctgccggccggcgaagaaggcctgatttatgcggaaattgatctgggcgcgattagcgtggcgaaagcggtgggcgatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 75 - protein, 344 aa, Synthetic construct
MNKPKFKAAAVQAAPVFLDLDKTIDKTIALMERAARNGAELIAFPETWLPGYPWYAWLDAPAMWLPQYTQRYFDNSLEVGTPQADRIRKAAKQNNIVVSMGLSEREGGSLYISQWFIDRDGDTIAKRRKLKPTHVERTIFGEGDGSDLAVYDLRLGRVGGLCCWEHLQPLSKYAMYAQNEQVHVAAWPSFSMYVAYAYALGHDVNTSASRIYAVEGQCFVLAPCATVSKEMVDEMCTTDVHKALLTTGGGYARIFGPDGRVLHEGLPAGEEGLIYAEIDLGLISVSKAVGDPAGHYSRPDVTQLIHDKAPRRAVVDTAPPSDEAPAVERPHGLDEPALPAMAEL

SEQ ID NO: 76 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctggttaccgggctatccgtggtatgcgtggctggatgcgccggcaatgtggttaccgcagtatacccagcgctattttgataacagcctggaagtgggcaccccgcaggcggatcgcattcgcaaagcggcgaaacagaacaacattgtggtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtatgatctgcgtttaggtcgcgttggcggcttatgctgctgggaacatctgcagccgctgagcaaatatgcgatgtatgcgcagaacgaacaggtgcatgtggcggcgtggccgagctttagcatgtatgtggcgtatgcgtatgcgttaggccatgatgtgaacaccagcgcgagcagaatttatgcggtggaaggccagtgctttgtgctggcgccgtgcgcaactgttagcaaagaaatggtggatgagatgtgcaccaccgatgtgcataaagcgctgttaaccaccggcggcggctatgcgcgcatttttggcccggatggccgcgtgttacatgaaggcctgccggccggcgaagaaggcctgatttatgcggaaattgatctgggcctgattagcgtgagcaaagcggtgggcgatccggcgggtcattatagccgcccagatgtgacccagttaattcatgataaagcgccgcgccgtgcagttgttgatactgcaccaccatctgacgaagcaccggcggtggaacgtccacatggtctggatgaaccggcattaccggcgatggcggaactg

SEQ ID NO: 77 - protein, 344 aa, Synthetic construct
MNKPKFKAAVVQAAPVFLDLDKTIDKTIALMERAARNGAELIAFPETWFPGYPWYAWLDAPAMWMPQYTQRYFDNSLEYGTPQAARIRKVAKDNNIHVSMGLSEREGSSLYISQWFIDRDGDTIAKRRKLKPTHVERTIFGEGDGSDLAVWDLRLGRVGGLCCWEHLQPLSKYAMYAQNEQVHVAAWPSFSMYEAYAYALGPDVNTHASRLYAVEGQCFVLAPCATVSKEMVDEMCTTDLKKALLKTGGGYARIFGPDGRVLHEGLPAGEEGLAYAEIDLGLISVSKAVSDPAGHYSRPDVTQLIHNKAPRRAVVDTAPPSDPAPAVERPHGADEPALPAMAEL

SEQ ID NO: 78 - DNA, 1032 bp, Synthetic construct
  source 1..1032: mol_type = other DNA
atgaacaaaccgaaatttaaagcggcggtggtgcaggcggcgccggtgtttctggatttagacaaaaccattgataagaccattgcgctgatggaacgcgcggcgcgcaacggcgcggaactgattgcgtttccggaaacctggtttccgggctatccgtggtatgcgtggctggatgcgccggcaatgtggatgccgcagtatacccagcgctattttgataacagcctggaatatggcaccccgcaggcggcgcgcattcgcaaagtggcgaaagataacaacattcatgtgagcatgggcctgagcgaacgcgaaggcagcagcctgtacatttctcagtggtttattgatcgcgatggcgataccattgcgaaacgccgcaaactgaaaccgacccatgtggaacgcaccatttttggcgaaggcgatggcagcgatctggcggtgtgggatctgcgtttaggtcgcgttggcggcttatgctgctgggaacatctgcagccgctgagcaaatatgcgatgtatgcgcagaacgaacaggtgcatgtggcggcgtggccgagctttagcatgtatgaagcgtatgcgtatgcgttaggcccggatgtgaacacccatgcgagcagactgtatgcggtggaaggccagtgctttgtgctggcgccgtgcgcaactgttagcaaagaaatggtggatgagatgtgcaccaccgatctgaaaaaagcgctgttaaaaaccggcggcggctatgcgcgcatttttggcccggatggccgcgtgttacatgaaggcctgccggccggcgaagaaggcctggcgtatgcggaaattgatctgggcctgattagcgtgagcaaagcggtgagcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 79 - protein, 344 aa, Synthetic construct
MNKPKFKAAAVQAAPVFLDLDKTIDKTIALIEDAARNGAELIAFPETWLPGYPWYAWLDAPAMWLPQYTKRYFDNSLEIGTPQAARIRKAAKDNNIYVSMGLSEREGGSLYISQWFIDRDGDTIAKRRKLKPTHVERTIFGEGDGSDLAVIDTRLGRVGGLCCWEHLQPLSKYAMYAQNEQVHVAAWPSFSVYGAYAYALGPIVNTSASRIYAVEGQCFVLAPCASVSKEMVDEMCTTDLRKALLRTGGASARIFGPDGRQLHEGLPAGEEGLIYAEIDLGLISVSKAVTDPAGHYSRPDVTQLIHNKAPRRAVMDTATPSDPAPAVERPHGADEPALPAMAEL

SEQ ID NO: 80 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgattgaagatgcggcgcgcaacggcgcggaactgattgcgtttccggaaacctggttaccgggctatccgtggtatgcgtggctggatgcgccggcaatgtggttaccgcagtataccaaacgctattttgataacagcctggaaattggcaccccgcaggcggcgcgcattcgcaaagcggcgaaagataacaacatttatgtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgattgatacccgtttaggtcgcgttggcggcttatgctgctgggaacatctgcagccgctgagcaaatatgcgatgtatgcgcagaacgaacaggtgcatgtggcggcgtggccgagctttagcgtgtatggcgcgtatgcgtatgcgttaggcccgattgtgaacaccagcgcgagcagaatttatgcggtggaaggccagtgctttgtgctggcgccgtgcgcaagcgttagcaaagaaatggtggatgagatgtgcaccaccgatctgcgcaaagcgctgttacgcaccggcggcgcgagcgcgcgcatttttggcccggatggccgccagttacatgaaggcctgccggccggcgaagaaggcctgatttatgcggaaattgatctgggcctgattagcgtgagcaaagcggtgaccgatccggcgggtcattatagccgcccagatgtgacccagttaattcataacaaagcgccgcgccgtgcagttatggatactgcaaccccatctgacccagcaccggcggtggaacgtccacatggtgcagatgaaccggcattaccggcgatggcggaactg

SEQ ID NO: 81 - protein, 344 aa, Synthetic construct
MNKPKFKAAAVQAAPVFLDLDKTIDKTIALMEDAARNGAELIAFPETWLPGYPWYAWLDAPAMWVPQYTQRYFDNSLEIGTPQAERIRKAAKDNNITVSMGLSEREGASLYISQWFIDRDGDTIAKRRKLKPTHGERTIFGEGDGSDLAVHDIRLGRVGGLCCWEHLQPLSKYAMYAQKEQVHVAAWPSFSLYEAYAYALGPEVNTSASRIYAVEGQCFVLAPCACVSKAMVDEMCGTDLQKALLKTGGVYASIFGPDGRPLHEGLPAGEEGLIYAEIDLGLISVSKAVGDPAGHYSRPDVTQLIHNKRPRRAVVDTAPPSDPAPTVERPHGADEPALPAMAEL

SEQ ID NO: 82 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtgggtgccgcagtatacccagcgctattttgataacagcctggaaattggcaccccgcaggcggaacgcattcgcaaagcggcgaaagataacaacattaccgtgagcatgggcctgagcgaacgcgaaggcgcgagcctgtacatttctcagtggtttattgatcgcgatggcgataccattgcgaaacgccgcaaactgaaaccgacccatggcgaacgcaccatttttggcgaaggcgatggcagcgatctggcggtgcatgatattcgcttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcatgcgttagcaaagcgatggtggatgagatgtgcggcaccgatctgcagaaagcgctgttaaaaaccggcggcgtgtatgcgagcatttttggcccggatggccgcccgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaacgcccgcgccgtgcagttgttgatactgcaccaccatctgacccagcaccgaccgtggaacgtccacatggtgcagatgaaccggcattaccggcgatggcggaactg

SEQ ID NO: 83 - protein, 344 aa, Synthetic construct
MNKPKFKAAAVQAAPVFLDLDKSIDKTIALMEDAARNGAELIAFPETWLPGYPWYAWLNAPAMWLPQYTQRYFDNSLEFGTPQAERIRKAAKKNNITVSMGLSEREGESLYISQWFIDRDGDTIARRRKLKPTHVERTIFGEGDGSDLAVYDTRLGRVGGLCCWEHLQPLSKYAMYAQKEQVHVAAWPSFSLYEAYAYALGPEVNTAASRIYAVEGQCFVLAPCALVSKEMVDEMCTTDLHKALLKTGGGAARIFGPDGRALHEGLPAGEEGLIYAEIDLGLISVAKAVNDPAGHYSRPDVTQLIHDKAPRRAVVDTAPPSDPAPAVERPHGADEPALPAMAEL

SEQ ID NO: 84 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagaccattgcgctgatggaagatgcggcgcgcaacggcgcggaactgattgcgtttccggaaacctggttaccgggctatccgtggtatgcgtggctgaacgcgccggcaatgtggttaccgcagtatacccagcgctattttgataacagcctggaatttggcaccccgcaggcggaacgcattcgcaaagcggcgaaaaaaaacaacattaccgtgagcatgggcctgagcgaacgcgaaggcgaaagcctgtacatttctcagtggtttattgatcgcgatggcgataccattgcgcgccgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacaccgcggcgagcagaatttatgcggtggaaggccagtgctttgtgctggcgccgtgcgcactggttagcaaagaaatggtggatgagatgtgcaccaccgatctgcataaagcgctgttaaaaaccggcggcggcgcggcgcgcatttttggcccggatggccgcgcgttacatgaaggcctgccggccggcgaagaaggcctgatttatgcggaaattgatctgggcctgattagcgtggcgaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 85 - protein, 344 aa, Synthetic construct
MNKPKFKAAAVQAAPVFLDLDKTIDKTIALMEEAARNGAELIAFPETWLPGYPWYAWLDAPAMWLPGYVQRYFDNSLEYGTPQAARIRKAAKDNNIVVSMGLSEREGGSLYISQWFIDRDGDTIAQRRKLKPTHVERTIFGEGDGSDLAVWDARLGRVGGLCCWEHLQPLSKYAMYAQNEQVHVAAWPSFSAYKAYAYALGSIVNTSASRIYAVEGQCFVLAPCATVSKDMVDEMCTTDDHKALLKTGGVYATIFGPDGRQLHEGLPAGEEGLAYAEIDLGLISVSKAVGDPAGHYSRPDVTQLIHNKAPRRAVVDTAAPSDPAPAVERPHGADQPALPAMAEL

SEQ ID NO: 86 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaccgggctatgtgcagcgctattttgataacagcctggaatatggcaccccgcaggcggcgcgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatgcgcgtttaggtcgcgttggcggcttatgctgctgggaacatctgcagccgctgagcaaatatgcgatgtatgcgcagaacgaacaggtgcatgtggcggcgtggccgagctttagcgcgtataaagcgtatgcgtatgcgttaggcagcattgtgaacaccagcgcgagcagaatttatgcggtggaaggccagtgctttgtgctggcgccgtgcgcaactgttagcaaagatatggtggatgagatgtgcaccaccgatgatcataaagcgctgttaaaaaccggcggcgtgtatgcgaccatttttggcccggatggccgccagttacatgaaggcctgccggccggcgaagaaggcctggcgtatgcggaaattgatctgggcctgattagcgtgagcaaagcggtgggcgatccggcgggtcattatagccgcccagatgtgacccagttaattcataacaaagcgccgcgccgtgcagttgttgatactgcagcgccatctgacccagcaccggcggtggaacgtccacatggtgcagatcagccggcattaccggcgatggcggaactg

SEQ ID NO: 87 - protein, 344 aa, Synthetic construct
MNKPKFKLAAVQAAPVFLDLDKTIDKTIALMEDAARNGAELIAFPETWLPGYPWYAWLDAPAMWLPQYTQRYFDNSLEFGTPQKHRIRKTAKDNNIVVSMGLSEREGASLYISQWIIDRDGDTIAQRRKLKPTHVERTIFGEGDGSDLAVWDTRLGRVGGLCCWEHLQPLSKYAMYAQDEQVHVAAWPSFSLYEAYAYALGPDVNTSASRIYAVEGQCFVLAPCACVSKEMVDEMCTTDVQKALLKTGGCYARIFGPDGRQLHEGLPAGEEGLIYAEIDLGAISVAKAVNDPAGHYSRPDVTQLIHDKAPRRAVVDTAPPSDPAPAVERPHGADELALPAMAEL

SEQ ID NO: 88 - DNA, 1032 bp, Synthetic construct
  source 1..1032: mol_type = other DNA
atgaacaaaccgaaatttaaactggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatttggcaccccgcagaaacatcgcattcgcaaaaccgcgaaagataacaacattgtggtgagcatgggcctgagcgaacgcgaaggcgcgagcctgtacatttctcagtggattattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcatgcgttagcaaagaaatggtggatgagatgtgcaccaccgatgtgcagaaagcgctgttaaaaaccggcggctgctatgcgcgcatttttggcccggatggccgccagttacatgaaggcctgccggccggcgaagaaggcctgatttatgcggaaattgatctgggcgcgattagcgtggcgaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaactggcattaccggcgatggcggaactg

SEQ ID NO: 89 - protein, 344 aa, Synthetic construct
MNKPKFKAAAVQAAPVFLDLDKTIDKTLALMEDAARNGAELIAFPETWLPGYPWYAWLDAPAMWGPQYTQRYFDNSLEYGTPQAERIRKVAKDNNIWMLMGLSEREGGSLYISQWHIDRDGDTIAQRRKLKPTHVERTIFGEGDGSDLAVYDIRLGRVGGLCCWEHLQPLSKYAMYAQNEQVHVAAWPSFSLYGAYAHALGPEVNTSASRIYAVEGSCFVLAPCALVSKEMVDEMCTTDLQKALLKTGGGYAAIFGPDGRPLHEGLPAGEEGLIYAEIDLGLISVSKAVADPAGHYSRPDVTQLIHHKAPRRAVVDTAPPSDPAPAVERPHGADEPALPAMAEL

SEQ ID NO: 90 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaagatgcggcgcgcaacggcgcggaactgattgcgtttccggaaacctggttaccgggctatccgtggtatgcgtggctggatgcgccggcaatgtggggcccgcagtatacccagcgctattttgataacagcctggaatatggcaccccgcaggcggaacgcattcgcaaagtggcgaaagataacaacatttggatgctgatgggcctgagcgaacgcgaaggcggcagcctgtacatttctcagtggcatattgatcgcgatggcgataccattgcgcagcgccgcaaactgaaaccgacccatgtggaacgcaccatttttggcgaaggcgatggcagcgatctggcggtgtatgatattcgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgcatgcgttaggcccggaagtgaacaccagcgcgagcagaatttatgcggtggaaggcagctgctttgtgctggcgccgtgcgcactggttagcaaagaaatggtggatgagatgtgcaccaccgatctgcagaaagcgctgttaaaaaccggcggcggctatgcggcgatttttggcccggatggccgcccgttacatgaaggcctgccggccggcgaagaaggcctgatttatgcggaaattgatctgggcctgattagcgtgagcaaagcggtggcggatccggcgggtcattatagccgcccagatgtgacccagttaattcatcataaagcgccgcgccgtgcagttgttgatactgcaccaccatctgacccagcaccggcggtggaacgtccacatggtgcagatgaaccggcattaccggcgatggcggaactg

SEQ ID NO: 91 - protein, 344 aa, Synthetic construct
MNKPKFKAAAVQAAPVFLDLDKTIDKTIALMEDAARNGAELIAFPETWLPGYPWYAWLDAPAMWLPAYTQRYFDNSLEYGTPQKERIRKDAKDHNIVVSMGLSEREGGSLYISQWFIDRDGDTLAKRRKLKPTHVERTIFGEGDGSDLAVHDLRLGRMGGLCCWEHLQPLSKYAMYAQHEQVHVAAWPSFSIYEAYAYALGPDVNTSASRIYAVEGTCFVLAPCALVSKEMVDEMCTTDGQKALLKTGGGYAVIFGPDGRQLHEGLPAGEEGLIYAEIDLGLISVSKAVGDPAGHYSRPDVTQLIHNKAVRRAVVDTAPPSDPAPAVERPHGADEPALPAMAEL

SEQ ID NO: 92 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccggcgtatacccagcgctattttgataacagcctggaatatggcaccccgcagaaagaacgcattcgcaaagatgcgaaagatcataacattgtggtgagcatgggcctgagcgaacgcgaaggcggcagcctgtacatttctcagtggtttattgatcgcgatggcgataccctggcgaaacgccgcaaactgaaaccgacccatgtggaacgcaccatttttggcgaaggcgatggcagcgatctggcggtgcatgatctgcgtttaggtcgcatgggcggcttatgctgctgggaacatctgcagccgctgagcaaatatgcgatgtatgcgcagcatgaacaggtgcatgtggcggcgtggccgagctttagcatttatgaagcgtatgcgtatgcgttaggcccggatgtgaacaccagcgcgagcagaatttatgcggtggaaggcacctgctttgtgctggcgccgtgcgcactggttagcaaagaaatggtggatgagatgtgcaccaccgatggccagaaagcgctgttaaaaaccggcggcggctatgcggtgatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcggtgcgccgtgcagttgttgatactgcaccaccatctgacccagcaccggcggtggaacgtccacatggtgcagatgaaccggcattaccggcgatggcggaactg

SEQ ID NO: 93 - protein, 344 aa, Synthetic construct
MNKPKFKAAAVQAAPVFLDLDKTIDKTIALMEKAARNGAELIAFPETWLPGYPWYAWLDAPAMWLPQYTARYFDNSLEYGTPQAERIRKSAKDNNIVVSMGLSEREGESLYISQWFIDRDGDTIAMRRKLKPTHVERTIFGEGDGSDLMVVDTRLGRVGGLCCWEHLQPLSKYAMYAQNEQVHVAAWPSFSLYDAYAYALGPDVNTSASRIYAVEGQCFVLAPCATVSKEMVDEMCTTDDHKALLKTGGGYAVIFGPDGRSLHEGLDAGEEGLIYAEIDLGLISVSKAVGDPAGHYSRPDVTQLIHNKAPRRRVVDTAAPSDPAPAVERPHGAEEPALPAMAEL

SEQ ID NO: 94 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtataccgcgcgctattttgataacagcctggaatatggcaccccgcaggcggaacgcattcgcaaaagcgcgaaagataacaacattgtggtgagcatgggcctgagcgaacgcgaaggcgaaagcctgtacatttctcagtggtttattgatcgcgatggcgataccattgcgatgcgccgcaaactgaaaccgacccatgtggaacgcaccatttttggcgaaggcgatggcagcgatctgatggtggtggatacccgtttaggtcgcgttggcggcttatgctgctgggaacatctgcagccgctgagcaaatatgcgatgtatgcgcagaacgaacaggtgcatgtggcggcgtggccgagctttagcctgtatgatgcgtatgcgtatgcgttaggcccggatgtgaacaccagcgcgagcagaatttatgcggtggaaggccagtgctttgtgctggcgccgtgcgcaactgttagcaaagaaatggtggatgagatgtgcaccaccgatgatcataaagcgctgttaaaaaccggcggcggctatgcggtgatttttggcccggatggccgcagcttacatgaaggcctggatgccggcgaagaaggcctgatttatgcggaaattgatctgggcctgattagcgtgagcaaagcggtgggcgatccggcgggtcattatagccgcccagatgtgacccagttaattcataacaaagcgccgcgccgtcgcgttgttgatactgcagcgccatctgacccagcaccggcggtggaacgtccacatggtgcagaagaaccggcattaccggcgatggcggaactg

SEQ ID NO: 95 - protein, 344 aa, Synthetic construct
MNKPKFKAAAVQAAPVFLDLDKTIDKTIALMEDAARNGAELIAFPETWLPGYPWYAWLGVPAMWLPAYTQRYFDNSLEYGTPQAERIRKDAKDNNIYVSMGLSEREGASLYISQWFIDRDGDTIAQRRKLKPTHVERTIFGEGDGSDLAVYDTRLGRLGGLCCWEHLQPLSKYAMYAQNEQVHVAAWPSFSLYEAYAYALGHEVNTSASRIYAVEGQCFVLAPCAIVSKEMVDEMCTTDARKALLKTGGGYASIFGPDGRLLHEGLPAGEEGLIYAEIDLGLIGVSKAVNDPAGHYSRPDVTQLIHNKAPRRAVVDTAPPSDPAPAVERPHGPDEPALPAMAEL

SEQ ID NO: 96 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgggcgtgccggcaatgtggttaccggcgtatacccagcgctattttgataacagcctggaatatggcaccccgcaggcggaacgcattcgcaaagatgcgaaagataacaacatttatgtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtatgatacccgtttaggtcgcctgggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggccatgaagtgaacaccagcgcgagcagaatttatgcggtggaaggccagtgctttgtgctggcgccgtgcgcaattgttagcaaagaaatggtggatgagatgtgcaccaccgatgcgcgcaaagcgctgttaaaaaccggcggcggctatgcgagcatttttggcccggatggccgcctgttacatgaaggcctgccggccggcgaagaaggcctgatttatgcggaaattgatctgggcctgattggcgtgagcaaagcggtgaacgatccggcgggtcattatagccgcccagatgtgacccagttaattcataacaaagcgccgcgccgtgcagttgttgatactgcaccaccatctgacccagcaccggcggtggaacgtccacatggtccggatgaaccggcattaccggcgatggcggaactg

SEQ ID NO: 97 - protein, 344 aa, Synthetic construct
MNKPKFKAAAVQAAPVFLDLDKTIDKTEALMEDAARNGAELIAFPETWLPGYPWYAWLDVPAMWLPQYTDRYFDNSLEYGTPQAKRIRKVAKENNIYMSMGLSEREGGSLYISQWFIDRDGDTIAQRRKLKPTHVERTIFGEGDGSDLAVRDIRLGRVGGLCCWEHLQPLSKYAMYAQNEQVHVAAWPSFSLYEAYAYALGPIVNTSASRIYAVEGQCFVLAPCATVSKSMVDEMCTTDSQKALLKTGGGYATIFGPDGRQLHEGLPAGEEGLIYAEIDLGLISVSKAVADPAGHYSRPDVTQLIHNKAPRRAVVDTAPPSDPAPGVERPHGKDEPALPAMAEL

SEQ ID NO: 98 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaagatgcggcgcgcaacggcgcggaactgattgcgtttccggaaacctggttaccgggctatccgtggtatgcgtggctggatgtgccggcaatgtggttaccgcagtataccgatcgctattttgataacagcctggaatatggcaccccgcaggcgaaacgcattcgcaaagtggcgaaagaaaacaacatttatatgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcgcgatattcgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccgattgtgaacaccagcgcgagcagaatttatgcggtggaaggccagtgctttgtgctggcgccgtgcgcaactgttagcaaaagcatggtggatgagatgtgcaccaccgatagccagaaagcgctgttaaaaaccggcggcggctatgcgaccatttttggcccggatggccgccagttacatgaaggcctgccggccggcgaagaaggcctgatttatgcggaaattgatctgggcctgattagcgtgagcaaagcggtggcggatccggcgggtcattatagccgcccagatgtgacccagttaattcataacaaagcgccgcgccgtgcagttgttgatactgcaccaccatctgacccagcaccgggcgtggaacgtccacatggtaaagatgaaccggcattaccggcgatggcggaactg

SEQ ID NO: 99 - protein, 344 aa, Synthetic construct
MNKPKFKLAAVQAAPVFLDLDKTIDKTIALMEEAARNGAELIAFPETWLPGYPWYAWLDAPAMWLPKYTQRYFDNSLEYGTPQAERIRKDAKDNNIWVSMGLSEREGGSLYISQWFIDRDGDTIAQRRKLKPTHVERTIFGEGDGSDLAVWDIRLGRVGGLCCWEHLQPLSKYAMYAQHEQVHVAAWPSFSLYEAYAYALGPIVNESASRIYAVEGQCFVLAPCATVSKEMVDEMCTTDDQKALLKTGGCYAVIFGPDGRQLHEGLPAGEEGLIYAEIDLGMISVSKAVGDPAGHYSRPDVTQLIHDKSPRRAVVDGAPPSDPAPAVERPHGADEPALPAMAEL

SEQ ID NO: 100 - DNA, 1032 bp, Synthetic construct
  source 1..1032: mol_type = other DNA
atgaacaaaccgaaatttaaactggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaccgaaatatacccagcgctattttgataacagcctggaatatggcaccccgcaggcggaacgcattcgcaaagatgcgaaagataacaacatttgggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagcatgaacaggtgcatgtggcggcgtggccgagctttagcctgtatgaagcgtatgcgtatgcgttaggcccgattgtgaacgaaagcgcgagcagaatttatgcggtggaaggccagtgctttgtgctggcgccgtgcgcaactgttagcaaagaaatggtggatgagatgtgcaccaccgatgatcagaaagcgctgttaaaaaccggcggctgctatgcggtgatttttggcccggatggccgccagttacatgaaggcctgccggccggcgaagaaggcctgatttatgcggaaattgatctgggcatgattagcgtgagcaaagcggtgggcgatccggcgggtcattatagccgcccagatgtgacccagttaattcatgataaaagcccgcgccgtgcagttgttgatggcgcaccaccatctgacccagcaccggcggtggaacgtccacatggtgcagatgaaccggcattaccggcgatggcggaactg

SEQ ID NO: 101 - protein, 344 aa, Synthetic construct
MNKPKFKAAIVQAAPVFLDLDKTIDKTIALMEDAARNGAELIAFPETWLPGYPWYAWLDAPAMWMPQYTQRYFDNSLEFGTPQADRIRKAAKDNNIVVSMGLSEREGGSLYISQWFIDRDGDTIAMRRKLKPTHVERTIFGEGDGSDLAVWDLRLGRVGGLCCWEHLQPLSKYAMYAQKEQVHVAAWPSFSAYGAYAYALGPEVNESASRIYAVEGQCFVLAPCATVSKDMVDEMCTTDLQKALLKTGGGYARIFGPDGRQLHEGLPAGAEGLIYAEIDLGLISVSKAVTDPAGHYSRPDVTQLIHEKKPRRAVSDTAPPSDPAPAVERPHGADEPALPAMAEL

SEQ ID NO: 102 - DNA, 1032 bp, Synthetic construct
  source 1..1032: mol_type = other DNA
atgaacaaaccgaaatttaaagcggcgattgtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggatgccgcagtatacccagcgctattttgataacagcctggaatttggcaccccgcaggcggatcgcattcgcaaagcggcgaaagataacaacattgtggtgagcatgggcctgagcgaacgcgaaggcggcagcctgtacatttctcagtggtttattgatcgcgatggcgataccattgcgatgcgccgcaaactgaaaccgacccatgtggaacgcaccatttttggcgaaggcgatggcagcgatctggcggtgtgggatctgcgtttaggtcgcgttggcggcttatgctgctgggaacatctgcagccgctgagcaaatatgcgatgtatgcgcagaaagaacaggtgcatgtggcggcgtggccgagctttagcgcgtatggcgcgtatgcgtatgcgttaggcccggaagtgaacgaaagcgcgagcagaatttatgcggtggaaggccagtgctttgtgctggcgccgtgcgcaactgttagcaaagatatggtggatgagatgtgcaccaccgatctgcagaaagcgctgttaaaaaccggcggcggctatgcgcgcatttttggcccggatggccgccagttacatgaaggcctgccggccggcgcggaaggcctgatttatgcggaaattgatctgggcctgattagcgtgagcaaagcggtgaccgatccggcgggtcattatagccgcccagatgtgacccagttaattcatgaaaaaaaaccgcgccgtgcagttagcgatactgcaccaccatctgacccagcaccggcggtggaacgtccacatggtgcagatgaaccggcattaccggcgatggcggaactg

SEQ ID NO: 103 - protein, 344 aa, Synthetic construct
MNKPKFKVAAVQAAPVFLDLDKTIDKTIALVEEAARNGAELIAFPETWLPGYPWYAWLNAPAMWLPQYTQRYFDNSLEYGTPQADRIRKDAKDNNIHVSMGLSEREGGSLYISQWFIDRDGDTIAQRRKLKPTHVERTIFGEGDGSDLAVHDPRLGRIGGLCCWEHLQPLSKYAMYAQNEQVHVAAWPSFSLYEAYAYALGPEVNTSASRIYAVEGQCFVLAPCATVSKEMVDEMCTTDLRKALLKTGGAYARIFGPDGRELHEGLPAGEEGLIYAEIDLGLISVSKTVGDPAGHYSRPDVTQLIHHKAPRRAVVDAAPPSDPAPAVERPHGADEPALPAMAEL

SEQ ID NO: 104 - DNA, 1032 bp, Synthetic construct
  source 1..1032: mol_type = other DNA
atgaacaaaccgaaatttaaagtggcggcggtgcaggcggcgccggtgtttctggatttagacaaaaccattgataagaccattgcgctggtggaagaagcggcgcgcaacggcgcggaactgattgcgtttccggaaacctggttaccgggctatccgtggtatgcgtggctgaacgcgccggcaatgtggttaccgcagtatacccagcgctattttgataacagcctggaatatggcaccccgcaggcggatcgcattcgcaaagatgcgaaagataacaacattcatgtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcatgatccgcgtttaggtcgcattggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggcgcgtatgcgcgcatttttggcccggatggccgcgaattacatgaaggcctgccggccggcgaagaaggcctgatttatgcggaaattgatctgggcctgattagcgtgagcaaaaccgtgggcgatccggcgggtcattatagccgcccagatgtgacccagttaattcatcataaagcgccgcgccgtgcagttgttgatgcggcaccaccatctgacccagcaccggcggtggaacgtccacatggtgcagatgaaccggcattaccggcgatggcggaactg

SEQ ID NO: 105 - protein, 344 aa, Synthetic construct
MNKPKFKAAAVQAAPVFLDLDKTIDKTIALMEDAARNGAELIAFPETWLPGYPWYAWLGAPAMWLPQYTHRYFDNSLEYGNPQARRIRKAAKDNNIVVSMGLSEREGGSLYISQWFIDRDGDTIATRRKLKPTHVERTIFGEGDGSDLAVWDLRLGRVGGLCCWEHVQPLSKYAMYAQNEQVHVAAWPSFSLYEAYAYALGPDVNTSASRTYAVEGQCFVLAPCATVSKEMVDEMCTTDSHKALLKTGGVYAAIFGPDGRQLHEGLPAGEEGLIYAEIDLGLISVSKAVSDPAGHYSRPDVTQLIHNKAPRRAVVDGAAPSDPAPAVERPHGADEPALPAMAEL

SEQ ID NO: 106 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgggcgcgccggcaatgtggttaccgcagtatacccatcgctattttgataacagcctggaatatggcaacccgcaggcgcgccgcattcgcaaagcggcgaaagataacaacattgtggtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgtgggatctgcgtttaggtcgcgttggcggcttatgctgctgggaacatgtgcagccgctgagcaaatatgcgatgtatgcgcagaacgaacaggtgcatgtggcggcgtggccgagctttagcctgtatgaagcgtatgcgtatgcgttaggcccggatgtgaacaccagcgcgagcagaacctatgcggtggaaggccagtgctttgtgctggcgccgtgcgcaactgttagcaaagaaatggtggatgagatgtgcaccaccgatagccataaagcgctgttaaaaaccggcggcgtgtatgcggcgatttttggcccggatggccgccagttacatgaaggcctgccggccggcgaagaaggcctgatttatgcggaaattgatctgggcctgattagcgtgagcaaagcggtgagcgatccggcgggtcattatagccgcccagatgtgacccagttaattcataacaaagcgccgcgccgtgcagttgttgatggcgcagcgccatctgacccagcaccggcggtggaacgtccacatggtgcagatgaaccggcattaccggcgatggcggaactg

SEQ ID NO: 107 - protein, 344 aa, Synthetic construct
MNKPKFKAAAVQAAPVFLDLDKTIDKTIALMEDAARNGAELIAFPETWLPGYPWYAWLDAPAMWLPAYTQRYFDNSLEYGTPQDERIRKAAKDNNITVSMGLSEREGSSLYISQWFIDSDGDTIARRRKLKPTHVERTIFGEGDGSDLHVHDTRLGRVGGLCCWEHFQPLSKYAMYAQNEQVHVAAWPSFSMYDAYAYALGGEVNTSASRIYAVEGQCFVLAPCATVSKSMVDEMCTTDLQKALLKTGGGYARILGPDGRSLHEGLPAGEEGLIYAEIDLGLISVSKAVTDPAGHYSRPDVTQLIHNKAPRRAVVDTAPPSDPAPAVERPHGADEPALPAMAEL

SEQ ID NO: 108 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccggcgtatacccagcgctattttgataacagcctggaatatggcaccccgcaggatgaacgcattcgcaaagcggcgaaagataacaacattaccgtgagcatgggcctgagcgaacgcgaaggcagcagcctgtacatttctcagtggtttattgatagcgatggcgataccattgcgcgccgccgcaaactgaaaccgacccatgtggaacgcaccatttttggcgaaggcgatggcagcgatctgcatgtgcatgatacccgtttaggtcgcgttggcggcttatgctgctgggaacattttcagccgctgagcaaatatgcgatgtatgcgcagaacgaacaggtgcatgtggcggcgtggccgagctttagcatgtatgatgcgtatgcgtatgcgttaggcggcgaagtgaacaccagcgcgagcagaatttatgcggtggaaggccagtgctttgtgctggcgccgtgcgcaactgttagcaaaagcatggtggatgagatgtgcaccaccgatctgcagaaagcgctgttaaaaaccggcggcggctatgcgcgcattctgggcccggatggccgcagcttacatgaaggcctgccggccggcgaagaaggcctgatttatgcggaaattgatctgggcctgattagcgtgagcaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 109 - protein, 344 aa, Synthetic construct
MNKPKFKAAAVQAAPVFLDLDKTIDKTIALMEDAARNGAELIAFPETWLPGYPWYAWLDAPAMWLPVYTRRYFDNSLEYGTPQAKRIRKSAKDNNIVVSSGLSEREGGSLYITQWFIDRDGDTIAQRRKLKPTHVERTIFGEGDGSDLAVWDTRLGRVGGLCCWEHLQPLSKYAMYAQNEQVHVAAWPSFSAYEAYAYALGPDVNTSASRIYAVEGQCFVLAPCATVSKAMVDEMCTTDSKKALLKTGGGYARIFGPDGRALHEGLPAGKEGLIYAEIDLGLISVSKAVGDPAGHYSRPDVTQLIHDKAPRRAVVDTAAPSDPAPAVERPHGVDEPALPAMAEL

SEQ ID NO: 110 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccggtgtatacccgccgctattttgataacagcctggaatatggcaccccgcaggcgaaacgcattcgcaaaagcgcgaaagataacaacattgtggtgagcagcggcctgagcgaacgcgaaggcggcagcctgtacattacccagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcgcgtatgaagcgtatgcgtatgcgttaggcccggatgtgaacaccagcgcgagcagaatttatgcggtggaaggccagtgctttgtgctggcgccgtgcgcaactgttagcaaagcgatggtggatgagatgtgcaccaccgatagcaaaaaagcgctgttaaaaaccggcggcggctatgcgcgcatttttggcccggatggccgcgcgttacatgaaggcctgccggccggcaaagaaggcctgatttatgcggaaattgatctgggcctgattagcgtgagcaaagcggtgggcgatccggcgggtcattatagccgcccagatgtgacccagttaattcatgataaagcgccgcgccgtgcagttgttgatactgcagcgccatctgacccagcaccggcggtggaacgtccacatggtgtggatgaaccggcattaccggcgatggcggaactg

SEQ ID NO: 111 - protein, 344 aa, Synthetic construct
MNKPKFKAAAVQAAPVFLDLDKTIDKTIALMEKAARNGAELIAFPETWLPGYPWYAWLGSPAMWLPQYTQRYFDNSLEYGTPQARRIRKAAKDNNIVVSMGLSEREGASLYISQWFIDRDGDTIAQRRKLKPTHVERTIFGEGDGSDLAVWDTRLGRVGGLCCWEHLQPLSKYAMYAQNEQVHVAAWPSFSLYRGYAYALGPIVNTSASRIYAVEGQCFVLAPCATVSKSMVDEMCTTDLKKALLTTGGGYARIFGPDGRALHEGLPAGEEGLIYAEIDLGAISVSKAVSDPAGHYSRPDVTQLIVDKAPRRAVVDTAPPSDPAPAVERPHGADEPALPAMAEL

SEQ ID NO: 112 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctgggcagcccggcaatgtggttaccgcagtatacccagcgctattttgataacagcctggaatatggcaccccgcaggcgcgccgcattcgcaaagcggcgaaagataacaacattgtggtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatcgcggctatgcgtatgcgttaggcccgattgtgaacaccagcgcgagcagaatttatgcggtggaaggccagtgctttgtgctggcgccgtgcgcaactgttagcaaaagcatggtggatgagatgtgcaccaccgatctgaaaaaagcgctgttaaccaccggcggcggctatgcgcgcatttttggcccggatggccgcgcgttacatgaaggcctgccggccggcgaagaaggcctgatttatgcggaaattgatctgggcgcgattagcgtgagcaaagcggtgagcgatccggcgggtcattatagccgcccagatgtgacccagttaattgtggataaagcgccgcgccgtgcagttgttgatactgcaccaccatctgacccagcaccggcggtggaacgtccacatggtgcagatgaaccggcattaccggcgatggcggaactg

SEQ ID NO: 113 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEVGTPQYERIRKDAKDNNIYVSMGLSEREGGSLYISQWFIDPDGDTIATRRKLKPTHVERTIFGEGDGSDLAVWDTRLGRVGGLCCWEHFQPLSKYAMYAQKEQVHVAAWPSFSLYEAYAYALGPEVNTSASRIYAVEGQCFVLAPCATVSKSMVDEMCETDEQKALLKTGGGYAAIFGPDGRSLHEGLPAGEEGLIYAEIDLGLISVSKAVGDPAGHYSRPDVTQLIHHKAPRRAVVDTAPPSDPAPTVERPHGADAPALPAMAEL

SEQ ID NO: 114 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaagtgggcaccccgcagtatgaacgcattcgcaaagatgcgaaagataacaacatttatgtgagcatgggcctgagcgaacgcgaaggcggcagcctgtacatttctcagtggtttattgatccggatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacattttcagccgctgagcaaatatgcgatgtatgcgcagaaagaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaaagcatggtggatgagatgtgcgaaaccgatgaacagaaagcgctgttaaaaaccggcggcggctatgcggcgatttttggcccggatggccgcagcttacatgaaggcctgccggccggcgaagaaggcctgatttatgcggaaattgatctgggcctgattagcgtgagcaaagcggtgggcgatccggcgggtcattatagccgcccagatgtgacccagttaattcatcataaagcgccgcgccgtgcagttgttgatactgcaccaccatctgacccagcaccgaccgtggaacgtccacatggtgcagatgcgccggcattaccggcgatggcggaactg

SEQ ID NO: 115 - protein, 344 aa, Synthetic construct
MNKPKFKAAAVQAAPVFLDLDKTIDKTIALMEKAARNGAELIAFPETWLPGYPWYAWLDSPAMWLPQYTDRYFDNSLEYGTPQAERIRKDAKDNNIVVSMGLSEREGGSLYISQWFIDRDGDTIATRRKLKPTHVERTIFGEGDGSDLAVRDARLGRVGGLCCWEHLQPLSKYAMYAQTEQVHVAAWPSFSLYEAYAYALGPEVNTSASRIYAVEGQCFVLAPCAIVSKSMVDEMCTTDLQKALLTTGGVYARIFGPDGRQLHEGLPAGEEGLICAEIDLGVISVSKAVTDPAGHYSRPDVTQLVHNKAPRRAVVDTAPPSDPAPAVERPHGADEPALPAMAEL

SEQ ID NO: 116 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatagcccggcaatgtggttaccgcagtataccgatcgctattttgataacagcctggaatatggcaccccgcaggcggaacgcattcgcaaagatgcgaaagataacaacattgtggtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgcgcgatgcgcgtttaggtcgcgttggcggcttatgctgctgggaacatctgcagccgctgagcaaatatgcgatgtatgcgcagaccgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaattgttagcaaaagcatggtggatgagatgtgcaccaccgatctgcagaaagcgctgttaaccaccggcggcgtgtatgcgcgcatttttggcccggatggccgccagttacatgaaggcctgccggccggcgaagaaggcctgatttgcgcggaaattgatctgggcgtgattagcgtgagcaaagcggtgaccgatccggcgggtcattatagccgcccagatgtgacccagttagtgcataacaaagcgccgcgccgtgcagttgttgatactgcaccaccatctgacccagcaccggcggtggaacgtccacatggtgcagatgaaccggcattaccggcgatggcggaactg

SEQ ID NO: 117 - protein, 344 aa, Synthetic construct
MNKPKFKAAAVQAAPVFLDLDKTIDKTRALMEEAARNGAELIAFPETWLPGYPWYAWLDAPAMWLPQYFQRYFDNSLEYGTPQAERIRKAAKDNNIVVSMGLSEREGASLYISQWFIDRDGDTIAQRRKLKPTHVERTIFGEGDGSDLAVIDTRLGRVGGLCCWEHLQPLSKYAMYAQNEQVHVAAWPSFSLYEAYAYALGPEVNTSASRIYAVEGQCFVLAPCATVSKEMVDEMCTTDLRKALLKTGGVSAAIFGPDGRQLHEGLPADEEGLIYAEIDLGLISVSKAVTDPAGHYSRPDVTQLIHHKAPRRAVVDTAPPSDNAPAVERPHGADEPALPAMAEL

SEQ ID NO: 118 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagaagcggcgcgcaacggcgcggaactgattgcgtttccggaaacctggttaccgggctatccgtggtatgcgtggctggatgcgccggcaatgtggttaccgcagtattttcagcgctattttgataacagcctggaatatggcaccccgcaggcggaacgcattcgcaaagcggcgaaagataacaacattgtggtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgattgatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggcgtgagcgcggcgatttttggcccggatggccgccagttacatgaaggcctgccggccgatgaagaaggcctgatttatgcggaaattgatctgggcctgattagcgtgagcaaagcggtgaccgatccggcgggtcattatagccgcccagatgtgacccagttaattcatcataaagcgccgcgccgtgcagttgttgatactgcaccaccatctgacaacgcaccggcggtggaacgtccacatggtgcagatgaaccggcattaccggcgatggcggaactg

SEQ ID NO: 119 - protein, 344 aa, Synthetic construct
MNKPKFKAAAVQAAPVFLDLDKTIDKTVALMEDAARKGAELIAFPETWLPGYPWYAWLDAPAMWLPQYTPRYFDNSLEYGNPQAERIRKAAKDNNIVVSMGLSEREGSSLYISQWFIDRDGDTIAMRRKLKPTHVERTIFGEGDGSDLAVWDTRLGRVGGLCCWEHLQPLSKYAMYAQNEQVHVAAWPSFSLYEPYANALGPIVNTSASRIYAVEGQCFVLAPCATVSKGMVDEMCTTDLQKALLKTGGAYASIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 120 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaagatgcggcgcgcaaaggcgcggaactgattgcgtttccggaaacctggttaccgggctatccgtggtatgcgtggctggatgcgccggcaatgtggttaccgcagtataccccgcgctattttgataacagcctggaatatggcaacccgcaggcggaacgcattcgcaaagcggcgaaagataacaacattgtggtgagcatgggcctgagcgaacgcgaaggcagcagcctgtacatttctcagtggtttattgatcgcgatggcgataccattgcgatgcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaaccgtatgcgaacgcgttaggcccgattgtgaacaccagcgcgagcagaatttatgcggtggaaggccagtgctttgtgctggcgccgtgcgcaactgttagcaaaggcatggtggatgagatgtgcaccaccgatctgcagaaagcgctgttaaaaaccggcggcgcgtatgcgagcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 121 - protein, 344 aa, Synthetic construct
MNKPKFKAAAVQAAPVFLDLDKTIDKTRALMEDAARNGAELIAFPETWLPGYPWYAWLDAPAMWLPQYFQRYFDNSLEYGTPQDERIRKAAKDNNIVVSMGLSEREGGSLYITQWFIDRDGDTIAQRRKLKPTHVERTIFGEGDGSDLAVWDTRLGRVGGLCCWEHFQPLSKYAMYAQNEQVHVAAWPSFSLYEAYAYALGPDVNTSASRIYAVEGQCFVLAPCATVSKSMVDEMCTTDARKALLKTGGGYAGIFGPDGRQLHEGLPHGEEGLIYAEIDLGLISVSKAVADPAGHYSRPDVTQLIHEKAPRRAVVDTAPPSDPAPAVERPHGADEPALPAMAEL

SEQ ID NO: 122 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagatgcggcgcgcaacggcgcggaactgattgcgtttccggaaacctggttaccgggctatccgtggtatgcgtggctggatgcgccggcaatgtggttaccgcagtattttcagcgctattttgataacagcctggaatatggcaccccgcaggatgaacgcattcgcaaagcggcgaaagataacaacattgtggtgagcatgggcctgagcgaacgcgaaggcggcagcctgtacattacccagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacattttcagccgctgagcaaatatgcgatgtatgcgcagaacgaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcaactgttagcaaaagcatggtggatgagatgtgcaccaccgatgcgcgcaaagcgctgttaaaaaccggcggcggctatgcgggcatttttggcccggatggccgccagttacatgaaggcctgccgcatggcgaagaaggcctgatttatgcggaaattgatctgggcctgattagcgtgagcaaagcggtggcggatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 123 - protein, 344 aa, Synthetic construct
MNKPKFKAAAVQAAPVFLDLDKTIDKTIALMEEAARNGAELIAFPETWLPGYPWYAWLDAPAMWLPQYTARYFDNSLEYNTPQARRIRKAAKDNNIVVSIGLSEREGGSLYISQWFIDRDGDTIATRRKLKPTHVERTIFGEGDGSDLAVWDTRLGRVGGLCCWEHLQPLSKYAMYAQNEQVHVAAWPSFSLYEAYAYALGPEVNTSASRIYAVEGQCFVLAPCALVSKDMVDEMCTTDLKKALLKEGGGYASIFGPDGRQLHEGLPAGEEGLIYAEIDLGLISVSKAVNDPAGHYSRPDVTQLIHNKAPRRAVVDVAPPSDPAPAVERPHGADEPALPAMAEL

SEQ ID NO: 124 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaccgcagtataccgcgcgctattttgataacagcctggaatataacaccccgcaggcgcgccgcattcgcaaagcggcgaaagataacaacattgtggtgagcattggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcactggttagcaaagatatggtggatgagatgtgcaccaccgatctgaaaaaagcgctgttaaaagaaggcggcggctatgcgagcatttttggcccggatggccgccagttacatgaaggcctgccggccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatgtggcaccaccatctgacccagcaccggcggtggaacgtccacatggtgcagatgaaccggcattaccggcgatggcggaactg

SEQ ID NO: 125 - protein, 344 aa, Synthetic construct
MNKPKFKAAIVQAAPVFLDLDKTIDKTVALMEDAARNGAELIAFPETWLPGYPWYAWLDAPAMWMPQYTQRYFDNSLEYGTPQAERIRKVAKDNNIVVSMGLSEREGGSLYISQWFIDRDGDTIATRRKLKPTHVERTIFGEGDGSDLAVWDTRLGRVGGLCCWEHLQPLSKYAMYAQDEQVHVAAWPSFSVYEAYAYALGPDVNTSASRIYAVEGQCFVLAPCATVSKDMVDEMCTTDLKKALLKTGGGYARILGPDGRQLHEGLPAGEEGLIYAEIDLGLISVSKAVGDPAGHYSRPDVTQLIHNKAPRRAVVDTAPPSDPAPAVERPHGEDAPALPAMAEL

SEQ ID NO: 126 - DNA, 1032 bp, Synthetic construct
  source 1..1032: mol_type = other DNA
atgaacaaaccgaaatttaaagcggcgattgtgcaggcggcgccggtgtttctggatttagacaaaaccattgataagaccgtggcgctgatggaagatgcggcgcgcaacggcgcggaactgattgcgtttccggaaacctggttaccgggctatccgtggtatgcgtggctggatgcgccggcaatgtggatgccgcagtatacccagcgctattttgataacagcctggaatatggcaccccgcaggcggaacgcattcgcaaagtggcgaaagataacaacattgtggtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcaggatgaacaggtgcatgtggcggcgtggccgagctttagcgtgtatgaagcgtatgcgtatgcgttaggcccggatgtgaacaccagcgcgagcagaatttatgcggtggaaggccagtgctttgtgctggcgccgtgcgcaactgttagcaaagatatggtggatgagatgtgcaccaccgatctgaaaaaagcgctgttaaaaaccggcggcggctatgcgcgcattctgggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgaagatgcgccggcattaccggcgatggcggaactg

SEQ ID NO: 127 - protein, 344 aa, Synthetic construct
MNKPKFKAAAVQAAPVFLDLDKTIDKTLALMEKAARNGAELIAFPETWLPGYPWYAWLDAPAMWLPQYTQRYFDNSLEYGTPQAERIRKVAKDNNIHVSMGLSEREGGSLYISQWFIDRDGDTIAQRRKLKPTHVERTIFGEGDGSDLAVRDTRLGRVGGLCCWEHLQPLSKYAMYAQNEQVHVAAWPSFSLYEAYAYALGHDVNTSASRIYAVEGQCFVLAPCAVVSKEMSDEMCTTDLQKALLRTGGVYARIFGPDGRLLHEGLPAGEEGLIYAEIDLGLISVGKAVGDPAGHYSRPDVTQLIHNKAPRRAVVDTAPPSDPAPAVERPHGADEPALPAMAEL

SEQ ID NO: 128 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcggaacgcattcgcaaagtggcgaaagataacaacattcatgtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcgcgatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggccatgatgtgaacaccagcgcgagcagaatttatgcggtggaaggccagtgctttgtgctggcgccgtgcgcagtggttagcaaagaaatgagcgatgagatgtgcaccaccgatctgcagaaagcgctgttacgcaccggcggcgtgtatgcgcgcatttttggcccggatggccgcctgttacatgaaggcctgccggccggcgaagaaggcctgatttatgcggaaattgatctgggcctgattagcgtgggcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 129 - protein, 344 aa, Synthetic construct
MNKPKFKAAAVQAAPVFLDLDKTIDKTIALMEDAARNGAELIAFPETWLPGYPWYAWLYAPAMWLPQYTQRYFDNSLEFGSPQAERIRKAAKDNNIWVSMGLSEREGASLYISQWFIDRDGDTIAQRRKLKPTHVERTIFGEGDGSDLAVWDTRLGRVGGLCCWEHLQPLSKYAMYAQNEQVHVAAWPSFSLYRAYAYALGPDVNTHASRIYAVEGQCFVLAPCATVSKEMVDEMCTTDLKKALLKTGGGYARIFGPDGRSLHEGLPPGEEGLIYAEIDLGLISVSKAVNDPAGHYSRPDVTQLIHNKAPRRAVVDTAAPSDPAPAVERPHGADEPALPAMAEL

SEQ ID NO: 130 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgtatgcgccggcaatgtggttaccgcagtatacccagcgctattttgataacagcctggaatttggcagcccgcaggcggaacgcattcgcaaagcggcgaaagataacaacatttgggtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatcgcgcgtatgcgtatgcgttaggcccggatgtgaacacccatgcgagcagaatttatgcggtggaaggccagtgctttgtgctggcgccgtgcgcaactgttagcaaagaaatggtggatgagatgtgcaccaccgatctgaaaaaagcgctgttaaaaaccggcggcggctatgcgcgcatttttggcccggatggccgcagcttacatgaaggcctgccgccgggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcagcgccatctgacccagcaccggcggtggaacgtccacatggtgcagatgaaccggcattaccggcgatggcggaactg

SEQ ID NO: 131 - protein, 344 aa, Synthetic construct
MNKPKFKAAAVQAAPVFLDLDKTIDKTIALMEEAARNGAELIAFPETWLPGYPWYAWLNAPAMWLPQYTQRYFDNSLEYGTPQAERIRKAAKDNNIHVSSGLSEREGGSLYISQWVIDRDGDTVAQRRKLKPTHVERTIFGEGDGSDLAVWDPRLGRVGGLCCWEHLQPLSKYAMYAQNEQVHVAAWPSFSLYGAYAHALGPDVNTSASRIYAVEGQCFVLAPCAIVSKEMVDEMCTTDLHKALLKTGGGYARIFGPDGRPLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 132 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctgaacgcgccggcaatgtggttaccgcagtatacccagcgctattttgataacagcctggaatatggcaccccgcaggcggaacgcattcgcaaagcggcgaaagataacaacattcatgtgagcagcggcctgagcgaacgcgaaggcggcagcctgtacatttctcagtgggtgattgatcgcgatggcgataccgtggcgcagcgccgcaaactgaaaccgacccatgtggaacgcaccatttttggcgaaggcgatggcagcgatctggcggtgtgggatccgcgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgcatgcgttaggcccggatgtgaacaccagcgcgagcagaatttatgcggtggaaggccagtgctttgtgctggcgccgtgcgcaattgttagcaaagaaatggtggatgagatgtgcaccaccgatctgcataaagcgctgttaaaaaccggcggcggctatgcgcgcatttttggcccggatggccgcccgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 133 - protein, 344 aa, Synthetic construct
MNKPKFKAAAVQAAPVFLDLDKTIDKTVALIEDAARNGAELIAFPETWLPGYPWYAWLDAPAMWLPRYTQRYFDNSLEYGTPQAKRIRKTAKDNNIVVSTGLSEREGGSLYISQWHIDRDGDTIAMRRKLKPTHVERTIFGEGDGSDLAVIDTRLGRVGGLCCWEHLQPLSKYAMYAQKEQVHVAAWPSFSAYDAYAYALGPIVNTSASRIYAVEGQCFVLAPCATVSKAMVDEMCDTDVQKALLKTGGGYATILGPDGRQLHEGLPTGEEGLIYAEIDLGLISVSKAVGDPAGHYSRPDVTQLIHNKAPRRAVVDTAPPSDPAPAVERPHGADEPALPAMAEL

SEQ ID NO: 134 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgattgaagatgcggcgcgcaacggcgcggaactgattgcgtttccggaaacctggttaccgggctatccgtggtatgcgtggctggatgcgccggcaatgtggttaccgcgctatacccagcgctattttgataacagcctggaatatggcaccccgcaggcgaaacgcattcgcaaaaccgcgaaagataacaacattgtggtgagcaccggcctgagcgaacgcgaaggcggcagcctgtacatttctcagtggcatattgatcgcgatggcgataccattgcgatgcgccgcaaactgaaaccgacccatgtggaacgcaccatttttggcgaaggcgatggcagcgatctggcggtgattgatacccgtttaggtcgcgttggcggcttatgctgctgggaacatctgcagccgctgagcaaatatgcgatgtatgcgcagaaagaacaggtgcatgtggcggcgtggccgagctttagcgcgtatgatgcgtatgcgtatgcgttaggcccgattgtgaacaccagcgcgagcagaatttatgcggtggaaggccagtgctttgtgctggcgccgtgcgcaactgttagcaaagcgatggtggatgagatgtgcgataccgatgtgcagaaagcgctgttaaaaaccggcggcggctatgcgaccattctgggcccggatggccgccagttacatgaaggcctgccgaccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 135 - protein, 344 aa, Synthetic construct
MNKPKFKAAAVQAAPVFLDLDKTIDKTIALMEDAARNGAELIAFPETWLPGYPWYAWLDAPAMWMPQYTQRYFDNSLEVGTPQARRIRKVAKDNNIVASMGLSEREGGSLYISQWFIDADGDTIATRRKLKPTHVERTIFGEGDGSDLAVYDTRLGRVGGLCCWEHLQPLSKYAMYAQNEQVHVAAWPSFSLYEAYAYALGPEVNTSASRIYAVEGQCFVLAPCASVSKEMQDEMCTTDSQKALLKTGGCYAGIFGPDGRLLTEGLPAGEEGLIYAEIDLGLISVSKAVTDPAGHYSRPDVTQLIHNKAPRRAVVDTAPPSDPAPGVERPHGASEPALPAMAEL

SEQ ID NO: 136 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggatgccgcagtatacccagcgctattttgataacagcctggaagtgggcaccccgcaggcgcgccgcattcgcaaagtggcgaaagataacaacattgtggcgagcatgggcctgagcgaacgcgaaggcggcagcctgtacatttctcagtggtttattgatgcggatggcgataccattgcgacccgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaagcgttagcaaagaaatgcaggatgagatgtgcaccaccgatagccagaaagcgctgttaaaaaccggcggctgctatgcgggcatttttggcccggatggccgcctgttaaccgaaggcctgccggccggcgaagaaggcctgatttatgcggaaattgatctgggcctgattagcgtgagcaaagcggtgaccgatccggcgggtcattatagccgcccagatgtgacccagttaattcataacaaagcgccgcgccgtgcagttgttgatactgcaccaccatctgacccagcaccgggcgtggaacgtccacatggtgcaagcgaaccggcattaccggcgatggcggaactg

SEQ ID NO: 137 - protein, 344 aa, Synthetic construct
MNKPKFKAAAVQAAPVFLDLDKTIDKTIALMEKAARNGAELIAFPETWFPGYPWYAWLDAPAMWLPQYVQRYFDNSLEYGTPQHHRIRKDAKDNNIYVSMGLSEREGGSLYISQWFIDDDGDTIAQRRKLKPTHVERTIFGEGDGSDLAVWDTRLGRVGGLCCWEHLQPLSKYAMYAQKEQVHVAAWPSFSLYEAYAYALGPDVNTSASRIYAVEGQCFVLAPCASVSKEMVDEMCTTDVKKALLKTGGVYARIYGPDGRALHEGLPAGEEGLVYAEIDLGLISVSKAVGDPAGHYSRPDVTQLIHDKAPRRAVVDTAPPSDAAPAVERPHGADEPALPAMAEL

SEQ ID NO: 138 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggtttccgggctatccgtggtatgcgtggctggatgcgccggcaatgtggttaccgcagtatgtgcagcgctattttgataacagcctggaatatggcaccccgcagcatcatcgcattcgcaaagatgcgaaagataacaacatttatgtgagcatgggcctgagcgaacgcgaaggcggcagcctgtacatttctcagtggtttattgatgatgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcaagcgttagcaaagaaatggtggatgagatgtgcaccaccgatgtgaaaaaagcgctgttaaaaaccggcggcgtgtatgcgcgcatttatggcccggatggccgcgcgttacatgaaggcctgccggccggcgaagaaggcctggtgtatgcggaaattgatctgggcctgattagcgtgagcaaagcggtgggcgatccggcgggtcattatagccgcccagatgtgacccagttaattcatgataaagcgccgcgccgtgcagttgttgatactgcaccaccatctgacgcggcaccggcggtggaacgtccacatggtgcagatgaaccggcattaccggcgatggcggaactg

SEQ ID NO: 139 - protein, 344 aa, Synthetic construct
MNKPKFKAAAVQAAPVYLDLDKTIDKTIALMEDAARNGAELIAFPETWLPGYPWYAWLYAPAMWLPQYTQRYFDNSLEFDTPQAARIRKDAKDNNIVVSMGLSEREGSSLYISQWFIDRDGDTIAQRRKLKPTHVERTIFGEGDGSDLAVWDTDLGRVGSLCCWEHLQPLSKYAMYAQDEQVHVAAWPSFSIYEAYAYALGPIVNTSASRIYAVEGQCFVLAPCATVSKEMVDEMCTTDEHKALLTTGGGYAGIFGPDGRALHEGLPAGEEGLIYAEIDLGAISVSKAVTDPAGHYSRPDVTQLIHNKAPRRAVVDTAPPSDPAPAVERPHGADEPALPAMAEL

SEQ ID NO: 140 - DNA, 1032 bp, Synthetic construct
  source 1..1032: mol_type = other DNA
atgaacaaaccgaaatttaaagcggcggcggtgcaggcggcgccggtgtatctggatttagacaaaaccattgataagaccattgcgctgatggaagatgcggcgcgcaacggcgcggaactgattgcgtttccggaaacctggttaccgggctatccgtggtatgcgtggctgtatgcgccggcaatgtggttaccgcagtatacccagcgctattttgataacagcctggaatttgataccccgcaggcggcgcgcattcgcaaagatgcgaaagataacaacattgtggtgagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtgtgggataccgatttaggtcgcgttggcagcttatgctgctgggaacatctgcagccgctgagcaaatatgcgatgtatgcgcaggatgaacaggtgcatgtggcggcgtggccgagctttagcatttatgaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatgaacataaagcgctgttaaccaccggcggcggctatgcgggcatttttggcccggatggccgcgcgttacatgaaggcctgccggccggcgaagaaggcctgatttatgcggaaattgatctgggcgcgattagcgtgagcaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 141 - protein, 344 aa, Synthetic construct
MNKPKFKAAAVQAAPVFLDLDKTIDKTIALMEDAARDGAELIAFPETWLPGYPWYAWLDAPAMWAPMYTQRYFDNSLEYDTPQAERIRKDAKDNNIVVSMGLSEREGGSLYISQWFIDRDGDTIAKRRKLKPTHVERTIFGEGDGSDLAVWDIRLGRVGGLCCWEHLQPLSKYAMYAQNEQVHVAAWPSFSIYEAYAYALGPDVNTGASRIYAVEGQCFVLAPCATVSKDMVDEMCTTDSKKALLKTGGGYATIFGPDGRQLHEGLPATKEGLIYAEIDLGLISVSKAVNDPAGHYSRPDVTQLVHHKAPRRAVVDTAPPSDPAPAVERPHGADEPALPAMAEL

SEQ ID NO: 142 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcgatggcgcggaactgattgcgtttccggaaacctggttaccgggctatccgtggtatgcgtggctggatgcgccggcaatgtgggcgccgatgtatacccagcgctattttgataacagcctggaatatgataccccgcaggcggaacgcattcgcaaagatgcgaaagataacaacattgtggtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaacgaacaggtgcatgtggcggcgtggccgagctttagcatttatgaagcgtatgcgtatgcgttaggcccggatgtgaacaccggcgcgagcagaatttatgcggtggaaggccagtgctttgtgctggcgccgtgcgcaactgttagcaaagatatggtggatgagatgtgcaccaccgatagcaaaaaagcgctgttaaaaaccggcggcggctatgcgaccatttttggcccggatggccgccagttacatgaaggcctgccggccaccaaagaaggcctgatttatgcggaaattgatctgggcctgattagcgtgagcaaagcggtgaacgatccggcgggtcattatagccgcccagatgtgacccagttagtgcatcataaagcgccgcgccgtgcagttgttgatactgcaccaccatctgacccagcaccggcggtggaacgtccacatggtgcagatgaaccggcattaccggcgatggcggaactg

SEQ ID NO: 143 - protein, 344 aa, Synthetic construct
MNKPKFKAAAVQAAPVFLDLDKTIDKTIALMEDAARNGAELIAFPETWLPGYPWYAWLDVPAMWVPQYTQRYFDNSLEYETPQKRRIRKAAKDNNIVVSMGLSEREGSSLYISQWFIDRDGDTIARRRKLKPTHVERTIFGEGDGSDLGVADTRLGRVGGLCCWEHLQPLSKYAMYAQNEQVHVAAWPSFSMYVAYAYALGPDVNSGASRIYAVEGQCFVLAPCATVSKEMVDEMCTEDLKKALLKTGGGYARIFGPDGRLLHEGLPAGEEGLIYAEIDLGLISVSKAVTDPAGHYSRPDVTQLIHEKAPRRAVVDTAPPSDPAPAVERPHGADEPALPAMAEL

SEQ ID NO: 144 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgtgccggcaatgtgggtgccgcagtatacccagcgctattttgataacagcctggaatatgaaaccccgcagaaacgccgcattcgcaaagcggcgaaagataacaacattgtggtgagcatgggcctgagcgaacgcgaaggcagcagcctgtacatttctcagtggtttattgatcgcgatggcgataccattgcgcgccgccgcaaactgaaaccgacccatgtggaacgcaccatttttggcgaaggcgatggcagcgatctgggcgtggcggatacccgtttaggtcgcgttggcggcttatgctgctgggaacatctgcagccgctgagcaaatatgcgatgtatgcgcagaacgaacaggtgcatgtggcggcgtggccgagctttagcatgtatgtggcgtatgcgtatgcgttaggcccggatgtgaacagcggcgcgagcagaatttatgcggtggaaggccagtgctttgtgctggcgccgtgcgcaactgttagcaaagaaatggtggatgagatgtgcaccgaagatctgaaaaaagcgctgttaaaaaccggcggcggctatgcgcgcatttttggcccggatggccgcctgttacatgaaggcctgccggccggcgaagaaggcctgatttatgcggaaattgatctgggcctgattagcgtgagcaaagcggtgaccgatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 145 - protein, 344 aa, Synthetic construct
MNKPKFKAAAVQAAPVFLDLDKSIDKTIALMERAARNGAELIAFPETWLPGYPWYAWLNAPAMWLPQYTQRYFDNSLIYGTPQAERIRKVAKDNNIVVSMGLSEREGGSLYISQWFIDRDGDTIAQRRKLKPTHVERTIFGEGDGSDLAVRDTRLGRVGGLCCWEHLQPLSKYAMYAQNEQVHVAAWPSFSLYGAYAHALGPDVNTSASRIYAVEGQCFVLAPCASVSKGMVDEMCTTDLKKALLKTGGCYAVIFGPDGRQLHEGLPAGEEGLIYAEIDLGLIGVSKAVSDPAGHYSRPDVTQLIHHKSPRRAVVDTATPSDPAPAVERPHGADEPALPAMAEL

SEQ ID NO: 146 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagaccattgcgctgatggaacgcgcggcgcgcaacggcgcggaactgattgcgtttccggaaacctggttaccgggctatccgtggtatgcgtggctgaacgcgccggcaatgtggttaccgcagtatacccagcgctattttgataacagcctgatttatggcaccccgcaggcggaacgcattcgcaaagtggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcgcgatacccgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgcatgcgttaggcccggatgtgaacaccagcgcgagcagaatttatgcggtggaaggccagtgctttgtgctggcgccgtgcgcaagcgttagcaaaggcatggtggatgagatgtgcaccaccgatctgaaaaaagcgctgttaaaaaccggcggctgctatgcggtgatttttggcccggatggccgccagttacatgaaggcctgccggccggcgaagaaggcctgatttatgcggaaattgatctgggcctgattggcgtgagcaaagcggtgagcgatccggcgggtcattatagccgcccagatgtgacccagttaattcatcataaaagcccgcgccgtgcagttgttgatactgcaaccccatctgacccagcaccggcggtggaacgtccacatggtgcagatgaaccggcattaccggcgatggcggaactg

SEQ ID NO: 147 - protein, 344 aa, synthetic construct
MNKPKFKAAAVQAAPVFLDLDKTIDKTIALMEDAARNGAELIAFPETYLPGYPWYAWLYAPAMWLFQYVPRYFDNSLEYGTPQAARIRKAAKDNNIVVSMGLSEREGSSLYISQWFIDRDGDTIAQRRKLKPTHVERTIFGEGDGSDLAVIDPRLGRVGTLCCWEHLQPLSKYAMYAQNEQVHVAAWPSFSVYEPYAYALGPDVNTSASRIYAVEGQCFVLAPCAIVSKGMVDEMCSTDLHKALLKTGGGYATIFGPDGRQLHEGLPAGEEGLIYAEIDLGLISVSKAVSDPAGHYSRPDVTQLIHNKAPRRAVVDTAPPSDPAPAVERPHGADEPALPAMAEL

SEQ ID NO: 148 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctatttaccgggctatccgtggtatgcgtggctgtatgcgccggcaatgtggttatttcagtatgtgccgcgctattttgataacagcctggaatatggcaccccgcaggcggcgcgcattcgcaaagcggcgaaagataacaacattgtggtgagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtgattgatccgcgtttaggtcgcgttggcaccttatgctgctgggaacatctgcagccgctgagcaaatatgcgatgtatgcgcagaacgaacaggtgcatgtggcggcgtggccgagctttagcgtgtatgaaccgtatgcgtatgcgttaggcccggatgtgaacaccagcgcgagcagaatttatgcggtggaaggccagtgctttgtgctggcgccgtgcgcaattgttagcaaaggcatggtggatgagatgtgcagcaccgatctgcataaagcgctgttaaaaaccggcggcggctatgcgaccatttttggcccggatggccgccagttacatgaaggcctgccggccggcgaagaaggcctgatttatgcggaaattgatctgggcctgattagcgtgagcaaagcggtgagcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 149 - protein, 344 aa, Synthetic construct
MNKPKFKLAAVQAAPVFLDLDKTIDKTIALMEDAARNGAELIAFPETWLPGYPWYAWLDAPAMWLMQYTARYFDNSLEYGTPQFHRIRKAAKDNNIVVSMGLSEREGSSLYISQWFIDRDGDTIAARRKLKPTHSERTIFGEGDGSDLAVADLRLGRVGGLCCWEHLQPLSKYAMYAQNEQVHVAAWPSFSLYVAYAYALGPEVNTSASRIYAVEGQCFVLAPCASVSKGMVDEMCTSDLQKALLKTGGCYAGIFGPDGRALHEGLPAGEEGLIYAEIDLGLISVSKAVGDPAGHYSRPDVTQLIHNKAARRAVVDTAPPSDPAPPVERPHGADEPALPAMAEL

SEQ ID NO: 150 - DNA, 1032 bp, Synthetic construct
  source 1..1032: mol_type = other DNA
atgaacaaaccgaaatttaaactggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaatgcagtataccgcgcgctattttgataacagcctggaatatggcaccccgcagtttcatcgcattcgcaaagcggcgaaagataacaacattgtggtgagcatgggcctgagcgaacgcgaaggcagcagcctgtacatttctcagtggtttattgatcgcgatggcgataccattgcggcgcgccgcaaactgaaaccgacccatagcgaacgcaccatttttggcgaaggcgatggcagcgatctggcggtggcggatctgcgtttaggtcgcgttggcggcttatgctgctgggaacatctgcagccgctgagcaaatatgcgatgtatgcgcagaacgaacaggtgcatgtggcggcgtggccgagctttagcctgtatgtggcgtatgcgtatgcgttaggcccggaagtgaacaccagcgcgagcagaatttatgcggtggaaggccagtgctttgtgctggcgccgtgcgcaagcgttagcaaaggcatggtggatgagatgtgcaccagcgatctgcagaaagcgctgttaaaaaccggcggctgctatgcgggcatttttggcccggatggccgcgcgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcggcgcgccgtgcagttgttgatactgcaccaccatctgacccagcaccgccggtggaacgtccacatggtgcagatgaaccggcattaccggcgatggcggaactg

SEQ ID NO: 151 - protein, 344 aa, Synthetic construct
MNKPKFKLAAVQAAPVFLDLDKTIDKTIALMEKAARNGAELIAFPETWLPGYPWYAWLDAPAMWLPQYTQRYFDNSLEIGTPQADRIRKVAKDNNIYVSMGLSEREGASLYISQWFIDRDGDTIAQRRKLKPTHVERTIFGEGDGSDLAVWDLRLGRVGGLCCWEHLQPLSKYAMYAQDEQVHVAAWPSFSMYEAYAYALGPDVNTSASRIYAVEGQCFVLAPCATVSKEMVDEMCTTEVKKALLTTGGGYARIFGPDGRQLHEGLPAGEEGLIYAEIDLGLISVSKAVTDPAGHYSRPDVTQLIVHKSPRRAVVDTAPPSDPAPAVERPHGAAEPALPAMAEL

SEQ ID NO: 152 - DNA, 1032 bp, Synthetic construct
  source 1..1032: mol_type = other DNA
atgaacaaaccgaaatttaaactggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaaattggcaccccgcaggcggatcgcattcgcaaagtggcgaaagataacaacatttatgtgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtgggatctgcgtttaggtcgcgttggcggcttatgctgctgggaacatctgcagccgctgagcaaatatgcgatgtatgcgcaggatgaacaggtgcatgtggcggcgtggccgagctttagcatgtatgaagcgtatgcgtatgcgttaggcccggatgtgaacaccagcgcgagcagaatttatgcggtggaaggccagtgctttgtgctggcgccgtgcgcaactgttagcaaagaaatggtggatgagatgtgcaccaccgaagtgaaaaaagcgctgttaaccaccggcggcggctatgcgcgcatttttggcccggatggccgccagttacatgaaggcctgccggccggcgaagaaggcctgatttatgcggaaattgatctgggcctgattagcgtgagcaaagcggtgaccgatccggcgggtcattatagccgcccagatgtgacccagttaattgtgcataaaagcccgcgccgtgcagttgttgatactgcaccaccatctgacccagcaccggcggtggaacgtccacatggtgcagcggaaccggcattaccggcgatggcggaactg

SEQ ID NO: 153 - protein, 344 aa, Synthetic construct
MNKPKFKAAAVQAAPVFLDLDKTIDKTIALMEEAARNGAELIAFPETWLPGYPWYAWLDAPAMWLFQYTQRYFDNSLEFDTPQARRIRKDAKDNNIWVSTGLSEREGGSLYISQWFIDRDGDTIATRRKLKPTHVERTIFGEGDGSDLGVADPRLGRVGGLCCWEHLQPLSKYAMYAQHEQVHVAAWPSFSIYVAYAYALGPEVNTSASRIYAVEGQCFVLAPCATVSKGMVDEMCTTDGQKALLKTGGGYARIFGPDGRQLHEGLPAGEEGLIYAEIDLGLIGVSKAVADPAGHYSRPDVTQLIHNKAPRRAVVDTAPPSDPAPAVERPHGADQPALPAMAEL

SEQ ID NO: 154 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttatttcagtatacccagcgctattttgataacagcctggaatttgataccccgcaggcgcgccgcattcgcaaagatgcgaaagataacaacatttgggtgagcaccggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctgggcgtggcggatccgcgtttaggtcgcgttggcggcttatgctgctgggaacatctgcagccgctgagcaaatatgcgatgtatgcgcagcatgaacaggtgcatgtggcggcgtggccgagctttagcatttatgtggcgtatgcgtatgcgttaggcccggaagtgaacaccagcgcgagcagaatttatgcggtggaaggccagtgctttgtgctggcgccgtgcgcaactgttagcaaaggcatggtggatgagatgtgcaccaccgatggccagaaagcgctgttaaaaaccggcggcggctatgcgcgcatttttggcccggatggccgccagttacatgaaggcctgccggccggcgaagaaggcctgatttatgcggaaattgatctgggcctgattggcgtgagcaaagcggtggcggatccggcgggtcattatagccgcccagatgtgacccagttaattcataacaaagcgccgcgccgtgcagttgttgatactgcaccaccatctgacccagcaccggcggtggaacgtccacatggtgcagatcagccggcattaccggcgatggcggaactg

SEQ ID NO: 155 - protein, 344 aa, Synthetic construct
MNKPKFKAAAVQAAPVFLDLDKTIDKTLALMEDAARNGAELIAFPETWLPGYPWYAWLDAPAMWLPQYTHRYFDNSLEYGTPQADRIRKDAKDNNIHASSGLSEREGGSLYISQWFIDRDGDTIAKRRKLKPTHVERTIFGEGDGSDLAVYDTRLGRVGGLCCWEHLQPLSKYAMYAQDEQVHVAAWPSFSLYVAYAYALGPEVNTSASRIYAVEGQCFVLAPCAIVSKEMVDEMCTTDGQKALLTTGGVYARIFGPDGRALHEGLPAGEEGLIYAEIDLGLIGVSKSVGDPAGHYSRPDVTQLVHEKAPRRAVVDTAPPSDPAPAVERPHGADEPALPAMAEL

SEQ ID NO: 156 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaagatgcggcgcgcaacggcgcggaactgattgcgtttccggaaacctggttaccgggctatccgtggtatgcgtggctggatgcgccggcaatgtggttaccgcagtatacccatcgctattttgataacagcctggaatatggcaccccgcaggcggatcgcattcgcaaagatgcgaaagataacaacattcatgcgagcagcggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcaggatgaacaggtgcatgtggcggcgtggccgagctttagcctgtatgtggcgtatgcgtatgcgttaggcccggaagtgaacaccagcgcgagcagaatttatgcggtggaaggccagtgctttgtgctggcgccgtgcgcaattgttagcaaagaaatggtggatgagatgtgcaccaccgatggccagaaagcgctgttaaccaccggcggcgtgtatgcgcgcatttttggcccggatggccgcgcgttacatgaaggcctgccggccggcgaagaaggcctgatttatgcggaaattgatctgggcctgattggcgtgagcaaaagcgtgggcgatccggcgggtcattatagccgcccagatgtgacccagttagtgcatgaaaaagcgccgcgccgtgcagttgttgatactgcaccaccatctgacccagcaccggcggtggaacgtccacatggtgcagatgaaccggcattaccggcgatggcggaactg

SEQ ID NO: 157 - protein, 344 aa, Synthetic construct
MNKPKFKAASVQAAPVFLDLDKTIDKTIALMEEAARNGAELIAFPETWLPGYPWYAWLDAPAMWLPQYTARYFDNSLIYDTPQARRIRKAAKDNNIHVSMGLSEREGESLYISQWFIDRDGDTAARRRKLKPTHVERTIFGEGDGSDLVVWDARLGRVGTLCCWEHLQPLSKYAMYAQNEQVHVAAWPSFSAYEAYAYALGPDVNTSASRIYAVEGQCFVLAPCATVSKSMVDEMCTTDARKALLKTGGVYAAIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 158 - DNA, 1032 bp, Synthetic construct
  source 1..1032: mol_type = other DNA
atgaacaaaccgaaatttaaagcggcgagcgtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaccgcagtataccgcgcgctattttgataacagcctgatttatgataccccgcaggcgcgccgcattcgcaaagcggcgaaagataacaacattcatgtgagcatgggcctgagcgaacgcgaaggcgaaagcctgtacatttctcagtggtttattgatcgcgatggcgataccgcggcgcgccgccgcaaactgaaaccgacccatgtggaacgcaccatttttggcgaaggcgatggcagcgatctggtggtgtgggatgcgcgtttaggtcgcgttggcaccttatgctgctgggaacatctgcagccgctgagcaaatatgcgatgtatgcgcagaacgaacaggtgcatgtggcggcgtggccgagctttagcgcgtatgaagcgtatgcgtatgcgttaggcccggatgtgaacaccagcgcgagcagaatttatgcggtggaaggccagtgctttgtgctggcgccgtgcgcaactgttagcaaaagcatggtggatgagatgtgcaccaccgatgcgcgcaaagcgctgttaaaaaccggcggcgtgtatgcggcgatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 159 - protein, 344 aa, Synthetic construct
MNKPKFKAAAVQAAPVFLDLDKTIDKTLALMEDAARNGAELIAFPETWLPGYPWYAWLDAPAMWLPQYTQRYFDNSLTYGTPQDHRIRKAAKDNNIYVSMGLSEREGGSLYISQWFIDRDGDTIATRRKLKPTHVERTIFGEGDGSDLAVVDTDLGRVGGLCCWEHLQPLSKYAMYAQNEQVHVAAWPSFSMYKAYAYALGPDVNTSASRIYAVEGQCFVLAPCAIVSKSMVDEMCTTDLKKALLKTGGGYATIFGPDGRQLHEGLAAGEEGLIYAEIDLGLISVSKSVGDPAGHYSRPDVTQLIHHKAPRRAVVDTATPSDDAPAVERPHGADEPALPAMAEL

SEQ ID NO: 160 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccctggcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctgacctatggcaccccgcaggatcatcgcattcgcaaagcggcgaaagataacaacatttatgtgagcatgggcctgagcgaacgcgaaggcggcagcctgtacatttctcagtggtttattgatcgcgatggcgataccattgcgacccgccgcaaactgaaaccgacccatgtggaacgcaccatttttggcgaaggcgatggcagcgatctggcggtggtggataccgatttaggtcgcgttggcggcttatgctgctgggaacatctgcagccgctgagcaaatatgcgatgtatgcgcagaacgaacaggtgcatgtggcggcgtggccgagctttagcatgtataaagcgtatgcgtatgcgttaggcccggatgtgaacaccagcgcgagcagaatttatgcggtggaaggccagtgctttgtgctggcgccgtgcgcaattgttagcaaaagcatggtggatgagatgtgcaccaccgatctgaaaaaagcgctgttaaaaaccggcggcggctatgcgaccatttttggcccggatggccgccagttacatgaaggcctggcggccggcgaagaaggcctgatttatgcggaaattgatctgggcctgattagcgtgagcaaaagcgtgggcgatccggcgggtcattatagccgcccagatgtgacccagttaattcatcataaagcgccgcgccgtgcagttgttgatactgcaaccccatctgacgatgcaccggcggtggaacgtccacatggtgcagatgaaccggcattaccggcgatggcggaactg

SEQ ID NO: 161 - protein, 344 aa, Synthetic construct
MNKPKFKVAAVQAAPVFLDLDKTIDKTIALMEDAARNGAELIAFPETWLPGYPWYAWLDAPAMWLPQYTQRYFDNSLEFGTPQFHRIRKSAKDNNIHLSMGLSEREGESLYISQWFIDRDGDTIAKRRKLKPTHVERTIFGEGDGSDLAVWDIRLGRVGGLCCWEHLQPLSKYAMYAQNEQVHVAAWPSFSAYEAYAYALGPIVNTSASRIYAVEGQCFVLAPCATVSKEMVDEMCTRDGRKALLLTGGGYAGIFGPDGRQLHEGLPAGEEGLIYAEIDLGLISVSKAVADPAGHYSRPDVTQLIHNKAPRRVVIDTAPPSDPAPAVERPHGADEPALPAMAEL

SEQ ID NO: 162 - DNA, 1032 bp, Synthetic construct
  source 1..1032: mol_type = other DNA
atgaacaaaccgaaatttaaagtggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatttggcaccccgcagtttcatcgcattcgcaaaagcgcgaaagataacaacattcatctgagcatgggcctgagcgaacgcgaaggcgaaagcctgtacatttctcagtggtttattgatcgcgatggcgataccattgcgaaacgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaacgaacaggtgcatgtggcggcgtggccgagctttagcgcgtatgaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcacccgcgatggccgcaaagcgctgttactgaccggcggcggctatgcgggcatttttggcccggatggccgccagttacatgaaggcctgccggccggcgaagaaggcctgatttatgcggaaattgatctgggcctgattagcgtgagcaaagcggtggcggatccggcgggtcattatagccgcccagatgtgacccagttaattcataacaaagcgccgcgccgtgtggttattgatactgcaccaccatctgacccagcaccggcggtggaacgtccacatggtgcagatgaaccggcattaccggcgatggcggaactg

SEQ ID NO: 163 - protein, 344 aa, Synthetic construct
MNKPKFKAAAVQAAPVYLDLDKTIDKTVALMEDAARNGAELIAFPETWLPGYPWYAWLDAPAMWAPQYTQRYFDNSLEYGTPQAERIRKAAKDNNIVVSMGLSEREGESLYIAQWFIDRDGDTIATRRKLKPTHVERTIFGEGDGSDLAVWDLRLGRVGGLCCWEHLQPLSKYAMYAQNEQVHVAAWPSFSIYEAYAYALGPDVNTSASRIYAVEGGCFVLAPCAIVSKGMVDEMCTTDLRKALLKTGGCYAGILGPDGRVLHEGLPAGEEGLIYAEIDLGLISVSKAVNDPAGHYSRPDVTQLIHNKAPRRAVVDTAPPSDPAPAVERPHGAPEPALPAMAEL

SEQ ID NO: 164 - DNA, 1032 bp, Synthetic construct
  source 1..1032: mol_type = other DNA
atgaacaaaccgaaatttaaagcggcggcggtgcaggcggcgccggtgtatctggatttagacaaaaccattgataagaccgtggcgctgatggaagatgcggcgcgcaacggcgcggaactgattgcgtttccggaaacctggttaccgggctatccgtggtatgcgtggctggatgcgccggcaatgtgggcgccgcagtatacccagcgctattttgataacagcctggaatatggcaccccgcaggcggaacgcattcgcaaagcggcgaaagataacaacattgtggtgagcatgggcctgagcgaacgcgaaggcgaaagcctgtacattgcgcagtggtttattgatcgcgatggcgataccattgcgacccgccgcaaactgaaaccgacccatgtggaacgcaccatttttggcgaaggcgatggcagcgatctggcggtgtgggatctgcgcttaggtcgcgttggcggcttatgctgctgggaacatctgcagccgctgagcaaatatgcgatgtatgcgcagaacgaacaggtgcatgtggcggcgtggccgagctttagcatttatgaagcgtatgcgtatgcgttaggcccggatgtgaacaccagcgcgagcagaatttatgcggtggaaggcggctgctttgtgctggcgccgtgcgcaattgttagcaaaggcatggtggatgagatgtgcaccaccgatctgcgcaaagcgctgttaaaaaccggcggctgctatgcgggcattctgggcccggatggccgcgtgttacatgaaggcctgccggccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacccagcaccggcggtggaacgtccacatggtgcaccggaaccggcattaccggcgatggcggaactg

SEQ ID NO: 165 - protein, 344 aa, Synthetic construct
MNKPKFKAAIVQAAPVFLDLDKTIDKTIALMEDAARNGAELIAFPETWLPGYPWYAWLYAPAMWLPQYTQRYFDNSLEFGTPQAHRIRKSAKDNNIVASTGLSEREGESLYISQWFIDRDGDTIAQRRKLKPTHVERTIFGEGDGSDLAVADTRLGRIGGLCCWEHLQPLSKYAMYAQHEQVHVAAWPSFSLYDAYAYALGPEVNTSASRIYAVEGQCFVLAPCALVSKEMVDEMCTTDLRKALLKTGGAYARIFGPDGRSLHEGLPASEEGLIYAEIDLGLISVSKAVSDPAGHYSRPDVTQLILHKAPRRAVVDTAPPSDPAPAVERPHGADEPALPAMAEL

SEQ ID NO: 166 - DNA, 1032 bp, Synthetic construct
  source 1..1032: mol_type = other DNA
atgaacaaaccgaaatttaaagcggcgattgtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgtatgcgccggcaatgtggttaccgcagtatacccagcgctattttgataacagcctggaatttggcaccccgcaggcgcatcgcattcgcaaaagcgcgaaagataacaacattgtggcgagcaccggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtggcggatacccgtttaggtcgcattggcggcttatgctgctgggaacatctgcagccgctgagcaaatatgcgatgtatgcgcagcatgaacaggtgcatgtggcggcgtggccgagctttagcctgtatgatgcgtatgcgtatgcgttaggcccggaagtgaacaccagcgcgagcagaatttatgcggtggaaggccagtgctttgtgctggcgccgtgcgcactggttagcaaagaaatggtggatgagatgtgcaccaccgatctgcgcaaagcgctgttaaaaaccggcggcgcgtatgcgcgcatttttggcccggatggccgcagcttacatgaaggcctgccggccagcgaagaaggcctgatttatgcggaaattgatctgggcctgattagcgtgagcaaagcggtgagcgatccggcgggtcattatagccgcccagatgtgacccagttaattctgcataaagcgccgcgccgtgcagttgttgatactgcaccaccatctgacccagcaccggcggtggaacgtccacatggtgcagatgaaccggcattaccggcgatggcggaactg

SEQ ID NO: 167 - protein, 344 aa, Synthetic construct
MNKPKFKAAAVQAAPVFLDLDKTIDKTIALAEDAARNGAELIAFPETWFPGYPWYAWLDAPAMWLPQYTDRYFDNSLEYGTPQDERIRKAAKDNNIWVSMGLSEREGGSLYISQWFIDRDGDTIAQRRKLKPTHVERTIFGEGDGSDLAVADTRLGRVGGLCCWEHIQPLSKYAMYAQDEQVHVAAWPSFSLYKAYAYALGPDVNTSASRAYAVEGQCFVLAPCACVSKAMVDEMCTTDLQKALLTTGGVYASIFGPDGRALHEGLPAGEEGLIYAEIDLGLISVSKAVADPAGHYSRPDVTQLIFEKAPRRAVVDTAPPSDPAPAVERPHGADEPALPAMAEL

SEQ ID NO: 168 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctggcggaagatgcggcgcgcaacggcgcggaactgattgcgtttccggaaacctggtttccgggctatccgtggtatgcgtggctggatgcgccggcaatgtggttaccgcagtataccgatcgctattttgataacagcctggaatatggcaccccgcaggatgaacgcattcgcaaagcggcgaaagataacaacatttgggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtggcggatacccgtttaggtcgcgttggcggcttatgctgctgggaacatattcagccgctgagcaaatatgcgatgtatgcgcaggatgaacaggtgcatgtggcggcgtggccgagctttagcctgtataaagcgtatgcgtatgcgttaggcccggatgtgaacaccagcgcgagcagagcgtatgcggtggaaggccagtgctttgtgctggcgccgtgcgcatgcgttagcaaagcgatggtggatgagatgtgcaccaccgatctgcagaaagcgctgttaaccaccggcggcgtgtatgcgagcatttttggcccggatggccgcgcgttacatgaaggcctgccggccggcgaagaaggcctgatttatgcggaaattgatctgggcctgattagcgtgagcaaagcggtggcggatccggcgggtcattatagccgcccagatgtgacccagttaatttttgaaaaagcgccgcgccgtgcagttgttgatactgcaccaccatctgacccagcaccggcggtggaacgtccacatggtgcagatgaaccggcattaccggcgatggcggaactg

SEQ ID NO: 169 - protein, 344 aa, Synthetic construct
MNKPKFKLAAVQAAPVFLDLDKTIDKTIALMEDAARNGAELIAFPETWLPGYPWYAWLYTPAMWLPQYTQRYFDNSLEYGTPQYERIRKAAKDNNIVVSMGLSEREGGSLYISQWIIDRDGDTIATRRKLKPTHVERTIFGEGDGSDLAVWDTRLGRVGGLCCWEHLQPLSKYAMYAQDEQVHVAAWPSFSLYRAYAYALGPIVNTSASRIYAVEGQCFVLAPCAVVSKGMVDEMCTTDSQKALLKTGGGYAVILGPDGRLLHEGLPHGEEGLIYAEIDLGLISVSKAVTDPAGHYSRPDVTQLIHHKAPRRAVIDTAPPSDPAPAVERPHGADEPALPAMAEL

SEQ ID NO: 170 - DNA, 1032 bp, Synthetic construct
  source 1..1032: mol_type = other DNA
atgaacaaaccgaaatttaaactggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgtataccccggcaatgtggttaccgcagtatacccagcgctattttgataacagcctggaatatggcaccccgcagtatgaacgcattcgcaaagcggcgaaagataacaacattgtggtgagcatgggcctgagcgaacgcgaaggcggcagcctgtacatttctcagtggattattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcaggatgaacaggtgcatgtggcggcgtggccgagctttagcctgtatcgcgcgtatgcgtatgcgttaggcccgattgtgaacaccagcgcgagcagaatttatgcggtggaaggccagtgctttgtgctggcgccgtgcgcagtggttagcaaaggcatggtggatgagatgtgcaccaccgatagccagaaagcgctgttaaaaaccggcggcggctatgcggtgattctgggcccggatggccgcctgttacatgaaggcctgccgcatggcgaagaaggcctgatttatgcggaaattgatctgggcctgattagcgtgagcaaagcggtgaccgatccggcgggtcattatagccgcccagatgtgacccagttaattcatcataaagcgccgcgccgtgcagttattgatactgcaccaccatctgacccagcaccggcggtggaacgtccacatggtgcagatgaaccggcattaccggcgatggcggaactg

SEQ ID NO: 171 - protein, 344 aa, Synthetic construct
MNKPKFKAAAVQAAPVFLDLDKTIDKTIALMEDAARNGAELIAFPETYLPGYPWYAWLDAPAMWLPQYTDRYFDNSLEYGTPQHRRIRKDAKDNNIVVSMGLSEREGGSLYISQWFIDRDGDTIAKRRKLKPTHVERTIFGEGDGSDLAVWDARLGRVGGLCCWEHLQPLSKYAMYAQTEQVHVAAWPSFSAYEAYAYALGGIVNTSASRIYAVEGGCFVLAPCAIVSKAMVDEMCTTDDQKALLKTGGAYARIFGPDGRLLSEGLDAGEEGLIYAEIDLGLIGVSKAVGDPAGHYSRPDVTQLIHNKAPRRAVVDTAPPSDPAPAVERPHGADEPALPAMAEL

SEQ ID NO: 172 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctatttaccgggctatccgtggtatgcgtggctggatgcgccggcaatgtggttaccgcagtataccgatcgctattttgataacagcctggaatatggcaccccgcagcatcgccgcattcgcaaagatgcgaaagataacaacattgtggtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtgggatgcgcgtttaggtcgcgttggcggcttatgctgctgggaacatctgcagccgctgagcaaatatgcgatgtatgcgcagaccgaacaggtgcatgtggcggcgtggccgagctttagcgcgtatgaagcgtatgcgtatgcgttaggcggcattgtgaacaccagcgcgagcagaatttatgcggtggaaggcggctgctttgtgctggcgccgtgcgcaattgttagcaaagcgatggtggatgagatgtgcaccaccgatgatcagaaagcgctgttaaaaaccggcggcgcgtatgcgcgcatttttggcccggatggccgcctgttaagcgaaggcctggatgccggcgaagaaggcctgatttatgcggaaattgatctgggcctgattggcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 173 - protein, 344 aa, Synthetic construct
MNKPKFKAAAVQAAPVFLDLDKTIDKTIALMEDAARNGAELIAFPETWLPGYPWYAWLTAPAMWAPQYTQRYFDNSLTYGTPQAKRIRKAAKDNNIVVSMGLSEREGESLYISQWFIDRDGDTIAQRRKLKPTHVERTIFGEGDGSDLAVRDIRLGRVGGLCCWEHLQPLSKYAMYAQDEQVHVAAWPSFSAYEAYAYALGPEVNVSASRIYAVEGQCFVLAPCALVSKDMVDEMCTTDLKKALLKTGGGYASIFGPDGRQLHEGLPAGEEGLIYAEIDLGLISVSKNVNDPAGHYSRPDVTQLIHNKAPRRRVVDTAPPSDPAPAVERPHGADELALPAMAEL

SEQ ID NO: 174 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgaccgcgccggcaatgtgggcgccgcagtatacccagcgctattttgataacagcctgacctatggcaccccgcaggcgaaacgcattcgcaaagcggcgaaagataacaacattgtggtgagcatgggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtgcgcgatattcgtttaggtcgcgttggcggcttatgctgctgggaacatctgcagccgctgagcaaatatgcgatgtatgcgcaggatgaacaggtgcatgtggcggcgtggccgagctttagcgcgtatgaagcgtatgcgtatgcgttaggcccggaagtgaacgtgagcgcgagcagaatttatgcggtggaaggccagtgctttgtgctggcgccgtgcgcactggttagcaaagatatggtggatgagatgtgcaccaccgatctgaaaaaagcgctgttaaaaaccggcggcggctatgcgagcatttttggcccggatggccgccagttacatgaaggcctgccggccggcgaagaaggcctgatttatgcggaaattgatctgggcctgattagcgtgagcaaaaacgtgaacgatccggcgggtcattatagccgcccagatgtgacccagttaattcataacaaagcgccgcgccgtcgcgttgttgatactgcaccaccatctgacccagcaccggcggtggaacgtccacatggtgcagatgaactggcattaccggcgatggcggaactg

SEQ ID NO: 175 - protein, 344 aa, Synthetic construct
MNKPKFKAAAVQAAPVFLDLDKTIDKTIALMEDAARNGAELIAFPETWLPGYPWYAWLDAPAMWLPQYTHRYFDNSLVYGTPQDHRIRKVAKDNNIVVSMGLSEREGSSLYISQWFIDRDGDTIAQRRKLKPTHVERTIFGEGDGSDLAVIDTRLGRVGGLCCWEHLQPLSKYAMYAQDEQVHVAAWPSFSMYEAYAYALGPEVNTSASRAYAVEGQCFVLAPCATVSKEMVDEMCTTDLQKALLKTGGAYASIFGPDGRSLHEGLPAGEEGLIYAEIDLGLISVSKTVTDPAGHYSRPDVTQLIHDKAPRRAVVDLATPSDPAPAVERPHGADEPALPAMAEL

SEQ ID NO: 176 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccatcgctattttgataacagcctggtgtatggcaccccgcaggatcatcgcattcgcaaagtggcgaaagataacaacattgtggtgagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtgattgatacccgtttaggtcgcgttggcggcttatgctgctgggaacatctgcagccgctgagcaaatatgcgatgtatgcgcaggatgaacaggtgcatgtggcggcgtggccgagctttagcatgtatgaagcgtatgcgtatgcgttaggcccggaagtgaacaccagcgcgagcagagcgtatgcggtggaaggccagtgctttgtgctggcgccgtgcgcaactgttagcaaagaaatggtggatgagatgtgcaccaccgatctgcagaaagcgctgttaaaaaccggcggcgcgtatgcgagcatttttggcccggatggccgcagcttacatgaaggcctgccggccggcgaagaaggcctgatttatgcggaaattgatctgggcctgattagcgtgagcaaaaccgtgaccgatccggcgggtcattatagccgcccagatgtgacccagttaattcatgataaagcgccgcgccgtgcagttgttgatctggcaaccccatctgacccagcaccggcggtggaacgtccacatggtgcagatgaaccggcattaccggcgatggcggaactg

SEQ ID NO: 177 - protein, 344 aa, Synthetic construct
MNKPKFKIASVQAAPVFLDLDKTIDKTIALMEDAARNGAELIAFPETWLPGYPWYAWLDAPAMWLPQYTQRYFDNSLEIGTPQHDRIRKDAKDNNIHVSMGLSEREGGSLYISQWLIDRDGDTIATRRKLKPTHVERTIFGEGDGSDLMVRDARLGRVGGLCCWEHLQPLSKYAMYAQNEQVHVAAWPSFSIYVAYAYALGPEVNTSASRIYAVEGQCFVLAPCATVSKEMVDEMCTTDEQKALLKTGGGYARIFGPDGRLLHEGLPAGEEGLIYAEIDLGLITVSKAVGDPAGHYSRPDVTQLIHEKAPRRAVVDTAPPSDPAPAVERPHGADEPALPAMAEL

SEQ ID NO: 178 - DNA, 1032 bp, Synthetic construct
  source 1..1032: mol_type = other DNA
atgaacaaaccgaaatttaaaattgcgagcgtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaaattggcaccccgcagcatgatcgcattcgcaaagatgcgaaagataacaacattcatgtgagcatgggcctgagcgaacgcgaaggcggcagcctgtacatttctcagtggctgattgatcgcgatggcgataccattgcgacccgccgcaaactgaaaccgacccatgtggaacgcaccatttttggcgaaggcgatggcagcgatctgatggtgcgcgatgcgcgtttaggtcgcgttggcggcttatgctgctgggaacatctgcagccgctgagcaaatatgcgatgtatgcgcagaacgaacaggtgcatgtggcggcgtggccgagctttagcatttatgtggcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatgaacagaaagcgctgttaaaaaccggcggcggctatgcgcgcatttttggcccggatggccgcctgttacatgaaggcctgccggccggcgaagaaggcctgatttatgcggaaattgatctgggcctgattaccgtgagcaaagcggtgggcgatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 179 - protein, 344 aa, Synthetic construct
MNKPKFKAAAVQAAPVFLDLDKTIDKTIALMEDAARNGAELIAFPETWLPGYPWYAWLDAPAMWAPQYTQRYFDNSLEYGTPQDERIRKAAKDNNITVSMGLSEREGGSLYISQWHIDRDGDTIARRRKLKPTHVERTIFGEGDGSDLAVVDARLGRVGGLCCWEHLQPLSKYAMYAQNEQVHVAAWPSFSVYEAYAYALGPDVNVSASRAYAVEGQCFVLAPCASVSKEMVDEMCTTDEQKALLKTGGAYARIFGPDGRPLHEGLPAGEEGLIYAEIDLGLITVGKAVGDPAGHYSRPDVTQLIHEKAPRRAVVDTAPPSDPAPAVERPHGADEPALPAMAEL

SEQ ID NO: 180 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtgggcgccgcagtatacccagcgctattttgataacagcctggaatatggcaccccgcaggatgaacgcattcgcaaagcggcgaaagataacaacattaccgtgagcatgggcctgagcgaacgcgaaggcggcagcctgtacatttctcagtggcatattgatcgcgatggcgataccattgcgcgccgccgcaaactgaaaccgacccatgtggaacgcaccatttttggcgaaggcgatggcagcgatctggcggtggtggatgcgcgtttaggtcgcgttggcggcttatgctgctgggaacatctgcagccgctgagcaaatatgcgatgtatgcgcagaacgaacaggtgcatgtggcggcgtggccgagctttagcgtgtatgaagcgtatgcgtatgcgttaggcccggatgtgaacgtgagcgcgagcagagcgtatgcggtggaaggccagtgctttgtgctggcgccgtgcgcaagcgttagcaaagaaatggtggatgagatgtgcaccaccgatgaacagaaagcgctgttaaaaaccggcggcgcgtatgcgcgcatttttggcccggatggccgcccgttacatgaaggcctgccggccggcgaagaaggcctgatttatgcggaaattgatctgggcctgattaccgtgggcaaagcggtgggcgatccggcgggtcattatagccgcccagatgtgacccagttaattcatgaaaaagcgccgcgccgtgcagttgttgatactgcaccaccatctgacccagcaccggcggtggaacgtccacatggtgcagatgaaccggcattaccggcgatggcggaactg

SEQ ID NO: 181 - protein, 344 aa, Synthetic construct
MNKPKFKAAAVQAAPVFLDLDKTIDKTIALMEDAARNGAELIAFPETWLPGYPWYAWLDAPAMWLPRYTQRYFDNSLEYGTPQFERIRKAAKNNNIVVSMGLSEREGESLYISQWVIDRDGDTIATRRKLKPTHVERTIFGEGDGSDLAVWDTRLGRVGGLCCWEHLQPLSKYAMYAQKEQVHVAAWPSFSAYEAYAYALGPIVNTSASRIYAVEGQCFVLAPCALVSKEMVDEMCTTDDKKALLKAGGCYATIFGPDGRQLHEGLAAGEEGLIYAEIDLGLISVSKAVSDPAGHYSRPDVTQLIINKAPRRAVVDTAPPSDPAPAVERPHGADEPALPAMAEL

SEQ ID NO: 182 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcgctatacccagcgctattttgataacagcctggaatatggcaccccgcagtttgaacgcattcgcaaagcggcgaaaaacaacaacattgtggtgagcatgggcctgagcgaacgcgaaggcgaaagcctgtacatttctcagtgggtgattgatcgcgatggcgataccattgcgacccgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcgcgtatgaagcgtatgcgtatgcgttaggcccgattgtgaacaccagcgcgagcagaatttatgcggtggaaggccagtgctttgtgctggcgccgtgcgcactggttagcaaagaaatggtggatgagatgtgcaccaccgatgataaaaaagcgctgttaaaagcgggcggctgctatgcgaccatttttggcccggatggccgccagttacatgaaggcctggcggccggcgaagaaggcctgatttatgcggaaattgatctgggcctgattagcgtgagcaaagcggtgagcgatccggcgggtcattatagccgcccagatgtgacccagttaattattaacaaagcgccgcgccgtgcagttgttgatactgcaccaccatctgacccagcaccggcggtggaacgtccacatggtgcagatgaaccggcattaccggcgatggcggaactg

SEQ ID NO: 183 - protein, 344 aa, Synthetic construct
MNKPKFKAAAVQAAPVFLDLDKTIDKTIALMEDAARNGAELIAFPETWLPGYPWYAWLYAPAMWLPQYTQRYFDNSLEIGTPQARRIRKVAKDNNIVVSTGLSEREGGSLYISQWFIDRDGDTIAMRRKLKPTHVERTIFGEGDGSDLAVYDTRLGRVGGLCCWEHLQPLSKYAMYAQKEQVHVAAWPSFSLYEAYAYALGPEVNTSASRIYAVEGQCFVLAPCATVSKEMHDEMCTTDSKKALLKTGGCYAVIFGPDGRQLHEGLPAGEEGLIYAEIDLGTISVGKAVADPAGHYSRPDVTQLIHEKAPRRAVVDTAPPSDPAPAVERPHGADEVALPAMAEL

SEQ ID NO: 184 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgtatgcgccggcaatgtggttaccgcagtatacccagcgctattttgataacagcctggaaattggcaccccgcaggcgcgccgcattcgcaaagtggcgaaagataacaacattgtggtgagcaccggcctgagcgaacgcgaaggcggcagcctgtacatttctcagtggtttattgatcgcgatggcgataccattgcgatgcgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatgcatgatgagatgtgcaccaccgatagcaaaaaagcgctgttaaaaaccggcggctgctatgcggtgatttttggcccggatggccgccagttacatgaaggcctgccggccggcgaagaaggcctgatttatgcggaaattgatctgggcaccattagcgtgggcaaagcggtggcggatccggcgggtcattatagccgcccagatgtgacccagttaattcatgaaaaagcgccgcgccgtgcagttgttgatactgcaccaccatctgacccagcaccggcggtggaacgtccacatggtgcagatgaagtggcattaccggcgatggcggaactg

SEQ ID NO: 185 - protein, 344 aa, Synthetic construct
MNKPKFKAAAVQAAPVFLDLDKTIDKTIALMEKAARNGAELIAFPETWLPGYPWYAWLDAPAMWLPQYTPRYFDNSLEYGTPQAERIRKAAKDNNIVVGMGLSEREGESLYISQWFIDRDGDTIAKRRKLKPTHVERTIFGEGDGSDLAVHDTRLGRVGGLCCWEHLQPLSKYAMYAQNEQVHVAAWPSFSIYEGYAYALGPDVNTSASRIYAVEGQCFVLAPCACVSKAMVDEMCTTDLHKALLKTGGCYAVIFGPDGRQLSEGLPAGEEGLIYAEIDLGLISVSKAVNDPAGHYSRPDVTQLWHNKAPRRAVVDTALPSDPAPAVERPHGADEPALPAMAEL

SEQ ID NO: 186 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtataccccgcgctattttgataacagcctggaatatggcaccccgcaggcggaacgcattcgcaaagcggcgaaagataacaacattgtggtgggcatgggcctgagcgaacgcgaaggcgaaagcctgtacatttctcagtggtttattgatcgcgatggcgataccattgcgaaacgccgcaaactgaaaccgacccatgtggaacgcaccatttttggcgaaggcgatggcagcgatctggcggtgcatgatacccgtttaggtcgcgttggcggcttatgctgctgggaacatctgcagccgctgagcaaatatgcgatgtatgcgcagaacgaacaggtgcatgtggcggcgtggccgagctttagcatttatgaaggctatgcgtatgcgttaggcccggatgtgaacaccagcgcgagcagaatttatgcggtggaaggccagtgctttgtgctggcgccgtgcgcatgcgttagcaaagcgatggtggatgagatgtgcaccaccgatctgcataaagcgctgttaaaaaccggcggctgctatgcggtgatttttggcccggatggccgccagttaagcgaaggcctgccggccggcgaagaaggcctgatttatgcggaaattgatctgggcctgattagcgtgagcaaagcggtgaacgatccggcgggtcattatagccgcccagatgtgacccagttatggcataacaaagcgccgcgccgtgcagttgttgatactgcactgccatctgacccagcaccggcggtggaacgtccacatggtgcagatgaaccggcattaccggcgatggcggaactg

SEQ ID NO: 187 - protein, 344 aa, Synthetic construct
MNKPKFKAAAVQAAPVFLDLDKTIDKTIALMEDAARNGAELIAFPETWLPGYPWYAWLYAPAMWLPQYTQRYFDNSLEYETPQDHRIRKAAKDNNIVVSMGLSEREGSSLYISQWFIDRDGDTIAQRRKLKPTHVERTIFGEGDGSDLAVWDARLGRVGALCCWEHLQPLSKYAMYAQNEQVHVAAWPSFSLYKAYAYALGPIVNTHASRIYAVEGQCFVLAPCATVSKDMVDEMCTTDGQKALLKTGGGYAVIFGPDGRELHEGLPTGEEGLIYAEIDLGLISVAKAVGDPAGHYSRPDVTQLIHDKAPRRAVVDTAPPSDPAPAVERPHGADEPALPAMAEL

SEQ ID NO: 188 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgtatgcgccggcaatgtggttaccgcagtatacccagcgctattttgataacagcctggaatatgaaaccccgcaggatcatcgcattcgcaaagcggcgaaagataacaacattgtggtgagcatgggcctgagcgaacgcgaaggcagcagcctgtacatttctcagtggtttattgatcgcgatggcgataccattgcgcagcgccgcaaactgaaaccgacccatgtggaacgcaccatttttggcgaaggcgatggcagcgatctggcggtgtgggatgcgcgtttaggtcgcgttggcgcgttatgctgctgggaacatctgcagccgctgagcaaatatgcgatgtatgcgcagaacgaacaggtgcatgtggcggcgtggccgagctttagcctgtataaagcgtatgcgtatgcgttaggcccgattgtgaacacccatgcgagcagaatttatgcggtggaaggccagtgctttgtgctggcgccgtgcgcaactgttagcaaagatatggtggatgagatgtgcaccaccgatggccagaaagcgctgttaaaaaccggcggcggctatgcggtgatttttggcccggatggccgcgaattacatgaaggcctgccgaccggcgaagaaggcctgatttatgcggaaattgatctgggcctgattagcgtggcgaaagcggtgggcgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 189 - protein, 344 aa, Synthetic construct
MNKPKFKAAAVQAAPVFLDLDKTIDKTVALMEDAARNGAELIAFPETWLPGYPWYAWLNAPAMWLPQYTQRYFDNSLEYGTPQAERIRKAAKDHNIVVAMGLSEREGGSLYISQWFIDRDGDTIAQRRKLKPTHVERTIFGEGDGSDLAVYDTRLGRVGGLCCWEHLQPLSKYAMYAQDEQVHVAAWPSFSIYEAYAYALGPIVNTSASRIYAVEGQCFVLAPCATVSKDMVDEMCTTDDRKALLKTGGGYATIFGPDGRELHEGLGAGEEGLIYAEIDLGLISVSKAVNDPAGHYSRPDVTQLIHEKAPRRAVVDQAPPSDNAPAVERPHGADEPALPAMAEL

SEQ ID NO: 190 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaagatgcggcgcgcaacggcgcggaactgattgcgtttccggaaacctggttaccgggctatccgtggtatgcgtggctgaacgcgccggcaatgtggttaccgcagtatacccagcgctattttgataacagcctggaatatggcaccccgcaggcggaacgcattcgcaaagcggcgaaagatcataacattgtggtggcgatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcaggatgaacaggtgcatgtggcggcgtggccgagctttagcatttatgaagcgtatgcgtatgcgttaggcccgattgtgaacaccagcgcgagcagaatttatgcggtggaaggccagtgctttgtgctggcgccgtgcgcaactgttagcaaagatatggtggatgagatgtgcaccaccgatgatcgcaaagcgctgttaaaaaccggcggcggctatgcgaccatttttggcccggatggccgcgaattacatgaaggcctgggcgccggcgaagaaggcctgatttatgcggaaattgatctgggcctgattagcgtgagcaaagcggtgaacgatccggcgggtcattatagccgcccagatgtgacccagttaattcatgaaaaagcgccgcgccgtgcagttgttgatcaggcaccaccatctgacaacgcaccggcggtggaacgtccacatggtgcagatgaaccggcattaccggcgatggcggaactg

SEQ ID NO: 191 - protein, 344 aa, Synthetic construct
MNKPKFKAAAVQAAPVFLDLDKTIDKTVALMEDAARNGAELIAFPETWLPGYPWYAWLDAPAMWVPQYTQRYFDNSLEYGTPQARRIRKVAKDNNIVVSMGLSEREGGSLYISQWFIDRDGDTIAQRRKLKPTHVERTIFGEGDGSDLAVWDIRLGRVGGLCCWEHLQPLSKYAMYAQNEQVHVAAWPSFSMYRAYAYALGHDVNTSASRIYAVEGQCFVLAPCATVSKEMLDEMCTTDGQKALLKTGGCYARIYGPDGRELHEGLPAGEEGLLYAEIDLGLISVAKAVTDPAGHYSRPDVTQLIHNKAPRRAVVDTAPPSDPAPAVERPHGADEPALPAMAEL

SEQ ID NO: 192 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaagatgcggcgcgcaacggcgcggaactgattgcgtttccggaaacctggttaccgggctatccgtggtatgcgtggctggatgcgccggcaatgtgggtgccgcagtatacccagcgctattttgataacagcctggaatatggcaccccgcaggcgcgccgcattcgcaaagtggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaacgaacaggtgcatgtggcggcgtggccgagctttagcatgtatcgcgcgtatgcgtatgcgttaggccatgatgtgaacaccagcgcgagcagaatttatgcggtggaaggccagtgctttgtgctggcgccgtgcgcaactgttagcaaagaaatgctggatgagatgtgcaccaccgatggccagaaagcgctgttaaaaaccggcggctgctatgcgcgcatttatggcccggatggccgcgaattacatgaaggcctgccggccggcgaagaaggcctgctgtatgcggaaattgatctgggcctgattagcgtggcgaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 193 - protein, 344 aa, Synthetic construct
MNKPKFKAAAVQAAPVFLDLDKTIDKTIALMEDAARNGAELIAFPETWLPGYPWYAWLDVPAMWLPVYTDRYFDNSLEYGSPQADRIRKVAKDNNIWVSMGLSEREGGSLYISQWFIDRDGDTIARRRKLKPTHVERTIFGEGDGSDLAVYDTRLGRVGGLCCWEHLQPLSKYAMYAQTEQVHVAAWPSFSLYEAYAHALGPIVNTSASRIYAVEGQCFVLAPCATVSKDMVDEMCTTDLQKALLHTGGCYARIFGPDGRQLHEGLPAGEEGLIYAEIDLGLISVSKAVADPAGHYSRPDVTQLIHNKAPRRAVSDTAPPSDPAPAVERPHGADEPALPAMAEL

SEQ ID NO: 194 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgtgccggcaatgtggttaccggtgtataccgatcgctattttgataacagcctggaatatggcagcccgcaggcggatcgcattcgcaaagtggcgaaagataacaacatttgggtgagcatgggcctgagcgaacgcgaaggcggcagcctgtacatttctcagtggtttattgatcgcgatggcgataccattgcgcgccgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagaccgaacaggtgcatgtggcggcgtggccgagctttagcctgtatgaagcgtatgcgcatgcgttaggcccgattgtgaacaccagcgcgagcagaatttatgcggtggaaggccagtgctttgtgctggcgccgtgcgcaactgttagcaaagatatggtggatgagatgtgcaccaccgatctgcagaaagcgctgttacataccggcggctgctatgcgcgcatttttggcccggatggccgccagttacatgaaggcctgccggccggcgaagaaggcctgatttatgcggaaattgatctgggcctgattagcgtgagcaaagcggtggcggatccggcgggtcattatagccgcccagatgtgacccagttaattcataacaaagcgccgcgccgtgcagttagcgatactgcaccaccatctgacccagcaccggcggtggaacgtccacatggtgcagatgaaccggcattaccggcgatggcggaactg

SEQ ID NO: 195 - protein, 344 aa, Synthetic construct
MNKPKFKAAAVQAAPVFLDLDKGIDKTIALMEDAARNGAELIAFPETWLPGYPWYAWLDAPAMWLPMYTQRYFDNSLEFGTPQAERIRKSAKENNIVVSMGLSEREGGSLYISQWFIDRDGDTIARRRKLKPTHVERTIFGEGDGSDLAVWDTRLGRLGGLCCWEHLQPLSKYAMYAQTEQVHVAAWPSFSLYEAYAYALGPDVNTSASRIYAVEGQCFVLAPCASVSKEMVDEMCTTDLKKALLKTGGAYAVIYGPDGRPLHEGLPAGEEGLIYAEIDLGLISVSKAVNDPAGHYSRPDVTQLIHNKAPRRAVVDTAPPSDPAPAVERPHGAEEPALPAMAEL

SEQ ID NO: 196 - DNA, 1032 bp, Synthetic construct
  source 1..1032: mol_type = other DNA
atgaacaaaccgaaatttaaagcggcggcggtgcaggcggcgccggtgtttctggatttagacaaaggcattgataagaccattgcgctgatggaagatgcggcgcgcaacggcgcggaactgattgcgtttccggaaacctggttaccgggctatccgtggtatgcgtggctggatgcgccggcaatgtggttaccgatgtatacccagcgctattttgataacagcctggaatttggcaccccgcaggcggaacgcattcgcaaaagcgcgaaagaaaacaacattgtggtgagcatgggcctgagcgaacgcgaaggcggcagcctgtacatttctcagtggtttattgatcgcgatggcgataccattgcgcgccgccgcaaactgaaaccgacccatgtggaacgcaccatttttggcgaaggcgatggcagcgatctggcggtgtgggatacccgtttaggtcgcctgggcggcttatgctgctgggaacatctgcagccgctgagcaaatatgcgatgtatgcgcagaccgaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcaagcgttagcaaagaaatggtggatgagatgtgcaccaccgatctgaaaaaagcgctgttaaaaaccggcggcgcgtatgcggtgatttatggcccggatggccgcccgttacatgaaggcctgccggccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacccagcaccggcggtggaacgtccacatggtgcagaagaaccggcattaccggcgatggcggaactg

SEQ ID NO: 197 - protein, 344 aa, Synthetic construct
MNKPKFKAAAVQAAPVFLDLDKTIDKTIALVEDAARNGAELIAFPETWLPGYPWYAWLDAPAMWVPQYTQRYFDNSLEFGTPQAERIRKAAKDNNIVVSMGLSEREGESLYISQWFIDRDGDTQARRRKLKPTHVERTIFGEGDGSDLAVADIRLGRVGGLCCWEHLQPLSKYAMYAQNEQVHVAAWPSFSLYGAYAYALGPIVNTSASRMYAVEGQCFVLAPCAIVSKDMVDEMCTTDGQKALLKTGGGYAAIFGPDGRQLHEGLPAGEEGLIYAEIDLGAISVSKAVGDPAGHYSRPDVTQLIHNKAPRRAVVDTAPPSDPAPAVERPHGADEPALPAMAEL

SEQ ID NO: 198 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctggtggaagatgcggcgcgcaacggcgcggaactgattgcgtttccggaaacctggttaccgggctatccgtggtatgcgtggctggatgcgccggcaatgtgggtgccgcagtatacccagcgctattttgataacagcctggaatttggcaccccgcaggcggaacgcattcgcaaagcggcgaaagataacaacattgtggtgagcatgggcctgagcgaacgcgaaggcgaaagcctgtacatttctcagtggtttattgatcgcgatggcgatacccaggcgcgccgccgcaaactgaaaccgacccatgtggaacgcaccatttttggcgaaggcgatggcagcgatctggcggtggcggatattcgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgtatgcgttaggcccgattgtgaacaccagcgcgagcagaatgtatgcggtggaaggccagtgctttgtgctggcgccgtgcgcaattgttagcaaagatatggtggatgagatgtgcaccaccgatggccagaaagcgctgttaaaaaccggcggcggctatgcggcgatttttggcccggatggccgccagttacatgaaggcctgccggccggcgaagaaggcctgatttatgcggaaattgatctgggcgcgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 199 - protein, 344 aa, Synthetic construct
MNKPKFKAAAVQAAPVFLDLDKTIDKTVALMEDAARNGAELIAFPETWLPGYPWYAWLDAPAMWVPQYTQRYFDNSLEYGTPQADRIRKAAKQNNIVVAMGLSEREGGSLYISQWFIDRDGDTIAKRRKLKPTHVERTIFGEGDGSDLAVWDTRLGRVGGLCCWEHLQPLSKYAMYAQNEQVHVAAWPSFSLYRAYAYALGPDVNTSASRIYAVEGQCFVLAPCAVVSKDMVDEMCTTDLQKALLKPGGGYATIFGPDGRALHEGLPAGEEGLIYAEIDLGLISVSKAVNDPAGHYSRPDVTQLIHEKAPRRAVVDTATPSDPAPPVERPHGADEPALPAMAEL

SEQ ID NO: 200 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgtggcgctgatggaagatgcggcgcgcaacggcgcggaactgattgcgtttccggaaacctggttaccgggctatccgtggtatgcgtggctggatgcgccggcaatgtgggtgccgcagtatacccagcgctattttgataacagcctggaatatggcaccccgcaggcggatcgcattcgcaaagcggcgaaacagaacaacattgtggtggcgatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatcgcgcgtatgcgtatgcgttaggcccggatgtgaacaccagcgcgagcagaatttatgcggtggaaggccagtgctttgtgctggcgccgtgcgcagtggttagcaaagatatggtggatgagatgtgcaccaccgatctgcagaaagcgctgttaaaaccgggcggcggctatgcgaccatttttggcccggatggccgcgcgttacatgaaggcctgccggccggcgaagaaggcctgatttatgcggaaattgatctgggcctgattagcgtgagcaaagcggtgaacgatccggcgggtcattatagccgcccagatgtgacccagttaattcatgaaaaagcgccgcgccgtgcagttgttgatactgcaaccccatctgacccagcaccgccggtggaacgtccacatggtgcagatgaaccggcattaccggcgatggcggaactg

SEQ ID NO: 201 - protein, 344 aa, Synthetic construct
MNKPKFKAAAVQAAPVFLDLDKTIDKTIALMERAARNGAELIAFPETWLPGYPWYAWLYAPAMWLPQYTQRYFDNSLEYGTPQAERIRKDAKDNNIWVSMGLSEREGGSLYISQWFIDRDGDTLAMRRKLKPTHVERTIFGEGDGSDLAVWDTRLGRVGGLCCWEHLQPLSKYAMYAQNEQVHVAAWPSFSMYEAYAYALGPEVNTSASRIYAVEGQCFVLAPCATVSKSMVDEMCTTPEHKALLKTGGVYASIFGPDGRQLHEGLPASEEGLIYAEIDLGLISVSKAVADPAGHYSRPDVTQLIVNKAPRRAVVDTAPPSDPAPAVERPHGKDEPALPAMAEL

SEQ ID NO: 202 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctggttaccgggctatccgtggtatgcgtggctgtatgcgccggcaatgtggttaccgcagtatacccagcgctattttgataacagcctggaatatggcaccccgcaggcggaacgcattcgcaaagatgcgaaagataacaacatttgggtgagcatgggcctgagcgaacgcgaaggcggcagcctgtacatttctcagtggtttattgatcgcgatggcgataccctggcgatgcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcatgtatgaagcgtatgcgtatgcgttaggcccggaagtgaacaccagcgcgagcagaatttatgcggtggaaggccagtgctttgtgctggcgccgtgcgcaactgttagcaaaagcatggtggatgagatgtgcaccaccccggaacataaagcgctgttaaaaaccggcggcgtgtatgcgagcatttttggcccggatggccgccagttacatgaaggcctgccggccagcgaagaaggcctgatttatgcggaaattgatctgggcctgattagcgtgagcaaagcggtggcggatccggcgggtcattatagccgcccagatgtgacccagttaattgtgaacaaagcgccgcgccgtgcagttgttgatactgcaccaccatctgacccagcaccggcggtggaacgtccacatggtaaagatgaaccggcattaccggcgatggcggaactg

SEQ ID NO: 203 - protein, 344 aa, Synthetic construct
MNKPKFKAAAVQAAPVYLDLDKTIDKTIALMEDAARNGAELIAFPETWLPGYPWYAWLTAPAMWGPQYFQRYFDNSLEYNTPQAERIRKDAKDNNITVGMGLSEREGGSLYISQWFIDRDGDTIAKRRKLKPTHVERTIFGEGDGSDLAVWDARLGRVGGLCCWEHLQPLSKYAMYAQDEQVHVAAWPSFSLYEAYAYALGPDVNTSASRIYAVEGQCFVLAPCATVSKEMVDEMCTTDAHKALLKTGGAYARIFGPDGRQLHEGLPAGEEGLIYAEIDLGLISVSKAVNDPAGHYSRPDVTQLIHNKAPRRAVVDTAPPSDPAPAVERPHGTDEPALPAMAEL

SEQ ID NO: 204 - DNA, 1032 bp, Synthetic construct
  source 1..1032: mol_type = other DNA
atgaacaaaccgaaatttaaagcggcggcggtgcaggcggcgccggtgtatctggatttagacaaaaccattgataagaccattgcgctgatggaagatgcggcgcgcaacggcgcggaactgattgcgtttccggaaacctggttaccgggctatccgtggtatgcgtggctgaccgcgccggcaatgtggggcccgcagtattttcagcgctattttgataacagcctggaatataacaccccgcaggcggaacgcattcgcaaagatgcgaaagataacaacattaccgtgggcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtgggatgcgcgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccggatgtgaacaccagcgcgagcagaatttatgcggtggaaggccagtgctttgtgctggcgccgtgcgcaactgttagcaaagaaatggtggatgagatgtgcaccaccgatgcgcataaagcgctgttaaaaaccggcggcgcgtatgcgcgcatttttggcccggatggccgccagttacatgaaggcctgccggccggcgaagaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacccagcaccggcggtggaacgtccacatggtaccgatgaaccggcattaccggcgatggcggaactg

SEQ ID NO: 205 - protein, 344 aa, Synthetic construct
MNKPKFKAAAVQAAPVFLDLDKTIDKTIALMERAARNGAELIAFPETFLPGYPWYAWLDAPAMWLPQYTPRYFDNSLEYGTPQADRIRKAAKDNNIVMSMGLSEREGGSLYISQWIIDRDGDTIAQRRKLKPTHVERTIFGEGDGSDLAVVDTRLGRVGGLCCWEHLQPLSKYAMYAQTEQVHVAAWPSFSLYEAYAYALGPIVNTSASRIYAVEGQCFVLAPCASVSKEMVDEMCTTDGRKALLKTGGGYAAIFGPDGRLLHEGLPAGEEGLIYAEIDLGLISVSKAVADPAGHYSRPDVTQLIHNKKPRRAVVDTAPPSDPAPAVERPHGPDEPALPAMAEL

SEQ ID NO: 206 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctttttaccgggctatccgtggtatgcgtggctggatgcgccggcaatgtggttaccgcagtataccccgcgctattttgataacagcctggaatatggcaccccgcaggcggatcgcattcgcaaagcggcgaaagataacaacattgtgatgagcatgggcctgagcgaacgcgaaggcggcagcctgtacatttctcagtggattattgatcgcgatggcgataccattgcgcagcgccgcaaactgaaaccgacccatgtggaacgcaccatttttggcgaaggcgatggcagcgatctggcggtggtggatacccgtttaggtcgcgttggcggcttatgctgctgggaacatctgcagccgctgagcaaatatgcgatgtatgcgcagaccgaacaggtgcatgtggcggcgtggccgagctttagcctgtatgaagcgtatgcgtatgcgttaggcccgattgtgaacaccagcgcgagcagaatttatgcggtggaaggccagtgctttgtgctggcgccgtgcgcaagcgttagcaaagaaatggtggatgagatgtgcaccaccgatggccgcaaagcgctgttaaaaaccggcggcggctatgcggcgatttttggcccggatggccgcctgttacatgaaggcctgccggccggcgaagaaggcctgatttatgcggaaattgatctgggcctgattagcgtgagcaaagcggtggcggatccggcgggtcattatagccgcccagatgtgacccagttaattcataacaaaaaaccgcgccgtgcagttgttgatactgcaccaccatctgacccagcaccggcggtggaacgtccacatggtccggatgaaccggcattaccggcgatggcggaactg

SEQ ID NO: 207 - protein, 344 aa, Synthetic construct
MNKPKFKAAAVQAAPVFLDLDKSIDKTVALMEDAARNGAELIAFPETWLPGYPWYAWLDAPAMWLPQYTQRYFDNSLEYGTPQFERIRKVAKDNNIVLSMGLSEREGGSLYISQWFIDRDGDTIAKRRKLKPTHVERTIFGEGDGSDLAVWDPRLGRLGGLCCWEHLQPLSKYAMYAQNEQVHVAAWPSFSAYGAYAYALGPEVNTSASRIYAVEGQCFVLAPCATVSKEMVDEMCTTDVQKALLKTGGCYAGIYGPDGRQLHEGLPAEEEGLIYAEIDLGLISVSKAVADPAGHYSRPDVTQLIHHKAPRRAVVDTAPPSDPAPAVERPHGADEPALPAMAEL

SEQ ID NO: 208 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagaccgtggcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcagtttgaacgcattcgcaaagtggcgaaagataacaacattgtgctgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgtgggatccgcgtttaggtcgcctgggcggcttatgctgctgggaacatctgcagccgctgagcaaatatgcgatgtatgcgcagaacgaacaggtgcatgtggcggcgtggccgagctttagcgcgtatggcgcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatgtgcagaaagcgctgttaaaaaccggcggctgctatgcgggcatttatggcccggatggccgccagttacatgaaggcctgccggccgaagaagaaggcctgatttatgcggaaattgatctgggcctgattagcgtgagcaaagcggtggcggatccggcgggtcattatagccgcccagatgtgacccagttaattcatcataaagcgccgcgccgtgcagttgttgatactgcaccaccatctgacccagcaccggcggtggaacgtccacatggtgcagatgaaccggcattaccggcgatggcggaactg

SEQ ID NO: 209 - protein, 344 aa, Synthetic construct
MNKPKFKAAAVQAAPVFLDLDKTIDKTIALAEDAARNGAELIAFPETWLPGYPWYAWLDAPAMWMPQYTHRYFDNSLEYGTPQAKRIRKSAKDNNIVVSMGLSEREGGSLYISQWFIDRDGDTIAQRRKLKPTHVERTIFGEGDGSDLAVADTRLGRVGGLCCWEHLQPLSKYAMYAQDEQVHVAAWPSFSLYEAYAYALGPEVNTHASRIYAVEGQCFVLAPCATVSKGMIDEMCTTDVRKALLKEGGVYASIFGPDGRQLHEGLPAGTEGLIYAEIDLGLISVSKAVTDPAGHYSRPDVTQLIHNKAPRRAVVDTAPPSDPAPAVERPHGADEPALPAMAEL

SEQ ID NO: 210 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctggcggaagatgcggcgcgcaacggcgcggaactgattgcgtttccggaaacctggttaccgggctatccgtggtatgcgtggctggatgcgccggcaatgtggatgccgcagtatacccatcgctattttgataacagcctggaatatggcaccccgcaggcgaaacgcattcgcaaaagcgcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtggcggatacccgtttaggtcgcgttggcggcttatgctgctgggaacatctgcagccgctgagcaaatatgcgatgtatgcgcaggatgaacaggtgcatgtggcggcgtggccgagctttagcctgtatgaagcgtatgcgtatgcgttaggcccggaagtgaacacccatgcgagcagaatttatgcggtggaaggccagtgctttgtgctggcgccgtgcgcaactgttagcaaaggcatgattgatgagatgtgcaccaccgatgtgcgcaaagcgctgttaaaagaaggcggcgtgtatgcgagcatttttggcccggatggccgccagttacatgaaggcctgccggccggcaccgaaggcctgatttatgcggaaattgatctgggcctgattagcgtgagcaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 211 - protein, 344 aa, Synthetic construct
MNKPKFKAAAVQAAPVFLDLDKTIDKTIALMEDAARNGAELIAFPETWLPGYPWYAWLDAPAMWLPVYTQRYFDNSLEYETPQFERIRKSAKDNNIVASMGLSEREGASLYISQWFIDRDGDTIAQRRKLKPTHVERTIFGEGDGSDLAVIDARLGRVGGLCCWEHLQPLSKYAMYAQKEQVHVAAWPSFSLYEAYAYALGPEVNTSASRIYAVEGQCFVLAPCASVSKEMVDEMCTTDLHKALLKTGGCYARIYGPDGRELHEGLPAGEEGLIYAEIDLGLISVSKAVGDPAGHYSRPDVTQLIHNKAPRRAVVDTAPPSDPAPEVERPHGADEPALPAMAEL

SEQ ID NO: 212 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccggtgtatacccagcgctattttgataacagcctggaatatgaaaccccgcagtttgaacgcattcgcaaaagcgcgaaagataacaacattgtggcgagcatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgattgatgcgcgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaagcgttagcaaagaaatggtggatgagatgtgcaccaccgatctgcataaagcgctgttaaaaaccggcggctgctatgcgcgcatttatggcccggatggccgcgaattacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggaagtggaacgtccacatggtgcagatgaaccggcattaccggcgatggcggaactg

SEQ ID NO: 213 - protein, 344 aa, Synthetic construct
MNKPKFKAAAVQAAPVFLDLDKTIDKTRALMEDAARNGAELIAFPETWFPGYPWYAWLDAPAMWLPMYTQRYFDNSLEYGSPQAARIRKDAKDNNIVVSMGLSEREGGSLYISQWFIDRDGDTIAMRRKLKPTHVERTIFGEGDGSDLAVWDPRLGRVGGLCCWEHLQPLSKYAMYAQKEQVHVAAWPSFSLYVAYAYALGPEVNTSASRIYAVEGQCFVLAPCATVSKEMVDEMCTTDLQKALLKTGGCAASIFGPDGRALHEGLPPGEEGLLYAEIDLGLISVSKAVGDPAGHYSRPDVTQLIHNKAPRRAVVDTAPPSDPAPAVERPHGADEPALPAMAEL

SEQ ID NO: 214 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagatgcggcgcgcaacggcgcggaactgattgcgtttccggaaacctggtttccgggctatccgtggtatgcgtggctggatgcgccggcaatgtggttaccgatgtatacccagcgctattttgataacagcctggaatatggcagcccgcaggcggcgcgcattcgcaaagatgcgaaagataacaacattgtggtgagcatgggcctgagcgaacgcgaaggcggcagcctgtacatttctcagtggtttattgatcgcgatggcgataccattgcgatgcgccgcaaactgaaaccgacccatgtggaacgcaccatttttggcgaaggcgatggcagcgatctggcggtgtgggatccgcgtttaggtcgcgttggcggcttatgctgctgggaacatctgcagccgctgagcaaatatgcgatgtatgcgcagaaagaacaggtgcatgtggcggcgtggccgagctttagcctgtatgtggcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggctgcgcggcgagcatttttggcccggatggccgcgcgttacatgaaggcctgccgccgggcgaagaaggcctgctgtatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 215 - protein, 344 aa, Synthetic construct
MNKPKFKAAAVQAAPVFLDLDKTIDKTIALMERAARNGAELIAFPETWLPGYPWYAWLDAPAMWVPQYTQRYFDNSLEYGTPQAERIRKAAKDNNIVVVMGLSEREGESLYISQWFIDRDGDTIAQRRKLKPTHVERTIFGEGDGSDLAVWDTRLGRVGGLCCWEHLQPLSKYAMYAQTEQVHVAAWPSFSAYRAYAYALGPEVNTSASRIYAVEGQCFVLAPCATVSKAMQDEMCTTDVQKALLKTGGVYATIFGPDGRQLHEGLPAGEEGLIYAEIDLGLIGVSKAVGDPAGHYSRPDVTQLIHHKAPRRAVVDTAQPSDPAPPVERPHGADEPALPAMAEL

SEQ ID NO: 216 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctggttaccgggctatccgtggtatgcgtggctggatgcgccggcaatgtgggtgccgcagtatacccagcgctattttgataacagcctggaatatggcaccccgcaggcggaacgcattcgcaaagcggcgaaagataacaacattgtggtggtgatgggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaccgaacaggtgcatgtggcggcgtggccgagctttagcgcgtatcgcgcgtatgcgtatgcgttaggcccggaagtgaacaccagcgcgagcagaatttatgcggtggaaggccagtgctttgtgctggcgccgtgcgcaactgttagcaaagcgatgcaggatgagatgtgcaccaccgatgtgcagaaagcgctgttaaaaaccggcggcgtgtatgcgaccatttttggcccggatggccgccagttacatgaaggcctgccggccggcgaagaaggcctgatttatgcggaaattgatctgggcctgattggcgtgagcaaagcggtgggcgatccggcgggtcattatagccgcccagatgtgacccagttaattcatcataaagcgccgcgccgtgcagttgttgatactgcacagccatctgacccagcaccgccggtggaacgtccacatggtgcagatgaaccggcattaccggcgatggcggaactg

SEQ ID NO: 217 - protein, 344 aa, Synthetic construct
MNKPKFKAAAVQAAPVFLDLDKTIDKTIALMEDAARNGAELIAFPETWLPGYPWYAWLNAPAMWLPQYTQRYFDNSLEIGTPQAERIRKAAKQNNIVVSMGLSEREGGSLYIAQWFIDRDGDTIAQRRKLKPTHVERTIFGEGDGSDLAVHDPRLGRVGGLCCWEHVQPLSKYAMYAQNEQVHVAAWPSFSMYDAYAYALGPEVNTSASRIYAVEGQCFVLAPCACVSKEMVDEMCTTDLHKALLKTGGGYARIYGPDGRPLVEGLPAGEEGLIYAEIDLGLISVSKAVNDPAGHYSRPDVTQLIHDKAPRRAVVDTAPPSDPAPAVERPHGADEPALPAMAEL

SEQ ID NO: 218 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctgaacgcgccggcaatgtggttaccgcagtatacccagcgctattttgataacagcctggaaattggcaccccgcaggcggaacgcattcgcaaagcggcgaaacagaacaacattgtggtgagcatgggcctgagcgaacgcgaaggcggcagcctgtacattgcgcagtggtttattgatcgcgatggcgataccattgcgcagcgccgcaaactgaaaccgacccatgtggaacgcaccatttttggcgaaggcgatggcagcgatctggcggtgcatgatccgcgtttaggtcgcgttggcggcttatgctgctgggaacatgtgcagccgctgagcaaatatgcgatgtatgcgcagaacgaacaggtgcatgtggcggcgtggccgagctttagcatgtatgatgcgtatgcgtatgcgttaggcccggaagtgaacaccagcgcgagcagaatttatgcggtggaaggccagtgctttgtgctggcgccgtgcgcatgcgttagcaaagaaatggtggatgagatgtgcaccaccgatctgcataaagcgctgttaaaaaccggcggcggctatgcgcgcatttatggcccggatggccgcccgttagtggaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 219 - protein, 344 aa, Synthetic construct
MNKPKFKAAAVQAAPVFLDLDKTIDKTIALMERAARNGAELIAFPETWLPGYPWYAWLDAPAMWLPQYTPRYFDNSLEYGTPQAARIRKAAKDNNIVVSMGLSEREGGSLYISQWFIDRDGDTIAQRRKLKPTHVERTIFGEGDGSDLAVADLRLGRVGGLCCWEHIQPLSKYAMYAQNEQVHVAAWPSFSIYEAYAYALGPEVNTSASRIYAVEGTCFVLAPCAVVSKGMVDEMCTTDLQKALLKTGGGYAGIFGPDGRSLHEGLPANREGLIYAEIDLGLISVSKAVNDPAGHYSRPDVTQLIHNKAPRRAVVDTAPPSDPAPGVERPHGADEPALPAMAEL

SEQ ID NO: 220 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaacgcgcggcgcgcaacggcgcggaactgattgcgtttccggaaacctggttaccgggctatccgtggtatgcgtggctggatgcgccggcaatgtggttaccgcagtataccccgcgctattttgataacagcctggaatatggcaccccgcaggcggcgcgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtggcggatctgcgtttaggtcgcgttggcggcttatgctgctgggaacatattcagccgctgagcaaatatgcgatgtatgcgcagaacgaacaggtgcatgtggcggcgtggccgagctttagcatttatgaagcgtatgcgtatgcgttaggcccggaagtgaacaccagcgcgagcagaatttatgcggtggaaggcacctgctttgtgctggcgccgtgcgcagtggttagcaaaggcatggtggatgagatgtgcaccaccgatctgcagaaagcgctgttaaaaaccggcggcggctatgcgggcatttttggcccggatggccgcagcttacatgaaggcctgccggccaaccgcgaaggcctgatttatgcggaaattgatctgggcctgattagcgtgagcaaagcggtgaacgatccggcgggtcattatagccgcccagatgtgacccagttaattcataacaaagcgccgcgccgtgcagttgttgatactgcaccaccatctgacccagcaccgggcgtggaacgtccacatggtgcagatgaaccggcattaccggcgatggcggaactg

SEQ ID NO: 221 - protein, 344 aa, Synthetic construct
MNKPKFKAAAVQAAPVFLDLDKTIDKTRALMEDAARNGAELIAFPETWLPGYPWYAWLDAPAMWLPGYTQRYFDNSLEYGTPQADRIRKAAKENNIHVSMGLSEREGGSLYISQWFIDRDGDTIAQRRKLKPTHVERTIFGEGDGSDLAVWDTRLGRVGGLCCWEHFQPLSKYAMYAQNEQVHVAAWPSFSLYVAYAFALGPDVNTSASRIYAVEGQCFVLAPCATVSKSMVDEMCITDVKKALLKTGGGYATIFGPDGRPLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 222 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagatgcggcgcgcaacggcgcggaactgattgcgtttccggaaacctggttaccgggctatccgtggtatgcgtggctggatgcgccggcaatgtggttaccgggctatacccagcgctattttgataacagcctggaatatggcaccccgcaggcggatcgcattcgcaaagcggcgaaagaaaacaacattcatgtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacattttcagccgctgagcaaatatgcgatgtatgcgcagaacgaacaggtgcatgtggcggcgtggccgagctttagcctgtatgtggcgtatgcgtttgcgttaggcccggatgtgaacaccagcgcgagcagaatttatgcggtggaaggccagtgctttgtgctggcgccgtgcgcaactgttagcaaaagcatggtggatgagatgtgcattaccgatgtgaaaaaagcgctgttaaaaaccggcggcggctatgcgaccatttttggcccggatggccgcccgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 223 - protein, 344 aa, Synthetic construct
MNKPKFKAAAVQAAPVFLDLDKTIDKTRALMEDAARNGAELIAFPETWLPGYPWYAWLDAPAMWLPAYTQRYFDNSLEYGTPQAERIRKDAKDNNIVVSMGLSEREGESLYISQWIIDRDGDTIAQRRKLKPTHVERTIFGEGDGSDLAVWDTRLGRVGGLCCWEHLQPLSKYAMYAQNEQVHVAAWPSFSVYDAYANALGPEVNTSASRIYAVEGQCFVLAPCAIVSKSMVDEMCTTDLHKALLKAGGGYAVIFGPDGRQLHEGLAAGEEGLIYAEIDLGLISVSKAVADPAGHYSRPDVTQLIHNKAPRRAVVDTAPPSDPAPAVERPHGADEVALPAMAEL

SEQ ID NO: 224 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagatgcggcgcgcaacggcgcggaactgattgcgtttccggaaacctggttaccgggctatccgtggtatgcgtggctggatgcgccggcaatgtggttaccggcgtatacccagcgctattttgataacagcctggaatatggcaccccgcaggcggaacgcattcgcaaagatgcgaaagataacaacattgtggtgagcatgggcctgagcgaacgcgaaggcgaaagcctgtacatttctcagtggattattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcgtgtatgatgcgtatgcgaacgcgttaggcccggaagtgaacaccagcgcgagcagaatttatgcggtggaaggccagtgctttgtgctggcgccgtgcgcaattgttagcaaaagcatggtggatgagatgtgcaccaccgatctgcataaagcgctgttaaaagcgggcggcggctatgcggtgatttttggcccggatggccgccagttacatgaaggcctggcggccggcgaagaaggcctgatttatgcggaaattgatctgggcctgattagcgtgagcaaagcggtggcggatccggcgggtcattatagccgcccagatgtgacccagttaattcataacaaagcgccgcgccgtgcagttgttgatactgcaccaccatctgacccagcaccggcggtggaacgtccacatggtgcagatgaagtggcattaccggcgatggcggaactg

SEQ ID NO: 225 - protein, 344 aa, Synthetic construct
MNKPKFKAAAVQAAPVWLDLDKTIDKTRALMEEAARNGAELIAFPETWLPGYPWYAWLDSPAMWLPQYTQRYFDNSLEYGTPQAARIRKAAKDNNIVVSMGLSEREGESLYISQWFIDRDGDTIAKRRKLKPTHVERTIFGEGDGSDLAVWDIRLGRVGGLCCWEHLQPLSKYAMYAQTEQVHVAAWPSFSVYEAYAYALGPEVNTSASRIYAVEGQCFVLAPCAIVSKAMVDEMCTTDLQKALLKAGGGYARIFGPDGRLLHEGLPAGEEGLIYAEIDLGLISVSKAVGDPAGHYSRPDVTQLILNKAPRRAVVDTAPPSDPAPAVERPHGADEEALPAMAEL

SEQ ID NO: 226 - DNA, 1032 bp, Synthetic construct
  source 1..1032: mol_type = other DNA
atgaacaaaccgaaatttaaagcggcggcggtgcaggcggcgccggtgtggctggatttagacaaaaccattgataagacccgcgcgctgatggaagaagcggcgcgcaacggcgcggaactgattgcgtttccggaaacctggttaccgggctatccgtggtatgcgtggctggatagcccggcaatgtggttaccgcagtatacccagcgctattttgataacagcctggaatatggcaccccgcaggcggcgcgcattcgcaaagcggcgaaagataacaacattgtggtgagcatgggcctgagcgaacgcgaaggcgaaagcctgtacatttctcagtggtttattgatcgcgatggcgataccattgcgaaacgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaccgaacaggtgcatgtggcggcgtggccgagctttagcgtgtatgaagcgtatgcgtatgcgttaggcccggaagtgaacaccagcgcgagcagaatttatgcggtggaaggccagtgctttgtgctggcgccgtgcgcaattgttagcaaagcgatggtggatgagatgtgcaccaccgatctgcagaaagcgctgttaaaagcgggcggcggctatgcgcgcatttttggcccggatggccgcctgttacatgaaggcctgccggccggcgaagaaggcctgatttatgcggaaattgatctgggcctgattagcgtgagcaaagcggtgggcgatccggcgggtcattatagccgcccagatgtgacccagttaattctgaacaaagcgccgcgccgtgcagttgttgatactgcaccaccatctgacccagcaccggcggtggaacgtccacatggtgcagatgaagaagcattaccggcgatggcggaactg

SEQ ID NO: 227 - protein, 344 aa, Synthetic construct
MNKPKFKAAAVQAAPVFLDLDKTIDKTIALMEDAARNGAELIAFPETWLPGYPWYAWLDSPAMWLPKYTHRYFDNSLEYGTPQAERIRKSAKDNNIVVSMGLSEREGGSLYISQWFIDRDGDTIAARRKLKPTHVERTIFGEGDGSDLAVWDARLGRVGGLCCWEHLQPLSKYAMYAQHEQVHVAAWPSFSLYEAYAYALGPEVNNSASRIYAVEGQCFVLAPCALVSKEMVDEMCTTDGQKALLRTGGVYARIFGPDGRLLHEGLPAGEEGLIYAEIDLGLISVSKAVGDPAGHYSRPDVTQLIHNKAPRRAVVDTAPPSDPAPAVERPHGLDEPALPAMAEL

SEQ ID NO: 228 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatagcccggcaatgtggttaccgaaatatacccatcgctattttgataacagcctggaatatggcaccccgcaggcggaacgcattcgcaaaagcgcgaaagataacaacattgtggtgagcatgggcctgagcgaacgcgaaggcggcagcctgtacatttctcagtggtttattgatcgcgatggcgataccattgcggcgcgccgcaaactgaaaccgacccatgtggaacgcaccatttttggcgaaggcgatggcagcgatctggcggtgtgggatgcgcgtttaggtcgcgttggcggcttatgctgctgggaacatctgcagccgctgagcaaatatgcgatgtatgcgcagcatgaacaggtgcatgtggcggcgtggccgagctttagcctgtatgaagcgtatgcgtatgcgttaggcccggaagtgaacaacagcgcgagcagaatttatgcggtggaaggccagtgctttgtgctggcgccgtgcgcactggttagcaaagaaatggtggatgagatgtgcaccaccgatggccagaaagcgctgttacgcaccggcggcgtgtatgcgcgcatttttggcccggatggccgcctgttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtctggatgaaccggcattaccggcgatggcggaactg

SEQ ID NO: 229 - protein, 344 aa, Synthetic construct
MNKPKFKAAAVQAAPVFLDLDKTIDKTIALMEEAARNGAELIAFPETWLPGYPWYAWLDAPAMWLPQYTKRYFDNSLEYGTPQAERIRKDAKDHNIVVSMGLSEREGGSLYISQWFIDRDGDTIAQRRKLKPTHVERTIFGEGDGSDLAVYDTRLGRVGGLCCWEHLQPLSKYAMYAQHEQVHVAAWPSFSLYDAYAYALGPEVNTSASRIYAVEGQCFVLAPCASVSKEMVDEMCTTDEQKALLTTGGGYARIFGPDGRELHEGLPEGEEGLIYAEIDLGLISVSKAVTDPAGHYSRPDVTQLIHNKAPRRAVVDTAPPSDPAPAVERPHGADLPALPAMAEL

SEQ ID NO: 230 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaccgcagtataccaaacgctattttgataacagcctggaatatggcaccccgcaggcggaacgcattcgcaaagatgcgaaagatcataacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagcatgaacaggtgcatgtggcggcgtggccgagctttagcctgtatgatgcgtatgcgtatgcgttaggcccggaagtgaacaccagcgcgagcagaatttatgcggtggaaggccagtgctttgtgctggcgccgtgcgcaagcgttagcaaagaaatggtggatgagatgtgcaccaccgatgaacagaaagcgctgttaaccaccggcggcggctatgcgcgcatttttggcccggatggccgcgaattacatgaaggcctgccggaaggcgaagaaggcctgatttatgcggaaattgatctgggcctgattagcgtgagcaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatctgccggcattaccggcgatggcggaactg

SEQ ID NO: 231 - protein, 344 aa, Synthetic construct
MNKPKFKAAAVQAAPVFLDLDKTIDKTEALMEDAARNGAELIAFPETWLPGYPWYAWLDAPAMWMPQYTQRYFDNSLEYGTPQADRIRKVAKDNNITVSIGLSEREGGSLYISQWFIDRDGDTIAQRRKLKPTHVERTIFGEGDGSDLAVYDTRLGRVGGLCCWEHLQPLSKYAMYAQNEQVHVAAWPSFSVYEAYAYALGPEVNTSASRIYAVEGQCFVLAPCACVSKEMVDEMCTTDLQKALLKTGGVYARIFGPDGRQLSEGLPAGEEGLIYAEIDLGLISVSKAVGDPAGHYSRPDVTQLILHKAPRRAVVDTATPSDPAPAVERPHGADEPALPAMAEL

SEQ ID NO: 232 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaagatgcggcgcgcaacggcgcggaactgattgcgtttccggaaacctggttaccgggctatccgtggtatgcgtggctggatgcgccggcaatgtggatgccgcagtatacccagcgctattttgataacagcctggaatatggcaccccgcaggcggatcgcattcgcaaagtggcgaaagataacaacattaccgtgagcattggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtatgatacccgtttaggtcgcgttggcggcttatgctgctgggaacatctgcagccgctgagcaaatatgcgatgtatgcgcagaacgaacaggtgcatgtggcggcgtggccgagctttagcgtgtatgaagcgtatgcgtatgcgttaggcccggaagtgaacaccagcgcgagcagaatttatgcggtggaaggccagtgctttgtgctggcgccgtgcgcatgcgttagcaaagaaatggtggatgagatgtgcaccaccgatctgcagaaagcgctgttaaaaaccggcggcgtgtatgcgcgcatttttggcccggatggccgccagttaagcgaaggcctgccggccggcgaagaaggcctgatttatgcggaaattgatctgggcctgattagcgtgagcaaagcggtgggcgatccggcgggtcattatagccgcccagatgtgacccagttaattctgcataaagcgccgcgccgtgcagttgttgatactgcaaccccatctgacccagcaccggcggtggaacgtccacatggtgcagatgaaccggcattaccggcgatggcggaactg

SEQ ID NO: 233 - protein, 344 aa, Synthetic construct
MNKPKFKAAAVQAAPVFLDLDKTIDKTIALMEDAARNGAELIAFPETWLPGYPWYAWLDAPAMWMPGYTQRYFDNSLEYGTPQAERIRKAAKDNNIVVSMGLSEREGASLYISQWFIDRDGDTIAQRRKLKPTHAERTIFGEGDGSDLAVRDPRLGRVGGLCCWEHLQPLSKYAMYAQNEQVHVAAWPSFSIYRAYAYALGPEVNTSASRIYAVEGQCFVLAPCAVVSKEMVDEMCTTDLQKALLKPGGVHAVIFGPDGRQLHEGLPAGEEGLIYAEIDLGLISVSKAVGDPAGHYSRPDVTQLLHNKAPRRAVVDTAPPSDPAPAVERPHGADEPALPAMAEL

SEQ ID NO: 234 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggatgccgggctatacccagcgctattttgataacagcctggaatatggcaccccgcaggcggaacgcattcgcaaagcggcgaaagataacaacattgtggtgagcatgggcctgagcgaacgcgaaggcgcgagcctgtacatttctcagtggtttattgatcgcgatggcgataccattgcgcagcgccgcaaactgaaaccgacccatgcggaacgcaccatttttggcgaaggcgatggcagcgatctggcggtgcgcgatccgcgtttaggtcgcgttggcggcttatgctgctgggaacatctgcagccgctgagcaaatatgcgatgtatgcgcagaacgaacaggtgcatgtggcggcgtggccgagctttagcatttatcgcgcgtatgcgtatgcgttaggcccggaagtgaacaccagcgcgagcagaatttatgcggtggaaggccagtgctttgtgctggcgccgtgcgcagtggttagcaaagaaatggtggatgagatgtgcaccaccgatctgcagaaagcgctgttaaaaccgggcggcgtgcatgcggtgatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttactgcataacaaagcgccgcgccgtgcagttgttgatactgcaccaccatctgacccagcaccggcggtggaacgtccacatggtgcagatgaaccggcattaccggcgatggcggaactg

SEQ ID NO: 235 - protein, 344 aa, Synthetic construct
MNKPKFKAAAVQAAPVFLDLDKTIDKTIALMEEAARNGAELIAFPETWLPGYPWYAWLDAPAMWLIGYTQRYFDNSLEYDTPQAERIRKAAKDNNITVLMGLSEREGASLYISQWFIDRDGDTIAQRRKLKPTHVERTIFGEGDGSDLAVWDTRLGRVGGLCCWEHLQPLSKYAMYAQNEQVHVAAWPSFSIYGAYAYALGPIVNTSASRIYAVEGQCFVLAPCATVSKEMVDEMCTTDLQKALLKTGGCYAAIFGPDGRQLHEGLPTGEEGLIYAEIDLGLISVSKAVTDPAGHYSRPDVTQLIHNKAPRRAVVDTAPPSDPAPAVERPHGADEPALPAMAEL

SEQ ID NO: 236 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagaagcggcgcgcaacggcgcggaactgattgcgtttccggaaacctggttaccgggctatccgtggtatgcgtggctggatgcgccggcaatgtggttaattggctatacccagcgctattttgataacagcctggaatatgataccccgcaggcggaacgcattcgcaaagcggcgaaagataacaacattaccgtgctgatgggcctgagcgaacgcgaaggcgcgagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggctgctatgcggcgatttttggcccggatggccgccagttacatgaaggcctgccgaccggcgaagaaggcctgatttatgcggaaattgatctgggcctgattagcgtgagcaaagcggtgaccgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 237 - protein, 344 aa, Synthetic construct
MNKPKFKAAAVQAAPVFLDLDKAIDKTIALMEEAARNGAELIAFPETFLPGYPWYAWLYAPAMWLPQYTQRYFDNSLEYGTPQAERIRKAAKDNNITVSMGLSEREGGSLYISQWFIDRDGDTIAKRRKLKPTHVERTIFGEGDGSDLAVIDIRLGRVGGLCCWEHLQPLSKYAMYAQNEQVHVAAWPSFSVYEAYAYALGPIVNTSASRIYAVEGQCFVLAPCACVSKEMVDEMCTTDLQKALLKTGGGYARIFGPDGRQLHEGLPAGEEGLIYAEIDLGLISVSKAVGDPAGHYSRPDVTQLIHDKARRRAVVDTAPPSDDAPAVERPHGADEPALPAMAEL

SEQ ID NO: 238 - DNA, 1032 bp, Synthetic construct
  source 1..1032: mol_type = other DNA
atgaacaaaccgaaatttaaagcggcggcggtgcaggcggcgccggtgtttctggatttagacaaagcgattgataagaccattgcgctgatggaagaagcggcgcgcaacggcgcggaactgattgcgtttccggaaacctttttaccgggctatccgtggtatgcgtggctgtatgcgccggcaatgtggttaccgcagtatacccagcgctattttgataacagcctggaatatggcaccccgcaggcggaacgcattcgcaaagcggcgaaagataacaacattaccgtgagcatgggcctgagcgaacgcgaaggcggcagcctgtacatttctcagtggtttattgatcgcgatggcgataccattgcgaaacgccgcaaactgaaaccgacccatgtggaacgcaccatttttggcgaaggcgatggcagcgatctggcggtgattgatattcgtttaggtcgcgttggcggcttatgctgctgggaacatctgcagccgctgagcaaatatgcgatgtatgcgcagaacgaacaggtgcatgtggcggcgtggccgagctttagcgtgtatgaagcgtatgcgtatgcgttaggcccgattgtgaacaccagcgcgagcagaatttatgcggtggaaggccagtgctttgtgctggcgccgtgcgcatgcgttagcaaagaaatggtggatgagatgtgcaccaccgatctgcagaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcatgataaagcgcgccgccgtgcagttgttgatactgcaccaccatctgacgatgcaccggcggtggaacgtccacatggtgcagatgaaccggcattaccggcgatggcggaactg

SEQ ID NO: 239 - protein, 344 aa, Synthetic construct
MNKPKFKAAAVQAAPVFLDLDKTIDKTEALMERAAREGAELIAFPETWLPGYPWYAWLDAPAMWLPQYTQRYFDNSLEYGTPQAERIRKTAKDNNIVVSMGLSEREGGSLYISQWLIDRDGDTAAQRRKLKPTHVERTIFGEGDGSDLAVWDTRLGRVGGLCCWEHLQPLSKYAMYAQTEQVHVAAWPSFSLYEAYAYALGPDVNTSASRIYAVEGQCFVLAPCATVSKEMVDEMCTTDLHKALLKTGGAYAVIFGPDGRQLHEGLDAGEEGLIYAEIDLGLISVSKAVADPAGHYSRPDVTQLIHDKAPRRAVVDTAPPSDPAPAVERPHGADEPALPAMAEL

SEQ ID NO: 240 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccgaagcgctgatggaacgcgcggcgcgcgaaggcgcggaactgattgcgtttccggaaacctggttaccgggctatccgtggtatgcgtggctggatgcgccggcaatgtggttaccgcagtatacccagcgctattttgataacagcctggaatatggcaccccgcaggcggaacgcattcgcaaaaccgcgaaagataacaacattgtggtgagcatgggcctgagcgaacgcgaaggcggcagcctgtacatttctcagtggctgattgatcgcgatggcgataccgcggcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaccgaacaggtgcatgtggcggcgtggccgagctttagcctgtatgaagcgtatgcgtatgcgttaggcccggacgtgaacaccagcgcgagcagaatttatgcggtggaaggccagtgctttgtgctggcgccgtgcgcaaccgtgagcaaagaaatggtggatgagatgtgcaccaccgatttacataaagcgctgttaaaaaccggcggcgcgtatgcggtgatttttggcccggatggccgccagttacatgaaggcctggatgccggcgaagaaggcctgatttatgcggaaattgatctgggcctgattagcgtgagcaaagcggtggcggatccggcgggtcattatagccgcccagatgtgacccagttaattcatgacaaagcgccgcgccgtgcagttgttgatactgcaccaccatctgacccagcaccggcggtggaacgtccacatggtgcagatgaaccggcattaccggcgatggcggaactg

SEQ ID NO: 241 - protein, 344 aa, Synthetic construct
MNKPKFKAAAVQAAPVFLDLDKTIDKTRALMEDAARNGAELIAFPETWLPGYPWYAWLYAPAMWVPVYTRRYFDNSLEYGTPQKERIRKAAKDNNIVVSMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYEAYAYALGPEVNTSASRIYAVEGQCFVLAPCATVSKEMVDEMCTTDLQKALLKTGGGYARIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 242 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaagatgcggcgcgcaacggcgcggaactgattgcgtttccggaaacctggttaccgggctatccgtggtatgcgtggctgtatgcgccggcaatgtgggtgccggtgtatacccgccgctattttgataacagcctggaatatggcaccccgcagaaagaacgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 243 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEYGTPQAHRIRKDAKDNNIYVSMGLSEREGGSLYISQWFIDRDGDTIAERRKLKPTHVERTIFGEGDGSDLAVWDTRLGRVGGLCCWEHLQPLSKYAMYAQNEQVHVAAWPSFSLYEAYAYALGPEVNTSASRIYAVEGQCFVLAPCACVSKEMVDEMCTTDVQKALLKTGGGYARIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 244 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcgcatcgcattcgcaaagatgcgaaagataacaacatttatgtgagcatgggcctgagcgaacgcgaaggcggcagcctgtacatttctcagtggtttattgatcgcgatggcgataccattgcggaacgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcatgcgttagcaaagaaatggtggatgagatgtgcaccaccgatgtgcagaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 245 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEYGTPQAERIRKAAKDNNIVVSMGLSEREGESLYISQWFIDRDGDTIAQRRKLKPTHVERTIFGEGDGSDLAVHDIRLGRVGGLCCWEHLQPLSKYAMYAQNEQVHVAAWPSFSLYEAYAYALGPEVNTSASRIYAVEGQCFVLAPCATVSKEMVDEMCTTDLRKALLKTGGCYASIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 246 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcggaacgcattcgcaaagcggcgaaagataacaacattgtggtgagcatgggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtgcatgatattcgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggctgctatgcgtctatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 247 - protein, 344 aa, Synthetic construct
MNKPKFKAAAVQAAPVFLDLDKTIDKTIALMEKAARNGAELIAFPETWLPGYPWYAWLDAPAMWLPQYTQRYFDNSLEYGTPQAERIRKAAKDNNIVVSMGLSEREGGSLYISQWFIDRDGDTIAQRRKLKPTHVERTIFGEGDGSDLAVWDTRLGRVGGLCCWEHLQPLSKYAMYAQKEQVHVAAWPSFSIYEAYAYALGPIVNTSASRIYAVEGQCFVLAPCATVSKEMVDEMCTTDLQKALLKTGGGYARIFGPDGRQLHEGLPAGEEGLIYAEIDLGLISVSKAVNDPAGHYSRPDVTQLIHDKAPRRAVVDTAPPSDPAPAVERPHGADEPALPAMAEL

SEQ ID NO: 248 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcggaacgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcatttatgaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 249 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEFGTPQAERIRKAAKDNNIVVSMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYGAYAYALGPEVNTSASRIYAVEGQCFVLAPCATVSKAMVDEMCTTDLQKALLKTGGGYARIFGPDGRELHEGLPAGEEGLIYAEIDLGLISVSKAVGDPAGHYSRPDVTQLIHNKAPRRAVVDTAPPSDPAPAVERPHGADEPALPAMAEL

SEQ ID NO: 250 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatttggcaccccgcaggcggaacgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgtatgcgttaggcccggaagtgaacaccagcgcgagcagaatttatgcggtggaaggccagtgctttgtgctggcgccgtgcgcaactgttagcaaagcgatggtggatgagatgtgcaccaccgatctgcagaaagcgctgttaaaaaccggcggcggctatgcgcgcatttttggcccggatggccgcgaattacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 251 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEYGTPQAERIRKDAKDNNIVVSMGLSEREGGSLYISQWFIDRDGDTIAERRKLKPTHVERTIFGEGDGSDLAVWDTRLGRVGGLCCWEHLQPLSKYAMYAQNEQVHVAAWPSFSIYEAYAYALGPIVNTSASRIYAVEGQCFVLAPCATVSKEMVDEMCTTDLQKALLKTGGGYARIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 252 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcggaacgcattcgcaaagatgcgaaagataacaacattgtggtgagcatgggcctgagcgaacgcgaaggcggcagcctgtacatttctcagtggtttattgatcgcgatggcgataccattgcggaacgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcatttatgaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 253 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEYGTPQAHRIRKAAKDNNIYVSMGLSEREGGSLYISQWFIDRDGDTIAQRRKLKPTHVERTIFGEGDGSDLAVWDIRLGRVGGLCCWEHLQPLSKYAMYAQNEQVHVAAWPSFSLYEAYAYALGPEVNTSASRIYAVEGQCFVLAPCATVSKEMVDEMCTTDLQKALLKTGGGYASIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 254 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcaggcgcatcgcattcgcaaagcggcgaaagataacaacatttatgtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgtctatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 255 - protein, 344 aa, Synthetic construct
MNKPKFKAAAVQAAPVFLDLDKTIDKTIALMEKAARNGAELIAFPETWLPGYPWYAWLDAPAMWLPVYTQRYFDNSLEYGTPQAERIRKAAKDNNIVVSMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYEAYAYALGPEVNTSASRIYAVEGQCFVLAPCATVSKEMVDEMCTTDLRKALLKTGGGYARIFGPDGRQLHEGLPAGEEGLIYAEIDLGLISVSKAVGDPAGHYSRPDVTQLIHDKAPRRAVVDTAPPSDPAPAVERPHGADEPALPAMAEL

SEQ ID NO: 256 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtggttaccggtgtatacccagcgctattttgataacagcctggaatatggcaccccgcaggcggaacgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcgcaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 257 - protein, 344 aa, Synthetic construct
MNKPKFKAAAVQAAPVFLDLDKTIDKTIALMEDAARNGAELIAFPETWLPGYPWYAWLDAPAMWVPQYTRRYFDNSLEYGTPQAERIRKAAKDNNIVVSMGLSERDGGSLYISQWFIDRDGDTIAQRRKLKPTHVERTIFGEGDGSDLAVWDIRLGRVGGLCCWEHLQPLSKYAMYAQKEQVHVAAWPSFSIYEAYAYALGPEVNTSASRIYAVEGQCFVLAPCATVSKEMVDEMCTTDLQKALLKTGGGYARIFGPDGRQLHEPLPAGEEGLIYAEIDLGLISVSKAVGDPAGHYSRPDVTQLIHNKAPRRAVVDTAPPSDPAPAVERPHGADEPALPAMAEL

SEQ ID NO: 258 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtgggtgccgcagtatacccgccgctattttgataacagcctggaatatggcaccccgcaggcggaacgcattcgcaaagcggcgaaagataacaacattgtggtgagcatgggcctgagcgaacgcgatggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatattcgtttaggtcgcgttggcggcttatgctgctgggaacatctgcagccgctgagcaaatatgcgatgtatgcgcagaaagaacaggtgcatgtggcggcgtggccgagctttagcatttatgaagcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgcgcatttttggcccggatggccgccagttacatgaaccgctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 259 - protein, 344 aa, Synthetic construct
MNKPKFKAAAVQAAPVFLDLDKTIDKTIALMEDAARNGAELIAFPETWLPGYPWYAWLDAPAMWLPQYTDRYFDNSLEYGTPQAHRIRKAAKDNNIVVSMGLSEREGGSLYISQWFIDRDGDTIAQRRKLKPTHVERTIFGEGDGSDLAVWDTRLGRVGGLCCWEHLQPLSKYAMYAQNEQVHVAAWPSFSLYEAYAYALGPIVNTSASRIYAVEGQCFVLAPCATVSKEMVDEMCTTDVQKALLKTGGGYASIFGPDGRELHEGLPAGEEGLIYAEIDLGLISVSKAVGDPAGHYSRPDVTQLIHNKAPRRAVVDTAPPSDPAPAVERPHGADEPALPAMAEL

SEQ ID NO: 260 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtataccgatcgctattttgataacagcctggaatatggcaccccgcaggcgcatcgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccgattgtgaacaccagcgcgagcagaatttatgcggtggaaggccagtgctttgtgctggcgccgtgcgcaactgttagcaaagaaatggtggatgagatgtgcaccaccgatgtgcagaaagcgctgttaaaaaccggcggcggctatgcgagcatttttggcccggatggccgcgaattacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 261 - protein, 344 aa, Synthetic construct
MNKPKFKAAAVQAAPVFLDLDKTIDKTIALMEKAARNGAELIAFPETWLPGYPWYAWLDAPAMWVPQYTQRYFDNSLEYGTPQAERIRKAAKDNNIYVSMGLSEREGESLYISQWFIDRDGDTIAQRRKLKPTHVERTIFGEGDGSDLAVWDTRLGRVGGLCCWEHLQPLSKYAMYAQNEQVHVAAWPSFSLYGAYAYALGPEVNTSASRIYAVEGQCFVLAPCATVSKEMVDEMCTTDLQKALLKTGGGYARIFGPDGRQLHEPLPAGEEGLIYAEIDLGLISVSKAVGDPAGHYSRPDVTQLIHDKAPRRAVVDTAPPSDPAPAVERPHGADEPALPAMAEL

SEQ ID NO: 262 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaaaaagcggcgcgcaacggcgcggaactgattgcgtttccggaaacctggttaccgggctatccgtggtatgcgtggctggatgcgccggcaatgtgggtgccgcagtatacccagcgctattttgataacagcctggaatatggcaccccgcaggcggaacgcattcgcaaagcggcgaaagataacaacatttatgtgagcatgggcctgagcgaacgcgaaggcgaaagcctgtacatttctcagtggtttattgatcgcgatggcgataccattgcgcagcgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaacgaacaggtgcatgtggcggcgtggccgagctttagcctgtatggcgcgtatgcgtatgcgttaggcccggaagtgaacaccagcgcgagcagaatttatgcggtggaaggccagtgctttgtgctggcgccgtgcgcaactgttagcaaagaaatggtggatgagatgtgcaccaccgatctgcagaaagcgctgttaaaaaccggcggcggctatgcgcgcatttttggcccggatggccgccagttacatgaaccgctgccggccggcgaagaaggcctgatttatgcggaaattgatctgggcctgattagcgtgagcaaagcggtgggcgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 263 - protein, 344 aa, Synthetic construct
MNKPKFKAAAVQAAPVFLDLDKTIDKTIALMEDAARNGAELIAFPETWLPGYPWYAWLDAPAMWLPVYTQRYFDNSLEFGTPQAERIRKAAKDNNIVVSMGLSEREGGSLYISQWFIDRDGDTIAQRRKLKPTHVERTIFGEGDGSDLAVHDTRLGRVGGLCCWEHLQPLSKYAMYAQNEQVHVAAWPSFSLYEAYAYALGPEVNTSASRIYAVEGQCFVLAPCATVSKAMVDEMCTTDLRKALLKTGGGYARIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 264 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccggtgtatacccagcgctattttgataacagcctggaatttggcaccccgcaggcggaacgcattcgcaaagcggcgaaagataacaacattgtggtgagcatgggcctgagcgaacgcgaaggcggcagcctgtacatttctcagtggtttattgatcgcgatggcgataccattgcgcagcgccgcaaactgaaaccgacccatgtggaacgcaccatttttggcgaaggcgatggcagcgatctggcggtgcatgatacccgtttaggtcgcgttggcggcttatgctgctgggaacatctgcagccgctgagcaaatatgcgatgtatgcgcagaacgaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcaactgttagcaaagcgatggtggatgagatgtgcaccaccgatctgcgcaaagcgctgttaaaaaccggcggcggctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 265 - protein, 344 aa, Synthetic construct
MNKPKFKAAAVQAAPVFLDLDKTIDKTIALMEDAARNGAELIAFPETWLPGYPWYAWLDAPAMWLPQYTQRYFDNSLEYGTPQKERIRKAAKDNNIVVSMGLSERDGGSLYISQWFIDRDGDTIAERRKLKPTHVERTIFGEGDGSDLAVWDTRLGRVGGLCCWEHLQPLSKYAMYAQKEQVHVAAWPSFSLYEAYAYALGPEVNTSASRIYAVEGQCFVLAPCACVSKEMVDEMCTTDLRKALLKTGGCYARIFGPDGRQLHEGLPAGEEGLIYAEIDLGLISVSKAVGDPAGHYSRPDVTQLIHNKAPRRAVVDTAPPSDPAPAVERPHGADEPALPAMAEL

SEQ ID NO: 266 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagaccattgcgctgatggaagatgcggcgcgcaacggcgcggaactgattgcgtttccggaaacctggttaccgggctatccgtggtatgcgtggctggatgcgccggcaatgtggttaccgcagtatacccagcgctattttgataacagcctggaatatggcaccccgcagaaagaacgcattcgcaaagcggcgaaagataacaacattgtggtgagcatgggcctgagcgaacgcgatggcggcagcctgtacatttctcagtggtttattgatcgcgatggcgataccattgcggaacgccgcaaactgaaaccgacccatgtggaacgcaccatttttggcgaaggcgatggcagcgatctggcggtgtgggatacccgtttaggtcgcgttggcggcttatgctgctgggaacatctgcagccgctgagcaaatatgcgatgtatgcgcagaaagaacaggtgcatgtggcggcgtggccgagctttagcctgtatgaagcgtatgcgtatgcgttaggcccggaagtgaacaccagcgcgagcagaatttatgcggtggaaggccagtgctttgtgctggcgccgtgcgcatgcgttagcaaagaaatggtggatgagatgtgcaccaccgatctgcgcaaagcgctgttaaaaaccggcggctgctatgcgcgcatttttggcccggatggccgccagttacatgaaggcctgccggccggcgaagaaggcctgatttatgcggaaattgatctgggcctgattagcgtgagcaaagcggtgggcgatccggcgggtcattatagccgcccagatgtgacccagttaattcataacaaagcgccgcgccgtgcagttgttgatactgcaccaccatctgacccagcaccggcggtggaacgtccacatggtgcagatgaaccggcattaccggcgatggcggaactg

SEQ ID NO: 267 - protein, 344 aa, Synthetic construct
MNKPKFKAASVQAAPVFLDLDKTIDKTRALMEKAARNGAELIAFPETWLPGYPWYAWLYAPAMWVMVYVRRYFDNSLIFGTPQKHRIRKDAKDNNIYVVMGLSEREGESLYITQWIIDRDGDTQAERRKLKPTHVERTIFGEGDGSDLTVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELAEGLPAGEEGLILAEIDLGLISVSKAVNDPAGHYSRPDVTQLIHDKAPRRAVEDTAPPSDPAPAVERPHGADEPALPAMAEL

SEQ ID NO: 268 - DNA, 1032 bp, Synthetic construct
  source 1..1032: mol_type = other DNA
atgaacaaaccgaaatttaaagcggcgagcgtgcaggcggcgccggtgtttctggatttagacaaaaccattgataagacccgcgcgctgatggaaaaagcggcgcgcaacggcgcggaactgattgcgtttccggaaacctggttaccgggctatccgtggtatgcgtggctgtatgcgccggcaatgtgggtgatggtgtatgtgcgccgctattttgataacagcctgatttttggcaccccgcagaaacatcgcattcgcaaagatgcgaaagataacaacatttatgtggtgatgggcctgagcgaacgcgaaggcgaaagcctgtacattacccagtggattattgatcgcgatggcgatacccaggcggaacgccgcaaactgaaaccgacccatgtggaacgcaccatttttggcgaaggcgatggcagcgatctgaccgtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattagcggaaggcctgccggccggcgaagaaggcctgattctggcggaaattgatctgggcctgattagcgtgagcaaagcggtgaacgatccggcgggtcattatagccgcccagatgtgacccagttaattcatgataaagcgccgcgccgtgcagttgaagatactgcaccaccatctgacccagcaccggcggtggaacgtccacatggtgcagatgaaccggcattaccggcgatggcggaactg

SEQ ID NO: 269 - protein, 344 aa, Synthetic construct
MNKPKFKAAAVQAAPVYLDLDKTIDKTRALMEKAARAGAELIAFPETWLPGYPWYAWLYAPAMWVPVYTRRYFDNSLEFDTPQKHRIRKDAKENNIYISSGLSEREGESLYISQWFIDRDGDTIAERRKLKPTHGERTIFGEGDGSDLSVHDIRLGRVGGLCCWEHLQPLSKYAMYAQKEQVHVAAWPSFSIYGGYAYALGPIVNTSASRIYAVEGQCFVLAPCACVSKAMVDEMCTTPVRKALLKTGGCYASIFGPDGRELHEGLPAGEEGLIMAEIDLGLISVSKAVNDPAGHYSRPDVTQLIHDKAPRRAVVDTAPPSDPAPAVERPHGADEPALPAMAEL

SEQ ID NO: 270 - DNA, 1032 bp, Synthetic construct
  source 1..1032: mol_type = other DNA
atgaacaaaccgaaatttaaagcggcggcggtgcaggcggcgccggtgtatctggatttagacaaaaccattgataagacccgcgcgctgatggaaaaagcggcgcgcgcgggcgcggaactgattgcgtttccggaaacctggttaccgggctatccgtggtatgcgtggctgtatgcgccggcaatgtgggtgccggtgtatacccgccgctattttgataacagcctggaatttgataccccgcagaaacatcgcattcgcaaagatgcgaaagaaaacaacatttatattagcagcggcctgagcgaacgcgaaggcgaaagcctgtacatttctcagtggtttattgatcgcgatggcgataccattgcggaacgccgcaaactgaaaccgacccatggcgaacgcaccatttttggcgaaggcgatggcagcgatctgagcgtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcggctatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccccggtgcgcaaagcgctgttaaaaaccggcggctgctatgcgagcatttttggcccggatggccgcgaattacatgaaggcctgccggccggcgaagaaggcctgattatggcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 271 - protein, 344 aa, Synthetic construct
MNKPKFKAAAVQAAPVWLDLDKTIDKTRALIEKAARNGAELIAFPETWLPGYPWYAWLYAPAMWVPVYFRRYFDNSLIFGNPQKHRIRKDAKDANIYVLMGLSEREGESLYISQWFIDADGDTIAERRKLKPTHVERTIFGEGDGSDLVVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIYGPDGRELHEGLPAGEEGLIYAEIDLGLISVSKSVNDPAGHYSRPDVTQLIIDKAPRRAVVDTAPPSDPAPAVERPHGADEPALPAMAEL

SEQ ID NO: 272 - DNA, 1032 bp, Synthetic construct
  source 1..1032: mol_type = other DNA
atgaacaaaccgaaatttaaagcggcggcggtgcaggcggcgccggtgtggctggatttagacaaaaccattgataagacccgcgcgctgattgaaaaagcggcgcgcaacggcgcggaactgattgcgtttccggaaacctggttaccgggctatccgtggtatgcgtggctgtatgcgccggcaatgtgggtgccggtgtattttcgccgctattttgataacagcctgatttttggcaacccgcagaaacatcgcattcgcaaagatgcgaaagatgcgaacatttatgtgctgatgggcctgagcgaacgcgaaggcgaaagcctgtacatttctcagtggtttattgatgcggatggcgataccattgcggaacgccgcaaactgaaaccgacccatgtggaacgcaccatttttggcgaaggcgatggcagcgatctggtggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttatggcccggatggccgcgaattacatgaaggcctgccggccggcgaagaaggcctgatttatgcggaaattgatctgggcctgattagcgtgagcaaaagcgtgaacgatccggcgggtcattatagccgcccagatgtgacccagttaattattgataaagcgccgcgccgtgcagttgttgatactgcaccaccatctgacccagcaccggcggtggaacgtccacatggtgcagatgaaccggcattaccggcgatggcggaactg

SEQ ID NO: 273 - protein, 344 aa, Synthetic construct
MNKPKFKAAIVQAAPVFLDLDKTIDKTRALMEKAARNGAELIAFPETWLPGYPWYAWLYAPAMWVLVYIRRYFDNSLSFNNPQKHRIRKDAKDNNIYVVMGLSEREGESLYITQWFIDADGDTIAERRKLKPTHVERTIFGEGDGSDLAVHDIRLGRVGTLCCWEHLQPLSKYAMYAQKEQVHVAAWPSFSIYGAYAYALGPIVNTSASRIYAVEGQCFVLAPCACVSKAMVDEMCTTNVRKALLKTGGCYASIFGPDGRELHEGLPAGEEGLIYAEIDLGLISVSKAVNDPAGHYSRPDVTQLLHDKAPRRAVVDTAPPSDPAPAVERPHGADEPALPAMAEL

SEQ ID NO: 274 - DNA, 1032 bp, Synthetic construct
  source 1..1032: mol_type = other DNA
atgaacaaaccgaaatttaaagcggcgattgtgcaggcggcgccggtgtttctggatttagacaaaaccattgataagacccgcgcgctgatggaaaaagcggcgcgcaacggcgcggaactgattgcgtttccggaaacctggttaccgggctatccgtggtatgcgtggctgtatgcgccggcaatgtgggtgctggtgtatattcgccgctattttgataacagcctgagctttaacaacccgcagaaacatcgcattcgcaaagatgcgaaagataacaacatttatgtggtgatgggcctgagcgaacgcgaaggcgaaagcctgtacattacccagtggtttattgatgcggatggcgataccattgcggaacgccgcaaactgaaaccgacccatgtggaacgcaccatttttggcgaaggcgatggcagcgatctggcggtgcatgatattcgtttaggtcgcgttggcaccttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccaacgtgcgcaaagcgctgttaaaaaccggcggctgctatgcgagcatttttggcccggatggccgcgaattacatgaaggcctgccggccggcgaagaaggcctgatttatgcggaaattgatctgggcctgattagcgtgagcaaagcggtgaacgatccggcgggtcattatagccgcccagatgtgacccagttactgcatgataaagcgccgcgccgtgcagttgttgatactgcaccaccatctgacccagcaccggcggtggaacgtccacatggtgcagatgaaccggcattaccggcgatggcggaactg

SEQ ID NO: 275 - protein, 344 aa, Synthetic construct
MNKPKFKAAAVQAAPVLLDLDKTIDKTRALMEKAARNGAELIAFPETWLPGYPWYAWLYAPAMWVMVYTRRYFDNSLEFGSPQKHRIRKDAKKNNIYASMGLSEREGESLYITQWHIDDDGDTVAERRKLKPTHVERTIFGEGDGSDLAVHDIRLGRVGGLCCWEHMQPLSKYAMYAQKEQVHVAAWPSFSIYGAYAYALGPIVNTSASRIYAVEGQCFVLAPCACVSKAMVDEMCTTDVRKALLRTGGCYASIFGPDGRELHEGLPAGEEGLIYAEIDLGLISVSKAVNDPAGHYSRPDVTQLIHDKAPRRPVVDTAPPSDPAPAVERPHGADEEALPAMAEL

SEQ ID NO: 276 - DNA, 1032 bp, Synthetic construct
  source 1..1032: mol_type = other DNA
atgaacaaaccgaaatttaaagcggcggcggtgcaggcggcgccggtgctgctggatttagacaaaaccattgataagacccgcgcgctgatggaaaaagcggcgcgcaacggcgcggaactgattgcgtttccggaaacctggttaccgggctatccgtggtatgcgtggctgtatgcgccggcaatgtgggtgatggtgtatacccgccgctattttgataacagcctggaatttggcagcccgcagaaacatcgcattcgcaaagatgcgaaaaaaaacaacatttatgcgagcatgggcctgagcgaacgcgaaggcgaaagcctgtacattacccagtggcatattgatgatgatggcgataccgtggcggaacgccgcaaactgaaaccgacccatgtggaacgcaccatttttggcgaaggcgatggcagcgatctggcggtgcatgatattcgtttaggtcgcgttggcggcttatgctgctgggaacatatgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttacgcaccggcggctgctatgcgagcatttttggcccggatggccgcgaattacatgaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtccggttgttgatactgcaccaccatctgacccagcaccggcggtggaacgtccacatggtgcagatgaagaagcattaccggcgatggcggaactg

SEQ ID NO: 277 - protein, 344 aa, Synthetic construct
MNKPKFKVAAVQAAPVFLDLDKTIDKTRALMEKAARNGAELIAFPETWLPGYPWYAWLYTPAMWVPVYTRRYFDNSLEFELPQKHRIRKDAKDNNIYVVMGLSEREGESLYISQWIIDNDGDTIAERRKLKPTHAERTIFGEGDGSDLSVHDIELGRVGGLCCWEHLQPLSKYAMYAQKEQVHVAAWPSFSIYGAYAYALGPIVNTSASRTYAVEGQCFVLAPCACVSKAMVDEMCTTDVRKALLKTGGCYASIFGPDGRELHEGLPAGEEGLIYAEIDLGLISVSKSVNDPAGHYSRPDVTQLIHDKAPRRAVVDTAPPSDPAPTVERPHGADEPALPAMAEL

SEQ ID NO: 278 - DNA, 1032 bp, Synthetic construct
  source 1..1032: mol_type = other DNA
atgaacaaaccgaaatttaaagtggcggcggtgcaggcggcgccggtgtttctggatttagacaaaaccattgataagacccgcgcgctgatggaaaaagcggcgcgcaacggcgcggaactgattgcgtttccggaaacctggttaccgggctatccgtggtatgcgtggctgtataccccggcaatgtgggtgccggtgtatacccgccgctattttgataacagcctggaatttgaactgccgcagaaacatcgcattcgcaaagatgcgaaagataacaacatttatgtggtgatgggcctgagcgaacgcgaaggcgaaagcctgtacatttctcagtggattattgataacgatggcgataccattgcggaacgccgcaaactgaaaccgacccatgcggaacgcaccatttttggcgaaggcgatggcagcgatctgagcgtgcatgatattgaattaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaacctatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccggcgaagaaggcctgatttatgcggaaattgatctgggcctgattagcgtgagcaaaagcgtgaacgatccggcgggtcattatagccgcccagatgtgacccagttaattcatgataaagcgccgcgccgtgcagttgttgatactgcaccaccatctgacccagcaccgaccgtggaacgtccacatggtgcagatgaaccggcattaccggcgatggcggaactg

SEQ ID NO: 279 - protein, 344 aa, Synthetic construct
MNKPKFKAAAVQAAPVFLDLDKAIDKTRALMEKAARNGAELIAFPETWIPGYPWYAWLYAPAMWVPVYVRRYFDNSLEFNLPQKHRIRKDAKNNNIYASFGLSEREGESLYISQWIIDRDGDTQAERRKLKPTHVERTIFGEGDGSDLAVHDIRLGRVGMLCCWEHLQPLSKYAMYAQKEQVHVAAWPSFSIYGAYAYALGPIVNTSASRLYAVEGSCFVLAPCACVSKAMVDEMCTTDVRKALLKTGGCYASIFGPDGRELHEGLAAGEEGLIYAEIDLGLISVSKAVNDPAGHYSRPDVTQLIHDKAPRRAVVDTAPPSDPAPAVERPHGADEPALPAMAEL

SEQ ID NO: 280 - DNA, 1032 bp, Synthetic construct
  source 1..1032: mol_type = other DNA
atgaacaaaccgaaatttaaagcggcggcggtgcaggcggcgccggtgtttctggatttagacaaagcgattgataagacccgcgcgctgatggaaaaagcggcgcgcaacggcgcggaactgattgcgtttccggaaacctggattccgggctatccgtggtatgcgtggctgtatgcgccggcaatgtgggtgccggtgtatgtgcgccgctattttgataacagcctggaatttaacctgccgcagaaacatcgcattcgcaaagatgcgaaaaacaacaacatttatgcgagctttggcctgagcgaacgcgaaggcgaaagcctgtacatttctcagtggattattgatcgcgatggcgatacccaggcggaacgccgcaaactgaaaccgacccatgtggaacgcaccatttttggcgaaggcgatggcagcgatctggcggtgcatgatattcgtttaggtcgcgttggcatgttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagactgtatgcggtggaaggcagctgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctggcggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 281 - protein, 344 aa, Synthetic construct
MNKPKFKVAAVQAAPVFLDLDKTIDKTRALMEKAAREGAELIAFPETWLPGYPWYAWLYAPAMWVPVYVRRYFDNSLTFGSPQKHRIRKDAKDNNIYLSLGLSEREGESLYITQWFIDRDGDTVAERRKLKPTHTERTIFGEGDGSDLAVHDIRLGRVGGLCCWEHLQPLSKYAMYAQKEQVHVAAWPSFSIYGAYAYALGPIVNTSASRIYAVEGTCFVLAPCACVSKAMVDEMCTTDVRKALLKTGGCAASIFGPDGRELHEGLPAGEEGLVMAEIDLGLISVSKAVNDPAGHYSRPDVTQLIHDKAPRRAVVDTAPPSDPAPAVERPHGADEPALPAMAEL

SEQ ID NO: 282 - DNA, 1032 bp, Synthetic construct
  source 1..1032: mol_type = other DNA
atgaacaaaccgaaatttaaagtggcggcggtgcaggcggcgccggtgtttctggatttagacaaaaccattgataagacccgcgcgctgatggaaaaagcggcgcgcgaaggcgcggaactgattgcgtttccggaaacctggttaccgggctatccgtggtatgcgtggctgtatgcgccggcaatgtgggtgccggtgtatgtgcgccgctattttgataacagcctgacctttggcagcccgcagaaacatcgcattcgcaaagatgcgaaagataacaacatttatctgagcctgggcctgagcgaacgcgaaggcgaaagcctgtacattacccagtggtttattgatcgcgatggcgataccgtggcggaacgccgcaaactgaaaccgacccataccgaacgcaccatttttggcgaaggcgatggcagcgatctggcggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggcacctgctttgtgctggcgccgtgcgcatgcgttagcaaagcgatggtggatgagatgtgcaccaccgatgtgcgcaaagcgctgttaaaaaccggcggctgcgcggcgagcatttttggcccggatggccgcgaattacatgaaggcctgccggccggcgaagaaggcctggtgatggcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 283 - protein, 344 aa, Synthetic construct
MNKPKFKIAAVQAAPVFLDLDKTIDKTRALMEKAARAGAELIAFPETWLPGYPWYAWLYAPAMWVPVYIRRYFDNSLEFGLPQKHRIRKDAKDNNIYAAFGLSEREGESLYISQWVIDRDGDTIAERRKLKPTHSERTIFGEGDGSDLGVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELHEGLPAGEEGLIYAEIDLGLISVSKNVNDPAGHYSRPDVTQLLHDKVPRRAVVDTAPPSDPAPAVERPHGADPPALPAMAEL

SEQ ID NO: 284 - DNA, 1032 bp, Synthetic construct
  source 1..1032: mol_type = other DNA
atgaacaaaccgaaatttaaaattgcggcggtgcaggcggcgccggtgtttctggatttagacaaaaccattgataagacccgcgcgctgatggaaaaagcggcgcgcgcgggcgcggaactgattgcgtttccggaaacctggttaccgggctatccgtggtatgcgtggctgtatgcgccggcaatgtgggtgccggtgtatattcgccgctattttgataacagcctggaatttggcctgccgcagaaacatcgcattcgcaaagatgcgaaagataacaacatttatgcggcgtttggcctgagcgaacgcgaaggcgaaagcctgtacatttctcagtgggtgattgatcgcgatggcgataccattgcggaacgccgcaaactgaaaccgacccatagcgaacgcaccatttttggcgaaggcgatggcagcgatctgggcgtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccggcgaagaaggcctgatttatgcggaaattgatctgggcctgattagcgtgagcaaaaacgtgaacgatccggcgggtcattatagccgcccagatgtgacccagttactgcatgataaagtgccgcgccgtgcagttgttgatactgcaccaccatctgacccagcaccggcggtggaacgtccacatggtgcagatccgccggcattaccggcgatggcggaactg

SEQ ID NO: 285 - protein, 344 aa, Synthetic construct
MNKPKFKAAAVQAAPVFLDLDKTIDKTRALMEKAARNGAELIAFPETWVPGYPWYAWLYAPAMWVLVYFRRYFDNSLEFDLPQKHRIRKDAKRNNIYMLMGLSEREGESLYISQWFIDRDGDTQAERRKLKPTHAERTIFGEGDGSDLAVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELHEGLPADEEGLIYAEIDLGLISVSKAVNDPAGHYSRPDVTQLIHDKAPRRAVVDTATPSDPAPTVERPHGADEGALPAMAEL

SEQ ID NO: 286 - DNA, 1032 bp, Synthetic construct
  source 1..1032: mol_type = other DNA
atgaacaaaccgaaatttaaagcggcggcggtgcaggcggcgccggtgtttctggatttagacaaaaccattgataagacccgcgcgctgatggaaaaagcggcgcgcaacggcgcggaactgattgcgtttccggaaacctgggtgccgggctatccgtggtatgcgtggctgtatgcgccggcaatgtgggtgctggtgtattttcgccgctattttgataacagcctggaatttgatctgccgcagaaacatcgcattcgcaaagatgcgaaacgcaacaacatttatatgctgatgggcctgagcgaacgcgaaggcgaaagcctgtacatttctcagtggtttattgatcgcgatggcgatacccaggcggaacgccgcaaactgaaaccgacccatgcggaacgcaccatttttggcgaaggcgatggcagcgatctggcggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccgatgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaaccccatctgacccagcaccgaccgtggaacgtccacatggtgcagatgaaggcgcattaccggcgatggcggaactg

SEQ ID NO: 287 - protein, 344 aa, Synthetic construct
MNKPKFKAAAVQAAPVFLDLDKSIDKTRALMEKAARNGAELIAFPETWVPGYPWYAWLYAPAMWVPVYTRRYFDNSLEFGTPQKHRIRKDAKDNNIYVVSGLSEREGESLYITQWFIDRDGDTIAERRKLKPTHTERTIFGEGDGSDLAVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELHEGLPAGEEGLIYAEIDLGLIAVSKAVNDPAGHYSRPDVTQLIHDKAPRRAVVDTAPPSDPAPAVERPHGLDEPALPAMAEL

SEQ ID NO: 288 - DNA, 1032 bp, Synthetic construct
  source 1..1032: mol_type = other DNA
atgaacaaaccgaaatttaaagcggcggcggtgcaggcggcgccggtgtttctggatttagacaaaagcattgataagacccgcgcgctgatggaaaaagcggcgcgcaacggcgcggaactgattgcgtttccggaaacctgggtgccgggctatccgtggtatgcgtggctgtatgcgccggcaatgtgggtgccggtgtatacccgccgctattttgataacagcctggaatttggcaccccgcagaaacatcgcattcgcaaagatgcgaaagataacaacatttatgtggtgagcggcctgagcgaacgcgaaggcgaaagcctgtacattacccagtggtttattgatcgcgatggcgataccattgcggaacgccgcaaactgaaaccgacccataccgaacgcaccatttttggcgaaggcgatggcagcgatctggcggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccggcgaagaaggcctgatttatgcggaaattgatctgggcctgattgcggtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtctggatgaaccggcattaccggcgatggcggaactg

SEQ ID NO: 289 - protein, 344 aa, Synthetic construct
MNKPKFKAAAVQAAPVWLDLDKTIDKTRALMEKAARNGAELIAFPETFLPGYPWYAWLYAPAMWVPVYTRRYFDNSLEFGTPQKHRIRKDAKDNNIYAGMGLSEREGESLYITQWFIDNDGDTIAERRKLKPTHVERTIFGEGDGSDLAVHDIRLGRVGVLCCWEHLQPLSKYAMYAQKEQVHVAAWPSFSIYGAYAYALGPIVNTSASRIYAVEGQCFVLAPCACVSKAMVDEMCTTDVRKALLKTGGCYASIFGPDGRELHEGLPAGEEGLIYAEIDLGLISVSKAVNDPAGHYSRPDVTQLIHDKAPRRAVVDTAPPSDPAPAVERPHGLDEPALPAMAEL

SEQ ID NO: 290 - DNA, 1032 bp, Synthetic construct
  source 1..1032: mol_type = other DNA
atgaacaaaccgaaatttaaagcggcggcggtgcaggcggcgccggtgtggctggatttagacaaaaccattgataagacccgcgcgctgatggaaaaagcggcgcgcaacggcgcggaactgattgcgtttccggaaacctttttaccgggctatccgtggtatgcgtggctgtatgcgccggcaatgtgggtgccggtgtatacccgccgctattttgataacagcctggaatttggcaccccgcagaaacatcgcattcgcaaagatgcgaaagataacaacatttatgcgggcatgggcctgagcgaacgcgaaggcgaaagcctgtacattacccagtggtttattgataacgatggcgataccattgcggaacgccgcaaactgaaaccgacccatgtggaacgcaccatttttggcgaaggcgatggcagcgatctggcggtgcatgatattcgtttaggtcgcgttggcgtgttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaaaaccggcggctgctatgcgagcatttttggcccggatggccgcgaattacatgaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtctggatgaaccggcattaccggcgatggcggaactg

SEQ ID NO: 291 - protein, 344 aa, Synthetic construct
MNKPKFKIAAVQAAPVFLDLDKTIDKTRALMEKAARNGAELIAFPETWVPGYPWYAWLYAPAMWVPVYTRRYFDNSLEFDSPQKHRIRKDAKNANIYVSMGLSEREGESLYIAQWFIDRDGDTLAERRKLKPTHSERTIFGEGDGSDLAVHDIRLGRVGGLCCWEHFQPLSKYAMYAQKEQVHVAAWPSFSIYGAYAYALGPIVNTSASRIYAVEGQCFVLAPCACVSKAMVDEMCSTDVRKALLKTGGCYASIFGPDGRELHEGLPHGEEGLIYAEIDLGLISVGKAVNDPAGHYSRPDVTQLIHDKAPRRAVVDTAPPSDPAPAVERPHGADEPALPAMAEL

SEQ ID NO: 292 - DNA, 1032 bp, Synthetic construct
  source 1..1032: mol_type = other DNA
atgaacaaaccgaaatttaaaattgcggcggtgcaggcggcgccggtgtttctggatttagacaaaaccattgataagacccgcgcgctgatggaaaaagcggcgcgcaacggcgcggaactgattgcgtttccggaaacctgggtgccgggctatccgtggtatgcgtggctgtatgcgccggcaatgtgggtgccggtgtatacccgccgctattttgataacagcctggaatttgatagcccgcagaaacatcgcattcgcaaagatgcgaaaaacgcgaacatttatgtgagcatgggcctgagcgaacgcgaaggcgaaagcctgtacattgcgcagtggtttattgatcgcgatggcgataccctggcggaacgccgcaaactgaaaccgacccatagcgaacgcaccatttttggcgaaggcgatggcagcgatctggcggtgcatgatattcgtttaggtcgcgttggcggcttatgctgctgggaacattttcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcagcaccgatgtgcgcaaagcgctgttaaaaaccggcggctgctatgcgagcatttttggcccggatggccgcgaattacatgaaggcctgccgcatggcgaagaaggcctgatttatgcggaaattgatctgggcctgattagcgtgggcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcagatgaaccggcattaccggcgatggcggaactg

SEQ ID NO: 293 - protein, 344 aa, Synthetic construct
MNKPKFKAAIVQAAPVFLDLDKTIDKTRALMEKAARNGAELIAFPETYLPGYPWYAWLYAPAMWVPVYTRRYFDNSLEFGLPQKHRIRKDAKQNNIYVSMGLSEREGESLYISQWFIDRDGDTQAERRKLKPTHSERTIFGEGDGSDLVVHDIRLGRVGGLCCWEHLQPLSKYAMYAQKEQVHVAAWPSFSIYGAYAYALGPIVNTSASRIYAVEGQCFVLAPCACVSKAMVDEMCTTDVRKALLTTGGCYASIFGPDGRELHEGLPAGEEGLIYAEIDLGLISVSKAVNDPAGHYSRPDVTQLIHDKAPRRAVVDTAPPSDPAPAVERPHGAPHPALPAMAEL

SEQ ID NO: 294 - DNA, 1032 bp, Synthetic construct
  source 1..1032: mol_type = other DNA
atgaacaaaccgaaatttaaagcggcgattgtgcaggcggcgccggtgtttctggatttagacaaaaccattgataagacccgcgcgctgatggaaaaagcggcgcgcaacggcgcggaactgattgcgtttccggaaacctatttaccgggctatccgtggtatgcgtggctgtatgcgccggcaatgtgggtgccggtgtatacccgccgctattttgataacagcctggaatttggcctgccgcagaaacatcgcattcgcaaagatgcgaaacagaacaacatttatgtgagcatgggcctgagcgaacgcgaaggcgaaagcctgtacatttctcagtggtttattgatcgcgatggcgatacccaggcggaacgccgcaaactgaaaccgacccatagcgaacgcaccatttttggcgaaggcgatggcagcgatctggtggtgcatgatattcgtttaggtcgcgttggcggcttatgctgctgggaacatctgcagccgctgagcaaatatgcgatgtatgcgcagaaagaacaggtgcatgtggcggcgtggccgagctttagcatttatggcgcgtatgcgtatgcgttaggcccgattgtgaacaccagcgcgagcagaatttatgcggtggaaggccagtgctttgtgctggcgccgtgcgcatgcgttagcaaagcgatggtggatgagatgtgcaccaccgatgtgcgcaaagcgctgttaaccaccggcggctgctatgcgagcatttttggcccggatggccgcgaattacatgaaggcctgccggccggcgaagaaggcctgatttatgcggaaattgatctgggcctgattagcgtgagcaaagcggtgaacgatccggcgggtcattatagccgcccagatgtgacccagttaattcatgataaagcgccgcgccgtgcagttgttgatactgcaccaccatctgacccagcaccggcggtggaacgtccacatggtgcaccgcatccggcattaccggcgatggcggaactg

(19) United States
(12) Patent Application Publication
(10) Pub. No.: US 2025/0084395 A1
(43) Pub. Date: Mar. 13, 2025

(54) ENGINEERED NITRILASES FOR BIOCATALYSIS

(71) Applicant: Kcat Enzymatic Private Limited, Bangalore (IN)

(72) Inventors: Pravin Kumar R, Bangalore (IN); Abhishek Kumar, Bangalore (IN); Naveen B K, Bangalore (IN); Gladstone Sigamani G, Bangalore (IN); Roopa L, Bangalore (IN); Pradeepa. A, Bangalore (IN); Sharal Priyanka Lewis, Bangalore (IN)

(73) Assignee: Kcat Enzymatic Private Limited, Bangalore, KA (IN)

(21) Appl. No.: 18/788,213

(22) Filed: Jul. 30, 2024

(30) Foreign Application Priority Data
Jun. 2, 2023 (IN) 202341031260

(51) Int. Cl. C12N 9/78; C12P 7/54; C12P 17/12

(52) U.S. Cl. CPC C12N 9/78; C12P 7/54; C12P 17/12; C12Y 305/05001

(57) ABSTRACT

The present invention provides an engineered nitrilase polypeptide capable of converting (1-cyanomethyl) cyclohexane-1-carbonitrile into (1-cyanocyclohexyl)-acetic acid. Utilizing advanced enzyme engineering techniques, the nitrilase exhibits enhanced stability and activity over natural variants. These engineered enzymes can hydrolyze a wide range of nitrile-containing compounds, including cyclic and aliphatic substrates. They handle higher substrate concentrations (200 g/L to 300 g/L), are thermostable above 50° C., and remain stable within a pH range of 5.5 to 8.0, making them suitable for various industrial applications. Their ability to convert substrates such as mandelonitrile, 2-(2-chlorophenyl)-2-hydroxyacetonitrile, 2-(6-methoxynaphthalen-2-yl)propanenitrile, and 2-[1-(aminomethyl)cyclohexyl]acetonitrile into corresponding carboxylic acids enables efficient and cost-effective production from diverse starting materials. This invention offers an engineered nitrilase enzyme as an alternative to alkaline or acid hydrolysis for converting nitrile substrates into carboxylic acids. It has applications in pharmaceuticals, agrochemicals, fine chemicals, waste treatment, and bioremediation, making these engineered polypeptides valuable for chemical production.

The instant application contains a Sequence Listing which has been submitted electronically in XML file format and is hereby incorporated by reference in its entirety. Said XML copy, created on Jul. 30, 2024, is named Nitrilase_SequenceListing.xml and is 4,56,752 bytes in size.

### FIELD OF THE INVENTION

The present invention relates to the field of Biology, Life Sciences, Biocatalysis, Chemistry, Enzymology and enzyme engineering.

### BACKGROUND OF THE INVENTION

Nitrile bonds can be seen across different molecules, nitriles are compounds that have a nitrogen atom triple-bonded with a carbon atom. Carbon-Nitrogen hydrolysis reaction was found in many plants, animals and fungi using Nitrilase enzymes.

Nitrilases are enzymes known for the condensation and hydrolysis of carbon-nitrogen bonds (Howden et. a., 2009, Silva et. all., 2021). Nitrilase enzymes hold unique structural features, they can form long filamentous structures consisting of spirals, turns and helices. The active site of a monomer is stabilized by the other 4 monomers where the C-terminal will form β sheets which stabilizes the monomeric structure and supports forming spiral conformation Mulelu, Andani E. et. al., 2019. The core catalytic pocket of a monomer is supported other two chains structurally. The residues from other chains provide structural stability and anchor the substrate entry and product exit. The catalytic residues responsible for nitrilase activity contain a Lys, two Glu and a Cys in the active site (NOVO, Carlos, et al 2002). Glu and Cys were first mutated to other amino acids inorder to confirm the catalytic involvement of these residues. This resulted in a loss of activity in each case. There was no longer any activity once Cys was mutated to Ser and Ala. Glu also became inactive as it mutated into Asp and Gln. Following Lys's transformation, Arg shows 200 times less activity than Lys. This implies that these four catalytic residues are essential for the hydrolysis of nitrile molecules. For the process to be completed, water molecules are required. The reaction is initiated by Glu, which takes a proton from water, and water abstracts a proton from catalytic Cys. The S⁻ of Cys nucleophilic attack on cyano carbon of the substrate results in the formation of a thioacyl-enzyme intermediate, meanwhile the nitrogen of the cyano group abstracts a proton from water resulting in the formation of NH. The HO⁻ now forms a bond with the carbon of the Cyano group where a thioacyl bond formed, during the event NH of the cyano group abstracts another proton from Lys resulting in the formation of NH₂. The Lys abstracts a proton from HO and Glu, meanwhile, NH₂ of the Cyano group takes hydrogen from Lys which leads to the shuffling of electrons resulting in the release of NH₃ from the active site pocket. Another water molecule enters into the active site to complete the reaction where Glu abstracts a proton from water, negatively charged HO⁻ attacks on the carbon of substrate where a thioacyl bond formed resulting in the breaking of thioacyl bond. The negatively charged S⁻ of Cys abstracts the proton from Glu which prepares the enzyme for the next reaction. The nitrilase enzymes as they form thioacyl intermediate any thio-bond forming molecules such as silver nitrate, and copper sulphate inhibit the reaction mechanism (Lay, Norman, et. all, 1998).

Nitrilase enzymes are highly selective that a given nitrilase catalyses the conversion of a specific nitrile substrate only (DeSantis, Grace, et al., 2002, Shen, Ji-Dong, et al., 2020, Gong, Jin-Song, et al., 2012) They typically show little or no activity on other types of substrates, restricting their activity to the specific substrate. The enzymatic bio-catalyzed degradation of cyanide or nitrile molecules is environmentally better compared to chemical hydrolysis which uses strong bases and acids with higher temperatures. There are reported nitrilases from various organisms such as Syntrophobacter fumaroxidans, Pyrococcus abyssi, Burkholderia xenovorans, Bradyrhizobium japonicum, Acidovorax facilis, Delftia tsuruhatensis ZJB-05174, Rhodococcus aetherivorans ZJB1208, Klebsiella pneumoniae sp. ozaenae and Arabidopsis thaliana, Bacillus pallidus Dac521, etc., These and many other reported nitrilases are classified based on the substrate scope of it as they shown higher substrate specificity. Nitrilase from different plant sources shows better activity towards nitrile compounds as a part of cyanide metabolism which finds majorly in herbicides.

Nitrilases enzymes are reported for synthesizing important pharmaceutical intermediates example, (1-cyanocyclohexyl)-acetic acid, which is an important intermediate for the synthesis of Gabapentin. Gabapentin is commonly used to treat and prevent seizures in people with epilepsy or to treat nerve pain (postherpetic neuralgia) that can occur after a viral infection called shingles. The importance of the drug made it to be synthesized by many different chemical methods, where hydrogenation is a major method used for the synthesis (CN102093237A). The chemical synthesis of the gabapentin intermediate is not industrially economical as there is low yield and low enantiomeric access due to unspecific regioselectivity. Due to the unspecific regioselectivity of chemical process an enzymatic and chemoenzymatic processes were preferred using the nitrilase enzyme. The chemoenzymatic synthesis of gabapentin is a known method where harbouring nitrilase in a whole cell system or free enzyme to produce above mentioned intermediate and then for the second step of the reaction proceeded using chemical methods (Shen, Qi, et al., 2020, Xue, Ya-Ping, Ying-Peng Wang, et al. 2015, Xue, Ya-Ping, et al., 2017). Nitrilases enzymes have been reported to produce an intermediate of gabapentin, that converts 1-(cyanomethyl)cyclohexane-1-carbonitrile into (1-cyanocyclohexyl)-acetic acid with limited activity and substrate load tolerance. Wong et. al., 2005 implemented a whole-cell system method to synthesize gabapentin intermediate (US20050009154A1), where Acedovorax facilis organism and its mutants were used, In another instance, a nitrilase from Bradyrhizobium japonicum strain USDA10 was tested for the same gabapentin reaction, yet its lack of stability at higher substrate concentrations and longer reaction period made it not eligible for the industrially viable enzyme (Zhu, Dunming, et al., 2007). To address the limitations of existing nitrilases, we have engineered a nitrilase with phenomenal improvements specifically for the biocatalytic synthesis of gabapentin intermediates, thereby enhancing its suitability for industrial applications.

Many plants store cyanide group-associated lipid molecules in the seed as the source of nitrogen. The breakdown of glucosinolate molecule produces 1-cyano-2,3epithiopropane, 3-butene nitrile etc., which will be reduced by the nitrilase enzymes. And the conversion of indole-3-acetonitrile to indole-3-acetic acid is a common involvement of nitrilase in plants as indole-3-acetic acid is a plant growth hormone (DeSantis, Grace, et al., 2002, Shen, Ji-Dong, et al., 2020, Gong, Jin-Song, et al., 2012, Hanschen, Franziska S, et al., 2017)

Nitrilase enzymes are unique in their substrate selection, and many studies have been reported explaining their capability to convert different nitrile compounds, but also nitrilase enzymes are unstable in industrial environments, higher temperatures, solvent conditions, higher substrate concentrations, variable pH conditions etc., The very first nitrilase reported with the name ricinine nitrilase, which is by the name used to act on recinine (4-methoxy-1-methyl-2-oxo-dihydropyridine-3-carbonitrile) molecule which is a central nervous system stimulant found majorly in castor oil seeds, which is also intoxicated animals. Nitrilase enzyme from Pseudomonas Sp. Was considered in this study where it showed activity for other molecules as well such as 3-cyano2-pyridone, 3-cyano-4-methoxy-2-pyridone, and N-methyl-3-cyano-2-pyridone etc., where it showed relative activity of 28%, 24% and 38% respectively (Robinson, William, and Robert Hook et. all., 1964). Nitrilase converts many different molecules and uses it as an energy source, growth factor etc., Harper et. al., 1976 reported a nitrilase from Nocardia sp. which uses benzonitrile as the sole carbon, nitrogen and energy source. They have tested other molecules as well such as benzonate, catechol, and benzamide but the O₂ update was better when benzonitrile was consumed. Nagasawa, Toru, et al 2000, reported nitrilase from R. rhodochrous J1 was tested against Benzonitrile and Acrylnitrile where the activity was tested with a different medium. Both benzonitrile and Acrylnitrile showed better activity when they tested in the media+50% (v/v) glycerol+10% saturated (NH₄)₂SO₄ₗ⁻ The same cells were recovered and tested for the second round of activity and compared with the purified enzymes in the presence of (NH₄)₂SO₄. For benzonitrile, 3-cyanopyridine, Acrylonitrile, methacrylonitrile, and crotononitrile resulted in the relative activity of 100%, 95.5%, 128%, 9%, and 27% by recovered cells, 100%, 75%, 58%, 7.45%, 11% by purified enzymes in the presence of (NH₄)₂SO₄. Piotrowski, Markus, et. al in 2001 reported nitrilase from Arabidopsis thaliana which is tested against a range of substrates such as b-Cyano-L-alanine, 3-Phenylpropionitrile, Phenylacetonitrile, (Methylthio)acetonitrile, Chloroacetonitrile, 3-Aminopropionitrile, Allylcyanide, and 4-Phenylbutyronitrile resulting in the relative activity of 100%, 0.75%, 0.23%, 0.21%, 0.07%, 0.06%, 0.03% and 0.02%, respectively. The activity reported shows poor selectivity of the substrate by the enzyme which is tested at pH of 7.5. In 2002, Osswald et al. tested the same nitrilase which resulted in better activity for 3-phenyl propionitrile, octanenitrile and butyronitrile only. The other substrates tested showed minute conversion. Other aromatic and aliphatic substrates tested were shown less than 50% conversion. The whole experiment was tested under different conditions the buffer system was changed from Tris/HCl to glycine/NaOH system with optimum pH at 8.5, and Higher or lower pH modifications were subjected to loss of activity.

Pregabalin is a long-acting medication used to treat various nervous system illnesses, including epilepsy, post-herpetic neuralgia, diabetic peripheral neuropathy, anxiety, and social phobia. It has fewer adverse effects compared to other medications and provides longer-lasting relief. Pregabalin is also occasionally prescribed at lower dosages. Its success in the market is evident from the $5.06 billion in pharmaceutical sales it generated in 2017, making it a blockbuster drug in many countries. As a result, the pharmaceutical industry has invested significant research efforts into the production of pregabalin (Steffens, David. Et al., 2008, Calandre, Elena P., et al. 2016, Tao, Junhua, and Jian-He Xu. et al., 2009). The production process involves several steps, isovaleraldehyde and diethyl malonate are condensed using the Knoevenagel method. This is followed by cyanation to create a cyano diester, which serves as the regulatory starting material (2-carboxyethyl-3-cyano-5-methylhexanoic acid ethyl ester, CNDE). Subsequently, racemic pregabalin is hydrolyzed, decarboxylated, hydrogenated, and then resolved using (S)-mandelic acid. However, this approach has a total yield of only approximately 20% due to the need for a final-stage resolution, with the unwanted enantiomer being unable to be recycled (Burk MJ. et al., 2003, Li, Xiao-Jun, et al., 2014. In the study by Zhang, Qin, et al., 2019 a new nitrilase, known as AaNIT, derived from Arabis alpina. This nitrilase was modified to produce the N258D mutant, which exhibited remarkable activity and enantioselectivity greater than 300% in the synthesis of (S)-3-cyano-5-methylhexanoic acid ((S)-CMHA), a vital intermediate in the production of pregabalin. Through the utilization of a one-pot bi-enzymatic cascade involving the N258D mutant and an amidase from Pantoea sp. the researchers effectively eliminated the undesired byproduct ((S)-CMHM), resulting in a conversion rate of 45.0% to (S)-CMHA with a purity of 99.3% in terms of enantiomeric excess. To overcome the product of unwanted products a chemoenzymatic method was implemented and to improve the yield of pregabalin. Zhang, Qin et al., 2019 developed a novel chemoenzymatic approach that uses a distinct Brassica rapa nitrilase enzyme to synthesize pregabalin from isobutylsuccinonitrile (IBSN). Escherichia coli cells immobilized with Brassica rapa nitrilase (BrNIT) hydrolyzed IBSN in a regio- and enantioselective manner to produce the crucial intermediate (S)-CMHA. The substrate concentration of 100 g/L showed conversion of (>41.1%) and enantioselectivity (E>150). Furthermore, the leftover (R)-IBSN was efficiently recycled, and the resultant (S)-CMHA was hydrogenated straight to generate pregabalin with remarkable optical and 99.6% purity. Another study by Zhang, Qin, Xia-Feng Lu, et al. 2019 developed an engineered nitrilase to overcome chemo-enzymatic method and to improve the pregabalin yield. Where they successfully developed a novel approach to engineering nitrilase enzymes through a combination of fragment swapping and semi-rational design. This method has led to the creation of a chimeric enzyme called BaNIT, which possesses 12 amino acid alterations in comparison to the nitrilases found in Arabis alpine and Brassica rapa. These specific modifications have had a profound impact on the enzyme's performance, significantly enhancing both its activity and enantioselectivity in the synthesis of the precursor of pregabalin. Through further refinement of the design, the scientists have successfully generated the BaNITL223Q/H263D/Q279E variant, which exhibits an astounding 5.4-fold increase in activity and an enantiomeric ratio surpassing greater than 300. This studies also produced unwanted by product, the same but it also produced an unwanted by-product. To refine this, BaNITM0 underwent further engineering to enhance its reaction specificity, enantioselectivity, and overall activity. The resulting variant, V82L/M127I/C237S (BaNITM2), achieved a remarkable enantioselectivity (E=515), a 5.4-fold increase in enzyme activity, and significantly less by-product formation. Structural analysis and simulations revealed that specific mutations led to these improvements by altering the enzyme's active site. Using Escherichia coli cells with BaNITM2, the hydrolysis of IBSN resulted in a high yield of (S)-CMHA with excellent enantiomeric purity and conversion rate. A molecular modification of the nitrilase enzyme by Chen, Zhi, et al., 2021 produced favourable effects. Its ability to generate rac-ISBN to (S)-CMHA, an essential step in the synthesis of optically pure pregabalin, was made possible by this transformation. It was possible to produce two mutants with high enantioselectivity (E>300), namely W57F/V134M and W57Y/V134M. Additionally, with substantial enantiomeric excess (i.e., >99.9%) and conversion ratios of 43.8% and 40.9%, respectively, these mutants accomplished the kinetic resolution of rac-ISBN to (S)-CMHA. Baclofen, also known as (3S)-3-(4-chlorophenyl)-4-cyanobutanoic acid, is a potent medication that effectively relaxes specific muscles in the human body. It provides relief from spasms, cramps, and muscle tightness caused by medical conditions such as multiple sclerosis or certain spinal injuries (Warren, Rachel L., and Steven M. Davis et. all, 2015). 2-chloronicotinic acid is a known pharmaceutically important intermediate for the synthesis of antibiotics, anti-cardiovascular drugs, and many herbicides. It is also a majorly known compound in the synthesis of pranoprofen and diffluence which is an anti-inflammatory drug and a well-known herbicide, respectively. Indole acetic acid or commonly known as IAA is an auxin, growth factor in plants. It can be found on the tip of the growing plants; it promotes the growth and development process in plants. R-mandelic acid and R-chloromandelic acid are anti-bacterial drugs which is also used widely in dermatological applications, also known for their role as cardiovascular drugs. The synthesis of these two drugs chemically is economically not feasible as maintaining the specific enantiomeric access is not easy. R-mandelic acid as an intermediate in clopidogrel drug is sold under the brand name Plavix which is prescribed to control heart diseases and stroke. Naproxen, with the chemical name (2S)-2-(6-methoxynaphthalen-2-yl) propanoic acid is a known drug prescribed for arthritis-related joint pains, tiredness, etc. As earlier explained, gabapentin is used for epilepsy, and nerve pain, and is used to treat brain stroke, bipolar disorder and borderline personality disorder etc, Ibuprofen is known for its wide role in treating fever, pain reliever, anti-inflammatory, etc., Herein the invention provides engineered nitrilases that can act on substrates to produce above mentioned and more other products (FIG. 1). The engineered nitrilase can accommodate a wide number of pharmaceutical nitrile substrates and convert them into corresponding carboxylic acids, with improved activity at conditions such as higher substrate concentration, broad pH and temperature range making it suitable for wider applications in a faster and cost-effective manner.

### OBJECTS OF THE INVENTION

The primary objective is to engineer a nitrilase polypeptide that exhibits enhanced specificity and efficiency for the conversion of 1-(cyanomethyl)cyclohexane-1-carbonitrile to (1-cyanocyclohexyl)-acetic acid, achieving 100% conversion within 24 hours with an enzyme load of less than 5% relative to the substrate concentration under defined reaction conditions. Further, the secondary objective is to engineer nitrilases capable of acting on a wide range of nitrile substrates with improved activity, including 3-(4-chlorophenyl)pentanedinitrile, 2-chloropyridine-3-carbonitrile, indole-3-acetonitrile, mandelonitrile, 2-(2-chlorophenyl)-2-hydroxyacetonitrile, 2-(6-methoxynaphthalen-2-yl)propanenitrile, 2-[1-(aminomethyl)cyclohexyl]acetonitrile, 2-[4-(2-methylpropyl)phenyl]propanenitrile, (+/−)-2-hydroxy-3-phenylpropanenitrile, (+/−)-3-(3-fluorophenyl)-2-hydroxypropanenitrile, (+/−)-2-hydroxy-3-(naphthalen-1-yl)propanenitrile, (+/−)-2-hydroxy-3-(pyridin-2-yl)propanenitrile, (+/−)-2-hydroxy-3-(thiophen-3-yl)propanenitrile, and (+/−)-isobutylsuccinonitrile to produce (3S)-3-(4-chlorophenyl)-4-cyanobutanoic acid, 2-chloronicotinic acid, indoleacetic acid, R-mandelic acid, (2-chlorophenyl)(hydroxy)acetic acid, S-naproxen, (1-cyanocyclohexyl)acetic acid, gabapentin, S-ibuprofen, S-phenyllactate, (2S)-3-(3-fluorophenyl)-2-hydroxypropanoic acid, (2S)-2-hydroxy-3-(naphthalen-1-yl)propanoic acid, (2S)-2-hydroxy-3-(pyridin-2-yl)propanoic acid, (2S)-2-hydroxy-3-(thiophen-3-yl)propanonitrile, and S-3-cyano-5-methylhexanoic acid, respectively, including engineering the active site to accommodate these substrates, increasing enzyme stability across a broader pH range and different temperatures, and developing a polynucleotide that encodes the engineered nitrilase, operably linked to one or more promoter sequences to facilitate the production of recombinant nitrilase in a recombinant host cell using an expression vector and expressing the engineered nitrilase in a recombinant host cell.

### SUMMARY OF THE INVENTION

The engineered nitrilases described here is specifically engineered for converting (1-cyanomethyl) cyclohexane-1-carbonitrile to (1-cyanocyclohexyl)-acetic acid. Additionally, the engineered nitrilases act on a wide variety of nitrile-containing compounds. They can hydrolyse both cyclic and aliphatic substrates. They are more stable than natural nitrilase in reactions with higher substrate concentrations as 200 g/L to 300 g/L which improves the efficiency of the reaction. They are thermostable withstanding a broad range of temperatures greater than 50° C. and are stable at acidic and basic conditions withstanding pH ranges of 5.5 to 8.0, allowing for their use in broad-temperature and pH applications. Here, in this invention the nitrilase enzyme is engineered in the active site to accommodate wide range of substrates such as 3-(4-chlorophenyl)pentanedinitrile, 2-chloropyridine-3-carbonitrile, indole-3-acetonitrile, mandelonitrile, 2-(2-chlorophenyl)-2-hydroxyacetonitrile, 2-(6-methoxynaphthalen-2-yl)propanenitrile, 1-(cyanomethyl) cyclohexane-1-carbonitrile, 2-[1-(aminomethyl) cyclohexyl]acetonitrile, 2-[4-(2-methylpropyl)phenyl]propanenitrile, (+/−)-2-hydroxy-3-phenylpropanenitrile, (+/−)-3-(3-fluorophenyl)-2-hydroxypropanenitrile, (+/−)-2-hydroxy-3-(naphthalen-1-yl)propanenitrile, (+/−)-2-hydroxy-3-(pyridin-2-yl)propanenitrile, (+/−)-2-hydroxy-3-(thiophen-3-yl)propanenitrile, and (+/−)-Isobutylsuccinonitrile to produce (3S)-3-(4-chlorophenyl)-4-cyanobutanoic acid, 2-chloronicotinic acid, indoleacetic acid, R-mandelic acid, (2-chlorophenyl)(hydroxy)acetic acid, S-naproxen, (1-cyanocyclohexyl)-acetic acid, gabapentin, S-ibuprofen, S-phenyllactate, (2S)-3-(3-fluorophenyl)-2-hydroxypropanoic acid, (2S)-2-hydroxy-3-(naphthalen-1-yl)propanoic acid, (2S)-2-hydroxy-3-(pyridin-2-yl)propanoic acid, (2S)-2-hydroxy-3-(thiophen-3-yl)propanoic acid, and S-3-cyano-5-methylhexanoic acid, respectively (FIG. 1). The active site engineering allowed these substrates to fit and be anchored by the substituted amino acids. The catalytic residues, two Glu, a Lys and a Cys were not disturbed to maintain the reaction mechanism. The reaction begins with Glu accepting a proton from water, while water removes a proton from catalytic Cys. The S⁻ of Cys then initiates a nucleophilic attack on the cyano carbon of the substrate, resulting in the formation of a thioacyl-enzyme intermediate. Simultaneously, the nitrogen of the cyano group removes a proton from water, forming NH₃. Next, HO⁻ forms a bond with the carbon of the cyano group, leading to the formation of a thioacyl bond. Throughout this process, NH₃ of the cyano group removes another proton from Lys, resulting in the creation of NH₂. Additionally, Lys removes a proton from HO⁻ and Glu, while NH₂ of the cyano group takes hydrogen from Lys. This electron rearrangement releases NH₃ from the active site pocket. Another water molecule enters the active site to complete the reaction. Glu removes a proton from water, and the negatively charged HO⁻ attacks the carbon of the substrate, forming a thioacyl bond and breaking the previous thioacyl bond. Lastly, the negatively charged S⁻ of Cys removes the proton from Glu, preparing the enzyme for the next reaction (FIG. 2). Having the reacting mechanism as a reference the docking studies were conducted for the above-mentioned substrates (FIG. 3-7). FIGS. 8 to 12 provide a detailed description of the method for deriving an engineered nitrilase enzyme. They explain how multiple natural nitrilase enzymes are used to engineer a new nitrilase enzyme, utilizing pLDDT_{F}-based scoring functions to identify the top-ranking substitutions. The engineered nitrilases exhibit greater activity compared to their natural counterparts, resulting in enhanced conversion rates and yields with fewer by-products. This reduction in by-products significantly decreases the number of downstream processing steps required. Nitrilases are increasingly used for the enzymatic conversion of various nitrile substrates to acid products, eliminating the need for many chemical synthetic procedures to produce key compounds. Biocatalytic conversions can employ whole cells expressing nitrilases, while purified enzymes are preferred when the presence of multiple nitrilases affects the specificity and yield of the desired product.

### BRIEF DESCRIPTION OF DRAWINGS

FIG. 1: Reaction showing conversion of nitrile substrate (i) 3-(4-chlorophenyl)pentanedinitrile; (ii) 2-chloropyridine-3-carbonitrile; (iii) indole-3-acetonitrile; (iv) Mandelonitrile; (v) 2-(2-chlorophenyl)-2-hydroxyacetonitrile; (vi) 2-(6-methoxynaphthalen-2-yl)propanenitrile; (vii) 1-(cyanomethyl)cyclohexane-1-carbonitrile; (viii) 2-[1-(aminomethyl)cyclohexyl]acetonitrile; (ix) 2-[4-(2-methylpropyl)phenyl]propanenitrile; (x) 2-hydroxy-3-phenylpropanenitrile; (xi) 3-(3-fluorophenyl)-2-hydroxypropanenitrile; (xii) 2-hydroxy-3-(naphthalen-1-yl)propanenitrile; (xiii) 2-hydroxy-3-(pyridin-2-yl)propanenitrile; (xiv) 2-hydroxy-3-(thiophen-3-yl)propanenitrile; and (xv) Isobutylsuccinonitrile to corresponding carboxylic acid product catalysed by an engineered Nitrilases; the reaction substrate and product carrying R group wherein the possible structures of “R” are given herewith which is indicative of the wide substrate scope of the engineered nitrilase.

FIG. 2: The reaction mechanism of nitrilase enzyme where the reaction was proceeded by the catalytic residues Cys, Lys and Glu. These residues initiate the reaction with the help of a water molecule where Glu abstracts a proton from water, while water removes a proton from catalytic Cys. The S⁻ of Cys then initiates a nucleophilic attack on the cyano carbon of the substrate, resulting in the formation of a thioacyl-enzyme intermediate. Simultaneously, the nitrogen of the cyano group removes a proton from water, forming NH₃. Next, HO⁻ forms a bond with the carbon of the cyano group, leading to the formation of a thioacyl bond. Throughout this process, NH₃ of the cyano group removes another proton from Lys, resulting in the creation of NH₂. Additionally, Lys removes a proton from HO⁻ and Glu, while NH₂ of the cyano group takes hydrogen from Lys. This electron rearrangement releases NH₃ from the active site pocket. Another water molecule enters the active site to complete the reaction. Glu removes a proton from water, and the negatively charged HO⁻ attacks the carbon of the substrate, forming a thioacyl bond and breaking the previous thioacyl bond. Lastly, the negatively charged S⁻ of Cys removes the proton from Glu, preparing the enzyme for the next reaction.

FIG. 3. Illustrates the near attack conformation of substrates 3-(4-chlorophenyl) pentanedinitrile, 2-chloropyridine-3-carbonitrile and Indole-3-acetonitrile in the active site of engineered nitrilase enzyme, where all the substrates were shown energetically feasible conformation with the distance less than 4° A to the catalytic Cys. The substrate molecules are shown in dark sticks and catalytic residues in light sticks FIG. 3a is near attack conformation of 3-(4-chlorophenyl) pentanedinitrile, FIG. 3b is a near attack conformation of 2-chloropyridine-3-carbonitrile FIG. 3c is a near attack conformation of Indole-3-acetonitrile.

FIG. 4. Illustrates the near attack conformation of substrates Mandelonitrile, 2-(2-chlorophenyl)-2-hydroxyacetonitrile and 2-(6-methoxynaphthalen-2-yl)propanenitrile in the active site of engineered nitrilase enzyme, where all the substrates were shown energetically feasible conformation with the distance less than 4° A to the catalytic Cys. The substrate molecules are shown in dark sticks and catalytic residues in light sticks FIG. 4a is near attack conformation of Mandelonitrile, FIG. 4b is a near attack conformation of 2-(2-chlorophenyl)-2-hydroxyacetonitrile FIG. 4c is a near attack conformation of 2-(6-methoxynaphthalen-2-yl)propanenitrile.

FIG. 5. Illustrates the near attack conformation of substrates 1-(cyanomethyl)cyclohexane-1-carbonitrile, 2-[1-(aminomethyl)cyclohexyl]acetonitrile and 2-[4-(2-methylpropyl)phenyl]propanenitrile in the active site of engineered nitrilase enzyme, where all the substrates were shown energetically feasible conformation with the distance less than 4° A to the catalytic Cys. The substrate molecules are shown in dark sticks and catalytic residues in light sticks FIG. 5a is near attack conformation of 1-(cyanomethyl)cyclohexane-1-carbonitrile, FIG. 5b is a near attack conformation of 2-[1-(aminomethyl)cyclohexyl]acetonitrile FIG. 5c is a near attack conformation of 2-[4-(2-methylpropyl)phenyl]propanenitrile.

FIG. 6. Illustrates the near attack conformation of substrates 2-hydroxy-3-phenylpropanenitrile, 3-(3-fluorophenyl)-2-hydroxypropanenitrile and 2-hydroxy-3-(naphthalen-1-yl)propanenitrile in the active site of engineered nitrilase enzyme, where all the substrates were shown energetically feasible conformation with the distance less than 4° A to the catalytic Cys. The substrate molecules are shown in dark sticks and catalytic residues in light sticks FIG. 6a is near attack conformation of 2-hydroxy-3-phenylpropanenitrile, FIG. 6b is a near attack conformation of 3-(3-fluorophenyl)-2-hydroxypropanenitrile FIG. 6c is a near attack conformation of 2-hydroxy-3-(naphthalen-1-yl)propanenitrile.

FIG. 7. Illustrates the near attack conformation of substrates 2-hydroxy-3-(pyridin-2-yl)propanenitrile, 2-hydroxy-3-(thiophen-3-yl)propanenitrile and 2-(2-methylpropyl)butanedinitrile in the active site of engineered nitrilase enzyme, where all the substrates were shown energetically feasible conformation with the distance less than 4° A to the catalytic Cys. The substrate molecules are shown in dark sticks and catalytic residues in light sticks FIG. 7a is near attack conformation of 2-hydroxy-3-(pyridin-2-yl)propanenitrile, FIG. 7b is a near attack conformation of 2-hydroxy-3-(thiophen-3-yl)propanenitrile FIG. 7c is a near attack conformation of 2-(2-methylpropyl)butanedinitrile.

FIG. 8: The flowchart describes the protocol for identifying the hotspots for engineering a nitrilase to convert (1-(cyanomethyl)cyclohexane-1-carbonitrile into (1-cyanocyclohexyl)-acetic acid using Step 1: using a natural nitrilase enzyme sequence as a template. Step2: The structure of natural nitrilase enzyme sequence was modelled using AlphaFold tool. Step 3: The modelled structure facilitates the identification of vdW contact residues for each amino acid in the enzyme, which are then catalogued as distinct fragments. For instance, the interaction details for the amino acid at the first position are extracted and designated as Fragment 1. This procedure is mirrored for the second position, which is archived as Fragment 2, and so on for all amino acids within the enzyme, ensuring that the contact residues and their associated fragment information are systematically documented. Additionally, 3D structural fragments for each residue of the nitrilase enzyme are constructed using the vdWcontacts method. These structural fragments are computed according to Equation 1 where the term ‘vdWcontact’ refers to the interaction between two atoms, (i) and (j), calculated as the sum of their Van der Waals (vdW) radii minus the distance between them, with a deduction for an allowance in cases of potential hydrogen-bonded atom pairs. This allowance is only applied to pairs consisting of a hydrogen bond donor (or the hydrogen atom attached to the donor) and an acceptor. For example, a position at X229 and its contacting residues X233, X232, X247, and X231 are collectively defined as fragment-229. Step 4: In the next step, the fragments were further analysed by extracting pLDDT scores of individual amino acids. The sum value of all the pLDDT scores of the residues in a fragment is used to obtain pLDDT_{F} value, the calculation of pLDDT_{F} is explained in Equation 2. Step 5: The pLDDT_{F} values with respect to its fragment were tabulated and fragments with lower pLDDT_{F} values were selected to predict hotspots. Step 6: Within the identified hotspots, certain ones may be essential for function, stability and evolutionary conservation. Multiple sequence alignment was generated using diverse natural nitrilase enzymes from the UniRef90 databases. Further, probability values were derived using MSA for each residue position using Equation 3. The non-conserved residues were selected from the fragments for subsequent engineering processes.

FIG. 9: The flowchart outlines the sequential process of enhancing the stability of selected nitrilase enzyme fragments. This is achieved by improving the pLDDT_{F} scores through strategic substitutions. The process begins with the tabulation of pLDDT_{F} scores for each fragment, as depicted in a previous flowchart (FIG. 8, step 5). A fragment exhibiting lower pLDDT_{F} scores is chosen for hotspot prediction. In step 6, non-conserved residues within the fragments are pinpointed based on probability values, deliberately avoiding highly conserved residues for hotspot designation. In Step 7 Substituting specific residues having low pLDDT_{F} scores with evolutionary information-based amino acids possessing higher pLDDT scores, as identified through multiple sequence alignment and sourced from the uniref90 database. In Step 8, Utilizing AlphaFold to model the substitutions and validate the enhanced pLDDT_{F} score of the modified fragment. Step 9, Computing the ΔpLDDT_{F} score for the fragment to confirm the ΔpLDDT_{F} score improvement at engineered sites. Step 10, Generating an engineered nitrilase by integrating substitutions at multiple identified hotspots. Further engineering protocols for engineering nitrilase (derived from steps 1 to 10) are explained in Steps 11a, 11b, and 11c.

FIG. 10: Flowchart showing a method of engineering of nitrilases in the active site for improving the selectivity of different substrates (1-cyanocyclohexyl)-acetic acid, 3-(4-chlorophenyl)pentanedinitrile, 2-chloropyridine-3-carbonitrile, indole-3-acetonitrile,

Mandelonitrile, 2-(2-chlorophenyl)-2-hydroxyacetonitrile, 2-(6-methoxynaphthalen-2-yl)propanenitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile, and Isobutylsuccinonitrile to corresponding carboxylic acid using site saturation mutagenesis. The starting (engineered nitrilase) point is considered from step 10 of FIG. 9. The protocol 11a onwards explains about site saturation mutagenesis of active site residues to improve binding affinity.

FIG. 11: Flowchart showing a method of engineering of nitrilase for improving the substrate load, pH and temperature tolerance against different substrates (1-cyanocyclohexyl)acetic acid, 3-(4-chlorophenyl)pentanedinitrile, 2-chloropyridine-3-carbonitrile, indole-3-acetonitrile, Mandelonitrile, 2-(2-chlorophenyl)-2-hydroxyacetonitrile, 2-(6-methoxynaphthalen-2-yl)propanenitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile, and Isobutylsuccinonitrile to corresponding carboxylic acid using 7D grid technology.

FIG. 12: Flowchart highlighting the method of engineering of nitrilase enzyme using funnel metadynamics simulations. The Collective variables were defined with respect to the funnel. The XY-plane and Z-axis of the funnel were prepared by considering hotspots selected for the engineering. The Funnel metadynamics will allow movement of the molecule or substrate of interest with the given boundary of XY-plane or within the funnel radius and explore the Gaussian wells. The deposited Gaussian wells provide observed free-energy convergence of the simulated E-S complex. The difference in free energy convergence of ENIT_{Mutant} and ENIT highlights the better or bad binding affinity of the substrate of interest. If the chosen hotspot and mutant are not improving the binding affinity of the substrate of interest the hotspots and substitutions will be selected in different combinations to generate optimised substrate-specific enzyme variants. In the figure, the grey box represents the funnel metadynamics simulation protocol.

FIG. 13: Generating van der Waals (vdW) contacts to create 3D structural fragments of the enzyme for every residue of the enzyme. The residue shown in dark grey sticks X229 is the central residue, while the residues shown in light grey are contact-making residues derived using vdWcontact; where ‘vdWcontact’ is defined between two atoms (i, j) as the sum of their vdW radii minus the distance between them and minus an allowance for potentially hydrogen-bonded pairs. The allowance is only subtracted for pairs comprised of a donor (or donor-borne hydrogen) and an acceptor.

FIG. 14: pLDDT matrix for introducing substitution using pLDDT score across the plurality of nitrilase for specific residue position.

FIG. 15: All the engineered Nitrilase polypeptides were induced and expressed in bacteria. SDS Gel images of 10 of the same are shown in the figure; Dark bands show the expression of engineered Nitrilase polypeptides.

FIG. 16a highlights the pET28a(+) plasmid construct with nitrilase gene insert region and FIG. 16b shows the Insertion of engineered nitrilase construct in the pET28a(+) plasmid between NdeI and BamHI restriction site.

FIG. 17. An example reaction of interest with respect to one of the engineered nitrilase polypeptides. (1-Cyanocyclohexaneacetonitrile is also known as 1-(cyanomethyl)cyclohexane-1-carbonitrile).

Table 1. Mutations on engineered nitrilase where multiple rounds of enzyme variants with different substitutions were generated and tested. The activity of the respective enzyme variants is given with the indication of “+”. The sequences were tested against all substrates listed in this embodiment and mainly for the conversion of (1-(cyanomethyl)cyclohexane-1-carbonitrile into (1-cyanocyclohexyl)-acetic acid. “++++” indicates the activity>98% and up to 100%, “+++” indicates the activity of >95% and <98%, “++” indicates >90% and <95%, and “+” indicates >85% and <90% activity obtained. The number of residues difference indicates the difference of substitutions incorporated with reference to SEQ ID NO: 1. Here, the SEQ ID NO is a combination of polypeptide SEQ ID followed by polynucleotide SEQ ID.

Table 2: Contains list of fragments selected for hotspots identification where pLDDT_{F} was calculated from the sum of pLDDT values of contacting residues of a position. The structure of natural nitrilase enzyme sequence modelling using AlphaFold tool. The contacting residues of individual amino acids were extracted and used to derive 3D structural fragments. The pLDDT values of the individual amino acids in a fragment extracted and calculated pLDDT_{F}. The table contains lower pLDDT_{F} 3D structural fragments which are chosen for engineering.

Table 3: Favourable substitution of each fragment, with the improved ΔpLDDT_{F} values in each considered fragment.

Table 4: Few natural nitrilase sequences used in this study.

Table 5: The probability values for individual amino acids of natural nitrilase enzyme sequence generated using the plurality of the nitrilases from table 5 using Equation 3. Each cell of the matrix represents the conservation percentage of a particular amino acids.

### BRIEF DESCRIPTION OF THE INVENTION

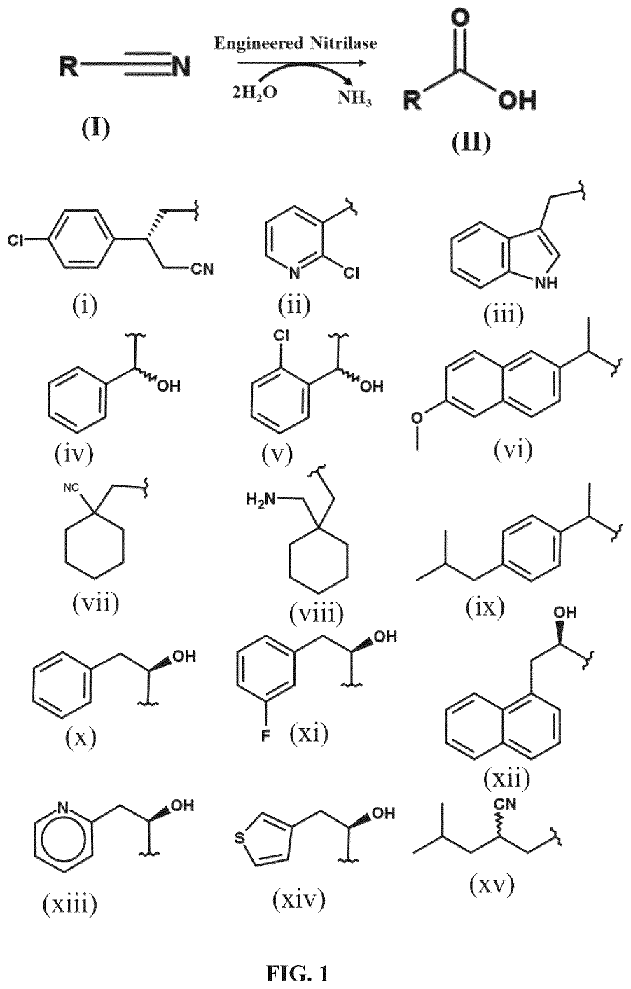

The invention presents engineered nitrilase polypeptides and their corresponding polynucleotides, optimized to convert 1-(cyanomethyl)cyclohexane-1-carbonitrile into (1-cyanocyclohexyl)-acetic acid. These polypeptides can achieve 100% catalytic activity under industrial conditions, operating efficiently within a temperature range of 20-65° C., a pH range of 5.5 to 8.0, a substrate load of 200 g/L, and with an enzyme load of less than 5% of the total weight of the substrate. The engineered nitrilase polypeptides containing multiple mutations were obtained using a step-by-step approach as described below (FIGS. 8, 9, 10, 11 and 12). The engineered nitrilase polypeptides are capable of converting 1-(cyanomethyl)cyclohexane-1-carbonitrile to (1-cyanocyclohexyl)-acetic acid and additionally other nitrile substrates into corresponding acid products as shown in FIG. 1.

The present disclosure provides engineered nitrilase polypeptides capable of converting nitrile substrates 3-(4-chlorophenyl)pentanedinitrile, 2-chloropyridine-3-carbonitrile, indole-3-acetonitrile, mandelonitrile, 2-(2-chlorophenyl)-2-hydroxyacetonitrile, 2-(6-methoxynaphthalen-2-yl)propanenitrile, 1-(cyanomethyl)cyclohexane-1-carbonitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile and Isobutylsuccinonitrile as shown in FIG. 1 to their corresponding acid products (3S)-3-(4-chlorophenyl)-4-cyanobutanoic acid, 2-chloronicotinic acid, indoleacetic acid, (R)-mandelic acid, (2-chlorophenyl)(hydroxy)-acetic acid, (S)-naproxen, (1-cyanocyclohexyl)acetic acid, gabapentin, (S)-ibuprofen, (S)-phenyllactate, (2S)-3-(3-fluorophenyl)-2-hydroxypropanoic acid, (2S)-2-hydroxy-3-(naphthalen-1-yl)propanoic acid, (2S)-2-hydroxy-3-(pyridin-2-yl)propanoic acid, (2S)-2-hydroxy-3-(thiophen-3-yl)propanoic acid, and (S)-3-cyano-5-methylhexanoic acid respectively. The nitrilase polypeptides comprise an amino acid sequence that is at least about 80% identical to SEQ ID NO:1 wherein residue corresponding to position X3 is a Lys; X88 is Cys or Arg; X106 is Glu; X122 is Asp; X196 is Tyr; X229 is Lys; X260 is Arg; and X265 is Gly.

The present disclosure also provides polynucleotides encoding the engineered nitrilase polypeptides given herein. The polynucleotide can include promoters and other regulatory elements useful for the expression of the encoded engineered nitrilase and can utilize codons optimized for specific desired expression systems.

The present disclosure provides host cells comprising the polynucleotides and/or expression vectors. The host cells may be E. coli or they may be a different organism. The host cells can be used for the expression and isolation of the engineered nitrilase enzymes, or, alternatively, they can be used directly for the conversion of the nitrile substrate to the corresponding acid product. Whether carrying out the conversion with whole cells, cell extracts or purified nitrilase enzymes, a single nitrilase enzyme may be used or, alternatively, mixtures of two or more other enzymes may be used.

### DESCRIPTION OF INVENTION IN DETAIL

### Terminologies Explained/Abbreviations

### Encode or Encoding

In this context, “encode” refers to the process of translating the information in polynucleotide sequences into proteins, which are made up of amino acids.

### Wild or Wild-Type

In this context, “wild” refers to polypeptide or polynucleotide sequence present in an organism that can be isolated from a source in nature.

Hotspots: Specific regions or residues in the enzyme that are targeted for modification to achieve desired changes in enzyme activity, stability, or specificity.

Engineering Hotspots: Regions within the enzyme structure that are particularly amenable to mutations and modifications.

Van der Waals Contacts or vdW contacts: Weak interactions between atoms that contribute to the overall structure and stability of a protein. These contacts are important for understanding the 3D conformation of the enzyme.

3D structure fragments: Portions of the enzyme's three-dimensional structure, which are analyzed separately to understand their individual contributions to the enzyme's function and stability.

pLDDT (Predicted Local Distance Difference Test): A metric used to assess the confidence in the predicted positions of residues within a protein structure. Higher pLDDT values indicate greater confidence in the accuracy of the predicted structure.

pLDDT_{F}: The sum of the pLDDT values for all residues within a 3D fragment is represented by pLDDT_{F}. This aggregate score helps identify which fragments of the enzyme have lower structural confidence and may be more flexible or amenable to modification.

Lower pLDDT_{F}: Fragments with amino acids having lower pLDDT scores are selected because they indicate regions of the enzyme with less structural confidence, which are more likely to be successfully engineered without disrupting the overall structure.

ΔpLDDT_{F} or higher pLDDT_{F}: The improved sum of the pLDDT score of a fragment after incorporating the substitution highlights the ΔpLDDT_{F} values.

Non-Conserved Residues: Amino acids within the enzyme that vary significantly among different species or variants of nitrilases. These residues are less likely to be essential for the enzyme's core function and are therefore good targets for engineering.

Probability Values for Residues: These values are derived from statistical analysis of multiple sequence alignments and indicate the likelihood of a particular residue being conserved or variable at a given position.

Multiple Sequence Alignment (MSA): A bioinformatics technique that aligns sequences from multiple proteins to identify conserved and variable regions. This alignment helps identify which residues are conserved (unchanged) and which are variable across different nitrilase sequences.

Plurality of Natural Nitrilases: A diverse set of naturally occurring nitrilase enzymes from various species.

### MSA

Multiple sequence alignment (MSA) is a method used in bioinformatics to align three or more biological sequences simultaneously to find conserved areas and patterns of similarity. It aids in understanding functional domains and evolutionary links.

The information provided by multiple sequence alignment (MSA) allows AI/ML algorithms to locate hotspots in the enzyme that are crucial for its function and are frequently targeted to increase the enzyme's activity or stability. AI/ML finds conserved areas and patterns of variation that are connected to enzyme function by aligning several sequences of similar enzymes. Based on the sequencing and structural characteristics of novel enzymes, this data is utilised to train AI/ML algorithms to identify and forecast the location of hotspots in those enzymes. Corresponding substitutions are derived from the Equation.

$$Pxi = \frac{f}{n} \times 100$$

Where, f is the frequency of amino acid at position i, n is total number of amino acids at position i, f/n is the relative frequency. (Raju et. al., 2022).

### Site Saturation Mutagenesis (SSM)

SSM is a method used to produce libraries of mutant enzymes with every possible amino acid substitution at a single site of interest.

### Site Directed Mutagenesis (SDM)

Site-directed mutagenesis is a molecular biology technique used to introduce specific, intentional changes or mutations in a DNA sequence. The technique allows to alter the genetic information of a gene at a precise location, allowing for the study of the effects of specific mutations on gene expression or protein function.

AI—AI or Artificial Intelligence can be explained logically as a field of computer science that is focused on developing computer systems that can perform tasks that typically require human intelligence. Here AI is used to do perform tasks like learning, problem-solving, decision-making, and understanding natural language. AI systems are designed to analyse data and learn from it, thereby improving their performance over time. These systems can work autonomously or in collaboration with human operators. They can also operate in real time, making decisions and taking actions based on real-time data inputs.

ML—Machine Learning, or ML, is a subfield of AI that focuses on developing computer systems that can improve their performance over time without being explicitly programmed for each task. Here, ML algorithms are trained using various techniques, such as supervised learning, unsupervised learning, and reinforcement learning, to recognize patterns in data and make predictions or decisions. Supervised learning involves training on labelled data, unsupervised learning involves finding patterns in unlabelled data, and reinforcement learning involves training on a reward-based system.

### Geometric Modelling

Enzyme geometric modelling entails the construction and analysis of the three-dimensional structure of enzymes using computational techniques. It includes techniques such as structure modelling, molecular docking, molecular dynamics simulations, and structural alignment.

### Molecular Dynamics

The movements and interactions of atoms and molecules in a system over time are studied by molecular dynamics simulations, this method offers insights into biological systems' dynamics and thermodynamics, including protein folding, substrate binding, diffusion, and product egress. The stability and adaptability of the enzymes are also studied using it.

### Substrate Binding Affinity

The degree of interaction between a substrate molecule and the binding site on an enzyme or receptor is referred to as substrate binding affinity. It influences the effectiveness of enzymatic reactions and is quantified by the dissociation constant (Kd).

### Percent Identity or Percentage Identical

In this context, the term percent identity or percentage identical are used to describe comparisons between polypeptides. To obtain this percentage, two sequences are optimally aligned over a comparison window, which may include gaps (i.e., deletions or additions) in the polypeptide sequence compared to the reference sequence, which does not contain gaps. The percentage is calculated by counting the number of positions in which the same nucleic acid base or amino acid residue appears in both sequences, dividing the number of matched positions by the total number of positions in the comparison window, and multiplying the result by 100 to obtain the percentage of sequence identity.

### Improved (Enzyme) Property

Improved (enzyme) property refers to a nitrilase polypeptide that exhibits an improvement in any enzyme property as compared to a reference nitrilase. For the engineered nitrilase polypeptides described herein, the comparison is generally made to the wild-type nitrilase enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

### Increased Enzymatic Activity

Increased enzymatic activity refers to an improved property of the engineered nitrilase polypeptides, which can be represented by an increase in specific activity (e.g., product produced/time/weight protein) or an increase in percent conversion of the substrate to the product (e.g., percent conversion of starting amount of substrate to product in a specified time period using a specified amount of nitrilase) as compared to the reference nitrilase enzyme.

“His-tag” refers to the Poly histidine tag, which is consists of 6 to 10 consecutive histidine residues, which is used for protein purification using Ni-NTA interaction, which is based on the selectivity and high affinity of Ni-NTA (nickel nitrilotriacetic acid).

### “Near Attack Conformation” “Enzyme-Substrate Complex” “Michaelis Complex”

Near attack conformation” “Enzyme-substrate complex” or “Michaelis complex” in this context refers to the association of a substrate with an enzyme that is an obligatory intermediate in the conversion of the substrate into the product of the enzymic reaction.

### Percentage Conversion

Conversion refers to the enzymatic conversion of the substrate to the corresponding product. “Percent conversion” refers to the percent of the substrate that is reduced to the product within a period of time under specified conditions. Thus, the “enzymatic activity” or “activity” of a nitrilase polypeptide can be expressed as the “percent conversion” of the substrate to the product.

### Stability at Broader Temperature or Thermostable

Stability at broader temperature or Thermostable refers to a nitrilase polypeptide that maintains similar activity (more than 60% to 80% or more, for example) after exposure to elevated temperatures (e.g., 40-80° C.) for a period of time (e.g., 0.5-24 hrs) compared to the untreated enzyme.

### Stability at Broader pH or pH Stable

Stability at broader pH or pH stable refers to a nitrilase polypeptide that maintains similar activity (more than, e.g., 60% to 80%) after exposure to high or low pH (e.g., 4.5-6 or 8 to 12)

“Amino acid” or “residue” as used in the context of the polypeptides disclosed herein refers to the specific monomer at a sequence position (e.g., F 116 indicates that the “amino acid” or “residue” at position 116 is a Phenylalanine.)

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to the loss of a hydrogen ion. Genetically encoded acidic amino acids include L-Glu I and L-Asp (D).

Basic amino acid or residue refers to a hydrophilic amino acid or residue having a side chain exhibiting a pKa value of greater than about 6 when the amino acid is included in a peptide or polypeptide. Basic amino acids typically have positively charged side chains at physiological pH due to their association with hydronium ions. Genetically encoded basic amino acids include L-Arg(R) and L-Lys (K).

Polar amino acid or residue refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S) and L-Thr (T).

Non-polar amino acid or residue refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M) and L-Ala (A).

Hydrophilic amino acid or residue refers to an amino acid or residue having a side chain exhibiting a hydrophobicity of less than zero according to the normalized consensus hydrophobicity scale. Genetically encoded hydrophilic amino acids include L-Thr (T), L-Ser (S), L-His (H), L-Glu (E), L-Asn (N), L-Gln (Q), L-Asp (D), L-Lys (K) L-Arg (R).

“Hydrophobic amino acid or residue” refers to an amino acid or residue having a side chain exhibiting a hydrophobicity of greater than zero according to the normalized consensus hydrophobicity scale. Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

Aromatic amino acid or residue refers to a hydrophilic or hydrophobic amino acid or residue having a side chain that includes at least one aromatic or heteroaromatic ring. Genetically encoded aromatic amino acids include L-Phe (F), L-Tyr (Y) and L-Trp (W). Although owing to the pKa of its heteroaromatic nitrogen atom L-His (H) it is sometimes classified as a basic residue, or as an aromatic residue as its side chain includes a heteroaromatic ring.

Aliphatic amino acid or residue refers to a hydrophobic amino acid or residue having an aliphatic hydrocarbon side chain. Genetically encoded aliphatic amino acids include L-Ala (A), L-Val (V), L-Leu (L) and L-Ile (I).

Amino acid difference or residue difference refers to a change in the residue at a specified position of a polypeptide sequence when compared to a reference sequence. For example, a residue difference at position X116, where the reference sequence has a phenylalanine, refers to a change of the residue at position X116 to any residue other than phenylalanine. As disclosed herein, an enzyme can include one or more residue differences relative to a reference sequence, where multiple residue differences typically are indicated by a list of the specified positions where changes are made relative to the reference sequence.

Codon optimized refers to changes in the codons of the polynucleotide encoding a protein to those preferentially used in a particular organism such that the encoded protein is efficiently expressed in the organism of interest. Although the genetic code is degenerate in that most amino acids are represented by several codons, it is well known that codon usage by particular organisms is non-random and biased towards particular codon triplets. This codon usage bias may be higher in reference to a given gene, genes of common function or ancestral origin, highly expressed proteins versus low copy number proteins and the aggregate protein coding regions of an organism's genome.

Reference sequence refers to a defined sequence to which another (e.g., altered) sequence is compared.

Conservative amino acid substitutions or mutations refer to the interchangeability of residues having similar side chains, and thus typically involve substitution of the amino acid in the polypeptide with amino acids within the same or similar defined class of amino acids.

Non-conservative substitution refers to the substitution or mutation of an amino acid in the polypeptide with an amino acid with significantly differing side chain properties.

“1-(cyanomethyl) cyclohexane-1-carbonitrile” also refers as “1-Cyanocyclohexane acetonitrile”.

### Method of Engineered Nitrilase Enzyme

Engineering of the nitrilase enzyme sequence is described below in a step-by-step manner (FIG. 8 to 12). Briefly, the hotspots for engineering the enzyme are identified by a method that employs van der Waals contacts to create 3D fragments of the enzyme, summing the pLDDT values of residues for each 3D fragment to generate a pLDDT_{F}, and further selecting fragments with lower pLDDT_{F} to identify residues that are relatively non-conserved within the fragment based on probability values derived for each residue position using multiple sequence alignments derived from a plurality of natural nitrilases each having sequences not more than 90% identical to each other. This method involves analysing the enzyme's 3D structure to understand the weak interactions between atoms, dividing the enzyme into smaller 3D fragments, and predicting the confidence in the position of each residue using pLDDT values. By summing these values for each fragment to generate a pLDDT_{F} score and selecting fragments with lower scores, regions with lower structural confidence that are more flexible and amenable to modification are identified. Relatively non-conserved residues, which vary among different nitrilase sequences and are identified through multiple sequence alignments, are targeted for engineering to improve the enzyme's activity, stability, or specificity without disrupting its core functions, ensuring the engineered nitrilase retains its overall structure while gaining enhanced properties for industrial or research applications. The hotspots were used by different methods explained below to incorporate multiple substitution and derive enzyme variants.

Step 1: A natural nitrilase enzyme sequence was selected and used as a starting point for the engineering the enzyme (Table 4, FIG. 8).

Step 2: The selected natural nitrilase enzyme sequence is modelled using AlphaFold, a homology-based approach with a scoring function using a predicted local distance difference test (pLDDT). The calculation of pLDDT is based on a per-residue confidence metric called the predicted local distance difference test (pLDDT) on a scale from 0 to 100. pLDDT estimates how well the prediction would agree with an experimental structure based on the local distance difference test Cα (1DDT-Cα) (FIG. 8).

Step 3: the modelled structure is used to identify the contacting residues of every amino acid present in the enzyme and stored as a fragment. For example, contacting residue details for the amino acid at 1^{st} position extracted and saved as fragment 1. Similarly, the 2nd position was extracted and saved as fragment 2. Likewise, for all the amino acids in the enzyme contact residues and fragment details were extracted and stored (FIG. 8).

The 3D structural fragment was generated using vdW_{contacts} for every residue of the nitrilase enzyme. The structural fragments were derived using Equation 1.

$$i . {vdW}_{contact} = {r}_{VDWi} + {r}_{VDWj} - {d}_{ij} - {allowance}_{ij} \qquad ( eq . 1 )$$

‘vdW_{contact}’ is defined between two atoms (i, j) as the sum of their vdW radii minus the distance between them and minus an allowance for potentially hydrogen-bonded pairs. The allowance is only subtracted for pairs comprised of a donor (or donor-borne hydrogen) and an acceptor. The schematic representation of one of the fragments is shown in FIG. 13.

Step 4: pLDDT_{F} score of the contacting residue in a fragment is obtained from the modelled 3D structure of the enzyme. The pLDDT score of each contacting residue in a fragment obtained from the modelled 3D structure of the enzyme is considered. For each fragment F, the pLDDT score of every amino acid residue xi present in the fragment is extracted. The pLDDT scores of all residues within the fragment are summed to calculate the pLDDT_{F} score of the fragment (Table 2, FIG. 8)) as given in Equation 2:

$${pLDDT}_{F} = {\sum }_{{x}_{i} ∈ F} pLDDT ( {x}_{i} ) \qquad ( eq . 2 )$$

This term represents the pLDDT_{F} score of a specific 3D structural fragment F.
It is calculated by summing the pLDDT scores of all residues within the fragment F.
Σ: Summation symbol indicating the summing operation.
xi∈F: Represents all residues xi that are part of the fragment F.
pLDDT(xi): Represents the predicted Local Distance Difference Test (pLDDT) score for residue xi, which is a confidence score for the accuracy of the predicted position of the residue in the protein structure.

Step 5: Fragments with lower pLDDT_{F} scores are selected for hotspot identification. Fragments with lower pLDDT_{F} scores indicate regions where the confidence in the predicted structure is lower, making these regions potential targets for engineering (FIG. 8).

Step 6: Fragments with lower pLDDT_{F} scores are further analyzed to identify residues that are relatively non-conserved within the fragment. This analysis uses probability values derived for each residue position through multiple sequence alignments of natural nitrilases with less than 90% sequence identity. To identify non-conserved amino acids, multiple sequence alignment method used and non-conserved residues were filtered (Table 5) from the fragment which are considered for further engineering. Multiple sequence alignments (MSA) were generated from A plurality of nitrilase enzymes from UniRef90 databases. The MSA information and annotated genetic variants were used as leverage for the learning methods to make a qualitative prediction about a mutation's effect on solubility fitness.

The identification of the non-conserved residues of the nitrilase is calculated using the probability values. The probability values were derived from the plurality of natural nitrilase sequences (Table 5, FIG. 8) using Equation 3.

$$Pxi = \frac{f}{n} \times 100 \qquad ( eq . 3 )$$

Where, ‘Pxi’ represents the probability of amino acids at specific position, ‘f’ is the frequency of amino acids at position ‘i’, ‘n’ is total number of amino acids at position ‘i’, f/n is the relative frequency.

The below steps are used to incorporate substitutions from the plurality of nitrilases, which are briefly explained in a step-by-step manner (FIG. 9).

Step 7: Introduce substitutions at specific residue positions from the information of various nitrilases, guided by pLDDT scores. Fragments with lower pLDDT_{F} scores are used to identify hotspots which are replaced with amino acids that have higher pLDDT values, sourced from evolutionary data in the UniRef90 database. These substitutions aim to enhance the fragment's stability within the protein (FIG. 9).

Step 8: Protein modelling studies are undertaken to assess the enhancements in the pLDDT_{F} score resulting from these substitutions (FIG. 9).

Step 9: Equations 1 and 2 are applied to the newly modelled protein, allowing us to extract the same fragment and verify the improvements in its pLDDT_{F} score. The improved sum of pLDDT_{F} score will be given as ΔpLDDT_{F} (FIG. 9). The results of engineered fragments are given in Table 4 and also in FIG. 14.

Step 10: An engineered nitrilase is generated by incorporating substitutions at multiple hotspots, resulting in a variant with improved activity and stability (FIG. 9).

### Method for Engineering Nitrilases Using Site Saturation Mutagenesis

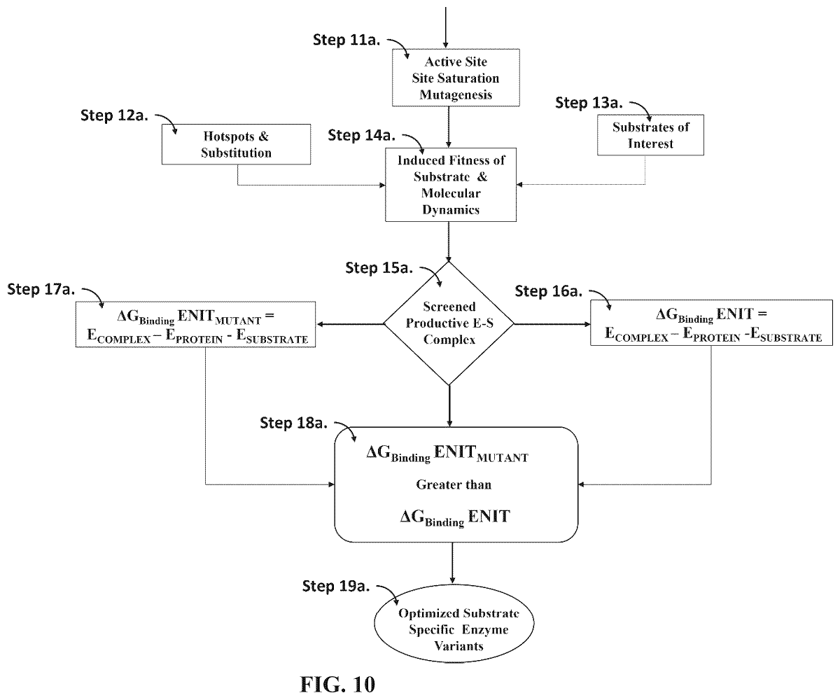

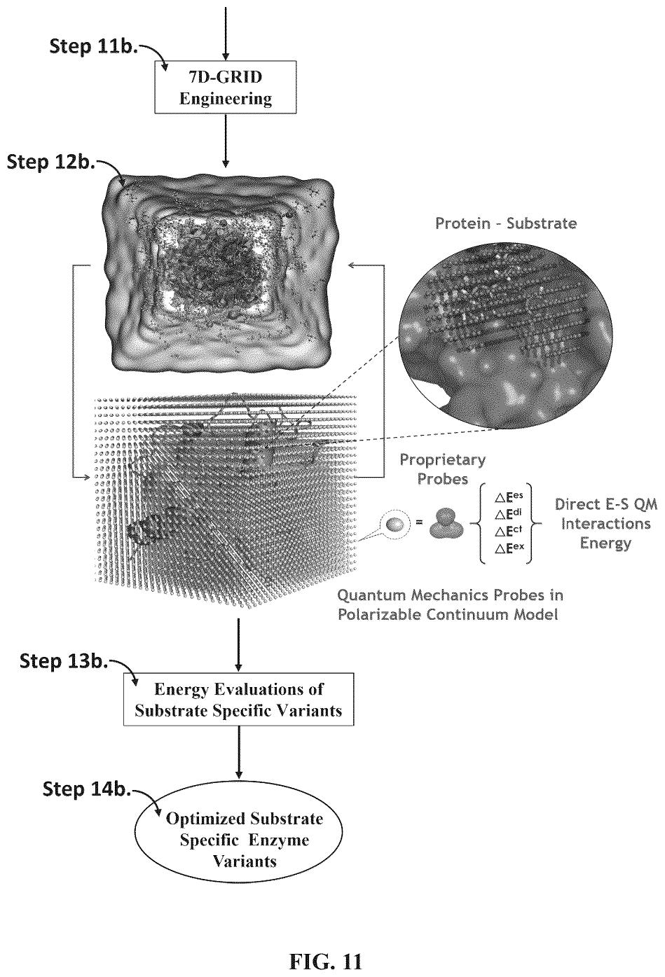

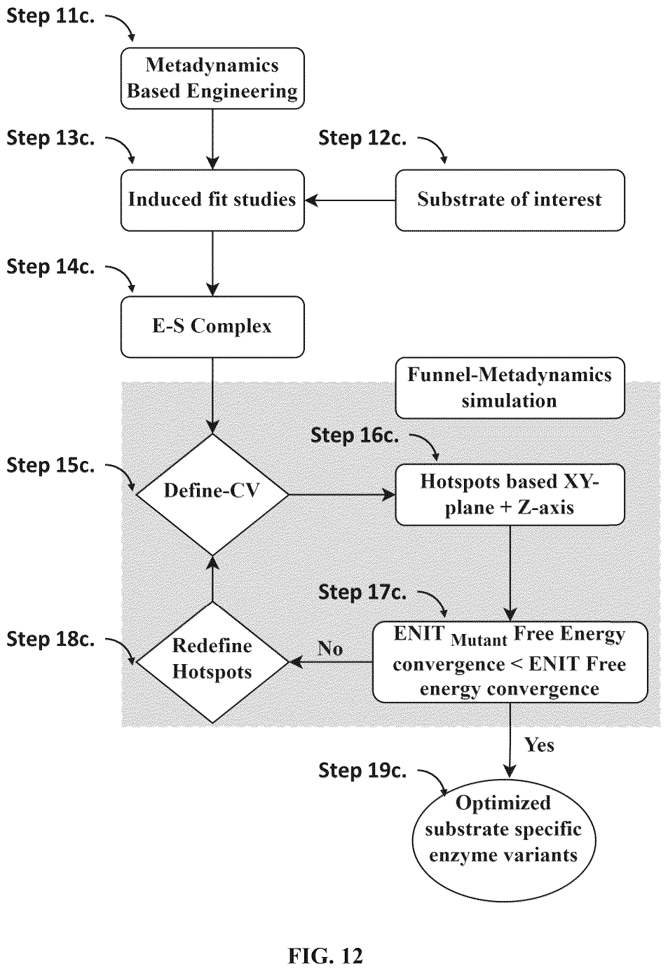

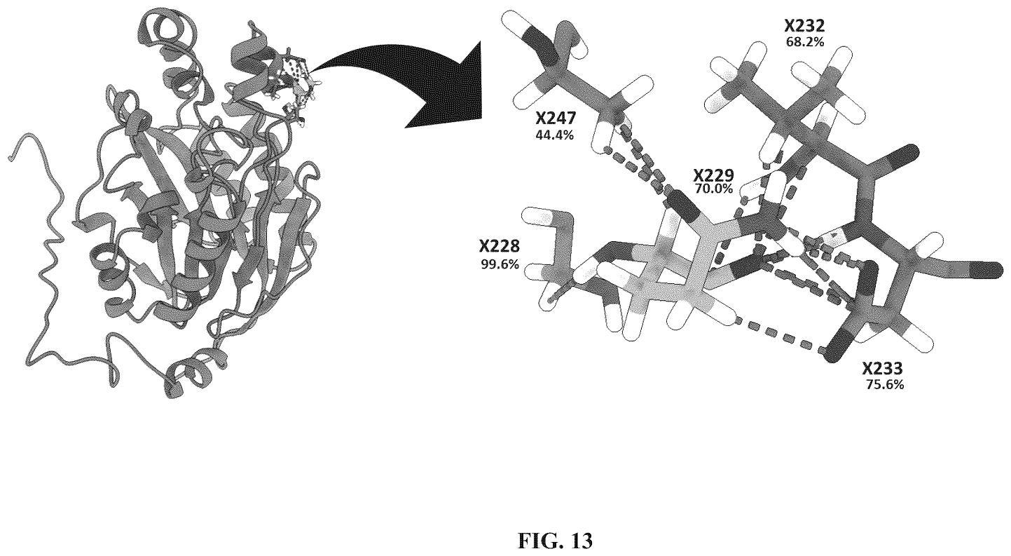

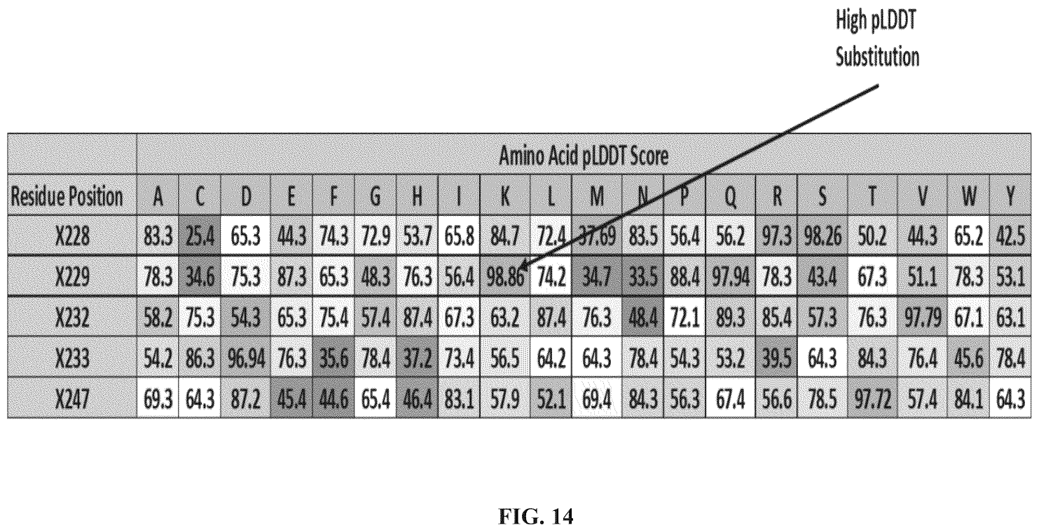

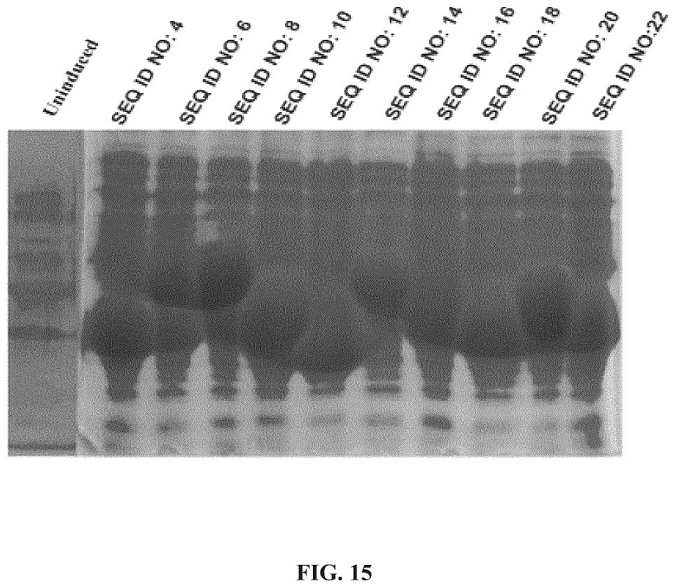

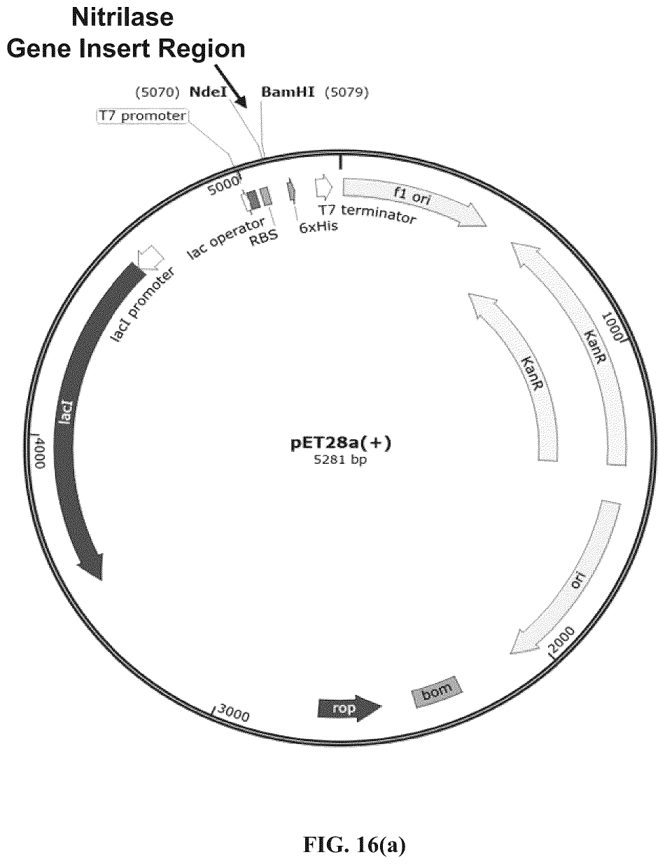

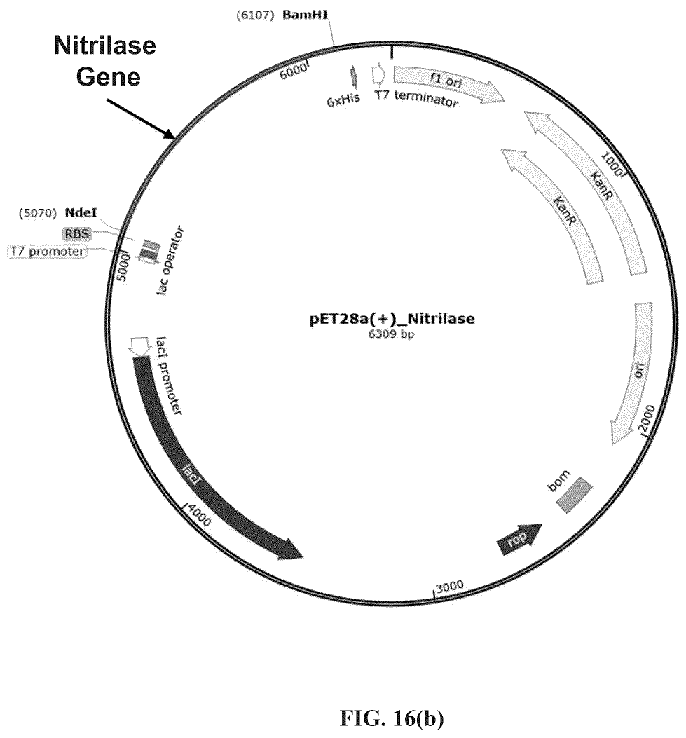

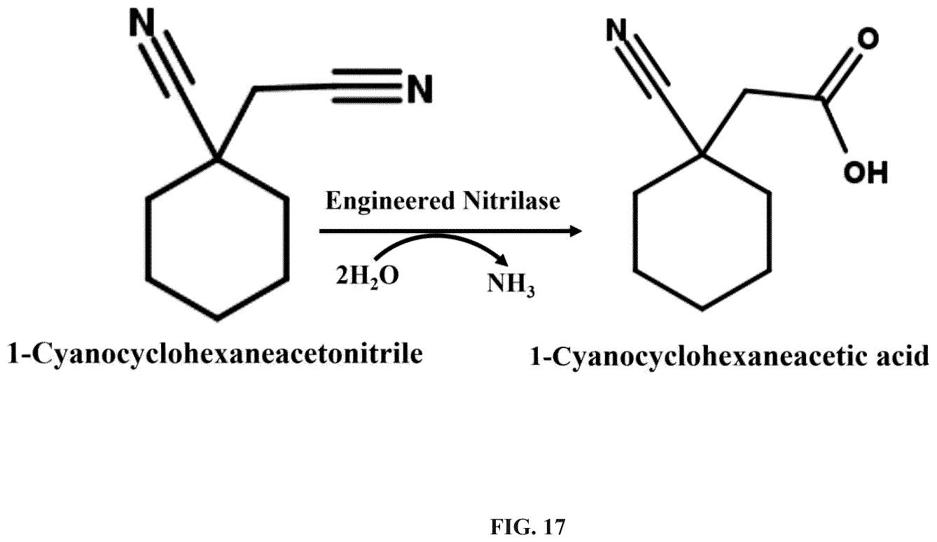

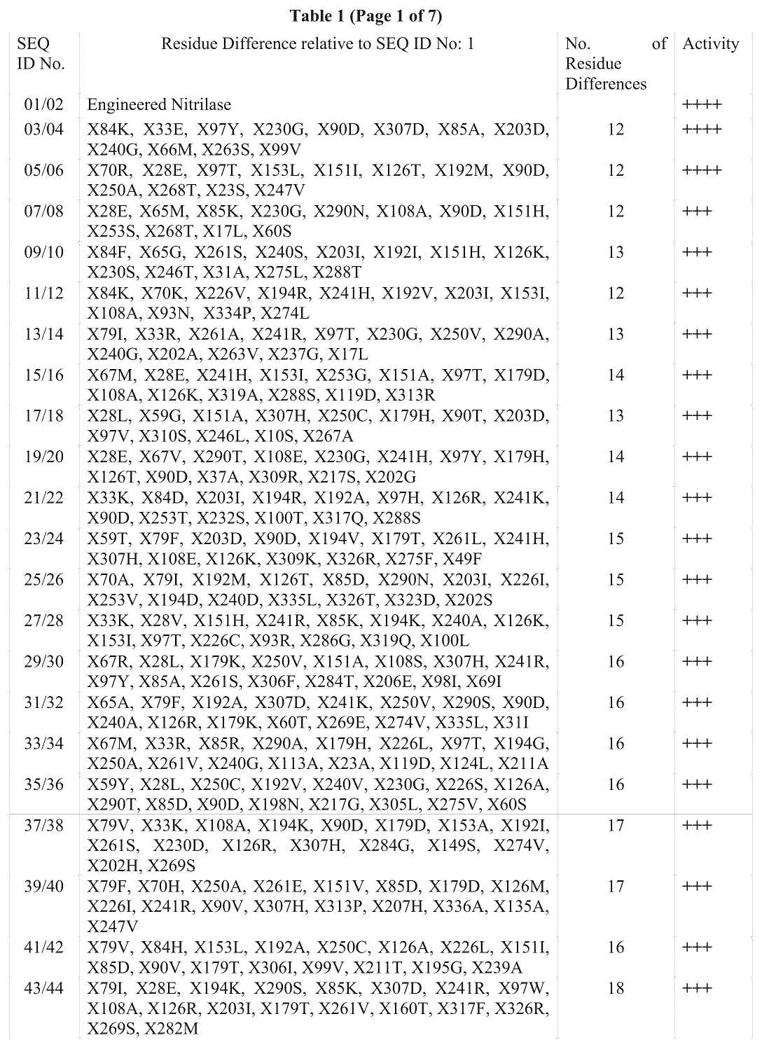

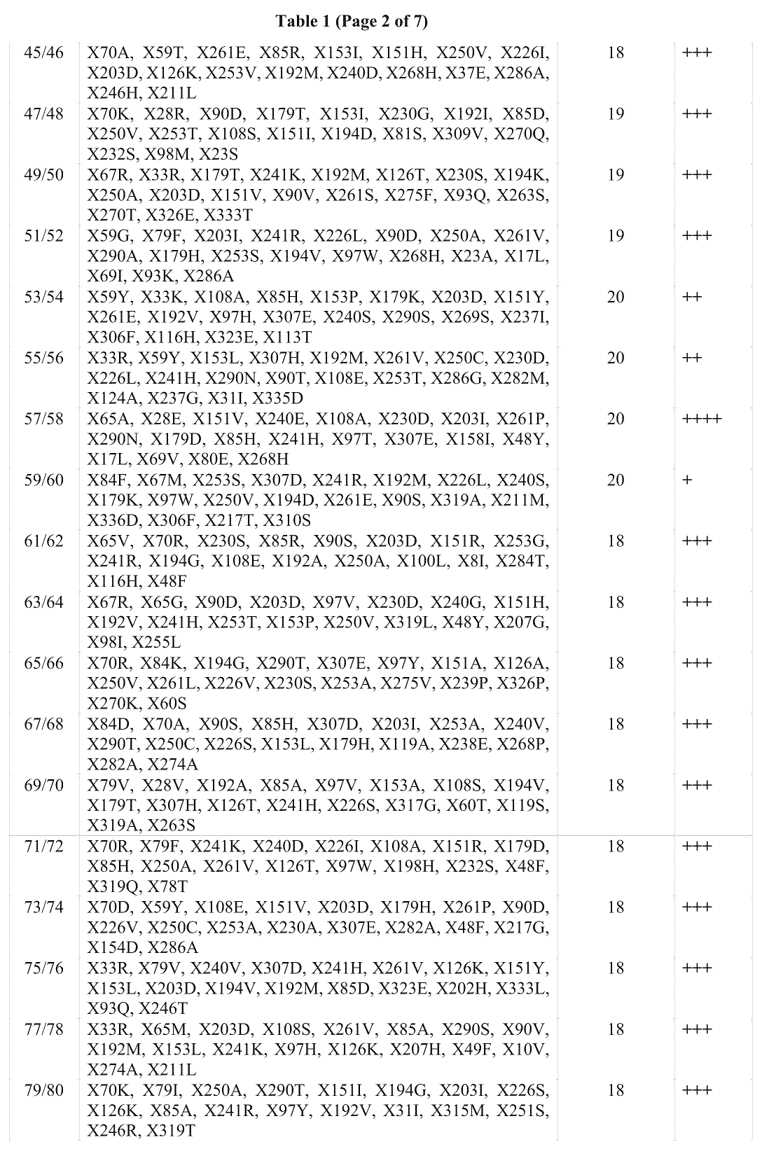

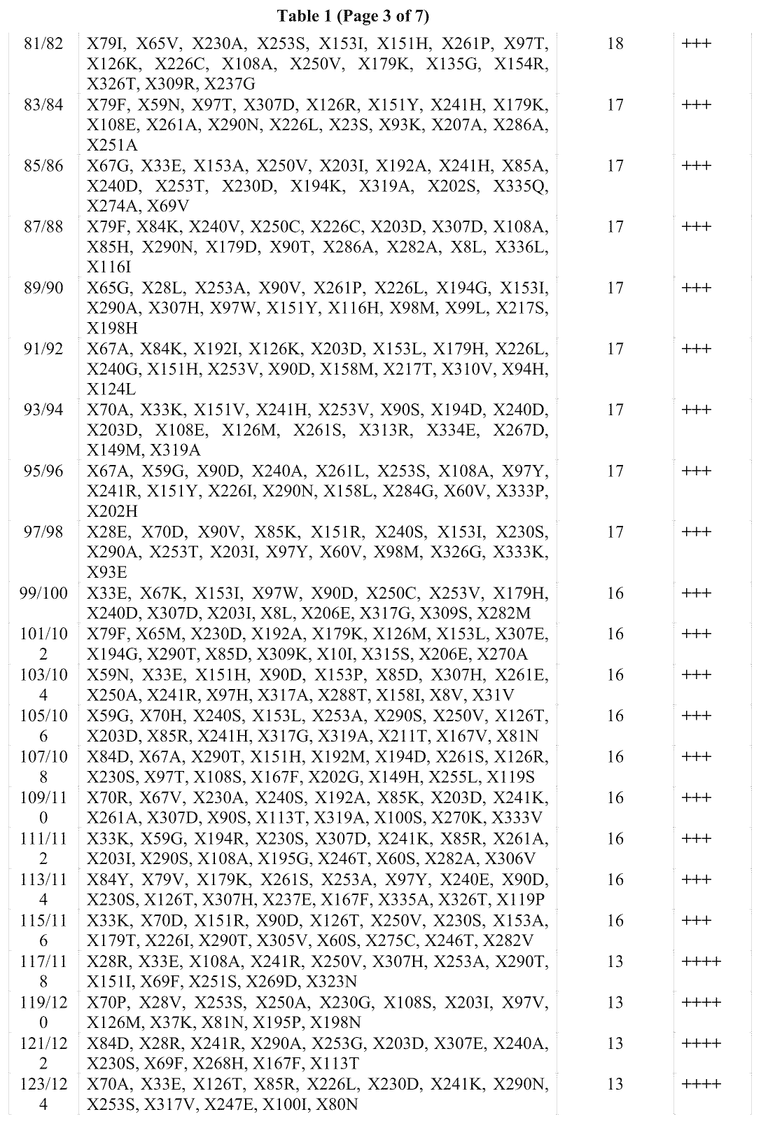

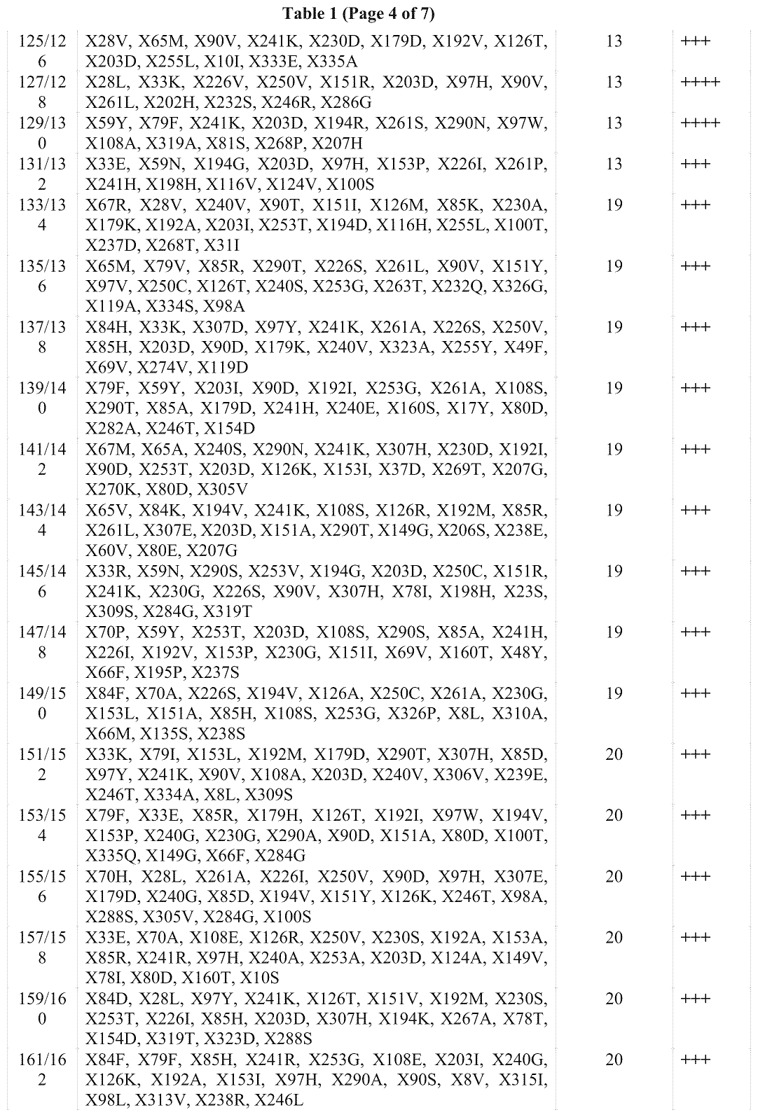

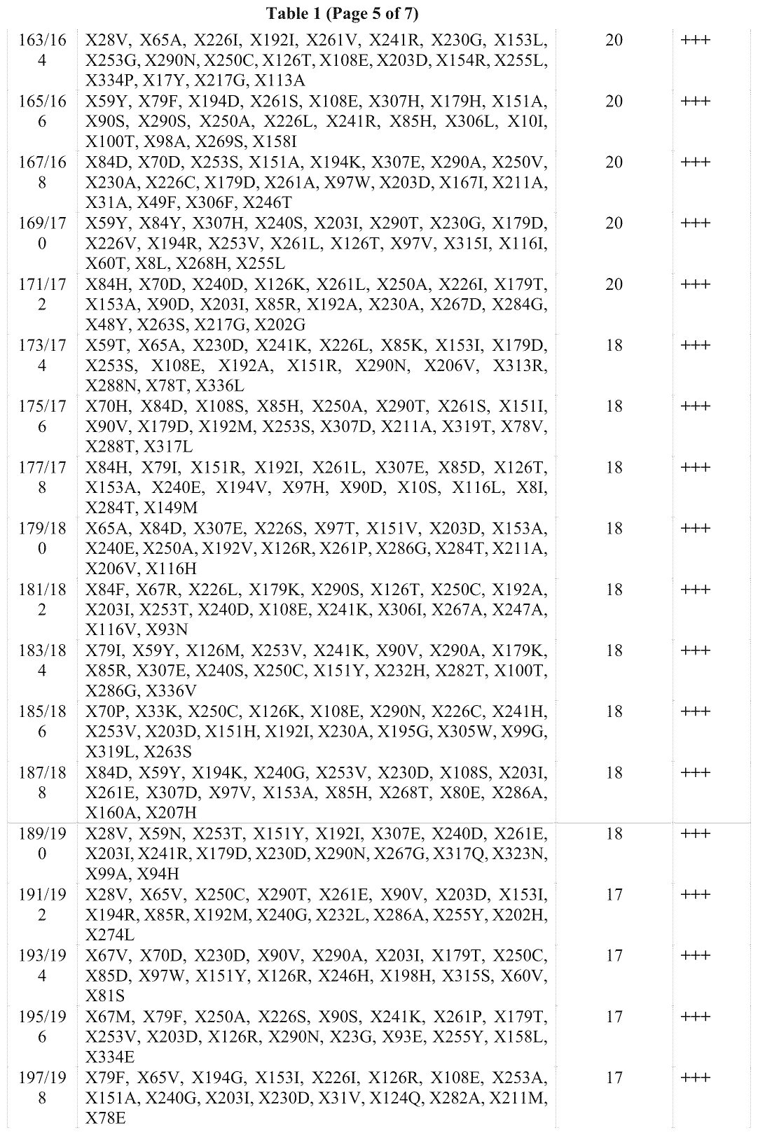

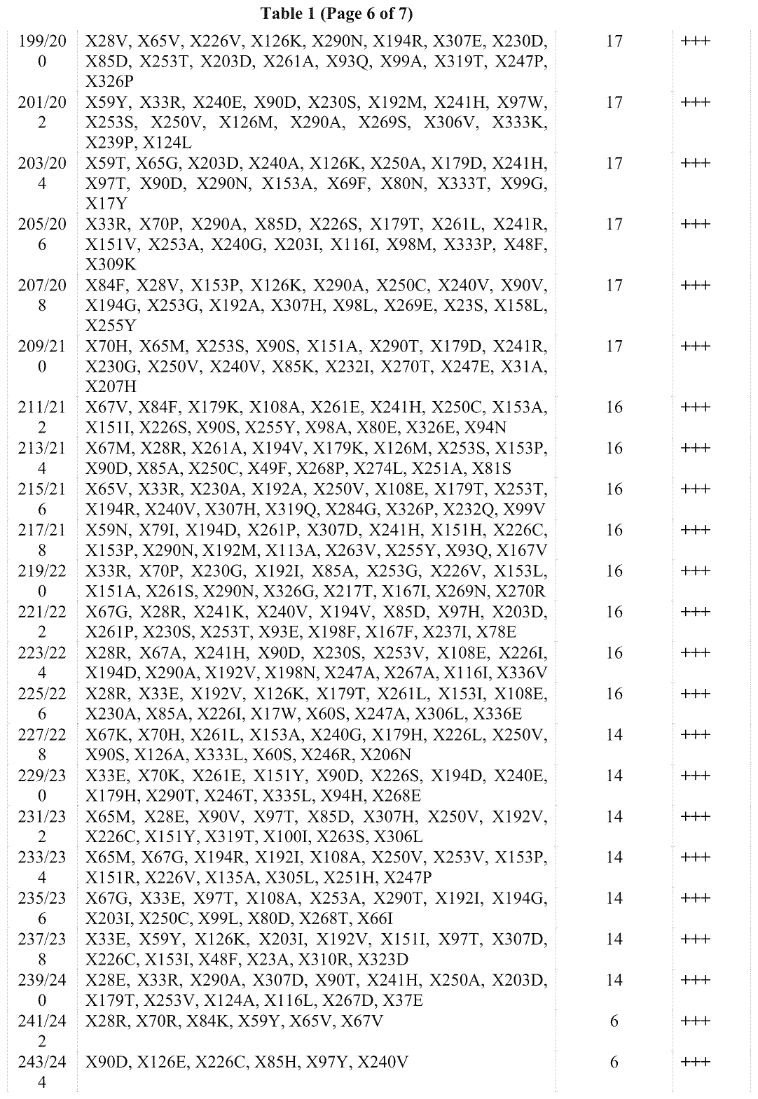

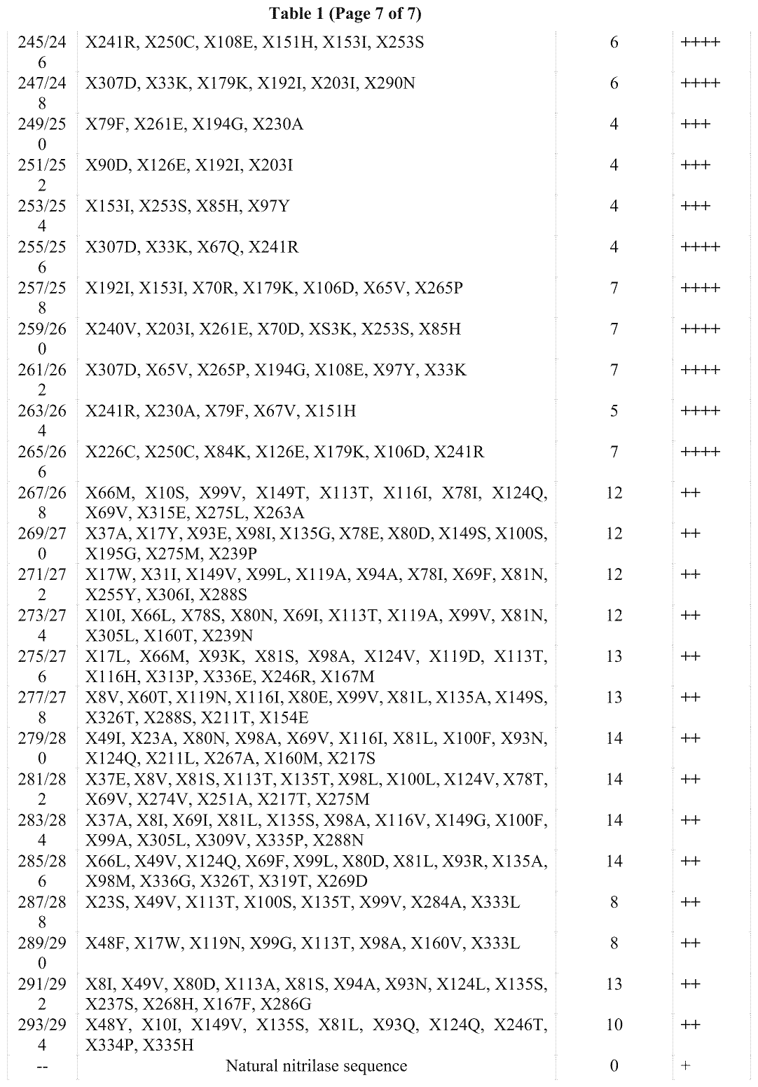

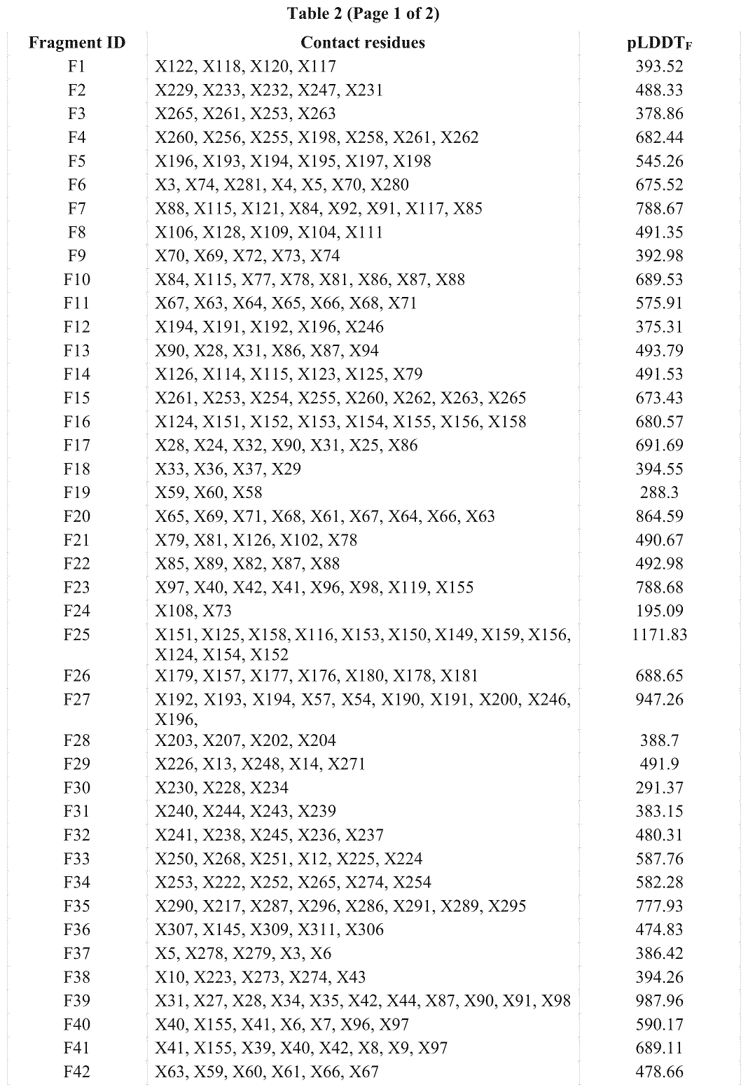

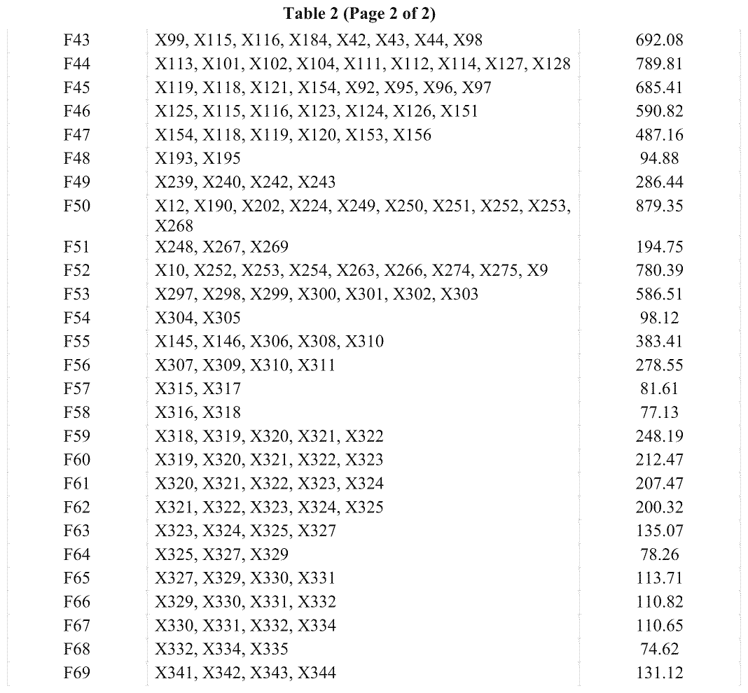

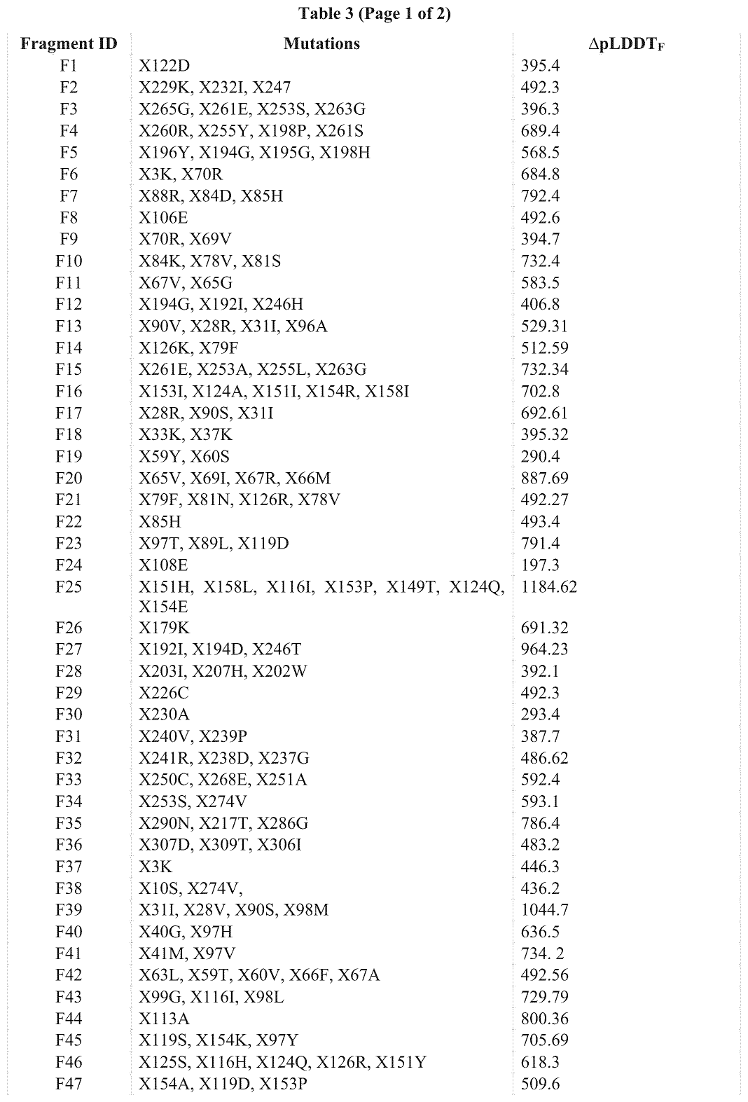

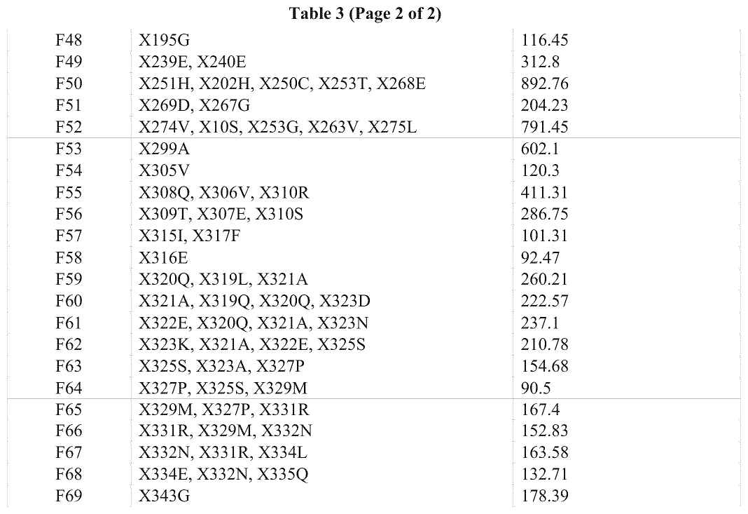

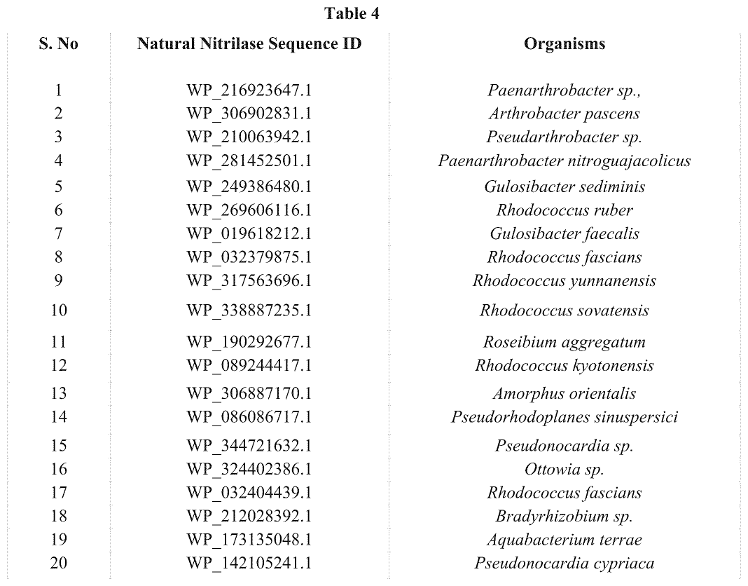

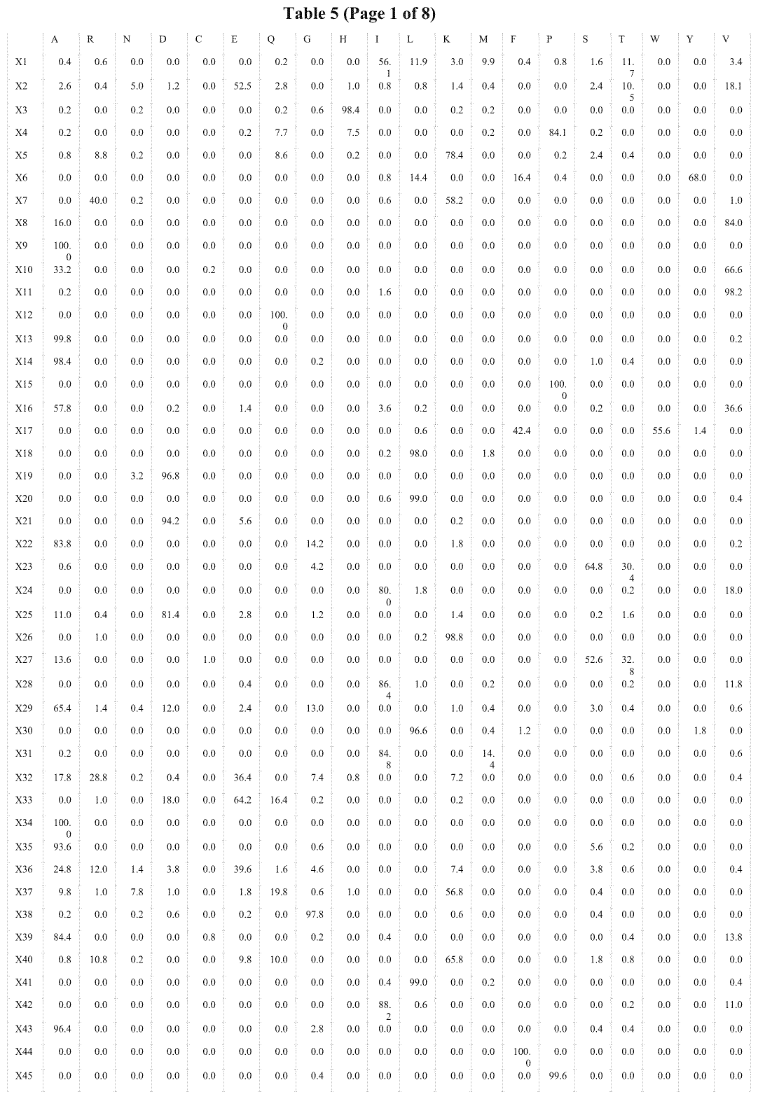

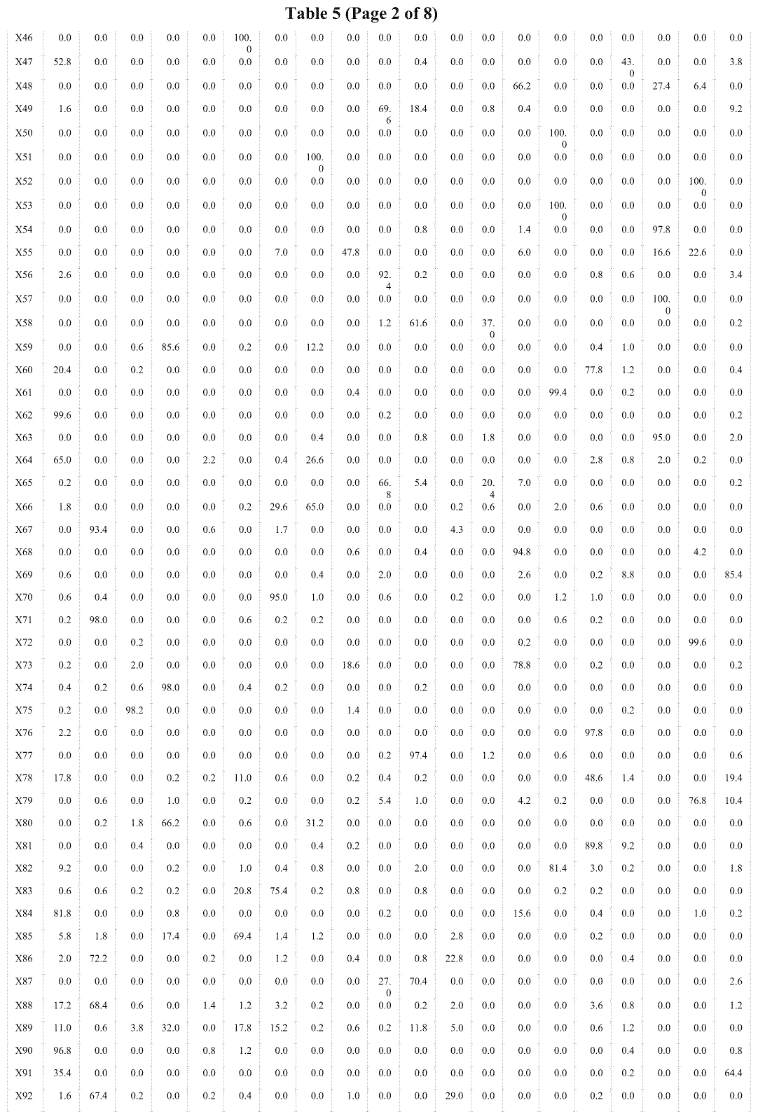

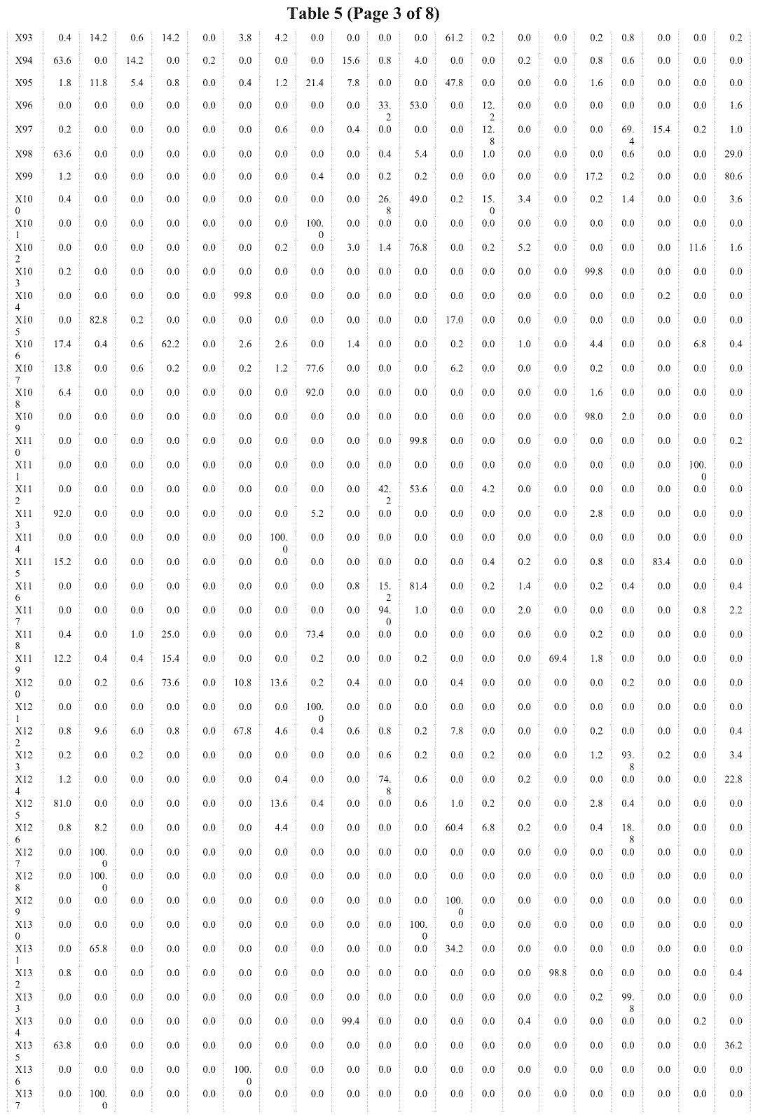

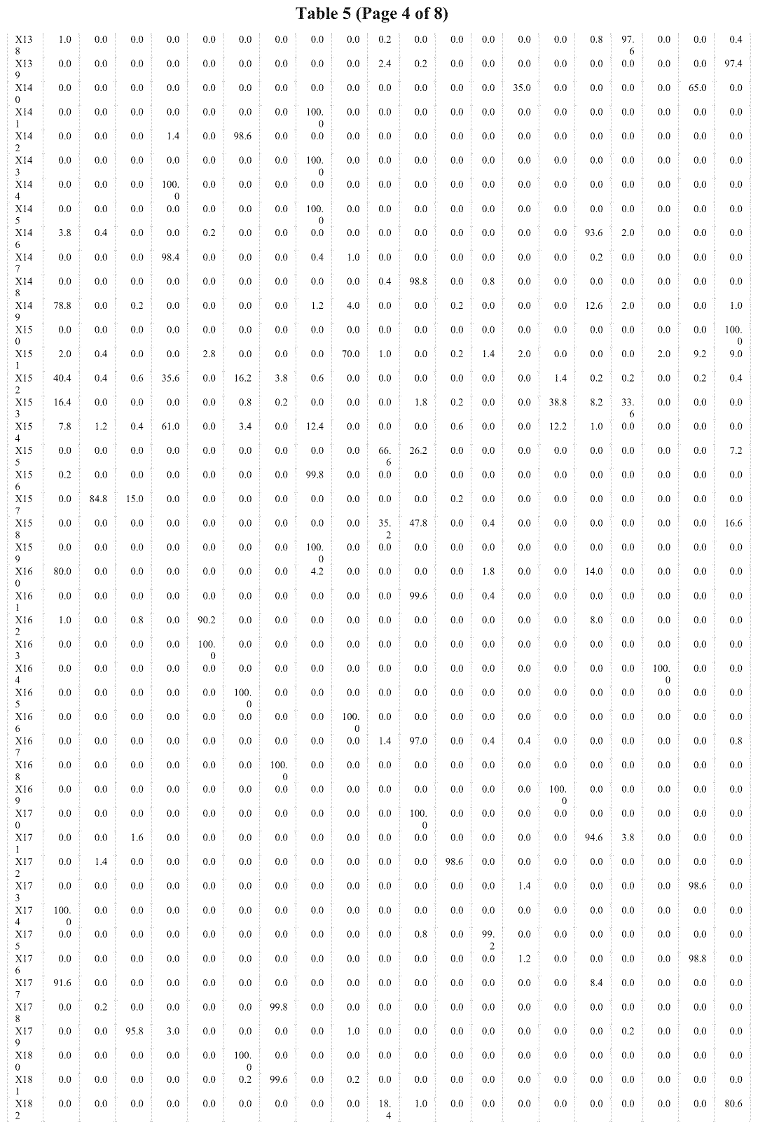

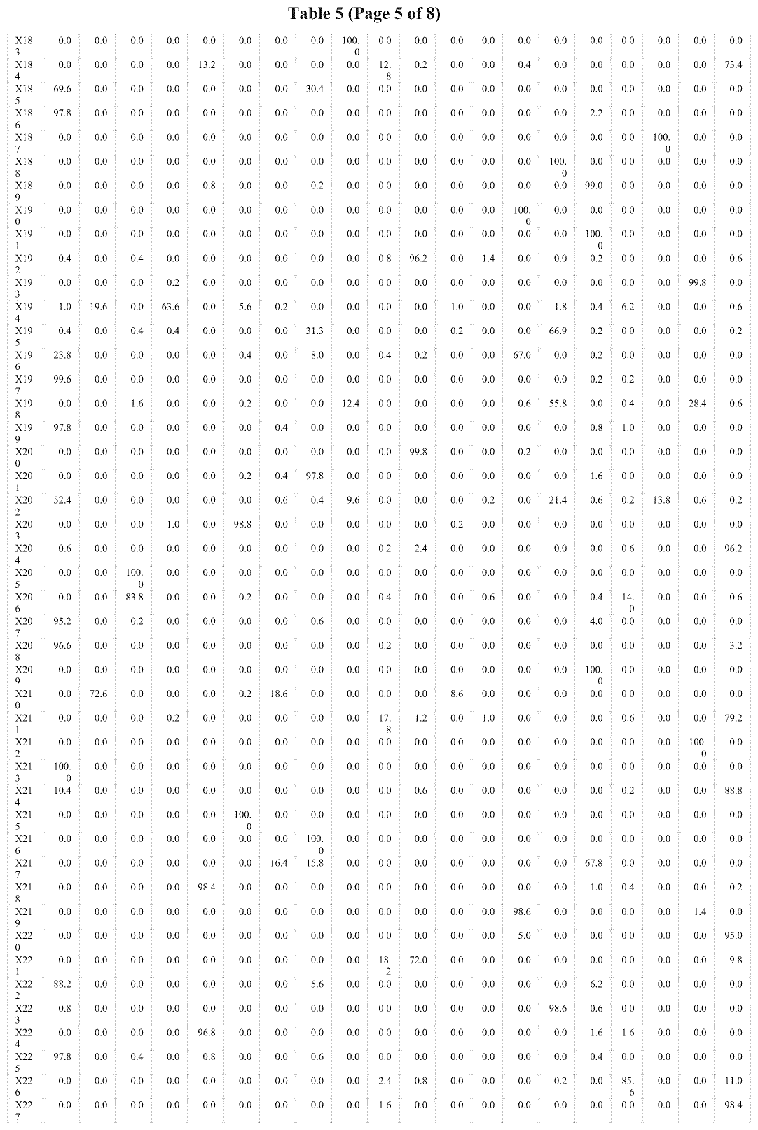

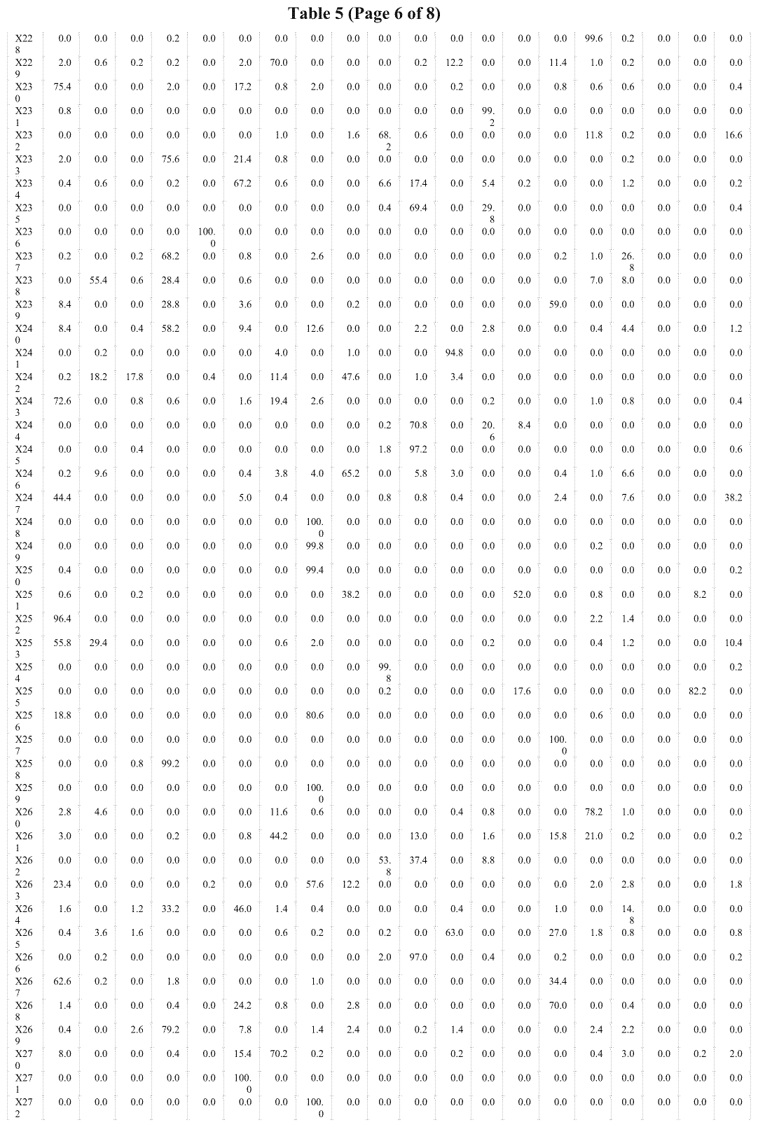

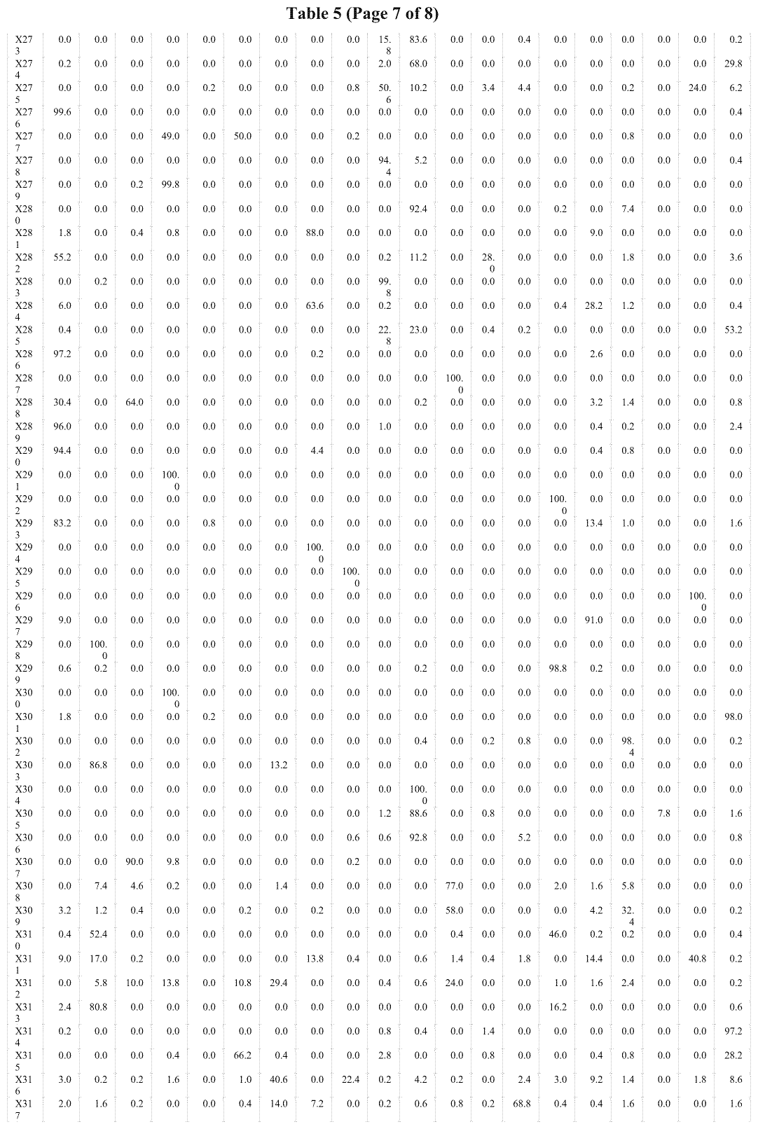

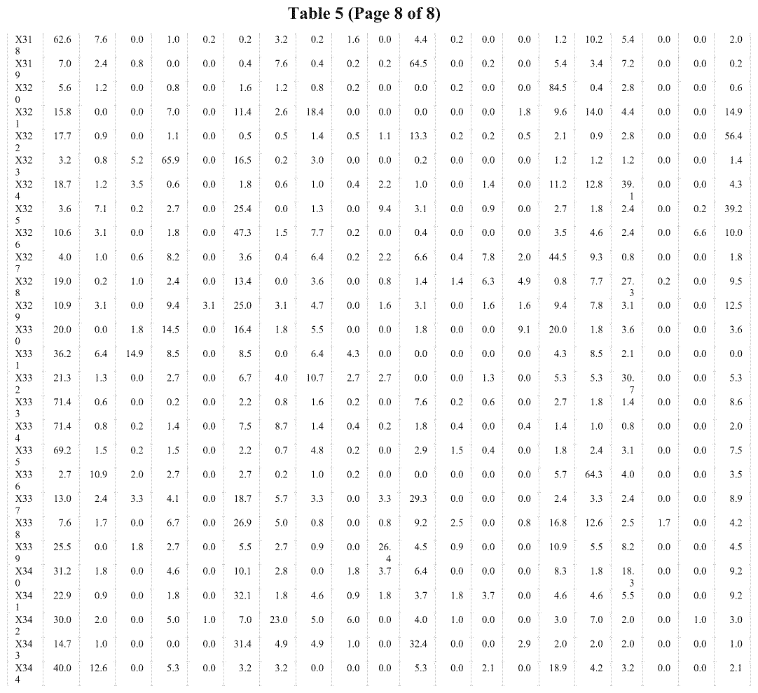

The engineered nitrilase enzyme sequence was further engineered to improve the selectivity for different substrates using active site, site saturation mutagenesis method with the steps mentioned in FIG. 10. A detailed stepwise description of the method is described below:

Step 11a: The active site, site saturation mutagenesis was used to mutate the identified hotspots into specific amino acids within the enzyme active site.

Step 12a: The hotspots were identified based on the non-conserved positions in the active site. Conserved positions are left due to the plausible evolutionary function. The substitution is suggested with all other possible amino acids on the selected hotspots position.

Step 13a and 14a: The substitution of amino acids at identified hotspots were screened against the different substrates (1-cyanocyclohexyl)acetic acid, 3-(4-chlorophenyl)pentanedinitrile, 2-chloropyridine-3-carbonitrile, indole-3-acetonitrile,

Mandelonitrile, 2-(2-chlorophenyl)-2-hydroxyacetonitrile, 2-(6-methoxynaphthalen-2-yl)propanenitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile, and Isobutylsuccinonitrile using induced fit and correspondingly simulated using molecular dynamics method to generate the all the plausible productive enzyme-substrate complex.

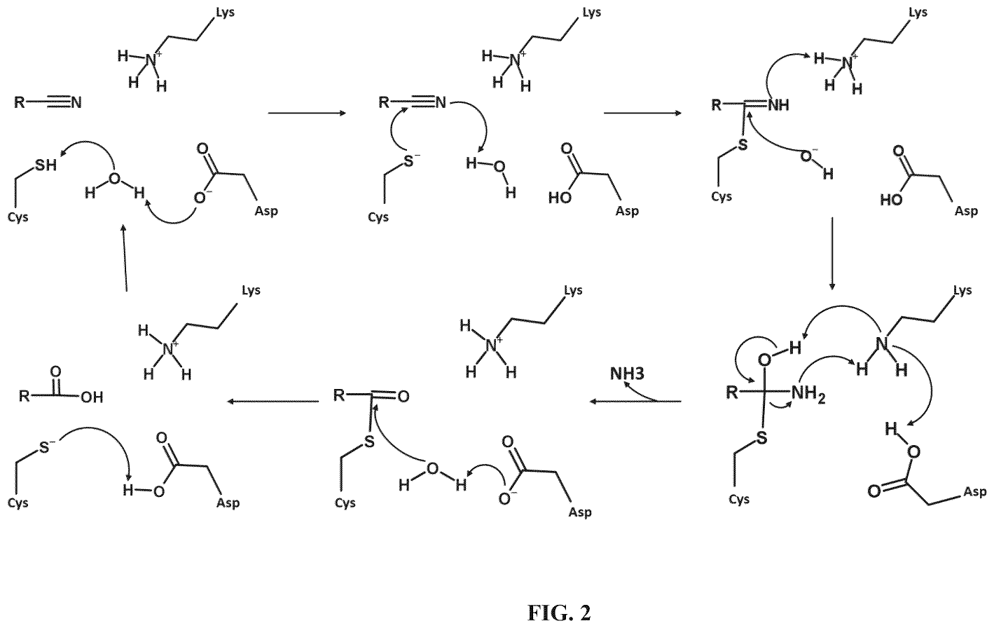

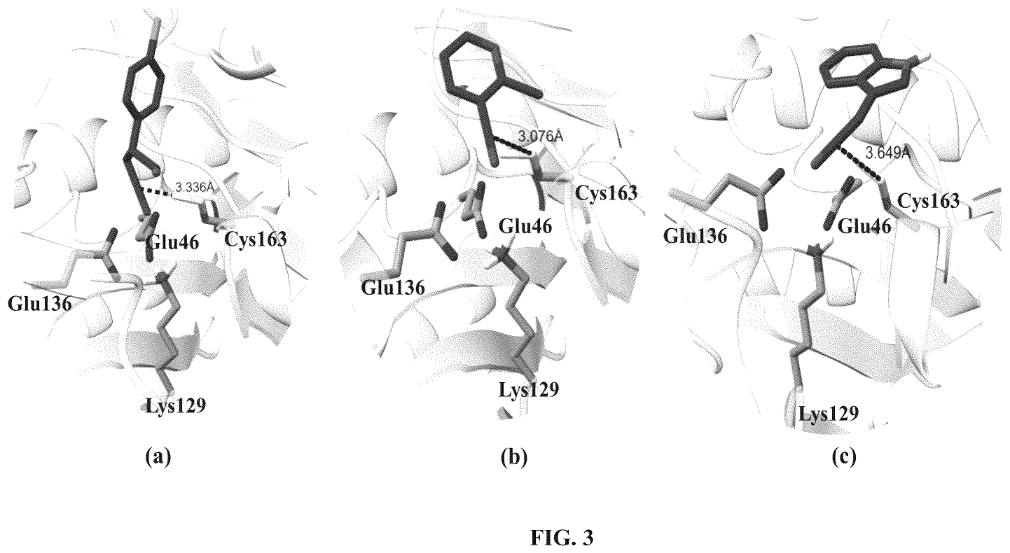

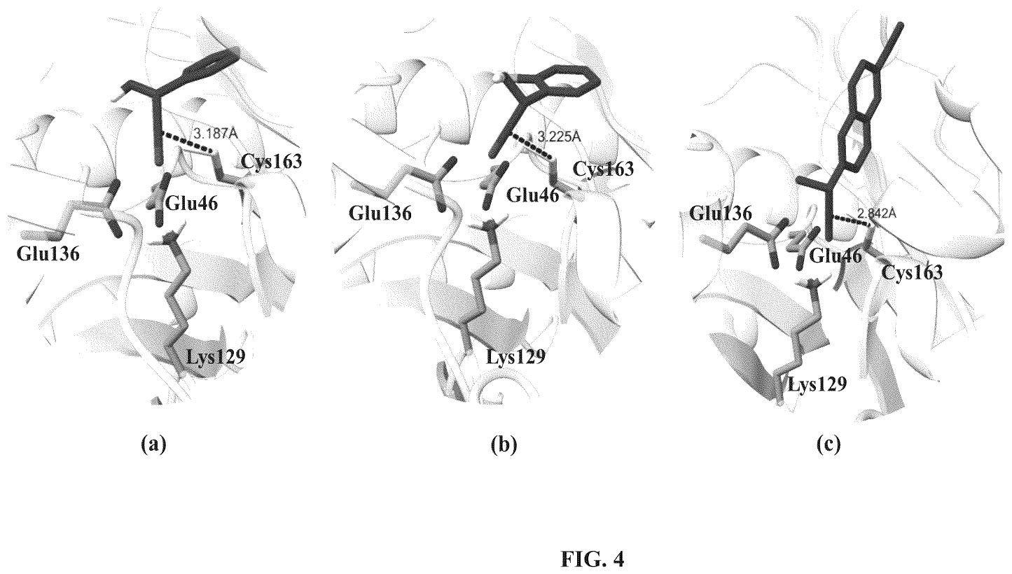

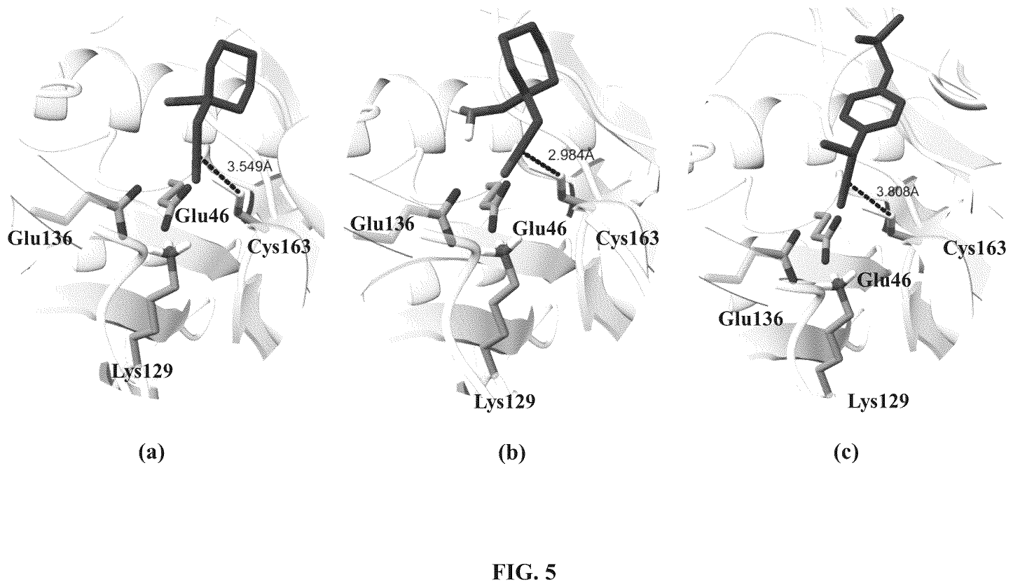

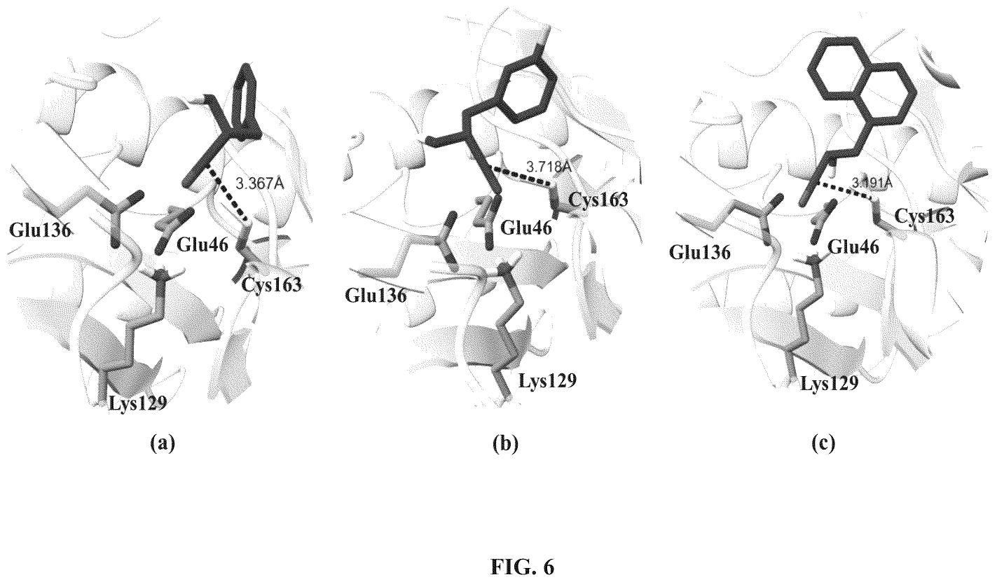

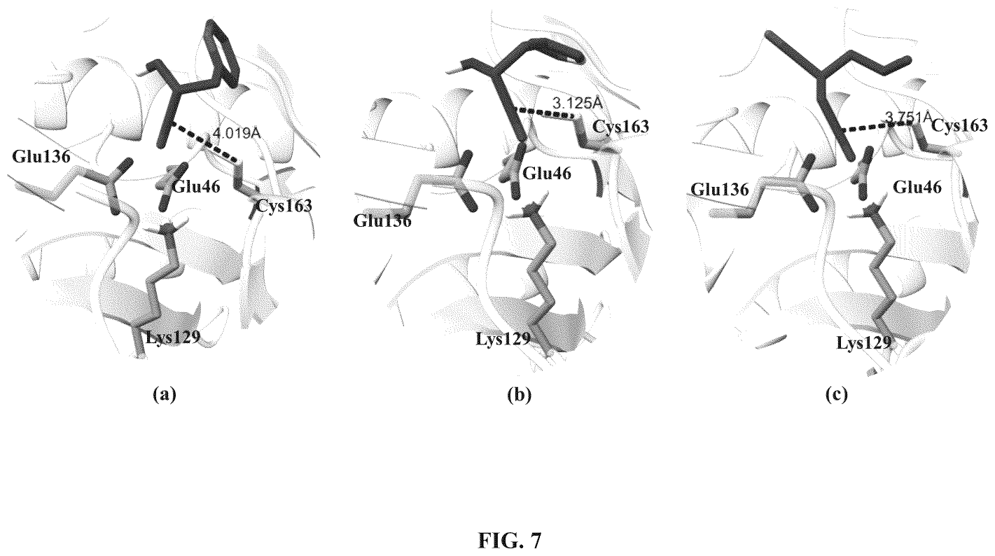

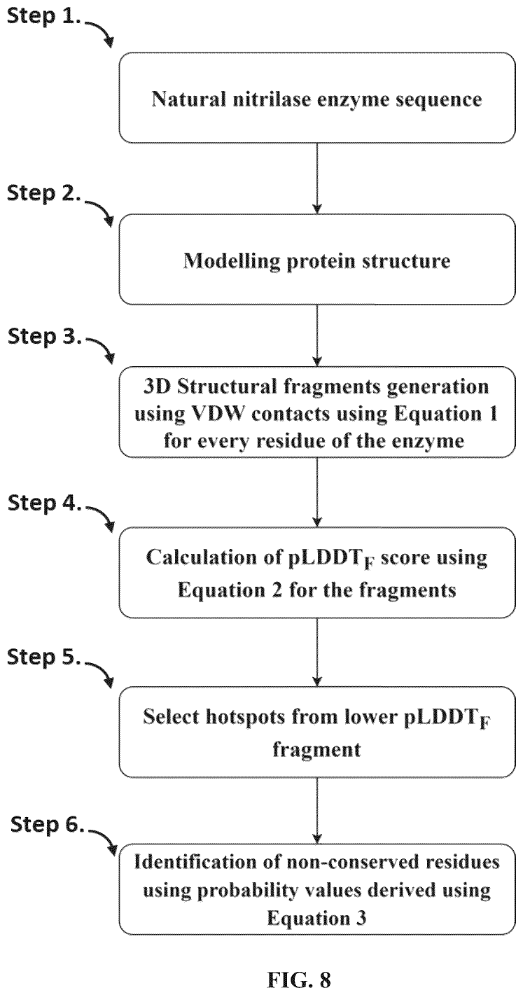

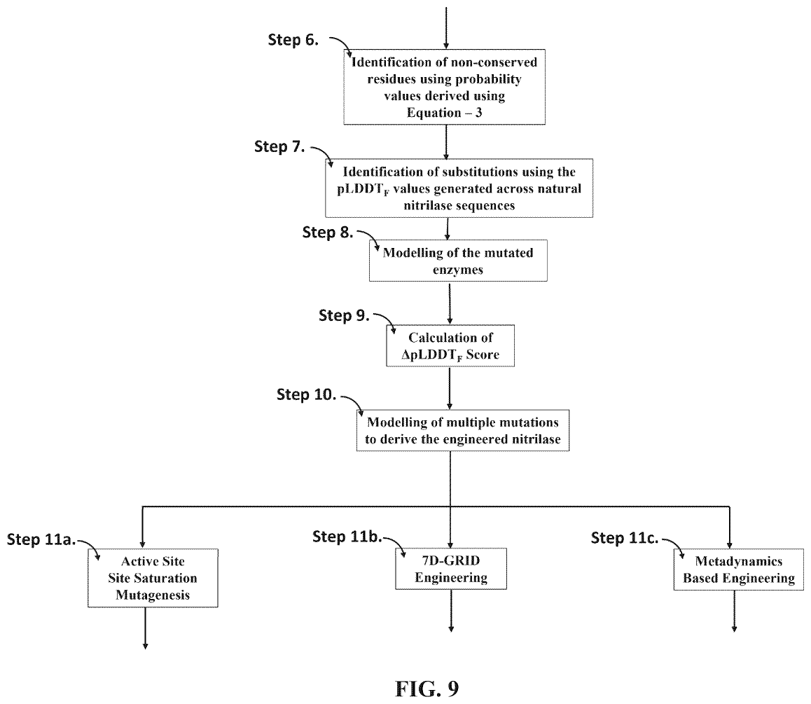

Step 15a: The generated enzyme-substrate complex are screened for the productive catalytic confrontation for all the substrates, 3-(4-chlorophenyl) pentanedinitrile (FIG. 3A), 2-chloropyridine-3-carbonitrile (FIG. 3B), Indole-3-acetonitrile (FIG. 3C), Mandelonitrile, (FIG. 4A), 2-(2-chlorophenyl)-2-hydroxyacetonitrile (FIG. 4B), 2-(6-methoxynaphthalen-2-yl)propanenitrile (FIG. 4C), 1-(cyanomethyl)cyclohexane-1-carbonitrile (FIG. 5A), 2-[1-(aminomethyl)cyclohexyl]acetonitrile (FIG. 5B), 2-[4-(2-methylpropyl)phenyl]propanenitrile (FIG. 5C), 2-hydroxy-3-phenylpropanenitrile (FIG. 6A), 3-(3-fluorophenyl)-2-hydroxypropanenitrile (FIG. 6B), 2-hydroxy-3-(naphthalen-1-yl)propanenitrile (FIG. 6C), 2-hydroxy-3-(pyridin-2-yl)propanenitrile (FIG. 7A), 2-hydroxy-3-(thiophen-3-yl)propanenitrile (FIG. 7B) and 2-(2-methylpropyl)butanedinitrile (FIG. 7C).

Step 16a & 17a: The screened productive Enzyme-substrate complexes are evaluated for binding affinity for the respective substrates against engineered nitrilase (ENIT) and ENITMUTANT. The assessment determines relative Km of the substrate in engineered variants and selectivity of the different substrates.

Step 18a: All the nitrilase variants were assessed with the relative binding affinity of the substrate against ENIT and ENIT_{MUTANT}. The higher binding affinity variants of nitrilases are considered with their active site substitution as an active variant for each substrate.

Step 19a: Optimized substrate specific nitrilase variants were obtained with improved selectivity. The optimized substrate specific enzyme variants are further synthesized and expressed in the E. coli BL21 using a pET28a(+) plasmid.

### Method for Engineering Nitrilase Using 7D-Grid Engineering.

The engineered nitrilase enzyme sequence was further engineered for improving the high substrate tolerance, broader pH and temperature for different substrates using 7D-GRID engineering method with the steps mentioned in FIG. 11. A detailed stepwise description of the method is described below:

Step 11b: 7D-GRID Technology uses Quantum Mechanic probes to “capture information across the enzymatic reaction and across the enzyme substrate system”. This information is used by AI methods, Convolutional Neural Networks, Support Vector Machines, to predict kinetic properties of the enzyme.

Step 12b: The 7D-GRID Technology uses a quantum mechanics probes in polarizable continuum model, with the proprietary probes that evaluates the direct enzyme-substrate QM interaction energy. This technology is a computer-implemented method for protein engineering that constructs a three-dimensional gridspace around a protein. Different probes, simulating the interactions of amino acids are iteratively placed within the grid. Pair Interaction Energy is calculated using FMO technique for probe-protein interactions. An algorithmic process calculates the sum of PIEs, extending from each grid point to its neighbours, until a higher cumulative PIE is obtained. Grid points are then grouped into patches and an alignment process matches a derived query probe pattern from each patch against existing patterns of an internal database. Mutations are made based on the highest PIE probe-amino acid pairs from the matched pattern, leading to a modified protein. This process can be iterated for generating optimal variants and can be localized to any part of the protein for identification of patches.

Step 13b: The generated enzyme variant library of engineered nitrilase enzymes were evaluated for the energetics for ranking of variants.

Step 14b: The optimized substrate-specific enzyme variants are further synthesized and expressed in the E. coli BL21 using a pET28a(+) plasmid.

### The Method of Engineering of Nitrilase Enzyme Using Funnel Metadynamics Simulations

The engineered nitrilase enzyme sequence was further engineered to improve the high substrate tolerance, broader pH and temperature for different substrates using the Metadynamics simulation-based engineering method with the steps mentioned in FIG. 12. A detailed stepwise description of the method is described below:

Step 11c: the method is used to engineer engineered nitrilase enzyme for improving the substrate specificity and activity at industrial conditions. The nitrilase enzyme engineered to fit nitrile substrates such as 3-(4-chlorophenyl)pentanedinitrile, 2-chloropyridine-3-carbonitrile, indole-3-acetonitrile, mandelonitrile, 2-(2-chlorophenyl)-2-hydroxyacetonitrile, 2-(6-methoxynaphthalen-2-yl)propanenitrile, 1-(cyanomethyl)cyclohexane-1-carbonitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile and Isobutylsuccinonitrile. These substrates were engineered individually using this method.

Step 12c: The substrate of interest from the above-mentioned list will be considered for further studies. The energy parameters were generated using the Amber force field and Gamess tools.

Step 13c: The engineered nitrilase and substrate of interest were used to conduct induced fit mode of studies yielding near-attack conformation of the Enzyme-Substrate complex.

Step 14c: The E-S complex is pre-processed for metadynamics simulations.

Step 15c: The funnel metadynamics simulations will be conducted from this step by defining proper collective variables (CVs) which form a funnel-shaped boundary to study energetically feasible minima. The substrate of interest is allowed to move through the narrow space of the funnel. The funnel will be defined with two different parameters in the funnel metadynamics where the XY plane defines the radius of the funnel curvature and Z-axis defines the narrow region of the funnel and also will be present in the centre of the funnel. The funnel restraint forces will be applied to the wall of the funnel which monitors the movement of the substrate of the interest within the boundary of the funnel.

Step 16c: The CVs in the metadynamics play an important role, which yields the results accordingly. The defined CVs involve the centre of mass (COM) the active site residues, surrounded pocket-forming residues (CV1) and the COM of the substrate (CV2). The funnel's XY plane and its curvature are defined to cover the complete active site pocket. The substitutions on any hotspots inside the funnel will make an impact in the centre of the mass of the funnel curvature.

Step 17c: After defining the CVs based on the hotspots, 200 ns of funnel Metadynamics simulations will be performed. The results of the Funnel Metadynamics should show highly impacting improvement in the convergence. If the free energy of ENIT mutant is not impactful or better than free of ENIT, that variant will not be considered for further testing.

Step 18c: The hotspot and its substitution which is not yielding better improvement is sent back to redefine the combinations and different substitutions.

Step 19c: When the free-energy convergence of ENIT mutant is lower than ENIT which is considered as an optimised variant for that particular substrate of interest. The optimised substrate-specific enzyme variants are further synthesised and expressed in the E. coli BL21 using a pET28a(+) plasmid. This cycle is repeated for all the substrates of interest to derive optimised substrate-specific ENIT enzyme variants.

The invention provides engineered Nitrilase polypeptides and respective polynucleotides that encode the same. The engineered Nitrilase polypeptides contain mutations at multiple positions obtained by the steps shown in FIGS. 8, 9, 10, 11 and 12. The engineered Nitrilase polypeptides are capable of converting 1-(cyanomethyl)cyclohexane-1-carbonitrile to (1-cyanocyclohexyl)acetic acid and additional nitrile substrates into corresponding acid products as shown in FIG. 1. The engineered Nitrilase polypeptides can catalyse these conversions with an activity that is equal to 100%, in industrial reaction conditions at a temperature range of 20-65° C. and pH range of 5.5-8.0.

The present disclosure provides engineered nitrilase polypeptides capable of converting nitrile substrates 3-(4-chlorophenyl)pentanedinitrile, 2-chloropyridine-3-carbonitrile, indole-3-acetonitrile, mandelonitrile, 2-(2-chlorophenyl)-2-hydroxyacetonitrile, 2-(6-methoxynaphthalen-2-yl)propanenitrile, 1-(cyanomethyl)cyclohexane-1-carbonitrile, 2-[1-(aminomethyl)cyclohexyl]acetonitrile, 2-[4-(2-methylpropyl)phenyl]propanenitrile, 2-hydroxy-3-phenylpropanenitrile, 3-(3-fluorophenyl)-2-hydroxypropanenitrile, 2-hydroxy-3-(naphthalen-1-yl)propanenitrile, 2-hydroxy-3-(pyridin-2-yl)propanenitrile, 2-hydroxy-3-(thiophen-3-yl)propanenitrile and Isobutylsuccinonitrile as shown in FIG. 1 to their corresponding acid products (3S)-3-(4-chlorophenyl)-4-cyanobutanoic acid, 2-chloronicotinic acid, indoleacetic acid, (R)-mandelic acid, (2-chlorophenyl)(hydroxy)acetic acid, (S)-naproxen, (1-cyanocyclohexyl)acetic acid, gabapentin, (S)-ibuprofen, (S)-phenyllactate, (2S)-3-(3-fluorophenyl)-2-hydroxypropanoic acid, (2S)-2-hydroxy-3-(naphthalen-1-yl)propanoic acid, (2S)-2-hydroxy-3-(pyridin-2-yl)propanoic acid, (2S)-2-hydroxy-3-(thiophen-3-yl)propanoic acid, and (S)-3-cyano-5-methylhexanoic acid respectively. The nitrilase polypeptides comprise an amino acid sequence that is at least about 80% identical to SEQ ID NO:1 wherein residue corresponding to position X3 is a Lys; X88 is Cys or Arg; X106 is Glu; X122 is Asp; X196 is Tyr; X229 is Lys; X260 is Arg; and X265 is Gly.

The engineered nitrilase involves the conversion of 1-(cyanomethyl) cyclohexane-1-carbonitrile to (1-cyanocyclohexyl)acetic acid, and it also efficiently converts the 2-(1-aminocyclohexyl)acetonitrile to (1-aminocyclohexyl)acetic acid. The synthesized product (1-cyanocyclohexyl)acetic acid and (1-aminocyclohexyl)acetic acid are common intermediate for commonly know drug Gabapentin, which is used as an anticonvulsant medication, primarily used to treat partial seizures and neuropathic pain.

Another substrate scope of engineered nitrilase involves the conversion of 2-(2-chlorophenyl)-2-hydroxyacetonitrile to (2-chlorophenyl) (hydroxy)acetic acid. The (2-chlorophenyl)(hydroxy)acetic acid is also called as (R)-o-chloromandelic acid which is a key precursor for the synthesis of the Clopidogrel, an antiplatelet medicine.

The another substrate scope of engineered nitrilase involves the conversion of Mandelonitrile to (R)-(−)-Mandelic acid, which has numerous potential applications in the pharmaceuticals industry as it is a an ideal starting material for the synthesis of antibiotics, antiobesity drugs and antitumor agents. It is also used in for dermatological purposes, for reducing acne, fine lines and wrinkles and improve the appearance of uneven skin.

Another substrate scope of engineered nitrilase involves the conversion of Isobutylsuccinonitrile to (S)-3-cyano-5-methylhexanoic acid, an key precursor for the (S)-pregabalin. (S)-pregabalin is a medication that is widely used for the treatment of nerve pain, and it is also used in the treatment of epilepsy by preventing and managing seizures.

The other substrate scope of engineered nitrilase involves the conversion of 3-(4-chlorophenyl)pentanedinitrile to (3S)-3-(4-chlorophenyl)-4-cyanobutanoic acid. Which is a key intermediate for pharmaceutically important drug Baclofen. It is used to treat muscle spasticity, manage reversible spasticity, particularly for the relief of flexor spasms, clonus, and concomitant pain, common sequelae of spinal cord lesions, and multiple sclerosis.

Another substrate scope of engineered nitrilase involves the conversion of 2-chloropyridine-3-carbonitrile to 2-chloronicotinic acid. Which is key intermediate for many important pharmaceutical drugs. It used in the synthesis of the anti-inflammatory and analgesic pralofen. It is an intermediate of the herbicides nicosulfuron and diflufenican. It is also used as pharmaceutical intermediates for the manufacture of mefenamic acid, niflumic acid, etc.

The another substrate scope of engineered nitrilase involves the conversion of Indole-3-acetonitrile to Indoleacetic acid (IAA). Indole-3-acetic acid is the most common plant hormone of the auxin class and regulates various plant growth processes.

The another substrate scope of engineered nitrilase involves the conversion of 2-(6-methoxynaphthalen-2-yl)propanenitrile to (S)-naproxen. It is a common drug used to treat pain and fever. It belongs to the class of medicines called non-steroidal anti-inflammatory drugs.

The another substrate scope of engineered nitrilase involves the conversion of 2-[4-(2-methylpropyl)phenyl]propanenitrile to (S)-ibuprofen. It is also called as Dexibuprofen, which is most commonly used as a non-narcotic analgesic and a non-steroidal anti-inflammatory drug.

Another substrate scope of engineered nitrilase involves the conversion of 2-hydroxy-3-phenylpropanenitrile to (S)-phenyllactate. (S)-phenyllactate is also called as (S)-phenyllactic acid which is used a versatile building blocks for the preparation of numerous biologically active compounds. Phenyllactic acid is a green compound used in biopreservation, and also acts as a broad-spectrum antimicrobial compounds.

Another substrate scope of engineered nitrilase involves the conversion of 3-(3-fluorophenyl)-2-hydroxypropanenitrile to (2S)-3-(3-fluorophenyl)-2-hydroxypropanoic acid.

The another substrate scope of engineered nitrilase involves the conversion of 2-hydroxy-3-(naphthalen-1-yl)propanenitrile to (2S)-2-hydroxy-3-(naphthalen-1-yl)propanoic acid.

The another substrate scope of engineered nitrilase involves the conversion of 2-hydroxy-3-(pyridin-2-yl)propanenitrile to (2S)-2-hydroxy-3-(pyridin-2-yl)propanoic acid.

The another substrate scope of engineered nitrilase involves the conversion of 2-hydroxy-3-(thiophen-3-yl)propanenitrile, to (2S)-2-hydroxy-3-(thiophen-3-yl)propanoic acid.

The improvement in enzyme activity is with respect to another engineered nitrilase, such as the polypeptide of SEQ ID NO:1 The improved activity on a nitrile substrate can be manifested by an increase in the amount of substrate converted to product (e.g., percent conversion) by the engineered enzyme relative to a reference enzyme (e.g., natural nitrilase sequence) under defined conditions. The improved activity can include an increased rate of product formation resulting in an increase in the conversion of nitrile substrate to acid product in a defined time under a defined condition. The increase in activity (e.g., increased percent conversion and/or conversion rate) may also be characterized by the conversion of substrate to the same amount of product with a lower amount of enzyme. The amount of product can be assessed by a variety of techniques, for example, separation of the reaction mixture (e.g., by chromatography) and detection of the separated product by UV absorbance or tandem mass spectroscopy (MS/MS).

In some embodiments, the improved enzymatic activity is also associated with other improvements in enzyme properties. In some embodiments, the improvement in enzyme property is with respect to thermal stability, such as at 450° C. or higher.

In some embodiments, the engineered nitrilase polypeptides of the present disclosure are capable of converting 3-(4-chlorophenyl)pentanedinitrile to (3S)-3-(4-chlorophenyl)-4-cyanobutanoic acid, 2-chloropyridine-3-carbonitrile to 2-chloronicotinic acid, Indole-3-acetonitrile to indoleacetic acid, Mandelonitrile to (R)-mandelic acid, 2-(2-chlorophenyl)-2-hydroxyacetonitrile to (2-chlorophenyl)(hydroxy)acetic acid, 2-(6-methoxynaphthalen-2-yl)propanenitrile to (S)- naproxen, 1-(cyanomethyl)cyclohexane-1-carbonitrile to (1-cyanocyclohexyl)acetic acid, 2-[1-(aminomethyl)cyclohexyl]acetonitrile to gabapentin, 2-[4-(2-methylpropyl)phenyl]propanenitrile to (S)-ibuprofen, 2-hydroxy-3-phenylpropanenitrile to (S)-phenyllactate, 3-(3-fluorophenyl)-2-hydroxypropanenitrile to (2S)-3-(3-fluorophenyl)-2-hydroxypropanoic acid, 2-hydroxy-3-(naphthalen-1-yl)propanenitrile to (2S)-2-hydroxy-3-(naphthalen-1-yl)propanoic acid, 2-hydroxy-3-(pyridin-2-yl)propanenitrile to (2S)-2-hydroxy-3-(pyridin-2-yl)propanoic acid, 2-hydroxy-3-(thiophen-3-yl)propanenitrile, to (2S)-2-hydroxy-3-(thiophen-3-yl)propanoic acid, Isobutylsuccinonitrile to (S)-3-cyano-5-methylhexanoic acid having improved activity with polypeptide sequence of SEQ ID NO: 1 as compared to any other reported nitrilase, and comprises an amino acid sequence that is at least 80% identical to engineered sequence SEQ ID NO: 1.

The other substrate scope of engineered nitrilase SEQ ID 267 involves the conversion of 3-(4-chlorophenyl)pentanedinitrile to (3S)-3-(4-chlorophenyl)-4-cyanobutanoic acid. Wherein the residue corresponding to X66 is M, the residue corresponding to X10 is S, the residue corresponding to X99 is V, the residue corresponding to X149 is T, the residue corresponding to X113 is T, the residue corresponding to X116 is I, the residue corresponding to X78 is I, the residue corresponding to X124 is Q, the residue corresponding to X69 is V, the residue corresponding to X315 is E, the residue corresponding to X275 is L, and the residue corresponding to X263 is A.

The other substrate scope of engineered nitrilase SEQ ID 269 involves the conversion of 2-chloropyridine-3-carbonitrileinto 2-chloronicotinic acid, Wherein the residue corresponding to X37 is A, the residue corresponding to X17 is Y, the residue corresponding to X93 is E, the residue corresponding to X98 is I, the residue corresponding to X135 is G, the residue corresponding to X78 is E, the residue corresponding to X80 is D, the residue corresponding to X149 is S, the residue corresponding to X100 is S, the residue corresponding to X195 is G, the residue corresponding to X275 is M, and the residue corresponding to X239 is P.

The other substrate scope of engineered nitrilase SEQ ID 271 involves the conversion of Indole-3-acetonitrile into indoleacetic acid (IAA). Wherein the residue corresponding to X17 is W, the residue corresponding to X31 is I, the residue corresponding to X149 is V, the residue corresponding to X99 is L, the residue corresponding to X119 is A, the residue corresponding to X94 is A, the residue corresponding to X78 is I, the residue corresponding to X69 is F, the residue corresponding to X81 is N, the residue corresponding to X255 is Y, the residue corresponding to X306 is I, and the residue corresponding to X288 is S.

The other substrate scope of engineered nitrilase SEQ ID 273 involves the conversion of Mandelonitrile is converted into (R)-mandelic acid. Wherein the residue corresponding to X10 is I, the residue corresponding to X66 is L, the residue corresponding to X78 is S, the residue corresponding to X80 is N, the residue corresponding to X69 is I, the residue corresponding to X113 is T, the residue corresponding to X119 is A, the residue corresponding to X99 is V, the residue corresponding to X81 is N, the residue corresponding to X305 is L, the residue corresponding to X160 is T, and the residue corresponding to X239 is N.

The other substrate scope of engineered nitrilase SEQ ID 275 involves the conversion of 2-(2-chlorophenyl)-2-hydroxyacetonitrile into (2-chlorophenyl)(hydroxy)acetic acid. Wherein the residue corresponding to X17 is L, the residue corresponding to X66 is M, the residue corresponding to X93 is K, the residue corresponding to X81 is S, the residue corresponding to X98 is A, the residue corresponding to X124 is V, the residue corresponding to X119 is D, the residue corresponding to X113 is T, the residue corresponding to X116 is H, the residue corresponding to X313 is P, the residue corresponding to X336 is E, the residue corresponding to X246 is R, and the residue corresponding to X167 is M.

The other substrate scope of engineered nitrilase SEQ ID 277 involves the conversion of 2-(6-methoxynaphthalen-2-yl)propanenitrile into (S)- naproxen. Wherein the residue corresponding to X8 is V, the residue corresponding to X60 is T, the residue corresponding to X119 is N, the residue corresponding to X116 is I, the residue corresponding to X80 is E, the residue corresponding to X99 is V, the residue corresponding to X81 is L, the residue corresponding to X135 is A, the residue corresponding to X149 is S, the residue corresponding to X326 is T, the residue corresponding to X288 is S, the residue corresponding to X211 is T, and the residue corresponding to X154 is E.

The other substrate scope of engineered nitrilase SEQ ID 279 involves the conversion of 2-[1-(aminomethyl)cyclohexyl]acetonitrile into gabapentin. Wherein the residue corresponding to X49 is I, the residue corresponding to X23 is A, the residue corresponding to X80 is N, the residue corresponding to X98 is A, the residue corresponding to X69 is V, the residue corresponding to X116 is I, the residue corresponding to X81 is L, the residue corresponding to X100 is F, the residue corresponding to X93 is N, the residue corresponding to X124 is Q, the residue corresponding to X211 is L, the residue corresponding to X267 is A, the residue corresponding to X160 is M, and the residue corresponding to X217S.

The other substrate scope of engineered nitrilase SEQ ID 281 involves the conversion of 2-[4-(2-methylpropyl)phenyl]propanenitrile into (S)-Ibuprofen. Wherein the residue corresponding to X37 is E, the residue corresponding to X8 is V, the residue corresponding to X81 is S, the residue corresponding to X113 is T, the residue corresponding to X135 is T, the residue corresponding to X98 is L, the residue corresponding to X100 is L, the residue corresponding to X124 is V, the residue corresponding to X78 is T, the residue corresponding to X69 is V, the residue corresponding to X274 is V, the residue corresponding to X251 is A, the residue corresponding to X217 is T, and the residue corresponding to X275 is M.

The other substrate scope of engineered nitrilase SEQ ID 283 involves the conversion of 2-hydroxy-3-phenylpropanenitrile into (S)-phenyllactate. Wherein the residue corresponding to X37 is A, the residue corresponding to X8 is I, the residue corresponding to X69 is I, the residue corresponding to X81 is L, the residue corresponding to X135 is S, the residue corresponding to X98 is A, the residue corresponding to X116 is V, the residue corresponding to X149 is G, the residue corresponding to X100 is F, the residue corresponding to X99 is A, the residue corresponding to X305 is L, the residue corresponding to X309 is V, the residue corresponding to X335 is P, and the residue corresponding to X288 is N.

The other substrate scope of engineered nitrilase SEQ ID 285 involves the conversion of 3-(3-fluorophenyl)-2-hydroxypropanenitrile into (2S)-3-(3-fluorophenyl)-2-hydroxypropanoic acid. Wherein the residue corresponding to X66 is L, the residue corresponding to X49 is V, the residue corresponding to X124 is Q, the residue corresponding to X69 is F, the residue corresponding to X99 is L, the residue corresponding to X80 is D, the residue corresponding to X81 is L, the residue corresponding to X93 is R, the residue corresponding to X135 is A, the residue corresponding to X98 is M, the residue corresponding to X336 is G, the residue corresponding to X326 is T, the residue corresponding to X319 is T, and the residue corresponding to X269 is D.

The other substrate scope of engineered nitrilase SEQ ID 287 involves the conversion of 2-hydroxy-3-(naphthalen-1-yl)propanenitrile into (2S)-2-hydroxy-3-(naphthalen-1-yl)propanoic acid. Wherein the residue corresponding to X23 is S, the residue corresponding to X49 is V, the residue corresponding to X113 is T, the residue corresponding to X100 is S, the residue corresponding to X135 is T, the residue corresponding to X99 is V, the residue corresponding to X284 is A, and the residue corresponding to X333 is L.

The other substrate scope of engineered nitrilase SEQ ID 289 involves the conversion of 2-hydroxy-3-(pyridin-2-yl)propanenitrile into (2S)-2-hydroxy-3-(pyridin-2-yl)propanoic acid. Wherein the residue corresponding to X48 is F, the residue corresponding to X17 is W, the residue corresponding to X119 is N, the residue corresponding to X99 is G, the residue corresponding to X113 is T, the residue corresponding to X98 is A, the residue corresponding to X160 is V, and the residue corresponding to X333 is L.

The other substrate scope of engineered nitrilase SEQ ID 291 involves the conversion of 2-hydroxy-3-(thiophen-3-yl)propanenitrile into (2S)-2-hydroxy-3-(thiophen-3-yl)propanoic acid. Wherein the residue corresponding to X8 is I, the residue corresponding to X49 is V, the residue corresponding to X80 is D, the residue corresponding to X113 is A, the residue corresponding to X81 is S, the residue corresponding to X94 is A, the residue corresponding to X93 is N, the residue corresponding to X124 is L, the residue corresponding to X135 is S, the residue corresponding to X237 is S, the residue corresponding to X268 is H, the residue corresponding to X167 is F, and the residue corresponding to X286 is G.

The other substrate scope of engineered nitrilase SEQ ID 293 involves the conversion of Isobutylsuccinonitrile into (S)-3-cyano-5-methylhexanoic acid. Wherein the residue corresponding to X48 is Y, the residue corresponding to X10 is I, the residue corresponding to X149 is V, the residue corresponding to X135 is S, the residue corresponding to X81 is L, the residue corresponding to X93 is Q, the residue corresponding to X124 is Q, the residue corresponding to X246 is T, the residue corresponding to X334 is P, and the residue corresponding to X335 is H.

In some embodiments, the engineered nitrilase polypeptides comprise an amino acid sequence that has one or more residue differences as compared to an engineered nitrilase sequence SEQ ID NO:1. The residue differences can be non-conservative substitutions, conservative substitutions, or a combination of non-conservative and conservative substitutions. With respect to the residue differences and the descriptions of residue positions, the nitrilases provided herein can be described in reference to the amino acid sequence of the engineered nitrilase of SEQ ID NO:1.

A specific substitution mutation, which is a replacement of the specific residue in a reference sequence with a different specified residue may be denoted by the conventional notation “X(number)Y”, where X is the single letter identifier of the residue in the reference sequence, “number” is the residue position in the reference sequence, and Y is the single letter identifier of the residue substitution in the engineered sequence.

In some embodiments, the residue differences can occur at one or more of the following residue positions: X3; X8; X10; X17; X23; X28; X31; X33; X37; X48; X49; X59; X60; X65; X66; X67; X69; X70; X78; X79; X80; X81; X84; X85; X88; X90; X93; X94; X97; X98; X99; X100; X106; X108; X113; X116; X119; X122; X124; X126; X135; X149; X151; X153; X154; X158; X160; X167; X179; X192; X194; X195; X196; X198; X202; X203; X206; X207; X211; X217; X226; X229; X230; X232; X237; X238; X239; X240; X241; X246; X247; X250; X251; X253; X255; X260; X261; X263; X265; X267; X268; X269; X270; X274; X275; X282; X284; X286; X288; X290; X305; X306; X307; X309; X310; X313; X315; X317; X319; X323; X326; X333; X334; X335; and X336. In some embodiments, the residue differences, or combinations thereof, are associated with the improved enzyme properties. In some embodiments, the engineered nitrilase polypeptides can have additionally 1, 2, 3, 4, 5, 6, 7, 8, 9, 10, 11, 12, 14, 15, 16, 18, 20, 22, 24, 25, 26, 27, 28, 29, 30, 31, 32, 33, 34, 35, and 36 residue differences at the other amino acid residue positions.

In some embodiments of the engineered nitrilases of the disclosure, the amino acid residues at a residue position can be defined in terms of the amino acid “features” (e.g., type or property of amino acids) that can appear at that position. Thus, in some embodiments the amino acid residues at the positions specified above can be selected from the following features: X3 is a Lys; X88 is Cys or Arg; X106 is Glu; X122 is Asp; X196 is Tyr; X229 is Lys; X260 is Arg; and X265 is Gly.

In some embodiments of the engineered nitrilases of the disclosure, the amino acid residues at a residue position can be defined in terms of the amino acid “features” (e.g., type or property of amino acids) that can appear at that position. Thus, in some embodiments the amino acid residues at the positions specified above can be selected from the following features: X14 is Cys; X28 is Arg; X33 is Lys; X59 is Tyr; X61 is Asn; X65 is Val; X67 is Lys or Val or Arg; X70 is Arg or Asp; X79 is Phe; X84 is Lys; X85 is His; X97 is Tyr; X107 is Ser; X108 is Glu; X121 is Cys; X151 is His; X153 is Ile; X160 is Trp; X179 is Lys; X184 is Cys; X192 is Ile; X194 is Gly; X197 is Cys; X199 is Trp; X203 is Ile; X206 is Asp; X226 is Cys; X228 is Cys; X230 is Ala; X235 is Leu; X237 is His; X240 is Val; X241 is Arg; X250 is Cys; X253 is Ser or Glu; X261 is Glu; X264 is Ser; X290 is Asn; X302 is Arg; and X307 is Asp.

In some embodiments of the engineered nitrilases of the disclosure, the amino acid residues at a residue position can be defined in terms of the amino acid “features” (e.g., type or property of amino acids) that can appear at that position. Thus, in some embodiments the amino acid residues at the positions specified above can be selected from the following features: X8 is Val or Leu or Ile; X10 is Val or Ser or Ile; X17 is Trp or Tyr or Leu; X23 is Ser or Gly or Ala; X31 is Ile or Val or Ala; X37 is Lys or Gln or Ala or Glu or His or Asp; X48 is Phe or Tyr; X49 is Ile or Val or Ala or Phe; X60 is Ser or Thr or Val; X66 is Ile or Met or Phe or Leu; X69 is Val or Ile or Phe; X78 is Ser or Val or Ile or Glu or Ile or Thr; X80 is Asp or Asn or Glu; X81 is Ser or Asn or Leu; X93 is Lys or Arg or Glu or Gln or Asn; X94 is Ala or His or Asn or Len; X98 is Ala or Len or Ile or Met; X99 is Val or Ala or Len or Gly; X100 is Len or Ile or Phe or Val or Thr or Ala or Ser; X113 is Ala or Thr; X116 is Len or Ile or Val or His; X119 is Pro or Asp or Ala or Ser or Asn; X124 is Val or Len or Ala or Gln; X135 is Ala or Gly or Ser or Thr; X149 is Ser or Thr or His or Gly or Val or Met; X154 is Asp or Gly or Arg or Lys or Glu; X158 is Ile or Len or Met; X160 is Ala or Ser or Met or Thr or Val; X167 is Ile or Val or Phe or Met; X195 is Gly or Pro; X198 is Pro or His or Asn or Phe; X202 is Ala or Trp or His or Gly or Ser; X206 is Asn or Val or Glu or Ser; X207 is Ala or Gly or His; X211 is Val or Len or Thr or Ala or Met; X217 is Ser or Thr or Gly; X232 is Ile or Ser or His or Gln or Leu; X237 is Asp or Gly or Glu or Ser or Ile; X238 is Arg or Asp or Ser or Glu; X239 is Pro or Ala or Glu or Asn; X246 is His or Arg or Len or Thr; X247 is Val or Ala or Glu or Pro; X251 is His or Phe or Ser or Ala; X255 is Tyr or Leu; X263 is Gly or Ala or Thr or Ser or Val; X267 is Ala or Asp or Gly; X268 is Pro or Glu or His or Thr; X269 is Asp or Glu or Asn or Thr or Ser; X270 is Gln or Ala or Thr or Val or Arg or Gly or Lys; X274 is Len or Val or Ala; X275 is Ile or Len or Val or Phe or Met or Cys; X282 is Ala or Met or Val or Thr; X284 is Gly or Ala or Thr; X286 is Ala or Gly; X288 is Asn or Ser or Thr; X305 is Len or Trp or Val or Met; X306 is Len or Phe or Val or Ile; X309 is Lys or Thr or Ser or Arg or Val; X310 is Arg or Ser or Val or Ala; X313 is Arg or Pro or Val; X315 is Glu or Ile or Met or Thr or Ser; X317 is Phe or Gln or Gly or Ala or Arg or Len or Val; X319 is Len or Gln or Thr or Ala; X323 is Asp or Glu or Asn or Ala; X326 is Gly or Ser or Thr or Glu or Pro or Arg; X333 is Thr or Val or Pro or Len or Glu or Lys; X334 is Glu or Ala or Pro or Len or Ser; X335 is Gln or Pro or Len or Ala or His or Asp or Ser; and X336 is Glu or Len or Ala or Gly or Val or Asp;

In some embodiments, the engineered nitrilase comprises an amino acid sequence that has a substitution or a plurality of amino acid substitutions, as described previously, with respect to the amino acid sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291 or 293 and having catalytic activity for the conversion of bulky substrates having nitrile group to corresponding carboxylic acid group under the suitable reaction conditions such as 200 mM substrate, in 100 mM phosphate buffer (pH 7.0) and incubation at 45° C.

Furthermore, a method for expression of recombinant nitrilase gene comprises preparing a host organism E. coli BL21(DE3) and introducing a plasmid pET28a(+), in which nitrilase gene is arranged under the control of an inducible promoter (FIG. 16a). Followed by culturing the host, inducing the expression of the nitrilase gene after the logarithmic growth phase, culturing the host at a temperature that is lower than the optimum temperature for the growth of the host cells and allowing the survival of the host, and thus causing the expression of the nitrilase within the host.

Furthermore, a method of fermentation of engineered nitrilase comprises the optimized medium composition as follows: peptone, 15 g/L; yeast extract, 12 g/L; NaCl, 10 g/L; Glycerol, 15 g/L; (NH₄)₂SO4, 5 g/L; K₂HPO₄.3H₂O 4.1 g/L, KH₂PO₄, 6.8 g/L; MgSO₄.7H₂O, 1.125 g/L (pH 7.0). Cells were firstly transferred to 750-ml flasks containing 150 mL of LB medium from the colony and incubated at 37° C. and 150 rpm. Kanamycin (50 g/L) was added to the medium at the beginning of inoculation. When cells were grown to the end of exponential growth phase, 900 mL of the culture broth was transferred to a 50-L fermentor containing 30L of LB medium. Cells were cultivated at 37° C. for 3 h with aeration at 1.1 vvm and agitation at 500 rpm. 15 L of the culture broth was then transformed to 500-L fermentator containing 300L of optimized fermentation medium. Fermentation was carried out at 37° C. with aeration at 1.4 vvm and agitation at 240 rpm for 4 h. The fermentation temperature was then decreased to 28° C., and lactose (12.5 g/L) was added to induce the nitrilase activity. After an 8 h fermentation, whole cells were harvested by centrifugation.

Furthermore, a method of immobilization with Ca-alginate embedding of whole cells comprises the resuspension of engineered nitrilase containing E. coli wet cells (5.0 g) were suspended in 100 mL of 3% (w/v) alginate solution. The cell-alginate solution was then cast into beeds by dropping into sterile 4% (w/v) CaCl₂ solution. The entrapped beads were hardened by keeping in the 4% CaCl₂ solution for 12 h at 4° C. These beads were washed with sterile distilled water 3 times and then stored at 4° C. in sterile distilled water. The activities of the beads were measured with Tris-HCl (100 mM, pH 7.0) as the buffer.

Furthermore, a method of immobilization with Carrageenan or agar embedding of whole cells comprises the resuspension of engineered nitrilase containing E. coli wet cells (5.0 g) were suspended in 20 ml of 3% (w/v) carrageenan or agar solution. Then the mixture was poured into a pretreated petri-plates and allowed to solidify. The solidified gel was cut into equal size cubes (0.5×0.5×0.5 cm), washed with sterile distilled water for 3-4 times. The cubes were stored at 4° C. in sterile distilled water until use.

Furthermore, a method of immobilization with Cell Cross-linking of whole cells comprises the resuspension of engineered nitrilase containing E. coli wet cells (5.0 g) were suspended in 100 mL of 100 mM sodium phosphate buffer (pH 7.0). 0.2% (v/v) cross-linking reagent and 0.3% (w/v) diatomite dosage was added to the suspended cells and then stirred by the magnetic stirrer at 25° C. for 30 min, then the cross-linked cells were recovered by filtration. For polyethyleneimine (PEI)/glutaraldehyde (GA) cross-linking, 0.2% (v/v) PEI and 0.3% (w/v) diatomite dosage were firstly added and stirred for 30 min for flocculation and sedimentation of cells. Then, 0.2% (v/v) glutaraldehyde was added, and the mixture was stirred by the magnetic stirrer for another 30 minutes. Finally, the cross-linked cell aggregates (CLCAs) were recovered by filtration. The CLCAs washed 3 to 4 times with sterile distilled water and stored at 4° C. in sterile distilled water until use.

Furthermore, a method of reusability of CLCAs engineered nitrilase comprises a typical reaction mixture (100 mL) containing 4.0 g wet cells or 17.5 g CLCAs (corresponding to 7.0 g free cells) and substrate (500 mM) suspended in 100 mM sodium phosphate buffer (pH 7.0) was incubated at 45° C. for 3 hr in a 250 ml 3-necked flask with a mechanical stirrer (200 rpm). After each cycle, the free cells were recovered by centrifugation at 10,000×g for 10 min and the CLCAs were recovered by filtration through two layers of gauze. The recovered free cells and CLCAs were then washed with 100 mM sodium phosphate buffer (pH 7.0) three times. The residual nitrilase activity of each batch was measured before the next cycle of biotransformation.

To create/facilitate the mutation of nitrilase described in the present invention, several of predetermined amino acid residues are substituted in the SEQ ID NO: 1. The mutations have significantly improved the nitrilase activity for bulky substrates with nitrile group and stability. Here “improved stability” means broader thermal stability (20-65° C.) and pH stability (5.5-8).

In some embodiments, the engineered nitrilase polypeptide works in the suitable process conditions where, the standard reaction was carried out by mixing the substrate of 200 mM with the purified or lyophilized enzyme in phosphate buffer at 100 mM with a pH of 7.0. The reaction mixture was incubated at 45° C. for 10 min with shaking at 150 rpm in the reciprocal shaker. The reactions were quenched by addition of 2M HCl.

In some embodiments, the suitable process conditions for nitrilase comprises a temperature range of 20° C. to about 65° C.

In some of embodiment the suitable process conditions for nitrilase comprises a pH of 5.5 to about 8.0.

In some of embodiment the suitable process conditions for nitrilase comprises the substrate compound at a loading of about 5 g/L to about 200 g/L.

In some of the embodiment the suitable process conditions for nitrilase comprises the engineered nitrilase at a concentration from 0.5 g/L to about 5 g/L.

Here, engineered nitrilase polypeptide is obtained by conventionally known methods for protein production. Specifically, a method for producing nitrilase gene comprises a preparation of a host organism E. coli BL21 (DE3) and introducing through an expression vector, in which engineered nitrilase gene is arranged under the control of inducible promoters between the restriction sites (FIG. 16b). Followed by culturing the host, inducing the expression of the engineered nitrilase gene after the logarithmic growth phase, culturing the host at a temperature that is lower than the optimum temperature for growth of the host cells and allows the survival of the host, and thus causing the expression of the engineered nitrilase within the host. As an inducible promoter to be used in the method for producing the engineered nitrilase, any other conventional promoters can also be used without limitations. As an illustration, when the expression host E. coli is utilized, an inducible promoter that can be activated by the presence of isopropyl-D-1-thiogalactopyranoside (IPTG) can activate transcription. The promoters Trp, Lac, Trc, and Tac are a few examples of this type.

The engineered nitrilase polypeptides can be expressed in bacterial host cell like E. coli, using the expression vector pET28a(+) on which the engineered nitrilase polynucleotides are constructed and cloned to the bacteria and cultured in a suitable medium. The host cells can be used for the expression and isolation of the engineered nitrilase polypeptides or alternatively, they can be used directly for the conversion of the nitrile substrate to product.

In another aspect, the engineered nitrilase polypeptides, in the form of whole cells, crude extracts, isolated polypeptides, or purified polypeptides, can be used individually or as a combination of different engineered nitrilase polypeptides.

For the purposes of the descriptions herein, the abbreviations used for the genetically encoded amino acids are conventional and are as follows:

| Amino Acid | Three-Letter Abbreviation | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (Ca).

### EXPERIMENTS

### Example 1: Expression Protocol for Engineered Nitrilase Polypeptides

The recombinant plasmid carrying the genes was transformed into BL21 competent cells and were grown at 37° C. in Luria bertani broth containing kanamycin (50 ug/ml). Enzyme production was induced by addition of 0.05 mM isopropyl-3-β-thiogalactoside (IPTG) when optical density of the culture broth reached 0.6 at OD 600 nm, and then incubated at 25-C for 16 hr. Culture was harvested by centrifugation at 4000 rpm for 15 min. The harvested cells were lysed with 1 mg/ml lysozyme and suspended in 100 mM Triethanolamine (pH-7.5) for sonication. The soluble protein fractions were obtained by centrifugation at 8000 rpm for 15 min at 4-C. The crude protein lysates were analyzed using 10% sodium dodecyl sulfate poly-acrylamide gel electrophoresis (SDS-PAGE). The gel pictures below show the expression of 10 of the many the engineered Nitrilases (Figure. 15).

### Biocatalytic Assay of Engineered Nitrilase Polypeptides

### Example 2: Engineered Nitrilase Gene Expression

The engineered nitrilase gene was introduced into the E. coli BL21 (DE3) strain. The bacteria were cultured in 500 mL Erlenmeyer flasks with 100 mL of fresh LB medium, consisting of 5 g/L yeast extract, 10 g/L tryptone, and 5 g/L NaCl. The culture was supplemented with 50 g/mL kanamycin and incubated at 37° C. until the OD600 reached 1.0. Then, the expression of the engineered nitrilase was induced by adding 0.6 mM isopropyl-beta-D-thiogalactopyranoside (IPTG) and incubating at 28° C. for 12 hours. The cells were harvested by centrifugation at 10,000×g for 15 minutes at 4° C. The resulting cell pellets were resuspended in 100 mM sodium phosphate buffer (pH 7.0) and sonicated on a water-ice bath for 5 minutes at 240 W. This was done using a is sonication followed by a 3s rest cycle, for a total of 5 minutes (75 cycles). The cell debris was removed by centrifugation at 12,000×g for 10 minutes. The soluble protein sample was loaded onto a Ni-NTA superflow column. The fractions containing pure engineered nitrilase were pooled and dialyzed against 100 mM sodium phosphate buffer (pH 7.0). The purified enzyme was then stored at 4° C.

The standard assays were carried out by mixing the substrate 1-(cyanocyclohexyl)acetonitrile (5 mg/mL to 200 mg/mL final concentration) with the purified enzyme in phosphate buffer (100 mM, pH 7.0). The reaction mixture (10 mL and 100 mL final volume) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of 1-(cyanocyclohexyl)-acetic acid formed in the reaction. One unit of enzyme activity was designed as the amount of enzyme producing 1 μmol of 1-(cyanocyclohexyl)-acetic acid per minute under the standard assay conditions (FIG. 17).

The concentration of 1-(cyanocyclohexyl)-acetic acid was determined by a C18 (5 μm×250 mm×4.6 mm) column (Elite Analytical Instruments Co., Ltd.). HPLC was used for the detection of product wherein, by UV detector was set at a wavelength of 215 nm, and each sample (20 L) was eluted at 40-C with 5 mM NH₄H2PO₄/15 mM sodium perchlorate (pH 1.8) treated with perchloric acid: acetonitrile=76:24 (v/v, 1.0 mL/min) as mobile phase.

The reaction mixture contained cell suspension (200 mg/mL) in 50 mM phosphate buffer (pH 7.8) and 120 mM substrate dissolved in DMSO. The biocatalysis reaction was carried out in an incubator shaker (37° C., 200 rpm) for 24 h. The cells were removed by centrifugation (7000×g) and the supernatant was extracted with ethyl acetate to recover the product.

Whole Cell Biocatalysis: 20 mM substrate concentration and 1 gm whole cell (dry cell weight) with 50 mM phosphate buffer (pH 7.8). 120 mM substrate (1-cyanocyclohexyl)acetonitrile) was used for the whole cell bio catalysis mediated product (1-cyanocyclohexyl)acetic acid) formation

### Example 3

Protocol for the conversion of 3-(4-chlorophenyl)pentanedinitrile to (3S)-3-(4-chlorophenyl)-4-cyanobutanoic acid using engineered nitrilase.

The standard assays were carried out by mixing the substrate 3-(4-chlorophenyl)pentanedinitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (3 S)-3-(4-chlorophenyl)-4-cyanobutanoic acid formed in the reaction. One unit of enzyme activity was designed as the amount of enzyme producing 1 mol of (3S)-3-(4-chlorophenyl)-4-cyanobutanoic acid per minute under the standard assay conditions. The concentration of (3S)-3-(4-chlorophenyl)-4-cyanobutanoic acid was determined by analytical methods.

### Example 4

Protocol for the conversion of 2-chloropyridine-3-carbonitrile to 2-chloronicotinic acid using engineered nitrilase.

The standard assays were carried out by mixing the substrate 2-chloropyridine-3-carbonitrile (5 mg/mL) with the purified enzyme in phosphate buffer (100 mM, pH 7.0). The reaction mixture (10 mL) was incubated at 35° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of 2-chloronicotinic acid formed in the reaction. One unit of enzyme activity was designed as the amount of enzyme producing 1 mol of 2-chloronicotinic acid per minute under the standard assay conditions. The concentration of 2-chloronicotinic acid was determined by analytical methods.

### Example 5

Protocol for the conversion of Indole-3-acetonitrile to indoleacetic acid using engineered nitrilase.

The standard assays were carried out by mixing the substrate Indole-3-acetonitrile (5 mg/mL) with the purified enzyme in phosphate buffer (100 mM, pH 7.5). The reaction mixture (10 mL) was incubated at 40° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of indoleacetic acid formed in the reaction. One unit of enzyme activity was designed as the amount of enzyme producing 1 mol of indoleacetic acid per minute under the standard assay conditions. The concentration of indoleacetic acid was determined by analytical method.

### Example 6

Protocol for the conversion of 2-(2-chlorophenyl)-2-hydroxyacetonitrile to (2-chlorophenyl)(hydroxy)acetic acid using engineered nitrilase.

The standard assays were carried out by mixing the substrate 2-(2-chlorophenyl)-2-hydroxyacetonitrile (5 mg/mL) with the purified enzyme in phosphate buffer (100 mM, pH 7.0). The reaction mixture (10 mL) was incubated at 30° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (2-chlorophenyl)(hydroxy)acetic acid formed in the reaction. One unit of enzyme activity was designed as the amount of enzyme producing 1 mol of (2-chlorophenyl)(hydroxy)acetic acid per minute under the standard assay conditions. The concentration of (2-chlorophenyl)(hydroxy)acetic acid was determined by analytical methods.

### Example 7

Protocol for the conversion of 2-(6-methoxynaphthalen-2-yl)propanenitrile to (S)-naproxen using engineered nitrilase.

The standard assays were carried out by mixing the substrate 2-(6-methoxynaphthalen-2-yl)propanenitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (S)-naproxen formed in the reaction. One unit of enzyme activity was designed as the amount of enzyme producing 1 mol of (S)-naproxen per minute under the standard assay conditions. The concentration of (S)-naproxen was determined by analytical methods.

### Example 8

Protocol for the conversion of 2-[1-(aminomethyl)cyclohexyl]acetonitrile to gabapentin using engineered nitrilase.

The standard assays were carried out by mixing the substrate 2-[1-(aminomethyl)cyclohexyl]acetonitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of gabapentin formed in the reaction. One unit of enzyme activity was designed as the amount of enzyme producing 1 mol of gabapentin per minute under the standard assay conditions. The concentration of gabapentin was determined by analytical methods.

### Example 9

Protocol for the conversion of 2-[4-(2-methylpropyl)phenyl]propanenitrile to (S)-ibuprofen using engineered nitrilase.

The standard assays were carried out by mixing the substrate 2-[4-(2-methylpropyl)phenyl]propanenitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (S)-ibuprofen formed in the reaction. One unit of enzyme activity was designed as the amount of enzyme producing 1 mol of (S)-ibuprofen per minute under the standard assay conditions. The concentration of (S)-ibuprofen was determined by analytical method.

### Example 10

Protocol for the conversion of 2-hydroxy-3-phenylpropanenitrile to (S)-phenyllactate using engineered nitrilase.

The standard assays were carried out by mixing the substrate 2-hydroxy-3-phenylpropanenitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (S)-phenyllactate formed in the reaction. One unit of enzyme activity was designed as the amount of enzyme producing 1 mol of (S)-phenyllactate per minute under the standard assay conditions. The concentration of (S)-phenyllactate was determined by analytical method.

### Example 11

Protocol for the conversion of 3-(3-fluorophenyl)-2-hydroxypropanenitrile to (2S)-3-(3-fluorophenyl)-2-hydroxypropanoic acid using engineered nitrilase.

The standard assays were carried out by mixing the substrate 3-(3-fluorophenyl)-2-hydroxypropanenitrile (5 mg/mL) with the purified enzyme in phosphate buffer (100 mM, pH 7.5). The reaction mixture (10 mL) was incubated at 40° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (2S)-3-(3-fluorophenyl)-2-hydroxypropanoic acid formed in the reaction. One unit of enzyme activity was designed as the amount of enzyme producing 1 mol of (2S)-3-(3-fluorophenyl)-2-hydroxypropanoic acid per minute under the standard assay conditions. The concentration of (2S)-3-(3-fluorophenyl)-2-hydroxypropanoic acid was determined by analytical method.

### Example 12

Protocol for the conversion of 2-hydroxy-3-(naphthalen-1-yl)propanenitrile to (2S)-2-hydroxy-3-(naphthalen-1-yl)propanoic acid using engineered nitrilase.

The standard assays were carried out by mixing the substrate 2-hydroxy-3-(naphthalen-1-yl)propanenitrile (5 mg/mL) with the purified enzyme in phosphate buffer (100 mM, pH 7.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (2S)-2-hydroxy-3-(naphthalen-1-yl)propanoic acid formed in the reaction. One unit of enzyme activity was designed as the amount of enzyme producing 1 mol of (2S)-2-hydroxy-3-(naphthalen-1-yl)propanoic acid per minute under the standard assay conditions. The concentration of (2S)-2-hydroxy-3-(naphthalen-1-yl)propanoic acid was determined by analytical method.

### Example 13

Protocol for the conversion of 2-hydroxy-3-(naphthalen-1-yl)propanenitrile to ((2S)-2-hydroxy-3-(pyridin-2-yl)propanoic acid using engineered nitrilase.

The standard assays were carried out by mixing the substrate 2-hydroxy-3-(pyridin-2-yl)propanenitrile (5 mg/mL) with the purified enzyme in phosphate buffer (100 mM, pH 8.0). The reaction mixture (10 mL) was incubated at 30° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (2S)-2-hydroxy-3-(pyridin-2-yl)propanoic acid formed in the reaction. One unit of enzyme activity was designed as the amount of enzyme producing 1 μmol of (2S)-2-hydroxy-3-(pyridin-2-yl)propanoic acid per minute under the standard assay conditions. The concentration of (2S)-2-hydroxy-3-(pyridin-2-yl)propanoic acid was determined by analytical method.

### Example 14

Protocol for the conversion of 2-hydroxy-3-(thiophen-3-yl)propanenitrile to (2S)-2-hydroxy-3-(thiophen-3-yl)propanoic acid using engineered nitrilase.

The standard assays were carried out by mixing the substrate 2-hydroxy-3-(thiophen-3-yl)propanenitrile (5 mg/mL) with the purified enzyme in phosphate buffer (100 mM, pH 8.0). The reaction mixture (10 mL) was incubated at 45° C. for 10 min to 60 min with shaking at 150 rpm in the reciprocal shaker. Aliquots (500 μL) were sampled, and the reactions were quenched by the addition of 500 μL HCl (2 M). The conversion was determined by measuring the amount of (2S)-2-hydroxy-3-(thiophen-3-yl)propanoic acid formed in the reaction. One unit of enzyme activity was designed as the amount of enzyme producing 1 μmol of (2S)-2-hydroxy-3-(thiophen-3-yl)propanoic acid per minute under the standard assay conditions. The concentration of (2S)-2-hydroxy-3-(thiophen-3-yl)propanoic acid was determined by analytical methods.

### Example 15

Enzyme assay protocol for the conversion of mandelonitrile to (R)-(−)-mandelic acid using engineered nitrilase

The continuous fed-batch process was used in 2 and 10L at 30° C. under mechanical agitation of 250 rpm in 5L and 10L double jacket glass reactors respectively. The reaction mixture contained 100 mM of mandelonitrile, 100 mM of phosphate sodium buffer (pH 8.0), 10 g/L of E. coli (wet cells), and 10% methanol. The control of the reaction temperature and pH, agitation of the reaction mixture, and substrate feeding were performed. The substrate feeding was conducted as it is on the 100 mL scale. The concentration of (R)-(−)-mandelic acid was determined analytical method.

### Example 16

Enzyme assay protocol for the conversion of Isobutylsuccinonitrile to (S)-3-cyano-5-methylhexanoic acid

Enzymatic hydrolysis of Isobutylsuccinonitrile was performed in a 5 L reactor containing 3L of Tris-HCl buffer (50 mM, pH 8.0), 300 g of Isobutylsuccinonitrile and 39.8 g of dry cell weight (DCW) whole cells at 30° C. with an agitation rate of 300 rpm for 6 h. The reaction samples (200 μL) were taken every 1 h and acidified by the addition of 50βL of 2.0 M HCl. The resulting mixtures were extracted with 800 μL of ethyl acetate and centrifuged at 12,000 rpm for 2 min. The organic layer was dried over anhydrous Na₂SO₄ for gas chromoatgraphy (GC) analysis. The resultant (S)-CMHA was separated from the reaction mixture by centrifugation, filtration and extraction. The concentration of (S)-CMHA was determined analytical method.

### Example 17

### Thermal and pH Inactivation Study of Nitrilase

The optimum temperature of Nitrilase was examined with 1-(cyanocyclohexyl)acetonitrile (200 mM) as the substrate in phosphate buffer pH 7.5 under controlled temperatures ranging from 10 to 65° C. during incubation of 20 min.

The optimum pH was determined using sodium acetate buffer at pH 4.0-6.0, sodium phosphate buffer at pH 6.0-8.0, Tris-Cl buffer at pH 8.0-9.0 and glycine-NaOH buffer at pH 9.0-10.0 in 100 mM concentrations for the 1-(cyanocyclohexyl)acetonitrile conversions. The reaction mixtures were incubated at 37° C. for 20 min and analyzed by HPLC. The enzyme solution in the phosphate buffer pH 7.5 was heated at 45 and 50° C. for different times separately. After heat treatment, 1-(cyanocyclohexyl)acetonitrile (100 mM) was added to the enzyme solution and incubated at 37° C. for 20 min. The resulting product mixture was analysed using analytical method.

### Example 18

### Preparations of Immobilized Enzymes

Two milliliters of nitrilase and nitrilase variants solution were mixed thoroughly with a 3% sodium alginate solution in the same volume. The enzyme/alginate mixture was dropwise added into a bath of a CaCl₂ (0.5M) solution by syringe. After stirring for an appropriate time on ice, the solution was decanted and the resulting beads (about 1 mm diameter) were washed once with 20 mL of tris buffer (50 mM Tris-HCl, 50 mM NaCl, pH 7.5). The beads were lyophilized for storage and stored at −20° C. Before use, the beads were re-swelled with tris-Cl buffer (50 mM, pH 7.5). For comparison, the nitrilase and nitrilase variant were also immobilized in similar ways to CLEA using glutaraldehyde (4%, w/v), polyethyleneimine (5%, w/v) or dextran polyaldehyde (5%, w/v) as cross-linking agents.

### ADVANTAGES

Nitrilases are a class of enzymes with the potential to be valuable catalysts in the synthesis of active pharmaceutical ingredients (APIs). The engineered nitrilase can catalyze the hydrolysis of nitriles to corresponding carboxylic acids, a reaction useful in producing a variety of APIs. Specifically, these engineered nitrilase enzymes efficiently and cost-effectively synthesize the pharmaceutical intermediate (1-cyanocyclohexyl)acetic acid, an important precursor for the synthesis of Gabapentin. Gabapentin is commonly used to treat and prevent seizures in people with epilepsy and to alleviate nerve pain (postherpetic neuralgia) following shingles.

The present engineered nitrilases offer several advantages over traditional chemical methods, including high selectivity, mild reaction conditions, and the ability to use renewable resources. By engineering the nitrilase enzyme's active site and surrounding regions, its substrate specificity and activity have been enhanced for a wide range of substrates. These engineered nitrilases can convert 15 different important pharmaceutical API intermediates with high conversion and selectivity, including those used as non-steroidal anti-inflammatories, anticonvulsants, plant growth hormones, antiplatelet agents, and anti-epileptics.

The ability to convert various nitrile substrates into corresponding carboxylic acids is particularly advantageous for industrial applications, enabling quick and cost-effective production of diverse products from different starting materials. Additionally, these nitrilases improve activity under conditions such as higher substrate concentrations, broad pH ranges, and wide temperature ranges. This invention introduces an engineered nitrilase enzyme as an alternative to alkaline or acid hydrolysis for converting nitrile substrates into carboxylic acids, also can be used in waste treatment and bioremediation.

What is claimed is:

1. An engineered nitrilase polypeptide and polynucleotide encoding the same, wherein;
a. The engineered nitrilase polypeptide exhibits enhanced specificity and efficiency for converting 1-(cyanomethyl)-cyclohexane-1-carbonitrile to (1-cyanocyclohexyl)-acetic acid, characterized by 100% conversion within 24 hours with an enzyme load of less than 5% of the total weight of the substrate.
b. the engineered nitrilase polypeptide and polynucleotide comprise an amino acid sequence and a nucleotide sequence that is at least 80% identical to SEQ ID NO:1 and 2 respectively and include the modifications of residue corresponding to X3 is Lysine (Lys); X265 is Glycine (Gly); X196 is Tyrosine (Tyr); X260 is Arginine (Arg); X229 is Lysine (Lys); X106 is Glutamic acid (Glu); X88 is Arginine (Arg); X122 is Aspartic acid (Asp).

2. The engineered nitrilase polypeptide of claim 1, wherein the amino acid residue difference at the residue positions is selected from the following:
X28 is Arg or Val or Glu or Leu;
X33 is Lys or Arg or Glu;
X59 is Tyr or Gly or Asn or Thr;
X65 is Val or Gly or Ala or Met;
X67 is Val or Arg or Gly or Lys or Met or Ala;
X70 is Arg or Asp or Lys or Pro or His or Ala;
X79 is Phe or Val or Ile;
X84 is Lys or Phe or Asp or His or Tyr;
X85 is His or Lys or Arg or Ala or Asp;
X90 is Val or Thr or Ser or Asp; Asp or;
X97 is Thr or Tyr or Trp or Val or His;
X108 is Glu or Ala or Ser;
X126 is Lys or Arg or Thr or Met or Ala;
X151 is His or Tyr or Val or Ala or Ile or Arg;
X153 is Ile or Leu or Pro or Ala;
X179 is Lys or Asp or His or Thr;
X192 is Ile or Met or Val or Ala;
X194 is Gly or Asp or Arg or Lys or Val;
X203 is Ile or Asp;
X226 is a Cys or Ser or Val or Ile or Leu;
X230 is Ala or Asp or Ser or Gly;
X240 is Val or Asp or Gly or Ala or Glu or Ser;
X241 is Arg or Lys or His;
X250 is a Cys or Val or Ala;
X253 is Ser or Ala or Val or Thr or Gly;
X261 is Glu or Ser or Pro or Len or Ala or Val;
X290 is Asn or Ala or Ser or Thr; and
X307 is Asp or Glu or His;
X8 is Val or Len or Ile;
X10 is Val or Ser or Ile;
X17 is Trp or Tyr or Leu;
X23 is Ser or Gly or Ala;
X31 is Ile or Val or Ala;
X37 is Lys or Gln or Ala or Glu or His or Asp;
X48 is Phe or Tyr;
X49 is Ile or Val or Ala or Phe;
X60 is Ser or Thr or Val;
X66 is Ile or Met or Phe or Leu;
X69 is Val or Ile or Phe;
X78 is Ser or Val or Ile or Glu or Ile or Thr;
X80 is Asp or Asn or Glu;
X81 is Ser or Asn or Leu;
X93 is Lys or Arg or Glu or Gln or Asn;
X94 is Ala or His or Asn or Len;
X98 is Ala or Len or Ile or Met;
X99 is Val or Ala or Len or Gly;
X100 is Len or Ile or Phe or Val or Thr or Ala or Ser;
X113 is Ala or Thr;
X116 is Len or Ile or Val or His;
X119 is Pro or Asp or Ala or Ser or Asn;
X124 is Val or Len or Ala or Gln or;
X135 is Ala or Gly or Ser or Thr;
X149 is Ser or Thr or His or Gly or Val or Met;
X154 is Asp or Gly or Arg or Lys or Glu;
X158 is Ile or Leu or Met;
X160 is Ala or Ser or Met or Thr or Val;
X167 is Ile or Val or Phe or Met;
X195 is Gly or Pro;
X198 is Pro or His or Asn or Phe;
X202 is Ala or Trp or His or Gly or Ser;
X206 is Asn or Val or Glu or Ser;
X207 is Ala or Gly or His;
X211 is Val or Leu or Thr or Ala or Met;
X217 is Ser or Thr or Gly;
X232 is Ile or Ser or His or Gln or Leu;
X237 is Asp or Gly or Glu or Ser or Ile;
X238 is Arg or Asp or Ser or Glu;
X239 is Pro or Ala or Glu or Asn;
X246 is His or Arg or Leu or Thr;
X247 is Val or Ala or Glu or Pro;
X251 is His or Phe or Ser or Ala;
X255 is Tyr or Leu;
X263 is Gly or Ala or Thr or Ser or Val;
X267 is Ala or Asp or Gly;
X268 is Pro or Glu or His or Thr;
X269 is Asp or Glu or Asn or Thr or Ser;
X270 is Gln or Ala or Thr or Val or Arg or Gly or Lys;
X274 is Leu or Val or Ala;
X275 is Ile or Leu or Val or Phe or Met or Cys;
X282 is Ala or Met or Val or Thr;
X284 is Gly or Ala or Thr;
X286 is Ala or Gly;
X288 is Asn or Ser or Thr;
X305 is Leu or Trp or Val or Met;
X306 is Leu or Phe or Val or Ile;
X309 is Lys or Thr or Ser or Arg or Val;
X310 is Arg or Ser or Val or Ala;
X313 is Arg or Pro or Val;
X315 is Glu or Ile or Met or Thr or Ser;
X317 is Phe or Gln or Gly or Ala or Arg or Len or Val;
X319 is Len or Gln or Thr or Ala;
X323 is Asp or Glu or Asn or Ala;
X326 is Gly or Ser or Thr or Glu or Pro or Arg;
X333 is Thr or Val or Pro or Len or Glu or Lys;
X334 is Glu or Ala or Pro or Len or Ser;
X335 is Gln or Pro or Len or Ala or His or Asp or Ser; and
X336 is Glu or Len or Ala or Gly or Val or Asp.

3. The engineered nitrilase polypeptide of claim 1 functions at higher temperature of
a. 68° C. while maintaining a conversion rate of greater than 98% wherein the engineered nitrilase polypeptide contains the following features:
the residue corresponding to X28 is Arg,
the residue corresponding to X 70 is Arg,
the residue corresponding to X84 is Lys,
the residue corresponding to X59 is Tyr,
the residue corresponding to X65 is Val and
the residue corresponding to X67 is Val
b. 67° C. while maintaining a conversion rate of greater than 98% wherein the engineered nitrilase polypeptide contains the following features:
the residue corresponding to X90 is Asp,
the residue corresponding to X126 is Glu,
the residue corresponding to X226 is Cys,
the residue corresponding to X85 is His,
the residue corresponding to X97 is Tyr, and
the residue corresponding to X240 is Val
c. 66° C. while maintaining a conversion rate of greater than 96% wherein the engineered nitrilase polypeptide contains the following features:
the residue corresponding to X241 is Arg,
the residue corresponding to X250 is Cys,
the residue corresponding to X108 is Glu,
the residue corresponding to X151 is His,
the residue corresponding to X153 is Ile and the residue corresponding to X253 is Ser
d. 68° C. while maintaining a conversion rate of greater than 96% wherein the engineered nitrilase polypeptide contains the following features:
the residue corresponding to X307 is Asp,
the residue corresponding to X33 is Lys,
the residue corresponding to X179 is Lys,
the residue corresponding to X192 is Ile,
the residue corresponding to X203 is Ile, and
the residue corresponding to X290 is Asn.

4. The engineered nitrilase polypeptide of claim 1 functions at different pH
a. pH 8 and maintains a conversion rate >99% wherein the engineered polypeptide contains the following features:
the residue corresponding to X79 is Phe,
the residue corresponding to X261 is Glu,
the residue corresponding to X194 is Gly, and
the residue corresponding to X230 is Ala
b. pH 7 and maintains a conversion rate >99.5% wherein the engineered polypeptide contains the following features:
the residue corresponding to X90 is Asp,
the residue corresponding to X126 is Glu,
the residue corresponding to X192 is Ile, and
the residue corresponding to X203 is Ile
c. pH 6.5 and maintains a conversion rate >99% wherein the engineered polypeptide contains the following features:
the residue corresponding to X153 is Ile,
the residue corresponding to X253 is Ser,
the residue corresponding to X85 is His, and
the residue corresponding to X97 is Tyr
d. pH 5.5 to 6 and maintains a conversion rate >99% wherein the engineered polypeptide contains the following features:
the residue corresponding to X307 is Asp,
the residue corresponding to X33 is Lys,
the residue corresponding to X67 is Val, and
the residue corresponding to X241 is Arg s.

5. The engineered nitrilase polypeptide of claim 1 wherein,
a. The conversion of nitrile substrate 1-(cyanomethyl)cyclohexane-1-carbonitrile converted to (1-cyanocyclohexyl)acetic acid is at least >99%, the nitrilase polypeptide sequence further comprises the following substitutions the residue corresponding to X153 is Ile;
the residue corresponding to X179 is Lys;
the residue corresponding to X106 is Asp;
the residue corresponding to X265 is Pro;
the residue corresponding to X65 is Val;
the residue corresponding to X70 is Arg;
b. The conversion of nitrile substrate 1-(cyanomethyl)cyclohexane-1-carbonitrile to (1-cyanocyclohexyl)acetic acid is at least >95%, the nitrilase polypeptide sequence further comprises the following substitutions:
the residue corresponding to X240 is Val;
the residue corresponding to X70 is Asp;
the residue corresponding to X3 is Lys;
the residue corresponding to X253 is Ser;
the residue corresponding to X85 is His;
the residue corresponding to X203 is Ile;
the residue corresponding to X261 is Glu;
c. The conversion of nitrile substrate 1-(cyanomethyl)cyclohexane-1-carbonitrile to (1-cyanocyclohexyl)acetic acid is at least >90%, the nitrilase polypeptide sequence further comprises the following substitutions:
the residue corresponding to X65 is Val;
the residue corresponding to X265 is Pro;
the residue corresponding to X108 is Glu;
the residue corresponding to X97 is Tyr;
the residue corresponding to X33 is Lys;
the residue corresponding to X307 is Asp;
the residue corresponding to X194 is Gly;
d. The conversion of nitrile substrate 1-(cyanomethyl)cyclohexane-1-carbonitrile to (1-cyanocyclohexyl)acetic acid is at least >85%, the nitrilase polypeptide sequence further comprises the following substitutions:
the residue corresponding to X241 is Arg;
the residue corresponding to X79 is Phe;
the residue corresponding to X67 is Val;
the residue corresponding to X151 is His;
the residue corresponding to X230 is Ala;
e. The conversion of nitrile substrate 1-(cyanomethyl)cyclohexane-1-carbonitrile to (1-cyanocyclohexyl)acetic acid is at least >80%, the nitrilase polypeptide sequence further comprises the following substitutions:
the residue corresponding to X84 is Lys;
the residue corresponding to X126 is Glu;
the residue corresponding to X179 is Lys;
the residue corresponding to X106 is Asp;
the residue corresponding to X226 is Cys;
the residue corresponding to X250 is Cys;
the residue corresponding to X241 is Arg;
f. The nitrile substrate 3-(4-chlorophenyl)pentanedinitrile is converted into (3S)-3-(4-chlorophenyl)-4-cyanobutanoic acid with >95% conversion, the nitrilase polypeptide additionally comprises the following substitutions:
the residue corresponding to X66 is Met,
the residue corresponding to X10 is Ser,
the residue corresponding to X99 is Val,
the residue corresponding to X149 is Thr,
the residue corresponding to X113 is Thr,
the residue corresponding to X116 is Ile,
the residue corresponding to X78 is Ile,
the residue corresponding to X124 is Gln,
the residue corresponding to X69 is Val,
the residue corresponding to X315 is Glu,
the residue corresponding to X275 is Leu, and
the residue corresponding to X263 is Ala.
g. the nitrile substrate 2-chloropyridine-3-carbonitrile is converted into 2-chloronicotinic acid with >98% conversion, the nitrilase polypeptide additionally comprises the following substitutions:
the residue corresponding to X37 is Ala,
the residue corresponding to X17 is Tyr,
the residue corresponding to X93 is Glu,
the residue corresponding to X98 is Ile,
the residue corresponding to X135 is Gly,
the residue corresponding to X78 is Glu,
the residue corresponding to X80 is Asp,
the residue corresponding to X149 is Ser,
the residue corresponding to X100 is Ser,
the residue corresponding to X195 is Gly,
the residue corresponding to X275 is Met, and
the residue corresponding to X239 is Pro
h. the nitrile substrate Indole-3-acetonitrile is converted into indoleacetic acid (IAA) with >99% conversion, the nitrilase polypeptide additionally comprises the following substitutions:
the residue corresponding to X17 is Trp,
the residue corresponding to X31 is Ile,
the residue corresponding to X149 is Val,
the residue corresponding to X99 is Leu,
the residue corresponding to X119 is Ala,
the residue corresponding to X94 is Ala,
the residue corresponding to X78 is Ile,
the residue corresponding to X69 is Phe,
the residue corresponding to X81 is Asn,
the residue corresponding to X255 is Tyr, and
the residue corresponding to X306 is Ile,
the residue corresponding to X288 is Ser
i. the nitrile substrate Mandelonitrile is converted into (R)-mandelic acid with >98.5% conversion, the nitrilase polypeptide additionally comprises the following substitutions:
the residue corresponding to X10 is Ile,
the residue corresponding to X66 is Leu,
the residue corresponding to X78 is Ser,
the residue corresponding to X80 is Asn,
the residue corresponding to X69 is Ile,
the residue corresponding to X113 is Thr,
the residue corresponding to X119 is Ala,
the residue corresponding to X99 is Val,
the residue corresponding to X81 is Asn,
the residue corresponding to X305 is Leu,
the residue corresponding to X160 is Thr, and
the residue corresponding to X239 is Asn
j. the nitrile substrate 2-(2-chlorophenyl)-2-hydroxyacetonitrile is converted into (2-chlorophenyl)(hydroxy)acetic acid with >99% conversion, the nitrilase polypeptide additionally comprises the following substitutions:
the residue corresponding to X17 is Leu,
the residue corresponding to X66 is Met,
the residue corresponding to X93 is Lys,
the residue corresponding to X81 is Ser,
the residue corresponding to X98 is Ala,
the residue corresponding to X124 is Val,
the residue corresponding to X119 is Asp,
the residue corresponding to X113 is Thr,
the residue corresponding to X116 is His,
the residue corresponding to X313 is Pro,
the residue corresponding to X336 is Glu,
the residue corresponding to X246 is Arg, and
the residue corresponding to X167 is Met
k. the nitrile substrate 2-(6-methoxynaphthalen-2-yl)propanenitrile is converted into (S)-naproxen with >99% conversion, the nitrilase polypeptide additionally comprises the following substitutions:
the residue corresponding to X8 is Val,
the residue corresponding to X60 is Thr,
the residue corresponding to X119 is Asn,
the residue corresponding to X116 is Ile,
the residue corresponding to X80 is Glu,
the residue corresponding to X99 is Val,
the residue corresponding to X81 is Leu,
the residue corresponding to X135 is Ala,
the residue corresponding to X149 is Ser,
the residue corresponding to X326 is Thr,
the residue corresponding to X288 is Ser,
the residue corresponding to X211 is Thr, and
the residue corresponding to X154 is Glu
l. the nitrile substrate 2-[1-(aminomethyl)cyclohexyl]acetonitrile is converted into gabapentin with >99% conversion, the nitrilase polypeptide additionally comprises the following substitutions:
the residue corresponding to X49 is Ile,
the residue corresponding to X23 is Ala,
the residue corresponding to X80 is Asn,
the residue corresponding to X98 is Ala,
the residue corresponding to X69 is Val,
the residue corresponding to X116 is Ile,
the residue corresponding to X81 is Leu,
the residue corresponding to X100 is Phe,
the residue corresponding to X93 is Asn,
the residue corresponding to X124 is Gln,
the residue corresponding to X211 is Leu,
the residue corresponding to X267 is Ala,
the residue corresponding to X160 is Met, and
the residue corresponding to X217 Ser;
m. the nitrile substrate 2-[4-(2-methylpropyl)phenyl]propanenitrile is converted into (S)-Ibuprofen with >99% conversion, the nitrilase polypeptide additionally comprises the following substitutions:
the residue corresponding to X37 is Glu,
the residue corresponding to X8 is Val,
the residue corresponding to X81 is Ser,
the residue corresponding to X113 is Thr,
the residue corresponding to X135 is Thr,
the residue corresponding to X98 is Leu,
the residue corresponding to X100 is Leu,
the residue corresponding to X124 is Val,
the residue corresponding to X78 is Thr,
the residue corresponding to X69 is Val,
the residue corresponding to X274 is Val,
the residue corresponding to X251 is Ala,
the residue corresponding to X217 is Thr, and
the residue corresponding to X275 is Met
n. substrate 2-hydroxy-3-phenylpropanenitrile is converted into (S)-phenyllactate with >90% conversion, the nitrilase polypeptide additionally comprises the following substitutions:
the residue corresponding to X37 is Ala,
the residue corresponding to X8 is Ile,
the residue corresponding to X69 is Ile,
the residue corresponding to X81 is Leu,
the residue corresponding to X135 is Ser,
the residue corresponding to X98 is Ala,
the residue corresponding to X116 is Val,
the residue corresponding to X149 is Gly,
the residue corresponding to X100 is Phe,
the residue corresponding to X99 is Ala,
the residue corresponding to X305 is Leu,
the residue corresponding to X309 is Val,
the residue corresponding to X335 is Pro, and
the residue corresponding to X288 is Asn
o. the nitrile substrate 3-(3-fluorophenyl)-2-hydroxypropanenitrile is converted into (2S)-3-(3-fluorophenyl)-2-hydroxypropanoic acid with >99% conversion, the nitrilase polypeptide additionally comprises the following substitutions:
the residue corresponding to X66 is Leu,
the residue corresponding to X49 is Val,
the residue corresponding to X124 is Gln,
the residue corresponding to X69 is Phe,
the residue corresponding to X99 is Leu,
the residue corresponding to X80 is Asp,
the residue corresponding to X81 is Leu,
the residue corresponding to X93 is Arg,
the residue corresponding to X135 is Ala,
the residue corresponding to X98 is Met,
the residue corresponding to X336 is Gly,
the residue corresponding to X326 is Thr,
the residue corresponding to X319 is Thr, and
the residue corresponding to X269 is Asp
p. the nitrile substrate 2-hydroxy-3-(naphthalen-1-yl)propanenitrile is converted into (2S)-2-hydroxy-3-(naphthalen-1-yl)propanoic acid with >99% conversion, the nitrilase polypeptide additionally comprises the following substitutions:
the residue corresponding to X23 is Ser,
the residue corresponding to X49 is Val,
the residue corresponding to X113 is Thr,
the residue corresponding to X100 is Ser,
the residue corresponding to X135 is Thr,
the residue corresponding to X99 is Val,
the residue corresponding to X284 is Ala, and
the residue corresponding to X333 is Leu
q. the nitrile substrate 2-hydroxy-3-(pyridin-2-yl)propanenitrile is converted into (2S)-2-hydroxy-3-(pyridin-2-yl)propanoic acid with >99% conversion, the nitrilase polypeptide additionally comprises the following substitutions:
the residue corresponding to X48 is Phe,
the residue corresponding to X17 is Trp,
the residue corresponding to X119 is Asn,
the residue corresponding to X99 is Gly,
the residue corresponding to X113 is Thr,
the residue corresponding to X98 is Ala,
the residue corresponding to X160 is Val, and
the residue corresponding to X333 is Leu
r. the nitrile substrate 2-hydroxy-3-(thiophen-3-yl)propanenitrile is converted into (2S)-2-hydroxy-3-(thiophen-3-yl)propanoic acid with >90% conversion, the nitrilase polypeptide additionally comprises the following substitutions:
the residue corresponding to X8 is Ile,
the residue corresponding to X49 is Val,
the residue corresponding to X80 is Asp,
the residue corresponding to X113 is Ala,
the residue corresponding to X81 is Ser,
the residue corresponding to X94 is Ala,
the residue corresponding to X93 is Asn,
the residue corresponding to X124 is Leu,
the residue corresponding to X135 is Ser,
the residue corresponding to X237 is Ser,
the residue corresponding to X268 is His,
the residue corresponding to X167 is Phe, and
the residue corresponding to X286 is Gly
s. the nitrile substrate Isobutylsuccinonitrile is converted into (S)-3-cyano-5-methylhexanoic acid with >95% conversion, the nitrilase polypeptide additionally comprises the following substitutions:
the residue corresponding to X48 is Thr,
the residue corresponding to X10 is Ile,
the residue corresponding to X149 is Val,
the residue corresponding to X135 is Ser,
the residue corresponding to X81 is Leu,
the residue corresponding to X93 is Gln,
the residue corresponding to X124 is Gln,
the residue corresponding to X246 is Thr,
the residue corresponding to X334 is Pro, and
the residue corresponding to X335 is His.

6. The engineered nitrilase polypeptide of claim 1, wherein
a. the amino acid sequence corresponds to the sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291 or 293.
b. the nucleotide sequence corresponds to the sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292 or 294.

7. The engineered nitrilase of claim 1 is used in a reaction system in the form of wet cells or immobilised wet cells or as purified nitrilase as a biocatalyst, with 1-cyanocyclohexylacetonitrile as a substrate in 100 mM phosphate buffer at pH 7.0 and at temperature of 45° C. for 10 minutes with shaking at 150 rpm, using a substrate concentration of greater than 200 g/L with an enzyme load of less than 5% and after the reaction, the solution is separated and purified to obtain 1-cyanocyclohexyl acetic acid wherein wet cells are obtained from the fermentation culture of genetically engineered E. coli containing the nitrilase mutant gene, and purified nitrilase is obtained by ultrasonic breaking of wet cells followed by His-tag affinity chromatography.

8. The engineered nitrilase polypeptide of claim 1 is expressed and prepared using the following method:
a. Inoculate a genetically engineered strain containing the nitrilase mutant gene in Luria Bertani broth with kanamycin (50 μg/mL) and culture at 37° C. for 8-10 hours. Transfer the inoculum (2% volume) to LB medium with kanamycin (50 mg/L) and culture at 37° C. until OD600 reaches 0.6-0.8. Add IPTG (0.1 mM) and incubate at 28° C. for 10 hours. Harvest the wet cells by centrifugation and wash with saline.
b. Resuspend the wet cells in 50 mM phosphate buffer (pH 8.0) with 300 mM NaCl, break the cells ultrasonically, and centrifuge to remove debris. Apply the supernatant (crude enzyme) to a Ni-NTA column, wash with equilibrium buffer, and elute with elution buffer to remove impurities. Elute and collect the target protein with protein elution buffer.
c. The recombinant engineered nitrilase polynucleotide construct, comprising the polynucleotide of claim 1, is operably linked to promoter sequences for expression in a recombinant host cell. The polynucleotide is expressed using the vector pET28a(+) in E. coli.

9. The engineered nitrilase polypeptide of claim 1 is active in solvents such as DMSO, EDTA, IPA, MTB, ethyl acetate, hexane, decane, methanol, and ethanol.

10. The engineered nitrilase of claim 1 is derived using the natural nitrilase enzyme sequence wherein hotspots for engineering the enzyme are identified by a method that employs van der Waals contacts to create 3D fragments of the enzyme, summing the pLDDT values of residues for the 3D fragment to generate a pLDDT_{F} and further selecting fragments with lower pLDDT_{F} to identify residues that are relatively non-conserved within the fragment based on probability values derived for each residue position using multiple sequence alignments derived from a plurality of natural nitrilases each having sequences not more than 90% identical to each other.
